SEQUENCE LISTING (50 sequences)

SEQ ID NO: 1 - DNA, 611 bp, Simian immunodeficiency virus
  source 1..611: mol_type = genomic DNA
ccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatat

SEQ ID NO: 2 - DNA, 16 bp, synthetic construct
  source 1..16: mol_type = other DNA
tgagtttaaggtaagt

SEQ ID NO: 3 - DNA, 10 bp, synthetic construct
  source 1..10: mol_type = other DNA
ctctccacag

SEQ ID NO: 4 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 5 - DNA, 760 bp, synthetic construct
  source 1..760: mol_type = other DNA
gtaagtatcaaggttacaagacaggtttaaggagaccaatagaaactgggcttgtcgagacagagaagactcttgcgtttctgataggcacgcggccgcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatgcggccgcctattggtcttactgacatccactttgcctttctctccacag

SEQ ID NO: 6 - DNA, 1257 bp, Homo sapiens
  source 1..1257: mol_type = genomic DNA
atgcccagctctgtgtcctggggcattctgctgctggctggcctgtgctgtctggtgcctgtgtccctggctgaggaccctcagggggatgctgcccagaaaacagacacctcccaccatgaccaggaccaccccaccttcaacaagatcacccccaacctggcagagtttgccttcagcctgtacagacagctggcccaccagagcaacagcaccaacatctttttcagccctgtgtccattgccacagcctttgccatgctgagcctgggcaccaaggctgacacccatgatgagatcctggaaggcctgaacttcaacctgacagagatccctgaggcccagatccatgagggcttccaggaactgctgagaaccctgaaccagccagacagccagctgcagctgacaacaggcaatgggctgttcctgtctgagggcctgaagctggtggacaagtttctggaagatgtgaagaagctgtaccactctgaggccttcacagtgaactttggggacacagaagaggccaagaaacagatcaatgactatgtggaaaagggcacccagggcaagattgtggaccttgtgaaagagctggacagggacactgtgtttgcccttgtgaactacatcttcttcaagggcaagtgggagaggccctttgaagtgaaggacactgaggaagaggacttccatgtggaccaagtgaccacagtgaaggtgccaatgatgaagagactggggatgttcaatatccagcactgcaagaaactgagcagctgggtgctgctgatgaagtacctgggcaatgctacagccatattctttctgcctgatgagggcaagctgcagcacctggaaaatgagctgacccatgacatcatcaccaaatttctggaaaatgaggacagaagatctgccagcctgcatctgcccaagctgagcatcacaggcacatatgacctgaagtctgtgctgggacagctgggaatcaccaaggtgttcagcaatggggcagacctgagtggagtgacagaggaagcccctctgaagctgtccaaggctgtgcacaaggcagtgctgaccattgatgagaagggcacagaggctgctggggccatgtttctggaagccatccccatgtccatccccccagaagtgaagttcaacaagccctttgtgttcctgatgattgagcagaacaccaagagccccctgttcatgggcaaggttgtgaaccccacccagaaatga

SEQ ID NO: 7 - DNA, 1257 bp, Homo sapiens
  source 1..1257: mol_type = genomic DNA
tacgggtcgagacacaggaccccgtaagacgacgaccgaccggacacgacagaccacggacacagggaccgactcctgggagtccccctacgacgggtcttttgtctgtggagggtggtactggtcctggtggggtggaagttgttctagtgggggttggaccgtctcaaacggaagtcggacatgtctgtcgaccgggtggtctcgttgtcgtggttgtagaaaaagtcgggacacaggtaacggtgtcggaaacggtacgactcggacccgtggttccgactgtgggtactactctaggaccttccggacttgaagttggactgtctctagggactccgggtctaggtactcccgaaggtccttgacgactcttgggacttggtcggtctgtcggtcgacgtcgactgttgtccgttacccgacaaggacagactcccggacttcgaccacctgttcaaagaccttctacacttcttcgacatggtgagactccggaagtgtcacttgaaacccctgtgtcttctccggttctttgtctagttactgatacaccttttcccgtgggtcccgttctaacacctggaacactttctcgacctgtccctgtgacacaaacgggaacacttgatgtagaagaagttcccgttcaccctctccgggaaacttcacttcctgtgactccttctcctgaaggtacacctggttcactggtgtcacttccacggttactacttctctgacccctacaagttataggtcgtgacgttctttgactcgtcgacccacgacgactacttcatggacccgttacgatgtcggtataagaaagacggactactcccgttcgacgtcgtggaccttttactcgactgggtactgtagtagtggtttaaagaccttttactcctgtcttctagacggtcggacgtagacgggttcgactcgtagtgtccgtgtatactggacttcagacacgaccctgtcgacccttagtggttccacaagtcgttaccccgtctggactcacctcactgtctccttcggggagacttcgacaggttccgacacgtgttccgtcacgactggtaactactcttcccgtgtctccgacgaccccggtacaaagaccttcggtaggggtacaggtaggggggtcttcacttcaagttgttcgggaaacacaaggactactaactcgtcttgtggttctcgggggacaagtacccgttccaacacttggggtgggtctttact

SEQ ID NO: 8 - protein, 419 aa, Homo sapiens
AEDPQGDAAQKTDTSHHDQDHPTFAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 9 - DNA, 5013 bp, Homo sapiens
  source 1..5013: mol_type = genomic DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 10 - DNA, 4425 bp, Homo sapiens
  source 1..4425: mol_type = genomic DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 11 - DNA, 5013 bp, Homo sapiens
  source 1..5013: mol_type = genomic DNA
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttgtcgtccgtggggtcgtggtccgtcttcgtcaagttacggtggtggtagggactcttactgtatctcttctgtctgggtaccaaacgggtggcctgggggtacgggttctaggtcttacactcgtcgagactggacgactacgacgactccgtctcggggtggggggtaccggactcggacagactggacgtcctccggttcatactttggaagagactactggggtcgggaccccggtaactgtcgttgttgtcggacagactctactgggtgaagtccggggtcgacgtggtgagacccctgtaccacaagtggggactcagaccggacgtcgactccgacttactcttcgacccgtggtgacgacggtgactcgacttcttcgacctgaagtttcagaggtcgtggtcgttgttggactagtcgtggtaggggagactgttggaccgacgaccgtgactgttgtggtcgtcggacccgggggggtcgtacggacacgtgatactgtcggtcgacctgtggtgggacaaaccgttcttctcgtcgggggactgactcagacccccgggggactcggacagactcctcttgttactgtcgttcgacgacctcagaccggactacttgtcggtcctctcgtcgaccccgttcttacactcgtcgtccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 12 - DNA, 4425 bp, Homo sapiens
  source 1..4425: mol_type = genomic DNA
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttacggtgattacacagattgttgtcgttgtggtcgttactgtcgttacacagagggggtcacgacttctccgtggtctccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 13 - protein, 1670 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 14 - protein, 1474 aa, Homo sapiens [sequence omitted]

SEQ ID NO: 15 - DNA, 4458 bp, Homo sapiens
  source 1..4458: mol_type = genomic DNA
gctagccacatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggccc

SEQ ID NO: 16 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 17 - DNA, 435 bp, Homo sapiens
  source 1..435: mol_type = genomic DNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 18 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 19 - DNA, 426 bp, Mus musculus
  source 1..426: mol_type = genomic DNA
atgtggctgcagaacctgctgttcctgggcattgtggtgtacagcctgtctgcccctacaagatcccctatcacagtgaccagaccttggaaacatgtggaagccatcaaagaggccctgaatctgctggatgacatgcctgtgacactgaatgaagaggtggaagtggtgtccaatgagttcagcttcaagaaactgacctgtgtgcagaccaggctgaagatttttgagcagggcctgagaggcaacttcaccaagctgaaaggggctctgaacatgacagccagctactaccagacctactgtcctcctacacctgagacagactgtgaaacccaagtgaccacctatgctgacttcattgacagcctcaagaccttcctgacagacatcccctttgagtgcaagaaacctggccagaagtga

SEQ ID NO: 20 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 21 - DNA, 1080 bp, Homo sapiens
  source 1..1080: mol_type = genomic DNA
atgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaa

SEQ ID NO: 22 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 23 - DNA, 1434 bp, Homo sapiens
  source 1..1434: mol_type = genomic DNA
atgtcggctgcgcccggcctcctgcaccaggagctgtcctgcccgctgtgcctgcagctgttcgacgcgcccgtgacagccgagtgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaagacacagctgccacagcagaaactgcagctgcaggaggcatgcatgcgcaaggagaagagtgtggctgtgctggagcatcagctggtggaggtggaggagacagtgcgtcagttccggggggccgtgggggagcagctgggcaagatgcgggtgttcctggctgcactggagggctccttggaccgcgaggcagagcgtgtacggggtgaggcaggggtcgccttgcgccgggagctggggagcctgaactcttacctggagcagctgcggcagatggagaaggtcctggaggaggtggcggacaagccgcagactgagttcctcatgaaatactgcctggtgaccagcaggctgcagaagatcctggcagagtctcccccacccgcccgtctggacatccagctgccaattatctcagatgacttcaaattccaggtgtggaggaagatgttccgggctctgatgccagcgctggaggagctgacctttgacccgagctctgcgcacccgagcctggtggtgtcttcctctggccgccgcgtggagtgctcggagcagaaggcgccgccggccggggaggacccgcgccagttcgacaaggcggtggcggtggtggcgcaccagcagctctccgagggcgagcactactgggaggtggatgttggcgacaagccgcgctgggcgctgggcgtgatcgcggccgaggccccccgccgcgggcgcctgcacgcggtgccctcgcagggcctgtggctgctggggctgcgcgagggcaagatcctggaggcacacgtggaggccaaggagccgcgcgctctgcgcagccccgagaggcggcccacgcgcattggcctttacctgagcttcggcgacggcgtcctctccttctacgatgccagcgacgccgacgcgctcgtgccgctttttgccttccacgagcgcctgcccaggcccgtgtaccccttcttcgacgtgtgctggcacgacaagggcaagaatgcccagccgctgctgctcgtgggtcccgaaggcgccgaggcctga

SEQ ID NO: 24 - protein, 477 aa, Homo sapiens
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 25 - DNA, 5115 bp, Homo sapiens
  source 1..5115: mol_type = genomic DNA
atggctgtgctcaggcagctggcgctcctcctctggaagaactacaccctgcagaagcggaaggtcctggtgacggtcctggaactcttcctgccattgctgttttctgggatcctcatctggctccgcttgaagattcagtcggaaaatgtgcccaacgccaccatctacccgggccagtccatccaggagctgcctctgttcttcaccttccctccgccaggagacacctgggagcttgcctacatcccttctcacagtgacgctgccaagaccgtcactgagacagtgcgcagggcacttgtgatcaacatgcgagtgcgcggctttccctccgagaaggactttgaggactacattaggtacgacaactgctcgtccagcgtgctggccgccgtggtcttcgagcaccccttcaaccacagcaaggagcccctgccgctggcggtgaaatatcacctacggttcagttacacacggagaaattacatgtggacccaaacaggctcctttttcctgaaagagacagaaggctggcacactacttcccttttcccgcttttcccaaacccaggaccaagggaacctacatcccctgatggcggagaacctgggtacatccgggaaggcttcctggccgtgcagcatgctgtggaccgggccatcatggagtaccatgccgatgccgccacacgccagctgttccagagactgacggtgaccatcaagaggttcccgtacccgccgttcatcgcagaccccttcctcgtggccatccagtaccagctgcccctgctgctgctgctcagcttcacctacaccgcgctcaccattgcccgtgctgtcgtgcaggagaaggaaaggaggctgaaggagtacatgcgcatgatggggctcagcagctggctgcactggagtgcctggttcctcttgttcttcctcttcctcctcatcgccgcctccttcatgaccctgctcttctgtgtcaaggtgaagccaaatgtagccgtgctgtcccgcagcgacccctccctggtgctcgccttcctgctgtgcttcgccatctctaccatctccttcagcttcatggtcagcaccttcttcagcaaagccaacatggcagcagccttcggaggcttcctctacttcttcacctacatcccctacttcttcgtggcccctcggtacaactggatgactctgagccagaagctctgctcctgcctcctgtctaatgtcgccatggcaatgggagcccagctcattgggaaatttgaggcgaaaggcatgggcatccagtggcgagacctcctgagtcccgtcaacgtggacgacgacttctgcttcgggcaggtgctggggatgctgctgctggactctgtgctctatggcctggtgacctggtacatggaggccgtcttcccagggcagttcggcgtgcctcagccctggtacttcttcatcatgccctcctattggtgtgggaagccaagggcggttgcagggaaggaggaagaagacagtgaccccgagaaagcactcagaaacgagtactttgaagccgagccagaggacctggtggcggggatcaagatcaagcacctgtccaaggtgttcagggtgggaaataaggacagggcggccgtcagagacctgaacctcaacctgtacgagggacagatcaccgtcctgctgggccacaacggtgccgggaagaccaccaccctctccatgctcacaggtctctttccccccaccagtggacgggcatacatcagcgggtatgaaatttcccaggacatggttcagatccggaagagcctgggcctgtgcccgcagcacgacatcctgtttgacaacttgacagtcgcagagcacctttatttctacgcccagctgaagggcctgtcacgtcagaagtgccctgaagaagtcaagcagatgctgcacatcatcggcctggaggacaagtggaactcacggagccgcttcctgagcgggggcatgaggcgcaagctctccatcggcatcgccctcatcgcaggctccaaggtgctgatactggacgagcccacctcgggcatggacgccatctccaggagggccatctgggatcttcttcagcggcagaaaagtgaccgcaccatcgtgctgaccacccacttcatggacgaggctgacctgctgggagaccgcatcgccatcatggccaagggggagctgcagtgctgcgggtcctcgctgttcctcaagcagaaatacggtgccggctatcacatgacgctggtgaaggagccgcactgcaacccggaagacatctcccagctggtccaccaccacgtgcccaacgccacgctggagagcagcgctggggccgagctgtctttcatccttcccagagagagcacgcacaggtttgaaggtctctttgctaaactggagaagaagcagaaagagctgggcattgccagctttggggcatccatcaccaccatggaggaagtcttccttcgggtcgggaagctggtggacagcagtatggacatccaggccatccagctccctgccctgcagtaccagcacgagaggcgcgccagcgactgggctgtggacagcaacctctgtggggccatggacccctccgacggcattggagccctcatcgaggaggagcgcaccgctgtcaagctcaacactgggctcgccctgcactgccagcaattctgggccatgttcctgaagaaggccgcatacagctggcgcgagtggaaaatggtggcggcacaggtcctggtgcctctgacctgcgtcaccctggccctcctggccatcaactactcctcggagctcttcgacgaccccatgctgaggctgaccttgggcgagtacggcagaaccgtcgtgcccttctcagttcccgggacctcccagctgggtcagcagctgtcagagcatctgaaagacgcactgcaggctgagggacaggagccccgcgaggtgctcggtgacctggaggagttcttgatcttcagggcttctgtggaggggggcggctttaatgagcggtgccttgtggcagcgtccttcagagatgtgggagagcgcacggtcgtcaacgccttgttcaacaaccaggcgtaccactctccagccactgccctggccgtcgtggacaaccttctgttcaagctgctgtgcgggcctcacgcctccattgtggtctccaacttcccccagccccggagcgccctgcaggctgccaaggaccagtttaacgagggccggaagggattcgacattgccctcaacctgctcttcgccatggcattcttggccagcacgttctccatcctggcggtcagcgagagggccgtgcaggccaagcatgtgcagtttgtgagtggagtccacgtggccagtttctggctctctgctctgctgtgggacctcatctccttcctcatccccagtctgctgctgctggtggtgtttaaggccttcgacgtgcgtgccttcacgcgggacggccacatggctgacaccctgctgctgctcctgctctacggctgggccatcatccccctcatgtacctgatgaacttcttcttcttgggggcggccactgcctacacgaggctgaccatcttcaacatcctgtcaggcatcgccaccttcctgatggtcaccatcatgcgcatcccagctgtaaaactggaagaactttccaaaaccctggatcacgtgttcctggtgctgcccaaccactgtctggggatggcagtcagcagtttctacgagaactacgagacgcggaggtactgcacctcctccgaggtcgccgcccactactgcaagaaatataacatccagtaccaggagaacttctatgcctggagcgccccgggggtcggccggtttgtggcctccatggccgcctcagggtgcgcctacctcatcctgctcttcctcatcgagaccaacctgcttcagagactcaggggcatcctctgcgccctccggaggaggcggacactgacagaattatacacccggatgcctgtgcttcctgaggaccaagatgtagcggacgagaggacccgcatcctggcccccagtccggactccctgctccacacacctctgattatcaaggagctctccaaggtgtacgagcagcgggtgcccctcctggccgtggacaggctctccctcgcggtgcagaaaggggagtgcttcggcctgctgggcttcaatggagccgggaagaccacgactttcaaaatgctgaccggggaggagagcctcacttctggggatgcctttgtcgggggtcacagaatcagctctgatgtcggaaaggtgcggcagcggatcggctactgcccgcagtttgatgccttgctggaccacatgacaggccgggagatgctggtcatgtacgctcggctccggggcatccctgagcgccacatcggggcctgcgtggagaacactctgcggggcctgctgctggagccacatgccaacaagctggtcaggacgtacagtggtggtaacaagcggaagctgagcaccggcatcgccctgatcggagagcctgctgtcatcttcctggacgagccgtccactggcatggaccccgtggcccggcgcctgctttgggacaccgtggcacgagcccgagagtctggcaaggccatcatcatcacctcccacagcatggaggagtgtgaggccctgtgcacccggctggccatcatggtgcaggggcagttcaagtgcctgggcagcccccagcacctcaagagcaagttcggcagcggctactccctgcgggccaaggtgcagagtgaagggcaacaggaggcgctggaggagttcaaggccttcgtggacctgacctttccaggcagcgtcctggaagatgagcaccaaggcatggtccattaccacctgccgggccgtgacctcagctgggcgaaggttttcggtattctggagaaagccaaggaaaagtacggcgtggacgactactccgtgagccagatctcgctggaacaggtcttcctgagcttcgcccacctgcagccgcccaccgcagaggaggggcgatga

SEQ ID NO: 26 - protein, 1704 aa, Homo sapiens
MAVLRQLALLLWKNYTLQKRKVLVTVLELFLPLLFSGILIWLRLKIQSENVPNATIYPGQSIQELPLFFTFPPPGDTWELAYIPSHSDAAKTVTETVRRALVINMRVRGFPSEKDFEDYIRYDNCSSSVLAAVVFEHPFNHSKEPLPLAVKYHLRFSYTRRNYMWTQTGSFFLKETEGWHTTSLFPLFPNPGPREPTSPDGGEPGYIREGFLAVQHAVDRAIMEYHADAATRQLFQRLTVTIKRFPYPPFIADPFLVAIQYQLPLLLLLSFTYTALTIARAVVQEKERRLKEYMRMMGLSSWLHWSAWFLLFFLFLLIAASFMTLLFCVKVKPNVAVLSRSDPSLVLAFLLCFAISTISFSFMVSTFFSKANMAAAFGGFLYFFTYIPYFFVAPRYNWMTLSQKLCSCLLSNVAMAMGAQLIGKFEAKGMGIQWRDLLSPVNVDDDFCFGQVLGMLLLDSVLYGLVTWYMEAVFPGQFGVPQPWYFFIMPSYWCGKPRAVAGKEEEDSDPEKALRNEYFEAEPEDLVAGIKIKHLSKVFRVGNKDRAAVRDLNLNLYEGQITVLLGHNGAGKTTTLSMLTGLFPPTSGRAYISGYEISQDMVQIRKSLGLCPQHDILFDNLTVAEHLYFYAQLKGLSRQKCPEEVKQMLHIIGLEDKWNSRSRFLSGGMRRKLSIGIALIAGSKVLILDEPTSGMDAISRRAIWDLLQRQKSDRTIVLTTHFMDEADLLGDRIAIMAKGELQCCGSSLFLKQKYGAGYHMTLVKEPHCNPEDISQLVHHHVPNATLESSAGAELSFILPRESTHRFEGLFAKLEKKQKELGIASFGASITTMEEVFLRVGKLVDSSMDIQAIQLPALQYQHERRASDWAVDSNLCGAMDPSDGIGALIEEERTAVKLNTGLALHCQQFWAMFLKKAAYSWREWKMVAAQVLVPLTCVTLALLAINYSSELFDDPMLRLTLGEYGRTVVPFSVPGTSQLGQQLSEHLKDALQAEGQEPREVLGDLEEFLIFRASVEGGGFNERCLVAASFRDVGERTVVNALFNNQAYHSPATALAVVDNLLFKLLCGPHASIVVSNFPQPRSALQAAKDQFNEGRKGFDIALNLLFAMAFLASTFSILAVSERAVQAKHVQFVSGVHVASFWLSALLWDLISFLIPSLLLLVVFKAFDVRAFTRDGHMADTLLLLLLYGWAIIPLMYLMNFFFLGAATAYTRLTIFNILSGIATFLMVTIMRIPAVKLEELSKTLDHVFLVLPNHCLGMAVSSFYENYETRRYCTSSEVAAHYCKKYNIQYQENFYAWSAPGVGRFVASMAASGCAYLILLFLIETNLLQRLRGILCALRRRRTLTELYTRMPVLPEDQDVADERTRILAPSPDSLLHTPLIIKELSKVYEQRVPLLAVDRLSLAVQKGECFGLLGFNGAGKTTTFKMLTGEESLTSGDAFVGGHRISSDVGKVRQRIGYCPQFDALLDHMTGREMLVMYARLRGIPERHIGACVENTLRGLLLEPHANKLVRTYSGGNKRKLSTGIALIGEPAVIFLDEPSTGMDPVARRLLWDTVARARESGKAIIITSHSMEECEALCTRLAIMVQGQFKCLGSPQHLKSKFGSGYSLRAKVQSEGQQEALEEFKAFVDLTFPGSVLEDEHQGMVHYHLPGRDLSWAKVFGILEKAKEKYGVDDYSVSQISLEQVFLSFAHLQPPTAEEGR

SEQ ID NO: 27 - DNA, 574 bp, Homo sapiens
  source 1..574: mol_type = genomic DNA
agatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagc

SEQ ID NO: 28 - DNA, 873 bp, Cytomegalovirus
  source 1..873: mol_type = genomic DNA
ccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagc

SEQ ID NO: 29 - DNA, 395 bp, Homo sapiens
  source 1..395: mol_type = genomic DNA
agatccatatccgcggcaattttaaaagaaagggaggaatagggggacagacttcagcagagagactaattaatataataacaacacaattagaaatacaacatttacaaaccaaaattcaaaaaattttaaattttagagccgcggagatcccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagc

SEQ ID NO: 30 - DNA, 7494 bp, synthetic construct
  source 1..7494: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactcttgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatttttttgtttcaagccctatcgaattcccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcctgccttctccctcctgtgagtttaaggtaagtatcaaggttacaagacaggtttaaggagaccaatagaaactgggcttgtcgagacagagaagactcttgcgtttctgataggcacgcggccgcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatgcggccgcctattggtcttactgacatccactttgcctttctctccacagctagccaccatgcccagctctgtgtcctggggcattctgctgctggctggcctgtgctgtctggtgcctgtgtccctggctgaggaccctcagggggatgctgcccagaaaacagacacctcccaccatgaccaggaccaccccaccttcaacaagatcacccccaacctggcagagtttgccttcagcctgtacagacagctggcccaccagagcaacagcaccaacatctttttcagccctgtgtccattgccacagcctttgccatgctgagcctgggcaccaaggctgacacccatgatgagatcctggaaggcctgaacttcaacctgacagagatccctgaggcccagatccatgagggcttccaggaactgctgagaaccctgaaccagccagacagccagctgcagctgacaacaggcaatgggctgttcctgtctgagggcctgaagctggtggacaagtttctggaagatgtgaagaagctgtaccactctgaggccttcacagtgaactttggggacacagaagaggccaagaaacagatcaatgactatgtggaaaagggcacccagggcaagattgtggaccttgtgaaagagctggacagggacactgtgtttgcccttgtgaactacatcttcttcaagggcaagtgggagaggccctttgaagtgaaggacactgaggaagaggacttccatgtggaccaagtgaccacagtgaaggtgccaatgatgaagagactggggatgttcaatatccagcactgcaagaaactgagcagctgggtgctgctgatgaagtacctgggcaatgctacagccatattctttctgcctgatgagggcaagctgcagcacctggaaaatgagctgacccatgacatcatcaccaaatttctggaaaatgaggacagaagatctgccagcctgcatctgcccaagctgagcatcacaggcacatatgacctgaagtctgtgctgggacagctgggaatcaccaaggtgttcagcaatggggcagacctgagtggagtgacagaggaagcccctctgaagctgtccaaggctgtgcacaaggcagtgctgaccattgatgagaagggcacagaggctgctggggccatgtttctggaagccatccccatgtccatccccccagaagtgaagttcaacaagccctttgtgttcctgatgattgagcagaacaccaagagccccctgttcatgggcaaggttgtgaaccccacccagaaatgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 31 - DNA, 9064 bp, synthetic construct
  source 1..9064: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccaccatgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattgaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 32 - DNA, 9886 bp, synthetic construct
  source 1..9886: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagactagtgacttggtgagtaggcttcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtactatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcagaaggctgcacaccctatgacattaatcagatgcttaatgtgctaggagatcatcaaggggcattacaaatagtgaaagagatcattaatgaagaagcagcccagtgggatgtaacacacccactacccgcaggacccctaccagcaggacagctcagggaccctcgcggctcagatatagcagggaccaccagctcagtacaagaacagttagaatggatctatactgctaacccccgggtagatgtaggtgccatctaccggagatggattattctaggacttcaaaagtgtgtcaaaatgtacaacccagtatcagtcctagacattaggcagggacctaaagagcccttcaaggattatgtggacagattttacaaggcaattagagcagaacaagcctcaggggaagtgaaacaatggatgacagaatcattactcattcaaaatgctaatccagattgtaaggtcatcctgaagggcctaggaatgcaccccacccttgaagaaatgttaacggcttgtcagggggtaggaggcccaagctacaaagcaaaagtaatggcagaaatgatgcagaccatgcaaaatcaaaacatggtgcagcagggaggtccaaaaagacaaagacccccactaagatgttataattgtggaaaatttggccatatgcaaagacaatgtccggaaccaaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagacagtgtatatagaaggggtccccattaaggcactgctagacacaggggcagatgacaccataattaaagaaaatgatttacaattatcaggtccatggagacccaaaattatagggggcataggaggaggccttaatgtaaaagaatataacgacagggaagtaaaaatagaagataaaattttgagaggaacaatattgttaggagcaactcccattaatataataggtagaaatttgctggccccggcaggtgcccggttagtaatgggacaattatcagaaaaaattcctgtcacacctgtcaaattgaaggaaggggctcggggaccctgtgtaagacaatggcctctctctaaagagaagattgaagctttacaggaaatatgttcccaattagagcaggaaggaaaaatcagtagagtaggaggagaaaatgcatacaataccccaatattttgcataaagaagaaggacaaatcccagtggaggatgctagtagactttagagagttaaataaggcaacccaagatttctttgaagtgcaattagggataccccacccagcaggattaagaaagatgagacagataacagttttagatgtaggagacgcctattattccataccattggatccaaattttaggaaatatactgcttttactattcccacagtgaataatcagggacccgggattaggtatcaattcaactgtctcccgcaagggtggaaaggatctcctacaatcttccaaaatacagcagcatccattttggaggagataaaaagaaacttgccagcactaaccattgtacaatacatggatgatttatgggtaggttctcaagaaaatgaacacacccatgacaaattagtagaacagttaagaacaaaattacaagcctggggcttagaaaccccagaaaagaaggtgcaaaaagaaccaccttatgagtggatgggatacaaactttggcctcacaaatgggaactaagcagaatacaactggaggaaaaagatgaatggactgtcaatgacatccagaagttagttgggaaactaaattgggcagcacaattgtatccaggtcttaggaccaagaatatatgcaagttaattagaggaaagaaaaatctgttagagctagtgacttggacacctgaggcagaagctgaatatgcagaaaatgcagagattcttaaaacagaacaggaaggaacctattacaaaccaggaatacctattagggcagcagtacagaaattggaaggaggacagtggagttaccaattcaaacaagaaggacaagtcttgaaagtaggaaaatacaccaagcaaaagaacacccatacaaatgaacttcgcacattagctggtttagtgcagaagatttgcaaagaagctctagttatttgggggatattaccagttctagaactcccgatagaaagagaggtatgggaacaatggtgggcggattactggcaggtaagctggattcccgaatgggattttgtcagcaccccacctttgctcaaactatggtacacattaacaaaagaacccatacccaaggaggacgtttactatgtagatggagcatgcaacagaaattcaaaagaaggaaaagcaggatacatctcacaatacggaaaacagagagtagaaacattagaaaacactaccaatcagcaagcagaattaacagctataaaaatggctttggaagacagtgggcctaatgtgaacatagtaacagactctcaatatgcaatgggaattttgacagcacaacccacacaaagtgattcaccattagtagagcaaattatagccttaatgatacaaaagcaacaaatatatttgcagtgggtaccagcacataaaggaataggaggaaatgaggagatagataaattagtgagtaaaggcattagaagagttttattcttagaaaaaatagaagaagctcaagaagagcatgaaagatatcataataattggaaaaacctagcagatacatatgggcttccacaaatagtagcaaaagagatagtggccatgtgtccaaaatgtcagataaagggagaaccagtgcatggacaagtggatgcctcacctggaacatggcagatggattgtactcatctagaaggaaaagtagtcatagttgcggtccatgtagccagtggattcatagaagcagaagtcatacctagggaaacaggaaaagaaacggcaaagtttctattaaaaatactgagtagatggcctataacacagttacacacagacaatgggcctaactttacctcccaagaagtggcagcaatatgttggtggggaaaaattgaacatacaacaggtataccatataacccccaatctcaaggatcaatagaaagcatgaacaaacaattaaaagagataattgggaaaataagagatgattgccaatatacagagacagcagtactgatggcttgccatattcacaattttaaaagaaagggaggaatagggggacagacttcagcagagagactaattaatataataacaacacaattagaaatacaacatttacaaaccaaaattcaaaaaattttaaattttagagtctactacagagaagggagagaccctgtgtggaaaggaccagcacaattaatctggaaaggggaaggagcagtggtcctcaaggacggaagtgacctaaaggttgtaccaagaaggaaagctaaaattattaaggattatgaacccaaacaaagagtgggtaatgagggtgacgtggaaggtaccaggggatctgataactaaatggcagggaatagtcagatattggatgagacaaagaaatttgaaatggaactattatatgcatcagctggcggccgcgaattcactagtgattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccaatcgaattcccgcggccgcaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 33 - DNA, 3384 bp, synthetic construct
  source 1..3384: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgattatgcccctaggaccagaagaaagaagattgcttcgcttgatttggctcctttacagcaccaatccatatccaccaagtggggaagggacggccagacaacgccgacgagccaggagaaggtggagacaacagcaggatcaaattagagtcttggtagaaagactccaagagcaggtgtatgcagttgaccgcctggctgacgaggctcaacacttggctatacaacagttgcctgaccctcctcattcagcttagaatcactagtgaattcacgcgtggtacctctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgtcgaccaattgttgtgtctcaaaatctctgatgttacattgcacaagataaaaatatatcatcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctgttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 34 - DNA, 6264 bp, synthetic construct
  source 1..6264: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgattgccatggcaacatatatccagagagtacagtgcatctcaacatcactactggttgttctcaccacattggtctcgtgtcagattcccagggataggctctctaacataggggtcatagtcgatgaagggaaatcactgaagatagctggatcccacgaatcgaggtacatagtactgagtctagttccgggggtagactttgagaatgggtgcggaacagcccaggttatccagtacaagagcctactgaacaggctgttaatcccattgagggatgccttagatcttcaggaggctctgataactgtcaccaatgatacgacacaaaatgccggtgctccccagtcgagattcttcggtgctgtgattggtactatcgcacttggagtggcgacatcagcacaaatcaccgcagggattgcactagccgaagcgagggaggccaaaagagacatagcgctcatcaaagaatcgatgacaaaaacacacaagtctatagaactgctgcaaaacgctgtgggggaacaaattcttgctctaaagacactccaggatttcgtgaatgatgagatcaaacccgcaataagcgaattaggctgtgagactgctgccttaagactgggtataaaattgacacagcattactccgagctgttaactgcgttcggctcgaatttcggaaccatcggagagaagagcctcacgctgcaggcgctgtcttcactttactctgctaacattactgagattatgaccacaatcaggacagggcagtctaacatctatgatgtcatttatacagaacagatcaaaggaacggtgatagatgtggatctagagagatacatggtcaccctgtctgtgaagatccctattctttctgaagtcccaggtgtgctcatacacaaggcatcatctatttcttacaacatagacggggaggaatggtatgtgactgtccccagccatatactcagtcgtgcttctttcttagggggtgcagacataaccgattgtgttgagtccagattgacctatatatgccccagggatcccgcacaactgatacctgacagccagcaaaagtgtatcctgggggacacaacaaggtgtcctgtcacaaaagttgtggacagccttatccccaagtttgcttttgtgaatgggggcgttgttgctaactgcatagcatccacatgtacctgcgggacaggccgaagaccaatcagtcaggatcgctctaaaggtgtagtattcctaacccatgacaactgtggtcttataggtgtcaatggggtagaattgtatgctaaccggagagggcacgatgccacttggggggtccagaacttgacagtcggtcctgcaattgctatcagacccgttgatatttctctcaaccttgctgatgctacgaatttcttgcaagactctaaggctgagcttgagaaagcacggaaaatcctctcggaggtaggtagatggtacaactcaagagagactgtgattacgatcatagtagttatggtcgtaatattggtggtcattatagtgatcatcatcgtgctttatagactcagaaggtgaaatcactagtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 35 - DNA, 6522 bp, synthetic construct
  source 1..6522: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagcatgtggtctgagttaaaaatcaggagcaacgacggaggtgaaggaccagaggacgccaacgacccccggggaaagggggtgcaacacatccatatccagccatctctacctgtttatggacagagggttagggatggtgataggggcaaacgtgactcgtactggtctacttctcctagtggtagcaccacaaaaccagcatcaggttgggagaggtcaagtaaagccgacacatggttgctgattctctcattcacccagtgggctttgtcaattgccacagtgatcatctgtatcataatttctgctagacaagggtatagtatgaaagagtactcaatgactgtagaggcattgaacatgagcagcagggaggtgaaagagtcacttaccagtctaataaggcaagaggttatagcaagggctgtcaacattcagagctctgtgcaaaccggaatcccagtcttgttgaacaaaaacagcagggatgtcatccagatgattgataagtcgtgcagcagacaagagctcactcagcactgtgagagtacgatcgcagtccaccatgccgatggaattgccccacttgagccacatagtttctggagatgccctgtcggagaaccgtatcttagctcagatcctgaaatctcattgctgcctggtccgagcttgttatctggttctacaacgatctctggatgtgttaggctcccttcactctcaattggcgaggcaatctatgcctattcatcaaatctcattacacaaggttgtgctgacatagggaaatcatatcaggtcctgcagctagggtacatatcactcaattcagatatgttccctgatcttaaccccgtagtgtcccacacttatgacatcaacgacaatcggaaatcatgctctgtggtggcaaccgggactaggggttatcagctttgctccatgccgactgtagacgaaagaaccgactactctagtgatggtattgaggatctggtccttgatgtcctggatctcaaagggagaactaagtctcaccggtatcgcaacagcgaggtagatcttgatcacccgttctctgcactataccccagtgtaggcaacggcattgcaacagaaggctcattgatatttcttgggtatggtggactaaccacccctctgcagggtgatacaaaatgtaggacccaaggatgccaacaggtgtcgcaagacacatgcaatgaggctctgaaaattacatggctaggagggaaacaggtggtcagcgtgatcatccaggtcaatgactatctctcagagaggccaaagataagagtcacaaccattccaatcactcaaaactatctcggggcggaaggtagattattaaaattgggtgatcgggtgtacatctatacaagatcatcaggctggcactctcaactgcagataggagtacttgatgtcagccaccctttgactatcaactggacacctcatgaagccttgtctagaccaggaaataaagagtgcaattggtacaataagtgtccgaaggaatgcatatcaggcgtatacactgatgcttatccattgtcccctgatgcagctaacgtcgctaccgtcacgctatatgccaatacatcgcgtgtcaacccaacaatcatgtattctaacactactaacattataaatatgttaaggataaaggatgttcaattagaggctgcatataccacgacatcgtgtatcacgcattttggtaaaggctactgctttcacatcatcgagatcaatcagaagagcctgaataccttacagccgatgctctttaagactagcatccctaaattatgcaaggccgagtcttaagcggccgcgcatgcgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtctattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 36 - DNA, 7349 bp, synthetic construct
  source 1..7349: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactcttgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatttttttgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcccagctctgtgtcctggggcattctgctgctggctggcctgtgctgtctggtgcctgtgtccctggctgaggaccctcagggggatgctgcccagaaaacagacacctcccaccatgaccaggaccaccccaccttcaacaagatcacccccaacctggcagagtttgccttcagcctgtacagacagctggcccaccagagcaacagcaccaacatctttttcagccctgtgtccattgccacagcctttgccatgctgagcctgggcaccaaggctgacacccatgatgagatcctggaaggcctgaacttcaacctgacagagatccctgaggcccagatccatgagggcttccaggaactgctgagaaccctgaaccagccagacagccagctgcagctgacaacaggcaatgggctgttcctgtctgagggcctgaagctggtggacaagtttctggaagatgtgaagaagctgtaccactctgaggccttcacagtgaactttggggacacagaagaggccaagaaacagatcaatgactatgtggaaaagggcacccagggcaagattgtggaccttgtgaaagagctggacagggacactgtgtttgcccttgtgaactacatcttcttcaagggcaagtgggagaggccctttgaagtgaaggacactgaggaagaggacttccatgtggaccaagtgaccacagtgaaggtgccaatgatgaagagactggggatgttcaatatccagcactgcaagaaactgagcagctgggtgctgctgatgaagtacctgggcaatgctacagccatattctttctgcctgatgagggcaagctgcagcacctggaaaatgagctgacccatgacatcatcaccaaatttctggaaaatgaggacagaagatctgccagcctgcatctgcccaagctgagcatcacaggcacatatgacctgaagtctgtgctgggacagctgggaatcaccaaggtgttcagcaatggggcagacctgagtggagtgacagaggaagcccctctgaagctgtccaaggctgtgcacaaggcagtgctgaccattgatgagaagggcacagaggctgctggggccatgtttctggaagccatccccatgtccatccccccagaagtgaagttcaacaagccctttgtgttcctgatgattgagcagaacaccaagagccccctgttcatgggcaaggttgtgaaccccacccagaaatgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 37 - DNA, 4391 bp, synthetic construct
  source 1..4391: mol_type = other DNA
atgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattga

SEQ ID NO: 38 - DNA, 1738 bp, synthetic construct
  source 1..1738: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccacc

SEQ ID NO: 39 - DNA, 600 bp, Mus musculus
  source 1..600: mol_type = genomic DNA
gggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagct

SEQ ID NO: 40 - DNA, 1182 bp, Homo sapiens
  source 1..1182: mol_type = genomic DNA
atgcaccaagcagggtacccaggctgcagaggtgccatggctgagtcacacctgctgcagtggctgctgctgctgctgcccacgctctgtggcccaggcactgctgcctggaccacctcatccttggcctgtgcccagggccctgagttctggtgccaaagcctggagcaagcattgcagtgcagagccctagggcattgcctacaggaagtctggggacatgtgggagccgatgacctatgccaagagtgtgaggacatcgtccacatccttaacaagatggccaaggaggccattttccaggacacgatgaggaagttcctggagcaggagtgcaacgtcctccccttgaagctgctcatgccccagtgcaaccaagtgcttgacgactacttccccctggtcatcgactacttccagaaccagactgactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggatctctccgagcagcaattccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaaggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaagtgcaagcaatttgtggagcagcacacgccccagctgctgaccctggtgcccaggggctgggatgcccacaccacctgccaggccctcggggtgtgtgggaccatgtccagccctctccagtgtatccacagccccgacctttga

SEQ ID NO: 41 - protein, 381 aa, Homo sapiens
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 42 - DNA, 4116 bp, Homo sapiens
  source 1..4116: mol_type = genomic DNA
atgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctactgggcggtgggagacttcggcccatgcagcgcctcctgtgggggtggcctgcgggagcggccagtgcgctgcgtggaggcccagggcagcctcctgaagacattgcccccagcccggtgcagagcaggggcccagcagccagctgtggcgctggaaacctgcaacccccagccctgccctgccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgcggcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgcccacctaggtggaaagtcatgtcccttggcccatgttcggccagctgtggccttggcactgctagacgctcggtggcctgtgtgcagctcgaccaaggccaggacgtggaggtggacgaggcggcctgtgcggcgctggtgcggcccgaggccagtgtcccctgtctcattgccgactgcacctaccgctggcatgttggcacctggatggagtgctctgtttcctgtggggatggcatccagcgccggcgtgacacctgcctcggaccccaggcccaggcgcctgtgccagctgatttctgccagcacttgcccaagccggtgactgtgcgtggctgctgggctgggccctgtgtgggacagggtgcctgtggcaggcagcaccttgagccaacaggaaccattgacatgcgaggcccagggcaggcagactgtgcagtggccattgggcggcccctcggggaggtggtgaccctccgcgtccttgagagttctctcaactgcagtgcgggggacatgttgctgctttggggccggctcacctggaggaagatgtgcaggaagctgttggacatgactttcagctccaagaccaacacgctggtggtgaggcagcgctgcgggcggccaggaggtggggtgctgctgcggtatgggagccagcttgctcctgaaaccttctacagagaatgtgacatgcagctctttgggccctggggtgaaatcgtgagcccctcgctgagtccagccacgagtaatgcagggggctgccggctcttcattaatgtggctccgcacgcacggattgccatccatgccctggccaccaacatgggcgctgggaccgagggagccaatgccagctacatcttgatccgggacacccacagcttgaggaccacagcgttccatgggcagcaggtgctctactgggagtcagagagcagccaggctgagatggagttcagcgagggcttcctgaaggctcaggccagcctgcggggccagtactggaccctccaatcatgggtaccggagatgcaggaccctcagtcctggaagggaaaggaaggaacctga

SEQ ID NO: 43 - protein, 1371 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 44 - protein, 84 aa, Simian immunodeficiency virus
MPLGPEERRLLRLIWLLYSTNPYPPSGEGTARQRRRARRRWRQQQDQIRVLVERLQEQVYAVDRLADEAQHLAIQQLPDPPHSA

SEQ ID NO: 45 - DNA, 22 bp, synthetic construct
  source 1..22: mol_type = other DNA
gggaaagtgatgtggtgtactg

SEQ ID NO: 46 - DNA, 20 bp, synthetic construct
  source 1..20: mol_type = other DNA
caatggacacagggctgaaa

SEQ ID NO: 47 - DNA, 17 bp, synthetic construct
  source 1..17: mol_type = other DNA
tgccttctccctcctgt

SEQ ID NO: 48 - DNA, 1013 bp, synthetic construct
  source 1..1013: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcaagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcagctctagagcctctgctaaccatgttcatgccttcttctttttcctacag

SEQ ID NO: 49 - DNA, 5824 bp, synthetic construct
  source 1..5824: mol_type = other DNA
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcaacagagatcgatctgtttccttgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatggaattcaccccaccagtgcaggctgcctatcagaaagtggtggctggtgtggctaatgccctggcccacaagtttcactaagctcgcttccttgctgtccaatttctattaaaggttccttggttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagtttggctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 50 - DNA, 355 bp, Human immunodeficiency virus 1
  source 1..355: mol_type = genomic DNA
agtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagt

(19) United States
(12) Patent Application Publication
(10) Pub. No.: US 2026/0357291 A1
(43) Pub. Date: Nov. 12, 2026

(54) RETROVIRAL VECTOR COMPRISING RRE INSERTED WITHIN AN INTRON

(71) Applicant: IMPERIAL COLLEGE INNOVATIONS LIMITED, London (GB)

(72) Inventors: Jack HICKMOTT, London (GB); Uta GRIESENBACH, London (GB); Eric ALTON, London (GB)

(21) Appl. No.: 19/114,349

(22) PCT Filed: Sep. 22, 2023
(86) PCT No.: PCT/GB2023/052462

§ 371 Date: Mar. 21, 2025

(30) Foreign Application Priority Data
Sep. 23, 2022 (GB) 2213936.4

(51) Int. Cl. C12N 15/86; C07K 14/805

(52) U.S. Cl. CPC C12N 15/86; C07K 14/805; C12N 2740/15043; C12N 2740/15045; C12N 2830/42; C12N 2830/48

(57) ABSTRACT

The present invention relates to retroviral vectors modified to improve transgene expression. In particular, the invention relates to retroviral vectors lacking an endogenous Rev response element (RRE) and comprising an intron, particularly a chimeric intron, into which an RRE has been inserted, as well as methods of production and uses thereof.

### FIELD OF THE INVENTION

The present invention relates to retroviral vectors modified to improve transgene expression. In particular, the invention relates to retroviral vectors lacking an endogenous Rev response element (RRE) and comprising an intron, particularly a chimeric intron into which an RRE has been inserted, as well as methods of production and uses thereof.

### BACKGROUND TO THE INVENTION

The use of nucleic acids as medicine, or gene therapy, is a promising new treatment modality. The reason many gene therapies currently in use or under development are not effective at curing diseases is because it is difficult to make sufficient protein to reach the therapeutic threshold needed to treat or cure the disease. As such, generating sufficient gene expression is a major barrier to the success of many gene therapies.

The current approach to reaching the large doses needed for gene therapy to be successful is to administer massive amounts of the gene therapy to the patient, over 1 trillion viruses per kg of body mass. For example, Zolgensma is given at 1.1×10¹⁴ viral genomes per kg of body mass. Producing so much virus is expensive, contributing to the $1,000,000 USD cost of gene therapies, and giving so much virus to a person can trigger immune responses that threaten the health of the patient and the efficacy of the therapy. To circumvent these problems, research to-date has focused on gain of function mutations resulting in more potent proteins. Such an approach has been used previously in the gene therapies for haemophilia B (the Padua mutation in Factor IX) and lipoprotein lipase deficiency (the S447X variant of lipoprotein lipase). However, such gain-of-function mutations are not available for most gene therapies. Furthermore, even with gain-of-function mutations, high doses of the gene therapy vector were still necessary to make the treatment effective. Therefore, such gain-of-function mutations alone do not adequately address the existing problems associated with producing sufficient quantities of vector, or the unwanted and clinically dangerous side effects associated with the large doses required.

The present inventors have previously developed a lentiviral vector, which has been pseudotyped with hemagglutinin-neuraminidase (HN) and fusion (F) proteins from a respiratory paramyxovirus, comprising a promoter and a transgene. Typically, the backbone of the vector is from a simian immunodeficiency virus (SIV), such as SIV1 or African green monkey SIV (SIV-AGM). Preferably the backbone of a viral vector of the invention is from SIV-AGM. The HN and F proteins function, respectively, to attach to sialic acids and mediate cell fusion for vector entry to target cells. The present inventors discovered that this specifically F/HN-pseudotyped lentiviral vector can efficiently transduce airway epithelium, resulting in transgene expression sustained for periods beyond the proposed lifespan of airway epithelial cells. Importantly, the present inventors also found that re-administration does not result in a loss of efficacy. These features make the vectors of the present invention attractive candidates for treating diseases via their use in expressing therapeutic proteins: (i) within the cells of the respiratory tract; (ii) secreted into the lumen of the respiratory tract; and (iii) secreted into the circulatory system. However, even using this state-of-the-art platform technology, the levels of transgene expressed are at the lower predicted threshold required for clinical efficacy.

There is therefore an unmet clinical need for new technologies to improve the potency of gene therapies. It is an object of the invention to address one or more of these problems. In particular, it is an object of the invention to provide new nucleic acid cassettes and gene therapy vectors which enable increased production of therapeutic proteins, allowing for lower doses of vector to be administered to patients.

### SUMMARY OF THE INVENTION

At present, there remains a pressing need for technology that more efficiently produces therapeutic proteins for gene therapy, including from the inventors' own lentiviral platform. To-date, other groups have focused on making each copy of a gene produce more of the therapeutic protein. For example, work on adeno-associated viral vectors (AAV vectors) have introduced an intron into the viral genome. The splicing of introns by a cell leads to the stabilization of mRNA, increasing the amount of mRNA in the cell and resulting in the production of more protein. However, this approach cannot readily be applied to viral vectors based on RNA viruses such as retrovirus and lentiviruses. This is because RNA genomes are subject to the same intron removal steps that mRNA is, such that introns are removed from RNA genomes during manufacturing, reducing the amount of protein an RNA viral vector can make.

The present inventors have for the first time demonstrated that it is possible to introduce an intron into a lentiviral genome, and that the introduction of such an intron can increase transgene expression. In particular, the inventors have found that by removal of the endogenous Rev response element of a Simian Immunodeficiency Virus (SIV vector) pseudotyped with a VSV-G or F/HN envelope, and introduction of a β-globin/IgG intron with a precisely inserted SIV RRV into the SIV.VSV-G or SIV.F/HN genome increased AAT transgene expression by 686-fold or 501-fold, respectively, compared with corresponding vectors lacking an intron. The inventors' innovative approach has the potential to provide several clinically important advantages: (i) allowing gene therapies to more easily reach the required therapeutic window, making them more efficacious; (ii) lowering the dose of a gene therapy agent required for administration to a patient, making the gene therapy safer; and/or (iii) lowering the production costs (as less vector is needed per patient), solving a major challenge for clinical trials, & pharmaceutical companies, and health care providers.

Accordingly, the present invention provides a retroviral vector comprising an intron; wherein: (a) the endogenous Rev response element (RRE) of the retroviral genome is deleted; and (b) a retroviral RRE is inserted into the intron within 100 bp 5′ of the splice acceptor's branch site. The retroviral RRE may be inserted within 20 bp 5′ of the splice acceptor's branch site. The intron may be a chimeric intron, optionally selected from a β-globin/IgG chimeric intron or a chimeric intron from the CAGGS promoter. The intron may be a viral intron, optionally selected from SV40 intron, CMV Intron A and adenovirus tripartite leader sequence intron.

The invention also preferably provides a retroviral vector comprising a chimeric intron; wherein: (a) the endogenous Rev response element (RRE) of the retroviral genome is deleted; and (b) a retroviral RRE is inserted into the chimeric intron.

According to any retroviral vector of the invention, the retroviral RRE inserted into the intron may be the endogenous RRE of the retroviral genome. The RRE may be a Simian immunodeficiency virus (SIV) RRE. The RRE may comprise or consist of a nucleic acid sequence having at least 90% identity to SEQ ID NO: 1.

According to any retroviral vector of the invention, the intron may be less than 1,000 bp in length, preferably less than 800 bp in length.

According to any retroviral vector of the invention, the chimeric intron may be a β-globin/IgG chimeric intron or a chimeric intron from the CAGGS promoter. The chimeric intron may be a β-globin/IgG chimeric intron and the RRE inserted between (i) a splice donor site comprising or consisting of a nucleic acid sequence of TGAGTTTAAGGTAAGT (SEQ ID NO: 2); and (ii) a splice acceptor site comprising or consisting of a nucleic acid sequence of CTCTCCACAG (SEQ ID NO: 3). The 3-globin/IgG chimeric intron may comprise or consist of a nucleic acid sequence having at least 90% identity to SEQ ID NO: 4. The intron may be a β-globin/IgG chimeric intron and the RRE an SIV RRE, and optionally wherein the chimeric intron comprising the RRE may comprise or consist of a nucleic acid sequence having at least 90% identity to SEQ ID NO: 5.

According to any retroviral vector of the invention, the intron may be between a promoter and a transgene operably linked to said promoter, wherein optionally the promoter is selected from the group consisting of a cytomegalovirus (CMV) promoter, elongation factor 1a (EF1a) promoter, and a hybrid human CMV enhancer/EF1a (hCEF) promoter, preferably a hCEF promoter. The transgene may encode a therapeutic protein, wherein optionally said therapeutic protein is selected from: (a) a secreted therapeutic protein, optionally Alpha-1 Antitrypsin (AAT), Factor VIII, Surfactant Protein B (SFTPB), ADAMTS13, Factor VII, Factor IX, Factor X, Factor XI, von Willebrand Factor, Granulocyte-Macrophage Colony-Stimulating Factor (GM-CSF), Surfactant Protein C (SP-C), decorin, an anti-inflammatory protein and a monoclonal antibody against an infectious agent; or (b) CFTR, ABCA3, DNAH5, DNAH11, DNA11, DNA12, CSF2RA, CSF2RB and TRIM-72.

Any retroviral vector of the invention may be a lentiviral vector. Said lentiviral vector may be selected from the group consisting of a Human immunodeficiency virus (HIV) vector, a Simian immunodeficiency virus (SIV) vector, a Feline immunodeficiency virus (FIV) vector, an Equine infectious anaemia virus (EIAV) vector, and a Visna/maedi virus vector.

Any retroviral vector of the invention may be pseudotyped with haemagglutinin-neuraminidase (HN) and fusion (F) proteins from a respiratory paramyxovirus or G glycoprotein from Vesicular Stomatitis Virus (G-VSV).

According to any retroviral vector of the invention, transgene expression may be increased by at least about 2-fold, preferably at least about 5-fold, more preferably at least about 10-fold compared with a corresponding vector which lacks a intron into which a retroviral RRE has been inserted.

The invention also provides a nucleic acid comprising or consisting of a intron into which a retroviral RRE has been inserted, wherein optionally (i) the intron; and/or (ii) the RRE are as defined herein.

The invention further provides a plasmid comprising a nucleic acid of the invention.

The invention also provides a retroviral vector, nucleic acid and/or plasmid of the invention, which is codon-optimised.

The invention further provides a composition comprising a retroviral vector, nucleic acid and/or plasmid of the invention, and a pharmaceutically-acceptable carrier.

The invention also provides a host cell comprising a retroviral vector, nucleic acid and/or plasmid of the invention.

The invention also provides a retroviral vector, nucleic acid plasmid or composition according as described herein for use in a method of treatment.

The invention also provides a method of producing a retroviral vector, said method comprising the following steps: (a) growing cells in suspension; (b) transfecting the cells with one or more plasmids; (c) adding a nuclease; (d) harvesting the lentivirus; (e) adding trypsin; and (f) purification; wherein the one or more plasmids comprises a vector genome plasmid which comprises a nucleic acid of the invention, and optionally (i) a promoter of the invention and/or (ii) a transgene of the invention.

The invention also provides a method of differentiating between a retroviral vector and a transgene expressed by said retroviral vector, said method comprising the steps of: (a1) transfecting cells with a retroviral vector of the invention; (b1) culturing the cells to allow transgene expression by the retrovirus; and (c1) quantifying RNA within the cells; or (a2) quantifying RNA within cells of a sample obtained from a patient who has undergone treatment with a retroviral vector, nucleic acid, plasmid or composition of the invention; wherein: (i) the amount of RNA comprising the chimeric intron into which a retroviral RRE has been inserted corresponds to the copy number of the retroviral vector; and (ii) the amount of RNA lacking the chimeric intron into which a retroviral RRE has been inserted corresponds to the amount of transgene mRNA; wherein optionally RNA is quantified by a PCR-based or in situ hybridisation-based assay.

### BRIEF DESCRIPTION OF THE DRAWINGS

FIG. 1: Rev response element (RRE) intron created by inserting rSIV RRE into a chimeric intron. (A) Schematic of the chimeric intron which is composed of the splice donor from an intron in Hemoglobin subunit B (black), and the splice acceptor from an intron in immunoglobulin gamma (grey). (B) Schematic of the RRE intron, where the rSIV RRE (light grey) was inserted between the splice donor and splice acceptor.

FIG. 2: A-H show schematic drawings of exemplary plasmids used for production of the vectors of the invention. (A) Shows a schematic of an intron containing lentiviral vector genome plasmid (pDNA1) encoding an Alpha-1-Antitrypsin transgene. An intron has been inserted between the promoter (hCEF) and the RRE has been moved to within the intron. (B) Shows a schematic of a plasmid encoding codon optimized SIV Gag and Pol (pDNA2a) for lentiviral production. (C) Shows a schematic of a plasmid encoding SIV Gag and Pol (pDNA2a) for lentiviral production. (D) Shows a schematic of a plasmid encoding SIV Rev for lentiviral production (pDNA2b). (E) Shows a schematic of a plasmid encoding the fusion protein from Sendai virus (pDNA3a) for lentiviral production. (F) Shows a schematic of a plasmid encoding the hemagglutinin-neuraminidase protein from Sendai virus (pDNA3b) for lentiviral production. (G) Shows a schematic of a no intron lentiviral vector genome plasmid encoding an Alpha-1-Antitrypsin transgene (pDNA1). The RRE element is 5′ of the promoter (hCEF) between the Partial GAG and cPPT sequences. (H) Shows a schematic of a plasmid encoding the VSV glycoprotein (pDNA3) for lentiviral production.

FIG. 3: RRE Intron enhances AAT expression 10.6x. HEK293T cells were transfected with a plasmid encoding an Alpha-1-Antitrypsin (AAT) transgene without (No Intron) or with (Intron) the RRE intron. Inclusion of the intron significantly enhanced AAT expression (p=0.0003). Neg—negative control. Each dot represents a different well of transduced HEK293T cells. A Mann-Whitney test was used for statistical analysis.

FIG. 4: RRE Intron is correctly spliced in HEK293T cells. DNA (A) and RNA (B) was extracted from HEK293Tcells transfected with plasmids encoding an Alpha-1-Antitrypsin (AAT) transgene without (pGM407—No Intron) or with (pGM991—Intron) the RRE intron. (A) PCR of DNA extracted from transfected cells verified that pGM991 contains the 760 bp RRE intron. (B) Reverse Transcriptase PCR of RNA extracted from transfected cells verified that the RRE intron was spliced during mRNA maturation.

FIG. 5: RRE intron is packaged into rSIV.VSV-G lentivirus. DNA was extracted from HEK293T cells transduced with rSIV.VSV-G lentivirus expressing AAT without (vGM290) and with (vGM291) an RRE intron. Non-transduced cells (NTC) were used as controls. PCR of the resulting DNA using primers that bind to either side of the intron reveal that the intron was packaged into vGM291, producing an 1127 bp product, whereas in the absence of an intron, vGM290 produces a smaller 371 bp product. A no template control (n) as well as the lentiviral transfer plasmids for vGM290 (pGM407) and vGM291 (pGM991) were included.

FIG. 6: RRE intron is successfully spliced by transduced HEK293T cells. RNA was extracted from HEK293T cells transduced with rSIV.VSV-G lentivirus expressing AAT without (vGM290) and with (vGM291) an RRE intron. Non-transduced cells (NTC) were used as controls. RT-PCR of the resulting RNA using primers that bind to either side of the intron reveal that the intron was spliced during expression of vGM291, producing the same 277 bp produced cells transduced with vGM290. A no template control (n) as well as the lentiviral transfer plasmids for vGM290 (pGM407) and vGM291 (pGM991) were included.

FIG. 7: RRE Intron enhances AAT expression 686-fold in HEK293T cells. HEK293Tcells were transduced with rSIV.VSV-G lentivirus expressing AAT without (No Intron) and with (Intron) an RRE intron. Compared to the state of the art no intron virus, the RRE containing virus significantly enhanced AAT expression (p=0.0037, Kruskal-Wallis test with Dunn's multiple comparison correction). Non-transduced cells (NTC) were used as controls.

FIG. 8: An RRE Intron increases transgene transcription in HEK293T cells. (A) RT-ddPCR was performed on RNA extracted from transfected HEK293T cells. Inclusion of the intron increased the amount of mRNA (WPRE copies). WPRE transcript copies were standardized to the house keeping gene Beta-2-Microglobulin (B2M) (B). Inclusion of an RRE intron increases the amount of mRNA produced per plasmid copy (quantified by ddPCR from DNA extracted from transfected HEK293T cells). Each dot represents a different well of transduced HEK293T cells. Statistical analysis was performed with a Mann-Whitney test.

FIG. 9: RRE Intron enhances AAT expression 501-fold in HEK293T cells. HEK293T cells were transduced with rSIV.F/HN lentivirus expressing AAT without (No Intron) and with (Intron) an RRE intron. Compared to the state of the art no intron virus, the RRE containing virus significantly enhanced AAT expression (p=0.0002, Mann-Whitney test). Non-transduced cells (NTC) were used as controls.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

A “vector” or “construct” (sometimes referred to as gene delivery or gene transfer “vehicle”) refers to a macromolecule or complex of molecules comprising a polynucleotide to be delivered to a host cell, either in vitro or in vivo. A vector can be a linear or a circular molecule. A vector of the invention may be viral or non-viral. All disclosure herein in relation vectors of the invention applies equally to viral and non-viral vectors unless otherwise stated. All disclosure in relation to viral vectors of the invention applies equally and without reservation to lentiviral (e.g. SIV) vectors, particularly to lentiviral (e.g. SIV) vectors that are pseudotyped with hemagglutinin-neuraminidase (HN) and fusion (F) proteins from a respiratory paramyxovirus (also referred to herein asSIV F/HN or SIV-FHN).

As used herein, the terms “viral vector”, “retroviral vector” and “retroviral F/HN vector” are used interchangeably to mean a retroviral vector comprising a retroviral RNA sequence and pseudotyped with hemagglutinin-neuraminidase (HN) and fusion (F) proteins from a respiratory paramyxovirus, unless otherwise stated. The terms “lentiviral vector” and “lentiviral F/HN vector” are used interchangeably to mean a lentiviral vector pseudotyped with hemagglutinin-neuraminidase (HN) and fusion (F) proteins from a respiratory paramyxovirus, unless otherwise stated. All disclosure herein in relation to retroviral vectors of the invention applies equally and without reservation to lentiviral vectors of the invention and to SIV vectors that are pseudotyped with hemagglutinin-neuraminidase (HN) and fusion (F) proteins from a respiratory paramyxovirus (also referred to herein asSIV F/HN or SIV-FHN).

The term “intron” as used herein refers to a nucleic acid sequence within a gene that is located between exons. Introns are transcribed along with the exons but are removed from the primary gene transcript by RNA splicing to leave mature mRNA. The removal of introns typically leads to the stabilization of mRNA, increasing the amount of mRNA in the cell.

Rev (regulator of virion) is a trans-acting nuclear protein whose functional expression are required for retroviral replication. Specifically, the rev gene products are required for processing and translation of the gag and env mRNAs, and thus rev regulates the expression of the viral structural proteins.

The term “Rev-responsive element” (RRE) refers to a cis-acting anti-repression sequence in env, which is responsive to the rev gene product. mRNAs that contain an RRE can be exported from the nucleus to the cytoplasm for translation and virion packaging. The terms “RRE” and “RRE sequence” are used interchangeably herein.

As used herein, the term “plasmid”, refers to a common type of non-viral vector. A plasmid is an extra-chromosomal DNA molecule separate from the chromosomal DNA which is capable of replicating independently of the chromosomal DNA. Preferably a plasmid is circular and may be double-stranded.

The terms “nucleic acid cassette”, “nucleic acid construct”, “expression cassette” and “nucleic acid expression cassette” are used interchangeably to mean a nucleic acid molecule that is capable of directing transcription. A nucleic acid cassette includes, at the least, a promoter or a structure functionally equivalent to a promoter and a nucleic acid sequence to be transcribed. Thus, a nucleic acid cassette includes, at the least, a promoter or a structure functionally equivalent to a promoter and a nucleic acid sequence encoding a protein of interest. In the present invention, a nucleic acid cassette includes, at the least, a promoter or a structure functionally equivalent to a promoter, a nucleic acid sequence encoding a signal peptide and a nucleic acid encoding a therapeutic protein. A nucleic acid cassette may include additional elements, such as an enhancer, and/or a transcription termination signal.

As used herein the terms “signal peptide”, “signal sequence”, “targeting sequence”, “leader sequence” and “secretory signal” are used interchangeably to mean heterogenous peptide sequences that are found at the N-terminus of secreted proteins that are instrumental in initiating the secretion process. In particular, signal peptides are found in proteins that are targeted to the endoplasmic reticulum and eventually destined to be either secreted or retained in the cell membrane of the cell, particularly as single-pass membrane proteins. Signal peptides are typically removed to produce the mature form of the protein. Signal peptides are normally short peptides, typically about 5 to about 40 amino acids in length, such as about 5 to about 35, or about 10 to about 35 amino acids in length, preferably about 10 to about 30 or about 15 to about 30 amino acids in length. A signal peptide may comprise a core of hydrophobic amino acids, said core typically being about 4 to about 20, such as about 5 to about 20, about 5 to about 16 or about 5 to about 15 amino acids in length). When present, a signal peptide is typically present at the N-terminus of a protein.

As used herein, the terms “transduced” and “modified” are used interchangeably to describe cells which have been modified to express a transgene of interest. Typically the modification occurs through transduction of the cells.

The term “endogenous,” when used in relation to a Rev response element (RRE) refers to the RRE which is from the same retroviral/lentiviral vector as the retroviral/lentiviral vector of the invention. A wildtype/unmodified vector will comprise an RRE within its genome, typically at a defined standard location. Viral vectors of the present invention typically have genomes which lack their endogenous RRE. The endogenous RRE may be inserted into an intron which is then itself introduced into the retroviral/lentiviral genome.

An exogenous RRE is from a different virus to the viral vector of the invention. By way of non-limiting example, the viral vector may be an HIV vector, and the RRE may be a SIV RRE.

As used herein, the terms “titre” and “yield” are used interchangeably to mean the amount of lentiviral (e.g. SIV) vector produced by a method of the invention. Titre is the primary benchmark characterising manufacturing efficiency, with higher titres generally indicating that more retroviral/lentiviral (e.g. SIV) vector is manufactured (e.g. using the same amount of reagents). Titre or yield may relate to the number of vector genomes that have integrated into the genome of a target cell (integration titre), which is a measure of “active” virus particles, i.e. the number of particles capable of transducing a cell. Transducing units (TU/mL also referred to as TTU/mL) is a biological readout of the number of host cells that get transduced under certain tissue culture/virus dilutions conditions, and is a measure of the number of “active” virus particles. The total number of (active+inactive) virus particles may also be determined using any appropriate means, such as by measuring either how much Gag is present in the test solution or how many copies of viral RNA are in the test solution. Assumptions are then made that a lentivirus particle contains either 2000 Gag molecules or 2 viral RNA molecules. Once total particle number and a transducing titre/TU have been measured, a particle:infectivity ratio calculated. Amino acids are referred to herein using the name of the amino acid, the three-letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

The transgenes of the present invention include nucleic acid sequences that have been removed from their naturally occurring environment, recombinant or cloned DNA isolates, and chemically synthesized analogues or analogues biologically synthesized by heterologous systems.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, lie, Phe or Val), (iii) a cysteine or proline is substituted for, or by, any other residue, or (iv) a residue having a bulky hydrophobic or aromatic side chain (e.g., Val, His, lie or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino Acid | Codons | Degenerate Codon |
|---|---|---|
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30 °C, typically in excess of 37 °C and preferably in excess of 45 °C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, or at least about 100%, or at least about 150%, or at least about 200%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. cystic fibrosis (CF) or any other disease described herein). Preferably said healthy individual(s) is not on medication affecting CF and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia

The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

### Retroviral and Lentiviral Vectors

The invention relates to a retroviral/lentiviral (e.g. SIV) vector. Retroviral/lentiviral vectors of the invention, can integrate into the genome of transduced cells and lead to long-lasting expression. The term “retrovirus” refers to any member of the Retroviridae family of RNA viruses that encode the enzyme reverse transcriptase. The term “lentivirus” refers to a family of retroviruses. Thus, all references herein to retroviral vectors of the invention apply equally and without reservation to lentiviral vectors. Further, all references herein to lentiviral vectors of the invention apply equally and without reservation to retroviral vectors.

Examples of retroviruses suitable for use in the present invention include gamma retroviruses such as murine leukaemia virus (MLV) and feline leukaemia virus (FLV). Examples of lentiviruses suitable for use in the present invention include Simian immunodeficiency virus (SIV), Human immunodeficiency virus (HIV), Feline immunodeficiency virus (FIV), Equine infectious anaemia virus (EIAV), and Visna/maedi virus. Preferably the invention relates to lentiviral vectors and the production thereof. A particularly preferred lentiviral vector is an SIV vector (including all strains and subtypes), such as a SIV-AGM (originally isolated from African green monkeys, Cercopithecus aethiops). Alternatively the invention relates to HIV vectors.

The retroviral/lentiviral (e.g. SIV) vectors of the present invention are typically pseudotyped with hemagglutinin-neuraminidase (HN) and fusion (F) proteins from a respiratory paramyxovirus, or with G glycoprotein from Vesicular Stomatitis Virus (referred to as VSV-G or G-VSV). Preferably the lentiviral (e.g. SIV) vectors of the present invention are pseudotyped with HN and F from a respiratory paramyxovirus. Particularly preferably the respiratory paramyxovirus is a Sendai virus (murine parainfluenza virus type 1). The retroviral/lentiviral (e.g. SIV) vectors of the present invention may be pseudotyped with proteins from another virus, provided that the pseudotyping proteins do not negatively impact the manufactured titre of the vector (or even result in an increased titre of the vector) and/or transgene expression (or even result in increased transgene expression). Non-limiting examples of other proteins that may be used to pseudotype retroviral/lentiviral (e.g. SIV) vectors of the present invention include severe acute respiratory syndrome coronavirus 2 (SARS-CoV-2) spike protein or modified forms thereof. VSV-G and SARS-Cov2 spike protein used for pseudotyping are as those described in UK Patent Application Nos. 2118685.3 and International Application No. PCT/GB2022/050933, each of which is herein incorporated by reference in its entirety.

A retroviral/lentiviral (e.g. SIV) vector for use according to the invention may be integrase-competent (IC). Alternatively, the lentiviral (e.g. SIV) vector may be integrase-deficient (ID).

Viral vectors of the invention, particularly retroviral/lentiviral (e.g. SIV) vectors as described herein may transduce one or more cell types as described herein to achieve long term transgene expression.

The retroviral/lentiviral (e.g. SIV) vectors of the present invention enable high levels of transgene expression. In particular, retroviral/lentiviral (e.g. SIV) vectors of the present invention typically result in high levels (therapeutic levels) of expression of a therapeutic protein.

The nucleic acid sequence encoding a therapeutic protein to be included in a viral vector of the invention, particularly a retroviral/lentiviral (e.g. SIV) vector of the invention may be modified to facilitate expression. For example, the transgene sequence may be in CpG-depleted (or CpG-fee) and/or codon-optimised form to facilitate gene expression. Standard techniques for modifying the transgene sequence in this way are known in the art. The genome of the retroviral/lentiviral (e.g. SIV) vector may be fully or partially CpG-depleted (or CpG-fee) and/or codon-optimised.

Retroviral/lentiviral (e.g. SIV) vectors, such as those of the invention, can integrate into the genome of transduced cells and lead to long-lasting expression, making them suitable for transduction of stem/progenitor cells. In the lung, several cell types with regenerative capacity have been identified as responsible for maintaining specific cell lineages in the conducting airways and alveoli. These include basal cells and submucosal gland duct cells in the upper airways, club cells and neuroendocrine cells in the bronchiolar airways, bronchioalveolar stem cells in the terminal bronchioles and type II pneumocytes in the alveoli. Therefore, and without being bound by theory, it is believed that said retroviral/lentiviral (e.g. SIV) vectors bring about long term gene expression of the transgene of interest by introducing the transgene into one or more long-lived airway epithelial cells or cell types, such as basal cells and submucosal gland duct cells in the upper airways, club cells and neuroendocrine cells in the bronchiolar airways, bronchioalveolar stem cells in the terminal bronchioles type II pneumocytes in the alveoli, submucosal acinar cells, ionocytes, and type I pneumocytes. As demonstrated herein, the integration of retroviral/lentiviral (e.g. SIV) vectors with modified retroviral/lentiviral (e.g. SIV) RNA sequences of the invention into target cell genomes is unexpectedly not negatively impacted, and in fact may even be increased.

Accordingly, the retroviral/lentiviral (e.g. SIV) vectors of the invention may transduce one or more cells or cell lines with regenerative potential within the lung (including the airways and respiratory tract) to achieve long term gene expression. For example, the retroviral/lentiviral (e.g. SIV) vectors may transduce basal cells, such as those in the upper airways/respiratory tract. Basal cells have a central role in processes of epithelial maintenance and repair following injury. In addition, basal cells are widely distributed along the human respiratory epithelium, with a relative distribution ranging from 30% (larger airways) to 6% (smaller airways).

The retroviral/lentiviral (e.g. SIV) vectors of the invention may be used to transduce isolated and expanded stem/progenitor cells ex vivo prior administration to a patient. Preferably, the retroviral/lentiviral (e.g. SIV) vectors of the invention are used to transduce cells within the lung (or airways/respiratory tract) in vivo.

The retroviral/lentiviral (e.g. SIV) vectors of the invention demonstrate remarkable resistance to shear forces with only modest reduction in transduction ability when passaged through clinically-relevant delivery devices such as bronchoscopes, spray bottles and nebulisers.

The retroviral/lentiviral (e.g. SIV) vectors of the present invention enable high levels of transgene expression, resulting in high levels (therapeutic levels) of expression of a therapeutic protein. The retroviral/lentiviral (e.g. SIV) vectors of the present invention typically provide high expression levels of a transgene when administered to a patient. The terms high expression and therapeutic expression are used interchangeably herein. Expression may be measured by any appropriate method (qualitative or quantitative, preferably quantitative), and concentrations given in any appropriate unit of measurement, for example ng/ml or nM.

Expression of a transgene of interest may be given relative to the expression of the corresponding endogenous (defective) gene in a patient. Expression may be measured in terms of mRNA or protein expression. The expression of the transgene of the invention, such as a functional CFTR gene, may be quantified relative to the endogenous gene, such as the endogenous (dysfunctional) CFTR genes in terms of mRNA copies per cell or any other appropriate unit.

Expression levels of a transgene and/or the encoded therapeutic protein of the invention may be measured in the lung tissue, epithelial lining fluid and/or serum/plasma as appropriate. A high and/or therapeutic expression level may therefore refer to the concentration in the lung, epithelial lining fluid and/or serum/plasma.

A retroviral/lentiviral (e.g. SIV) vector of the invention enables long-term transgene expression, resulting in long-term expression of a therapeutic protein. As described herein, the phrases “long-term expression”, “sustained expression”, “long-lasting expression” and “persistent expression” are used interchangeably. The retroviral/lentiviral (e.g. SIV) vectors of the present invention enable long-term transgene expression, resulting in long-term expression of a therapeutic protein, particularly by airway cells, as described herein. Long-term expression according to the present invention means expression of a therapeutic gene and/or protein, preferably at therapeutic levels, for at least 45 days, at least 60 days, at least 90 days, at least 120 days, at least 180 days, at least 250 days, at least 360 days, at least 450 days, at least 730 days or more. Preferably long-term expression means expression for at least 90 days, at least 120 days, at least 180 days, at least 250 days, at least 360 days, at least 450 days, at least 720 days or more, more preferably at least 360 days, at least 450 days, at least 720 days or more. This long-term expression may be achieved by repeated doses or by a single dose.

In particular, a retroviral/lentiviral (e.g. SIV) vector of the invention may drive (increased) long-lasting expression of a therapeutic protein in an airway cell in vivo in a patient. Preferably, a retroviral/lentiviral (e.g. SIV) vector of the invention drives expression of a therapeutic protein in an airway cell for at least 45 days, more preferably at least 90 days.

Repeated doses may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated.

The retroviral/lentiviral (e.g. SIV) vectors of the invention exhibit enhanced expression of the therapeutic protein. Accordingly, the retroviral/lentiviral (e.g. SIV) vectors of the invention are capable of producing long-lasting, repeatable, high-level expression, particularly in airway cells, without inducing an undue immune response.

Preferably, the invention relates to F/HN retroviral/lentiviral vectors comprising a promoter and a transgene, particularly SIV F/HN vectors.

The viral vectors of the invention may be made using any suitable process known in the art. In particular, retroviral/lentiviral (e.g. SIV) vectors of the invention may be made using the methods disclosed in International Application No. PCT/GB2022/050524 which is herein incorporated by reference in its entirety.

The viral vectors of the invention, particularly the retroviral/lentiviral (e.g. SIV) vectors of the invention may comprise a central polypurine tract (cPPT) and/or the Woodchuck hepatitis virus posttranscriptional regulatory elements (WPRE). An exemplary WPRE sequence is provided by SEQ ID NO: 39.

A retroviral/lentiviral (e.g. SIV) vector of the invention has been modified to (i) delete the endogenous RRE as described herein; and (ii) to introduce one or more intron into which a retroviral/lentiviral (e.g. SIV) RRE has been inserted. In other words, a retroviral/lentiviral (e.g. SIV) vector of the invention has a genome that has been modified to (i) delete the endogenous RRE as described herein; and (ii) to introduce one or more intron into which a retroviral/lentiviral (e.g. SIV) RRE has been inserted. Any reference herein to a retroviral/lentiviral (e.g. SIV) vector of the invention which comprises an intron with an RRE inserted into the intron applies equally and without reservation to a retroviral/lentiviral (e.g. SIV) vector of the invention genome which comprises an intron with an RRE inserted into the intron.

As described herein, typically an intron comprising an RRE which is introduced into a retroviral/lentiviral (e.g. SIV) vector of the invention is appropriately spliced by target cells, aiding in the maturation of a stable mRNA molecule. This results in an increased expression of the coding regions of the retroviral/lentiviral (e.g. SIV) genome, including the transgene. Thus, the introduction of an intron comprising an RRE within retroviral/lentiviral (e.g. SIV) vector results in increased expression of the transgene, which may encode a therapeutic protein. The introduction of an intron comprising an RRE into a retroviral/lentiviral (e.g. SIV) vector may increase expression of the therapeutic protein compared with expression of the therapeutic protein from a corresponding retroviral/lentiviral (e.g. SIV) vector without the RRE-comprising intron. Thus, a retroviral/lentiviral (e.g. SIV) vector of the invention comprising an RRE-comprising intron typically exhibits increased transgene expression compared with transgene expression from a corresponding retroviral/lentiviral (e.g. SIV) vector lacking said RRE-comprising intron. By way of non-limiting example, a retroviral/lentiviral (e.g. SIV) vector of the invention comprising an AAT transgene (SERPINA1) and 3-globulin/IgG chimeric intron with an inserted RRE (e.g. the RRE-comprising β-globulin/IgG chimeric intron of SEQ ID NO: 5) may increase AAT expression compared with a corresponding retroviral/lentiviral (e.g. SIV) vector which comprises the AAT transgene but which lacks the 3-globulin/IgG chimeric intron with an inserted RRE (e.g. the RRE-comprising β-globulin/IgG chimeric intron of SEQ ID NO: 5).

The increase in expression of the therapeutic protein by a retroviral/lentiviral (e.g. SIV) vector of the invention comprising an RRE-comprising intron may be as defined herein. In particular, the increase in expression of the therapeutic protein by a retroviral/lentiviral (e.g. SIV) vector of the invention comprising an RRE-comprising intron may an increase of at least about 5-fold, an increase of at least about 10-fold, an increase of at least about 50-fold, an increase of at least about 100-fold, an increase of at least about 200-fold, an increase of at least about 500-fold, an increase of at least about 600-fold or more, typically compared with expression of the therapeutic protein from a corresponding retroviral/lentiviral (e.g. SIV) vector without the RRE-comprising intron. Preferably, the increase in expression of the therapeutic protein by a retroviral/lentiviral (e.g. SIV) vector of the invention comprising an RRE-comprising intron is at least about 10-fold, more preferably at least about 100-fold, even more preferably at least about 500-fold, typically compared with expression of the therapeutic protein from a corresponding retroviral/lentiviral (e.g. SIV) vector without the RRE-comprising intron. By way of non-limiting example, when the RRE-comprising intron is a β-globulin/IgG chimeric intron comprising an RRE, such as the β-globulin/IgG chimeric RRE-comprising intron of SEQ ID NO: 5, said intron may increase transgene expression by a retroviral/lentiviral (e.g. SIV) vector of the invention comprising said β-globulin/IgG chimeric RRE-comprising intron by at least 600-fold, such as by about 686-fold compared with the expression of the transgene by a corresponding retroviral/lentiviral (e.g. SIV) vector without said β-globulin/IgG chimeric RRE-comprising intron.

The increase in expression of the therapeutic protein by a retroviral/lentiviral (e.g. SIV) vector of the invention comprising an RRE-comprising intron may be as defined herein. In particular, the increase in expression of the therapeutic protein by a retroviral/lentiviral (e.g. SIV) vector of the invention comprising an RRE-comprising intron may an increase of at least about 100%, an increase of at least about 500%, an increase of at least about 1000%, an increase of at least about 5000%, an increase of at least about 10000%, an increase of at least about 20000%, an increase of at least about 50000%, an increase of at least about 60000% or more, typically compared with expression of the therapeutic protein from a corresponding retroviral/lentiviral (e.g. SIV) vector without the RRE-comprising intron. Preferably, the increase in expression of the therapeutic protein by a retroviral/lentiviral (e.g. SIV) vector of the invention comprising an RRE-comprising intron is at least about 1000%, more preferably at least about 10000%, even more preferably at least about 50000%, typically compared with expression of the therapeutic protein from a corresponding retroviral/lentiviral (e.g. SIV) vector without the RRE-comprising intron. By way of non-limiting example, when the RRE-comprising intron is a β-globulin/IgG chimeric intron comprising an RRE, such as the β-globulin/IgG chimeric RRE-comprising intron of SEQ ID NO: 5, said intron may increase transgene expression by a retroviral/lentiviral (e.g. SIV) vector of the invention comprising said β-globulin/IgG chimeric RRE-comprising intron by at least 60000%, such as by about 68600% compared with the expression of the transgene by a corresponding retroviral/lentiviral (e.g. SIV) vector without said β-globulin/IgG chimeric RRE-comprising intron.

As described herein, typically an intron comprising an RRE which is introduced into a retroviral/lentiviral (e.g. SIV) vector of the invention is appropriately spliced by target cells, aiding in the maturation of a stable mRNA molecule. This results in an increased number of mRNA molecules (i.e. increased mRNA copy number) produced by the retroviral/lentiviral (e.g. SIV) vector and/or vector genome plasmid. Thus, the introduction of an intron comprising an RRE within retroviral/lentiviral (e.g. SIV) vector results in increased expression of the transgene, which may encode a therapeutic protein. The introduction of an intron comprising an RRE into a retroviral/lentiviral (e.g. SIV) vector may increase the number of mRNA molecules produced by the retroviral/lentiviral (e.g. SIV) vector and/or vector genome plasmid compared with the number of mRNA molecules produced from a corresponding retroviral/lentiviral (e.g. SIV) vector and/or vector genome plasmid without the RRE-comprising intron. Thus, a retroviral/lentiviral (e.g. SIV) vector of the invention comprising an RRE-comprising intron typically results in an increase in the number of mRNA molecules produced by the retroviral/lentiviral (e.g. SIV) vector and/or vector genome plasmid compared with the number of mRNA molecules produced from a corresponding retroviral/lentiviral (e.g. SIV) vector and/or vector genome plasmid lacking said RRE-comprising intron. By way of non-limiting example, a retroviral/lentiviral (e.g. SIV) vector of the invention comprising an AAT transgene (SERPINA1) and β-globulin/IgG chimeric intron with an inserted RRE (e.g. the RRE-comprising @3-globulin/IgG chimeric intron of SEQ ID NO: 5) may result in an increased number of mRNA molecules produced by the retroviral/lentiviral (e.g. SIV) vector and/or vector genome plasmid compared with a corresponding retroviral/lentiviral (e.g. SIV) vector and/or vector genome plasmid which comprises the AAT transgene but which lacks the β-globulin/IgG chimeric intron with an inserted RRE (e.g. the RRE-comprising β-globulin/IgG chimeric intron of SEQ ID NO: 5).

The increase in number of mRNA molecules produced by the retroviral/lentiviral (e.g. SIV) vector and/or vector genome plasmid may be as defined herein. In particular, the increase in expression of the therapeutic protein by a retroviral/lentiviral (e.g. SIV) vector of the invention comprising an RRE-comprising intron may an increase of at least about 2-fold, at least about 5-fold, at least about 7-fold, at least about 10-fold, at least about 12-fold, at least about 15-fold or more, typically compared with the number of mRNA molecules produced by a corresponding retroviral/lentiviral (e.g. SIV) vector without the RRE-comprising intron. Preferably, the increase in number of mRNA molecules produced by the retroviral/lentiviral (e.g. SIV) vector and/or vector genome plasmid is least about 10-fold, more preferably at least about 12-fold, even more preferably at least about 13-fold, typically compared with the number of mRNA molecules produced by a corresponding retroviral/lentiviral (e.g. SIV) vector and/or vector genome plasmid without the RRE-comprising intron. By way of non-limiting example, when the RRE-comprising intron is a @3-globulin/IgG chimeric intron comprising an RRE, such as the β-globulin/IgG chimeric RRE-comprising intron of SEQ ID NO: 5, said intron may increase the number of mRNA molecules produced by the retroviral/lentiviral (e.g. SIV) vector and/or vector genome plasmid by at least 12-fold, such as by about 13.7-fold compared with the number of mRNA molecules produced by a corresponding retroviral/lentiviral (e.g. SIV) vector and/or vector genome plasmid without said β-globulin/IgG chimeric RRE-comprising intron.

As described herein, typically an intron comprising an RRE which is introduced into a retroviral/lentiviral (e.g. SIV) vector of the invention is appropriately spliced by target cells, aiding in the maturation of a stable mRNA molecule. This results in an increased number of mRNA molecules (i.e. increased mRNA copy number) produced per copy of retroviral/lentiviral (e.g. SIV) vector and/or vector genome plasmid. Thus, the introduction of an intron comprising an RRE within retroviral/lentiviral (e.g. SIV) vector results in increased expression of the transgene, which may encode a therapeutic protein. The introduction of an intron comprising an RRE into a retroviral/lentiviral (e.g. SIV) vector may increase the number of mRNA molecules produced per copy of retroviral/lentiviral (e.g. SIV) vector and/or vector genome plasmid compared with the number of mRNA molecules produced per copy of retroviral/lentiviral (e.g. SIV) vector and/or vector genome plasmid from a corresponding retroviral/lentiviral (e.g. SIV) vector and/or vector genome plasmid without the RRE-comprising intron. Thus, a retroviral/lentiviral (e.g. SIV) vector of the invention comprising an RRE-comprising intron typically results in an increase in the number of mRNA molecules produced per copy of retroviral/lentiviral (e.g. SIV) vector and/or vector genome plasmid compared with the number of mRNA molecules produced from a corresponding retroviral/lentiviral (e.g. SIV) vector and/or vector genome plasmid lacking said RRE-comprising intron. By way of non-limiting example, a retroviral/lentiviral (e.g. SIV) vector of the invention comprising an AAT transgene (SERPINA1) and β-globulin/IgG chimeric intron with an inserted RRE (e.g. the RRE-comprising @3-globulin/IgG chimeric intron of SEQ ID NO: 5) may result in an increased number of mRNA molecules produced per copy of retroviral/lentiviral (e.g. SIV) vector and/or vector genome plasmid compared with a corresponding retroviral/lentiviral (e.g. SIV) vector and/or vector genome plasmid which comprises the AAT transgene but which lacks the β-globulin/IgG chimeric intron with an inserted RRE (e.g. the RRE-comprising β-globulin/IgG chimeric intron of SEQ ID NO: 5).

The increase in number of mRNA molecules produced per copy of retroviral/lentiviral (e.g. SIV) vector and/or vector genome plasmid may be as defined herein. In particular, the increase in number of mRNA molecules produced per copy of retroviral/lentiviral (e.g. SIV) vector and/or vector genome plasmid of the invention comprising an RRE-comprising intron may an increase of at least about 10-fold, at least about 20-fold, at least about 30-fold, at least about 40-fold, at least about 45-fold or more, typically compared with the number of mRNA molecules produced per copy of retroviral/lentiviral (e.g. SIV) vector and/or vector genome plasmid from a corresponding retroviral/lentiviral (e.g. SIV) vector without the RRE-comprising intron. Preferably, the increase in number of mRNA molecules produced per copy of retroviral/lentiviral (e.g. SIV) vector and/or vector genome plasmid is least about 20-fold, more preferably at least about 30-fold, even more preferably at least about 40-fold, typically compared with the number of mRNA molecules produced per copy of retroviral/lentiviral (e.g. SIV) vector and/or vector genome plasmid from a corresponding retroviral/lentiviral (e.g. SIV) vector and/or vector genome plasmid without the RRE-comprising intron. By way of non-limiting example, when the RRE-comprising intron is a β-globulin/IgG chimeric intron comprising an RRE, such as the β-globulin/IgG chimeric RRE-comprising intron of SEQ ID NO: 5, said intron may increase the number of mRNA molecules produced per copy of retroviral/lentiviral (e.g. SIV) vector and/or vector genome plasmid by at least 40-fold, such as by about 42.2-fold compared with the number of mRNA molecules produced per copy of retroviral/lentiviral (e.g. SIV) vector and/or vector genome plasmid by a corresponding retroviral/lentiviral (e.g. SIV) vector and/or vector genome plasmid without said β-globulin/IgG chimeric RRE-comprising intron.

The invention also provides host cells comprising a retroviral/lentiviral (e.g. SIV) vector of the invention. Typically a host cell is a mammalian cell, particularly a human cell or cell line. Non-limiting examples of host cells include HEK293 cells (such as HEK293F or HEK293T cells) and 293T/17 cells. Commercial cell lines suitable for the production of virus are also readily available (as described herein).

### Rev Response Elements (RRE)

The retroviral/lentiviral (e.g. SIV) vectors of the invention have been designed such that the (RNA) genome of said retroviral/lentiviral (e.g. SIV) vector comprises an intron that is not removed during manufacture of the vector, such that the final retroviral/lentiviral (e.g. SIV) vector comprises said intron, resulting in increased expression of the transgene upon transduction of target cells.

To facilitate retention of the intron within the genome of the retroviral/lentiviral (e.g. SIV) vector, the endogenous RRE of the retroviral/lentiviral (e.g. SIV) vector is deleted from its position within the wildtype/unmodified retroviral/lentiviral (e.g. SIV) genome and an RRE inserted into the intron sequence.

Deletion of the endogenous RRE from its position within the wildtype/unmodified retroviral/lentiviral (e.g. SIV) genome may be complete or a partial deletion, provided that if the deletion is partial, the activity of the remaining RRE sequence is decreased or completely ablated. Without being bound by theory, it is believed that partial deletion of the RRE sequence is sufficient provided that the activity of the remaining RRE sequence is insufficient for gene expression from the retroviral/lentiviral (e.g. SIV) genome, placing pressure on the retroviral/lentiviral (e.g. SIV) vector to rely on the activity of the RRE within the intron, and hence to retain the RRE inserted into the intron. Reference herein to deletion of the endogenous RRE therefore encompasses both complete and partial deletion of the endogenous RRE. Standard techniques are known in the art for the deletion of nucleic acid sequences from a nucleic acid (e.g. plasmid), and may be readily used by one of ordinary skill in the art to delete the endogenous RRE.

Any RRE may be inserted into the intron to be included in the genome of the retroviral/lentiviral (e.g. SIV) vector, provided said RRE is able to facilitate retroviral/lentiviral (e.g. SIV) gene expression in the absence of the endogenous RRE in its standard position within the wildtype/unmodified retroviral/lentiviral (e.g. SIV) genome. Typically the inserted RRE is a viral RRE, particularly a retroviral RRE, even more particularly a lentiviral RRE. Standard techniques are known in the art for the insertion of nucleic acid sequences into a nucleic acid (e.g. plasmid), and may be readily used by one of ordinary skill in the art to insert an RRE into an intron according to the present invention.

The RRE to be inserted into an intron may be the endogenous RRE of the retroviral/lentiviral (e.g. SIV) vector. Thus, the endogenous RRE may be deleted from within the wildtype/unmodified retroviral/lentiviral (e.g. SIV) genome and inserted into an intron which is itself introduced into a retroviral/lentiviral (e.g. SIV) vector of the invention. In other words, the endogenous RRE is moved from its position within the wildtype/unmodified retroviral/lentiviral (e.g. SIV) genome, and inserted into an intron. By way of non-limiting example, in an SIV vector of the invention, the endogenous SIV RRE has been deleted from within the wildtype/unmodified SIV genome, and an intron into which the endogenous SIV RRE has been inserted is itself introduced into the SIV vector.

The RRE to be inserted into an intron may be an exogenous RRE. In the case of a retroviral vector of the invention, an exogenous RRE may be an RRE from a different retrovirus. In the case of a lentiviral vector of the invention, an exogenous RRE may be an RRE from a different lentivirus. By way of non-limiting example, an HIV vector of the invention may have its endogenous HIV RRE deleted and an intron comprising a SIV RRE introduced into the HIV genome.

Preferably the RRE sequence inserted into an intron within a retroviral/lentiviral (e.g. SIV) vector of the invention is the same as the endogenous RRE sequence which is deleted from the retroviral/lentiviral (e.g. SIV) genome. Thus, preferably, the RRE is from the same virus as the viral vector, but is inserted into the (chimeric) intron, rather than being present in its standard location within the viral genome. By way of non-limiting example, the viral vector may be an SIV vector in which the SIV RRE has been deleted from the genome and a (chimeric) intron introduced into which an SIV RRE has been inserted. By way of further non-limiting example, the viral vector may be an HIV vector, and the RRE may be an HIV RRE, but the HIV RRE is inserted within a (chimeric) intron, rather than in the standard location of the HIV RRE within the HIV genome.

The RRE to be inserted into the intron may be less than 1,000 bp, such as less than 900 bp or less than 800 bp may be preferred. Without being bound by theory, it is believed that smaller RRE-comprising introns may be more suitable for general applicability, allowing for greater flexibility in terms of the additional elements to be included within the retroviral/lentiviral (e.g. SIV) genome. Particularly preferred are RRE of between about 750 bp to about 800 bp, such as about 760 bp, such as the exemplified SIV RRE of the invention.

The RRE to be inserted into the intron may be an SIV RRE. The SIV RRE may comprise or consist of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 1. Preferably, the SIV RRE comprises or consists of nucleic acid sequence having at least 90%, at least 95%, or at least 99% sequence identity to SEQ ID NO: 1. More preferably, the SIV RRE consists of nucleic acid sequence having at least 90%, at least 95%, or at least 99% sequence identity to SEQ ID NO: 1. Still more preferably, the SIV RRE comprises or consists, particularly consists of, the nucleic acid sequence of SEQ ID NO: 1.

The RRE to be inserted into the intron may be an HIV RRE. The HIV RRE may comprise or consist of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 50. Preferably, the HIV RRE comprises or consists of nucleic acid sequence having at least 90%, at least 95%, or at least 99% sequence identity to SEQ ID NO: 50. More preferably, the HIV RRE consists of nucleic acid sequence having at least 90%, at least 95%, or at least 99% sequence identity to SEQ ID NO: 50. Still more preferably, the HIV RRE comprises or consists, particularly consists of, the nucleic acid sequence of SEQ ID NO: 50.

According to the invention, typically the RRE inserted into the intron does not form part of the mature mRNA expressed within the host/target cells, as the RRE will be spliced out as part of the intron.

### Introns

A retroviral/lentiviral (e.g. SIV) vector of the invention has been modified to (i) delete the endogenous RRE as described herein; and (ii) to introduce one or more intron into which a retroviral/lentiviral (e.g. SIV) RRE has been inserted.

A retroviral/lentiviral (e.g. SIV) vector of the invention may comprise one or more intron, such as one, two, three, four, five or more introns. Typically a retroviral/lentiviral (e.g. SIV) vector of the invention comprises one or two introns, preferably one intron.

Wildtype/unmodified retroviral/lentiviral (e.g. SIV) vector genomes do not comprise introns. Therefore any and all references herein to retroviral/lentiviral (e.g. SIV) vectors/vector genomes comprising one or more introns refers to retroviral/lentiviral (e.g. SIV) vectors/vector genomes into which one or more introns have been introduced. In other words, any intron comprised in a retroviral/lentiviral (e.g. SIV) vector/vector genome of the invention is an intron which has been introduced into said retroviral/lentiviral (e.g. SIV) vector/vector genome, as described herein.

The size of the one or more intron to be inserted is not particularly limited, provided that the retroviral/lentiviral (e.g. SIV) vector/vector genome comprising the one or more intron is within the packing limit for the retroviral/lentiviral (e.g. SIV) vector/vector genome. As the retroviral/lentiviral (e.g. SIV) vector/vector genome will comprise other elements (including the genome backbone, transgene and transgene promoter) in addition to the intron, the upper size limit of the one or more intron to be inserted may be calculated as:

ULᵢ=VPL−GE
where UL, is the upper size limit of intron, VPL is the retroviral/lentiviral (e.g. SIV) packing limit, and GE is the total size of other retroviral/lentiviral (e.g. SIV) genome elements.

Retroviral/lentiviral (e.g. SIV) vectors typically have a packing limit of approximately 10 kb. Therefore, typically the size of the one or more intron to be inserted is less than about 5,000 bp, accounting for the other elements that must be present within the retroviral/lentiviral (e.g. SIV) genome.

Introns of less than 1,000 bp, such as less than 900 bp or less than 800 bp may be preferred. Without being bound by theory, it is believed that smaller introns of this type may be more suitable for general applicability, allowing for greater flexibility in terms of the additional elements to be included within the retroviral/lentiviral (e.g. SIV) genome. Particularly preferred are introns of between about 750 bp to about 800 bp, such as about 770 bp, such as the exemplified β-globulin/IgG chimeric intron of the invention.

The one or more intron may be introduced at any position within the retroviral/lentiviral (e.g. SIV) genome. Typically the one or more intron is introduced at a position within the retroviral/lentiviral (e.g. SIV) genome that does not disrupt the function of the retroviral/lentiviral (e.g. SIV) genome or any part thereof. By way of non-limiting example, the one or more intron may be inserted at any position within the retroviral/lentiviral (e.g. SIV) genome, provided that translation of the transgene is not decreased. Preferably the intron is introduced between a transgene and the promoter operably linked to said transgene. Thus, preferably a retroviral/lentiviral (e.g. SIV) vector/genome of the invention comprises an intron between the transgene and the promoter operably linked to said transgene. According to the invention, the RRE-comprising intron does not comprise the transgene to be expressed.

The sequence of the intron to be introduced is not particularly limited. Indeed, as exemplified herein, the present inventors have shown that the retroviral RRE remains functional in different contexts, and thus that RRE function does not depend on the specific intron sequence. Further, it has been known the art for decades that non-chimeric introns can be divided and combined with other DNA sequences (see for example, Choi, T. et al. (1990) Molecular and Cellular Biology, 11:6, 3070-3074, which is herein incorporated by reference). Thus, any appropriate intron may be used according to the present invention. In other words, any intron may have an RRE sequence inserted, and said intron/RRE introduced into a retroviral/lentiviral (e.g. SIV) vector/genome of the invention. The intron may be a naturally occurring, recombinant, or artificial, such as a chimeric intron. The intron may be a viral intron. Non-limiting examples of introns include: SV40 intron, Ef1-α intron 1, CMB intron A and adenovirus tripartite leader sequence intron. The intron may be a chimeric intron or a non-chimeric intron. Preferably the intron is a chimeric intron, with the chimeric β-globulin/IgG intron exemplified herein being particularly preferred.

Typically the intron is a chimeric intron, such that retroviral/lentiviral (e.g. SIV) vectors of the invention comprise a chimeric intron. A chimeric intron is an artificial intron which comprises or consists of sequences from two or more different introns. Non-limiting examples of chimeric introns include a β-globulin/IgG chimeric intron and the chimeric intron from the CAGGS promoter. The latter comprises the splice donor from chicken @3-actin and the splice acceptor from rabbit β-globulin. Preferably, a chimeric intron according to the present invention is a β-globulin/IgG chimeric intron, such as that exemplified herein. Particularly preferred is a β-globulin/IgG chimeric intron which comprises or consists of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 4. Preferably, the β-globulin/IgG chimeric intron comprises or consists of a nucleic acid sequence having at least 90%, at least 95%, or at least 99% sequence identity to SEQ ID NO: 4. More preferably, the β-globulin/IgG chimeric intron consists of a nucleic acid sequence having at least 90%, at least 95%, or at least 99% sequence identity to SEQ ID NO: 4. Still more preferably, the β-globulin/IgG chimeric intron comprises or consists, particularly consists of, the nucleic acid sequence of SEQ ID NO: 4.

A chimeric intron from the CAGGS promoter may comprise or consist of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 48. Preferably, the chimeric intron from the CAGGS promoter comprises or consists of a nucleic acid sequence having at least 90%, at least 95%, or at least 99% sequence identity to SEQ ID NO: 48. More preferably, the chimeric intron from the CAGGS promoter consists of a nucleic acid sequence having at least 90%, at least 95%, or at least 99% sequence identity to SEQ ID NO: 48. Still more preferably, the chimeric intron from the CAGGS promoter comprises or consists, particularly consists of, the nucleic acid sequence of SEQ ID NO: 48.

The RRE is introduced at a position within the intron which does not disrupt the splice donor and/or acceptor sites of the intron, to allow for the intron to be spliced correctly within a target cell. The splice donor and/or acceptor sites of a particular intron may be readily determined using routine methods and techniques, for example as described in Desmet et al. (Nucleic Acids Res. 2009 May; 37(9): e67) and Baten et al. (BMC Bioinformatics volume 7, Article number: S15 (2006)), both of which are herein incorporated by reference in their entirety.

In particular, the inventors have found that placing an RRE within 200 bp 5′ of the splice acceptor's branch site (and polypyrimidine tract), such as within 100 bp 5′ of the splice acceptor's branch site, within 50 bp 5′ of the splice acceptor's branch site or within 20 bp 5′ of the splice acceptor's branch site, is advantageous as it allows the RRE to be inserted whilst allowing efficient splicing of the intron to produce retroviral/lentiviral (e.g. SIV) mRNA. In particular, and as exemplified herein, an RRE may preferably be inserted within 20 bp 5′ of the splice acceptor's branch site (and polypyrimidine tract), such 20 bp 5′ of the splice acceptor's branch site, 19 bp 5′ of the splice acceptor's branch site, 18 bp 5′ of the splice acceptor's branch site or less, with 18 bp 5′ of the splice acceptor's branch site being particularly preferred.

Typically at least there is a space of at least 5 bp, at least 10 bp or at least 15 bp between the RRE insertion site and the splice acceptor's branch site, to ensure that this branch site is not disrupted by RRE insertion. Thus, an RRE may be inserted at a location between about 5-20 bp 5′ of the splice acceptor's branch site (and polypyrimidine tract), such as between about 10-20 bp 5′ of the splice acceptor's branch site or between about 15-20 bp 5′ of the splice acceptor's branch site, with RRE insertion 18 bp 5′ of the splice acceptor's branch site being preferred.

The RRE insertion site devised by the present inventors differs from insertion sites attempted in the art, which are typically closer to the splice donor site than the splice acceptor site.

The RRE insertion site of the invention is also typically designed such that any other regulatory sequences within the intron are not disrupted by RRE insertion.

When the intron is a chimeric intron, the RRE may be inserted at a junction between the sequences from the two or more different introns. By way of non-limiting example, where the chimeric intron is a β-globulin/IgG chimeric intron, the RRE may be inserted at the junction between the β-globulin intron sequence (the 5′ portion of the chimeric β-globulin/IgG chimeric intron) and the IgG intron sequence (the 3′ portion of the β-globulin/IgG chimeric intron).

In preferred embodiments relating to the use of a β-globulin/IgG chimeric intron, the RRE may be inserted between a splice donor site and a splice acceptor site, wherein (a) the splice donor site comprises or consists of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 2 (from β-globulin); and/or (b) the splice acceptor site comprises or consists of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 3 (from IgG). Preferably (a) the splice donor site comprises or consists of a nucleic acid sequence having at least 90%, at least 95%, or at least 99% sequence identity to SEQ ID NO: 2; and/or (b) the splice acceptor site comprises or consists of a nucleic acid sequence having at least 90%, at least 95%, or at least 99% sequence identity to SEQ ID NO: 3. More preferably (a) the splice donor site consists of a nucleic acid sequence having at least 90%, at least 95%, or at least 99% sequence identity to SEQ ID NO: 2; and/or (b) the splice acceptor site consists of a nucleic acid sequence having at least 90%, at least 95%, or at least 99% sequence identity to SEQ ID NO: 3. Still more preferably (a) the splice donor site comprises or consists, particularly consists of, the nucleic acid sequence of SEQ ID NO: 2; and/or (b) the splice acceptor site comprises or consists, particularly consists of, the nucleic acid sequence of SEQ ID NO: 3.

The intron may be introduced into the retroviral/lentiviral (e.g. SIV) vector/genome in either forward or reverse orientation. Preferably, the intron is introduced into the retroviral/lentiviral (e.g. SIV) vector/genome in the forward orientation.

Preferably, and as exemplified herein, the intron is a β-globin/IgG chimeric intron and the RRE is an SIV RRE. The β-globin/IgG chimeric intron comprising a SIV RRE may comprise or consist of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 5. Preferably, the @3-globin/IgG chimeric intron comprising a SIV RRE comprises or consists of nucleic acid sequence having at least 90%, at least 95%, or at least 99% sequence identity to SEQ ID NO: 5. More preferably, the 3-globin/IgG chimeric intron comprising a SIV RRE consists of nucleic acid sequence having at least 90%, at least 95%, or at least 99% sequence identity to SEQ ID NO: 5. Still more preferably, the β-globin/IgG chimeric intron comprising a SIV RRE comprises or consists, particularly consists of, the nucleic acid sequence of SEQ ID NO: 5.

An intron into which an RRE has been inserted according to the present invention may be referred to interchangeably herein as an “RRE-comprising intron”, an “intron comprising an RRE”, “an intron with an inserted RRE”, and an “intron with an introduced RRE”.

Typically the intron comprising an RRE within a retroviral/lentiviral (e.g. SIV) vector/genome is appropriately spliced by target cells, aiding in the maturation of a stable mRNA molecule. This results in an increased expression of the coding regions of the retroviral/lentiviral (e.g. SIV) genome, including the transgene. Thus, the introduction of an intron comprising an RRE within retroviral/lentiviral (e.g. SIV) vector/genome results in increased expression of the transgene, which may encode a therapeutic protein.

When inserting an RRE into an intron according to the present invention, it is important that the position at which the RRE is inserted into the intron is carefully defined and controlled. This is because it is necessary to insert the RRE at such a position that the RRE is still able to function, and the intron is still recognised as such during transcription of the retroviral/lentiviral (e.g. SIV) genome. Provided that the position at which the RRE is inserted into the intron is carefully defined and controlled as described herein, then the precise sequence of the intron is not limiting.

Accordingly, also provided is a method of designing and/or producing an RRE-comprising intron according to the invention. Said method may comprise or consist of the steps of (a) identifying the splice donor and splice acceptor sequences within the intron; and (b) inserting the RRE into the intron such that the splice donor and split acceptor sequences remain intact. The method may further comprise one or more steps to delete the endogenous RRE from the retroviral/lentiviral (e.g. SIV) genome. Standard techniques insert and/or delete nucleic acid sequences from a nucleic acid (e.g. plasmid) are known in the art, and may be used by one of ordinary skill to insert the RRE-comprising intron and/or to delete the endogenous RRE according to the invention.

### Transgenes and Promoters

A retroviral/lentiviral (e.g. SIV) vector the invention typically comprises a transgene encoding for a therapeutic protein. A therapeutic protein is one which has potential utility in the treatment or prevention of a disease or condition, such as those describe herein. Thus, a retroviral/lentiviral (e.g. SIV) vector of the invention comprises a transgene encoding a protein which has a therapeutic effect on a disease or condition to be treated.

A retroviral/lentiviral (e.g. SIV) vector of the invention may comprise a transgene encoding a therapeutic protein which is a functional or wild-type form of a protein which is present in a patient to be treated in a dysfunctional form (whether the dysfunction is inherent or acquired). As used herein, the phrase “inherent dysfunction” refers to a protein which is innately dysfunctional due to genetic factors and the phrase “acquired dysfunction” refers to a protein which is dysfunctional due to environmental or other factors after birth. By way of non-limiting example, CFTR is an example of a protein which is inherently dysfunctional in patients with cystic fibrosis.

Thus, a retroviral/lentiviral (e.g. SIV) vector of the invention may comprise a transgene encoding a therapeutic protein which is a functional or wild-type form of a protein which is present in a patient, but which that has become dysfunctional due to a genetic disease, such as a genetic respiratory disease.

The retroviral/lentiviral (e.g. SIV) vectors of the present invention are useful in the treatment of diseases via their use in expressing therapeutic proteins in target cells, wherein the therapeutic protein exerts its therapeutic effects: (i) within the target cells; (ii) by secretion from said cells into the surrounding tissue; or (iii) for secretion from said cells into the circulatory system.

A retroviral/lentiviral (e.g. SIV) vector of the present invention may be pseudotyped to target airway cells of the respiratory tract (e.g. by pseudotyping with F and HN proteins from a respiratory paramyxovirus such as a Sendai virus), as described herein. Such retroviral/lentiviral (e.g. SIV) vectors of the invention are useful in the treatment of diseases via their use in expressing therapeutic proteins in airway cells: (i) within the respiratory tract; (ii) for secretion from said cells into the lumen of the respiratory tract; and (iii) for secretion from said cells into the circulatory system.

The therapeutic protein may be selected from: (a) a secreted therapeutic protein, optionally alpha-1-antitrypsin (AAT), Factor VIII, Surfactant Protein B (SFTPB), Factor VII, Factor IX, Factor X, Factor XI, von Willebrand Factor, Granulocyte-Macrophage Colony-Stimulating Factor (GM-CSF), Surfactant Protein C (SP-C), an anti-inflammatory protein (e.g. IL-10 or TGGβ) or monoclonal antibody, an anti-inflammatory decoy and a monoclonal antibody against an infectious agent; or (b) CFTR, CSF2RA, CSF2RB and ATP-binding cassette sub-family member A (ABCA3). Preferred examples of therapeutic proteins include AAT, GM-CSF, FVIII, CFTR, decorin, TRIM72 and ABCA3.

The transgene may encode: (i) a therapeutic protein that is secreted into epithelial lining fluid and/or blood); (ii) a therapeutic protein that is secreted into blood); or (iii) a therapeutic membrane protein). Preferred examples of these classes of transgenes include (i) AAT; (ii) FVIII; and (iii) CFTR.

In some embodiments, the therapeutic protein is not an antibody, particularly not a monoclonal antibody and/or not a β-globulin gene. In such embodiments, the therapeutic protein may be selected from: (a) a secreted therapeutic protein, optionally alpha-1-Antitrypsin (AAT), Factor VIII, Surfactant Protein B (SFTPB), Factor VII, Factor IX, Factor X, Factor XI, von Willebrand Factor, Granulocyte-Macrophage Colony-Stimulating Factor (GM-CSF), Surfactant Protein C (SP-C), an anti-inflammatory protein (e.g. IL-10, TGGβ, or TNF-alpha) and an anti-inflammatory decoy; or (b) CFTR, CSF2RA, CSF2RB and ATP-binding cassette sub-family member A (ABCA3).

The retroviral/lentiviral (e.g. SIV) vectors of the invention are particularly efficient at driving the expression, secretion and/or membrane insertion of proteins (e.g. therapeutic proteins as described herein) by airway cells. This is particularly the case when the retroviral/lentiviral (e.g. SIV) vectors are F/HN pseudotyped viral vectors of the invention (as described herein), which are efficient at targeting cells in the airway epithelium.

As such, for therapeutic applications the retroviral/lentiviral (e.g. SIV) vectors of the invention are typically delivered to cells of the respiratory tract, including the cells of the airway epithelium. In other words, the retroviral/lentiviral (e.g. SIV) vectors of the invention are typically delivered to airway cells as described herein. Accordingly, the retroviral/lentiviral (e.g. SIV) vectors of the invention are particularly suited for treatment of diseases or disorders of the airways, respiratory tract, or lung. Typically, the retroviral/lentiviral (e.g. SIV) vectors of the invention may be used for the treatment of a genetic respiratory disease.

A retroviral/lentiviral (e.g. SIV) vector of the invention may comprise a transgene encoding a polypeptide or protein that is therapeutic for the treatment of such diseases, particularly a disease or disorder of the airways, respiratory tract, or lung. The transgene and therapeutic protein of the invention are not limited, one of ordinary skill in the art will be able to identify therapeutic proteins which may be usefully delivered according to the invention, particularly in the context of genetic diseases, particularly genetic respiratory diseases and diseases or disorders of the airways, respiratory tract, or lung such as those described herein.

Accordingly, a retroviral/lentiviral (e.g. SIV) vectors of the invention may comprise a nucleic acid sequence encoding a therapeutic protein selected from: (a) a secreted therapeutic protein, optionally alpha-1-antitrypsin (AAT), Factor VIII, Surfactant Protein B (SFTPB), ADAMTS13, Factor VII, Factor IX, Factor X, Factor XI, von Willebrand Factor, Granulocyte-Macrophage Colony-Stimulating Factor (GM-CSF), Surfactant Protein C (SP-C), an anti-inflammatory protein (e.g. IL-10, TGGβ, or TNF-alpha) or monoclonal antibody, an anti-inflammatory decoy and a monoclonal antibody against an infectious agent; or (b) CFTR, CSF2RA, CSF2RB, ATP-binding cassette sub-family member A (ABCA3), DNAH5, DNAH11, DNAI1, and DNA12. Other examples of therapeutic proteins that may be encoded by a transgene comprised in a retroviral/lentiviral (e.g. SIV) vector of the invention include genes related to or associated with other surfactant deficiencies. Preferred examples of therapeutic proteins include AAT, GM-CSF, FVIII, CFTR, ADAMTS13, SFTPB, decorin, TRIM72 and ABCA3.

The therapeutic protein encoded by a retroviral/lentiviral (e.g. SIV) vector of the invention may be an AAT. An example of an AAT therapeutic transgene (SERPINA1) is provided by SEQ ID NO: 6, or by the complementary sequence of SEQ ID NO: 7. SEQ ID NO: 6 is a codon-optimized CpG depleted AAT transgene (SERPINA1) previously designed by the present inventors to enhance translation in human cells. Such optimisation has been shown to enhance gene expression by up to 15-fold. Variants of same sequence (as defined herein) which possess the same technical effect of enhancing translation compared with the unmodified (wild-type) AAT gene sequence are also encompassed by the present invention. The therapeutic protein encoded by said AAT transgene, may be exemplified by the polypeptide of SEQ ID NO: 8. Variants thereof (as described therein) are also included, particularly variants with at least 90% (such as at least 90, 92, 94, 95, 96, 97, 98, 99 or 100%) to any one of SEQ ID NO: 6, 7 or 8.

The therapeutic protein encoded by a retroviral/lentiviral (e.g. SIV) vector of the invention may be an FVIII. Examples of a FVIII therapeutic transgene are provided by SEQ ID NOs: 9 and 10, or by the respective complementary sequences of SEQ ID NO: 11 and 12. The polypeptide encoded by the FVIII transgene, may be exemplified by the polypeptide of SEQ ID NO: 13 or 14. Variants thereof (as described therein) are also included, particularly variants with at least 90% (such as at least 90, 92, 94, 95, 96, 97, 98, 99 or 100%) to any one of SEQ ID NOs: 9 to 14.

Preferably, the therapeutic protein encoded by a retroviral/lentiviral (e.g. SIV) vector of the invention is a CFTR. An example of a CFTR transgene is provided by SEQ ID NO: 15. The polypeptide encoded by said CFTR transgene, may be exemplified by the polypeptide of SEQ ID NO: 16. Variants thereof (as described therein) are also included, particularly variants with at least 90% (such as at least 90, 92, 94, 95, 96, 97, 98, 99 or 100%) to SEQ ID NO: 15 or 16.

The therapeutic protein encoded by a retroviral/lentiviral (e.g. SIV) vector of the invention may be GM-CSF. A GM-CSF transgene may comprise or consist of SEQ ID NO: 17 (human). The polypeptide encoded by the GM-CSF transgene may be exemplified by the polypeptide of SEQ ID NO: 18 (human). Variants thereof (as described therein) are also included, particularly variants with at least 90% (such as at least 90, 92, 94, 95, 96, 97, 98, 99 or 100%) to any one of SEQ ID NOs: 17 and 18.

The transgene may encode decorin. An example of a DCN transgene is provided by SEQ ID NO: 21. The polypeptide encoded by said DCN transgene, may be exemplified by the polypeptide of SEQ ID NO: 22. Variants thereof (as described therein) are also included, particularly variants with at least 90% (such as at least 90, 92, 94, 95, 96, 97, 98, 99 or 100%) to SEQ ID NO: 21 or 22.

The transgene may encode TRIM72. An example of a TRIM72 transgene is provided by SEQ ID NO: 23. The polypeptide encoded by said TRIM72 transgene, may be exemplified by the polypeptide of SEQ ID NO: 24. Variants thereof (as described therein) are also included, particularly variants with at least 90% (such as at least 90, 92, 94, 95, 96, 97, 98, 99 or 100%) to SEQ ID NO: 23 or 24.

The transgene may encode ABCA3. An example of a ABCA3 transgene is provided by SEQ ID NO: 25. The polypeptide encoded by said ABCA3 transgene, may be exemplified by the polypeptide of SEQ ID NO: 26. Variants thereof (as described therein) are also included, particularly variants with at least 90% (such as at least 90, 92, 94, 95, 96, 97, 98, 99 or 100%) to SEQ ID NO: 25 or 26.

The transgene may encode SFTPB. An example of a SFTPB transgene is provided by SEQ ID NO: 40. The polypeptide encoded by said SFTPB transgene, may be exemplified by the polypeptide of SEQ ID NO: 41. Variants thereof (as described therein) are also included, particularly variants with at least 90% (such as at least 90, 92, 94, 95, 96, 97, 98, 99 or 100%) to SEQ ID NO: 40 or 41.

The transgene may encode ADAMTS13. An example of a ADAMTS13 transgene is provided by SEQ ID NO: 42. The polypeptide encoded by said ADAMTS13 transgene, may be exemplified by the polypeptide of SEQ ID NO: 43. Variants thereof (as described therein) are also included, particularly variants with at least 90% (such as at least 90, 92, 94, 95, 96, 97, 98, 99 or 100%) to SEQ ID NO: 42 or 43.

The therapeutic protein encoded by a retroviral/lentiviral (e.g. SIV) vector of the invention may be encoded by any one of SFTPB, SFTPC, ADAMTS13, Factor V, Factor VII, Factor IX, Factor X and/or Factor XI, von Willebrand Factor, GM-CSF, ABCA3, TRIM72 or DCN, or other known related gene.

When the respiratory tract epithelium is targeted for delivery of the retroviral/lentiviral (e.g. SIV) vectors of the invention, the therapeutic protein may be AAT, SFTPB, or GM-CSF. The therapeutic protein may be a monoclonal antibody (mAb) against an infectious agent (bacterial, fungal or viral, e.g. the SARS-Co-V2 virus). The therapeutic protein may be anti-TNF alpha. The therapeutic protein may be one implicated in an inflammatory, immune or metabolic condition.

A retroviral/lentiviral (e.g. SIV) vector of the invention may be delivered to the cells of the respiratory tract to allow production of proteins to be secreted into circulatory system. In such embodiments, the therapeutic protein may be any one of Factor VII, Factor VIII, Factor IX, Factor X, Factor XI and/or von Willebrand's factor. Such a retroviral/lentiviral (e.g. SIV) vector of the invention may be used in the treatment of diseases, particularly cardiovascular diseases and blood disorders, preferably blood clotting deficiencies such as haemophilia. Again, the therapeutic protein may be an mAb against an infectious agent or a protein implicated in an inflammatory, immune or metabolic condition, such as, lysosomal storage disease.

The retroviral/lentiviral (e.g. SIV) vector comprises a promoter operably linked to a transgene, enabling expression of the transgene. Typically the promoter is a hybrid human CMV enhancer/EF1a (hCEF) promoter. This hCEF promoter may lack the intron corresponding to nucleotides 570-709 and the exon corresponding to nucleotides 728-733 of the hCEF promoter. A preferred example of an hCEF promoter sequence of the invention is provided by SEQ ID NO: 27. The promoter may be a CMV promoter. An example of a CMV promoter sequence is provided by SEQ ID NO: 28. The promoter may be a human elongation factor 1a (EF1a) promoter. An example of a EF1a promoter is provided by SEQ ID NO: 29. Other promoters for transgene expression are known in the art and their suitability for the retroviral/lentiviral (e.g. SIV) vectors of the invention determined using routine techniques known in the art. Non-limiting examples of other promoters include UBC and UCOE. As described herein, the promoter may be modified to further regulate expression of the transgene of the invention.

The promoter included in the retroviral/lentiviral (e.g. SIV) vector of the invention may be specifically selected and/or modified to further refine regulation of expression of the therapeutic gene. Again, suitable promoters and standard techniques for their modification are known in the art. As a non-limiting example, a number of suitable (CpG-free) promoters suitable for use in the present invention are described in Pringle et al. (J. Mol. Med. Berl. 2012, 90(12): 1487-96), which is herein incorporated by reference in its entirety. Preferably, the retroviral/lentiviral vectors (particularly SIV F/HN vectors) of the invention comprise a hCEF promoter having low or no CpG dinucleotide content. The hCEF promoter may have all CG dinucleotides replaced with any one of AG, TG or GT. Thus, the hCEF promoter may be CpG-free. A preferred example of a CpG-free hCEF promoter sequence of the invention is provided by SEQ ID NO: 27. The absence of CpG dinucleotides typically further improves the performance of retroviral/lentiviral (e.g. SIV) vectors of the invention and in particular in situations where it is not desired to induce an immune response against an expressed antigen or an inflammatory response against the delivered expression construct. The elimination of CpG dinucleotides reduces the occurrence of flu-like symptoms and inflammation which may result from administration of constructs, particularly when administered to the airways.

The retroviral/lentiviral (e.g. SIV) vector of the invention may be modified to allow shut down of gene expression. Standard techniques for modifying the vector in this way are known in the art. As a non-limiting example, Tet-responsive promoters are widely used.

A retroviral/lentiviral (e.g. SIV) vector of the invention may comprise a hCEF promoter and a CFTR transgene, including those described herein.

A retroviral/lentiviral (e.g. SIV) vector of the invention may comprise a hCEF promoter and an AAT transgene (SERPINA1), including those described herein.

A retroviral/lentiviral (e.g. SIV) vector of the invention may comprise a hCEF or CMV promoter and an FVIII transgene, including those described herein.

A retroviral/lentiviral (e.g. SIV) vector of the invention may comprise a hCEF or CMV promoter and an DCN transgene, including those described herein.

A retroviral/lentiviral (e.g. SIV) vector of the invention may comprise a hCEF or CMV promoter and an TRIM72 transgene, including those described herein.

A retroviral/lentiviral (e.g. SIV) vector of the invention may comprise a hCEF or CMV promoter and an ABCA3 transgene, including those described herein.

A retroviral/lentiviral (e.g. SIV) vector of the invention may comprise a hCEF or CMV promoter and an SFTPB transgene, including those described herein.

A retroviral/lentiviral (e.g. SIV) vector of the invention may comprise a hCEF or CMV promoter and an ADAMTS13 transgene, including those described herein.

The retroviral/lentiviral (e.g. SIV) vector of the invention comprises a nucleic acid encoding a therapeutic protein (said nucleic acid is referred to interchangeably herein as a transgene). The nucleic acid sequence encodes a gene product, e.g., a protein, particularly a therapeutic protein.

For example, the retroviral/lentiviral (e.g. SIV) vector may comprise a transgene encoding an AAT, GM-CSF, FVIII, SFTPB, ADAMTS13, CFTR, decorin, TRIM72 or ABCA3 and said transgene comprises (or consists of) a nucleic acid sequence having at least 90% (such as at least 90, 92, 94, 95, 96, 97, 98, 99 or 100%) sequence identity to the AAT, GM-CSF, FVIII, SFTPB, ADAMTS13, CFTR, decorin, TRIM72 or ABCA3 transgene respectively, examples of which are described herein. In a further embodiment, the transgene encoding AAT, GM-CSF, FVIII, SFTPB, ADAMTS13, CFTR, decorin, TRIM72 or ABCA3 comprises (or consists of) a nucleic acid sequence having at least 95% (such as at least 95, 96, 97, 98, 99 or 100%) sequence identity to the AAT, GM-CSF, FVIII, SFTPB, ADAMTS13, CFTR, decorin, TRIM72 or ABCA3 nucleic acid sequence respectively, examples of which are described herein. The nucleic acid sequence encoding CFTR may be provided by SEQ ID NO: 15, the nucleic acid sequence encoding AAT may be provided by SEQ ID NO: 6, or by the complementary sequence of SEQ ID NO: 7 and/or the nucleic acid sequence encoding FVIII may be provided by SEQ ID NO: 11 or 12, or by the respective complementary sequences of SEQ ID NO: 13 or 14, and/or the nucleic acid sequence encoding SFTPB may be provided by SEQ ID NO: 40, and/or the nucleic acid sequence encoding ADAMTS13 may be provided by SEQ ID NO: 42, and/or the nucleic acid sequence encoding GM-CSF may be provided by SEQ ID NO: 17, the nucleic acid sequence encoding decorin may be provided by SEQ ID NO: 21, the nucleic acid sequence encoding TRIM72 may be provided by SEQ ID NO: 23, and/or the nucleic acid sequence encoding ABCA3 may be provided by SEQ ID NO: 25, or variants thereof.

The amino acid sequence of the therapeutic protein may be a functional variant having at least 95% (such as at least 95, 96, 97, 98, 99 or 100%) sequence identity to the functional protein. For example, an AAT, FVIII, SFTPB, ADAMTS13, CFTR, GM-CSF, decorin, TRIM72 and/or ABCA3 polypeptide encoded by the respective AAT, FVIII, SFTPB, ADAMTS13, CFTR, CSF2, DCN, TRIM72, and/or ABCA3 transgene may comprise (or consist of) an amino acid sequence having at least 95% (such as at least 95, 96, 97, 98, 99 or 100%) sequence identity to the functional AAT, FVIII, SFTPB, ADAMTS13, CFTR, GM-CSF, decorin, TRIM72 and/or ABCA3 polypeptide sequence respectively.

The transgene encoding for a therapeutic protein may include a nucleic acid sequence encoding for the endogenous signal peptide of the therapeutic protein, or may exclude a nucleic acid sequence encoding for this signal peptide. All disclosure herein relates to both transgenes and therapeutic proteins including and excluding endogenous signal peptides unless explicitly stated. By way of non-limiting example, sequence identity of variants, and/or lengths of fragments may be based on the sequence with or without a signal peptide.

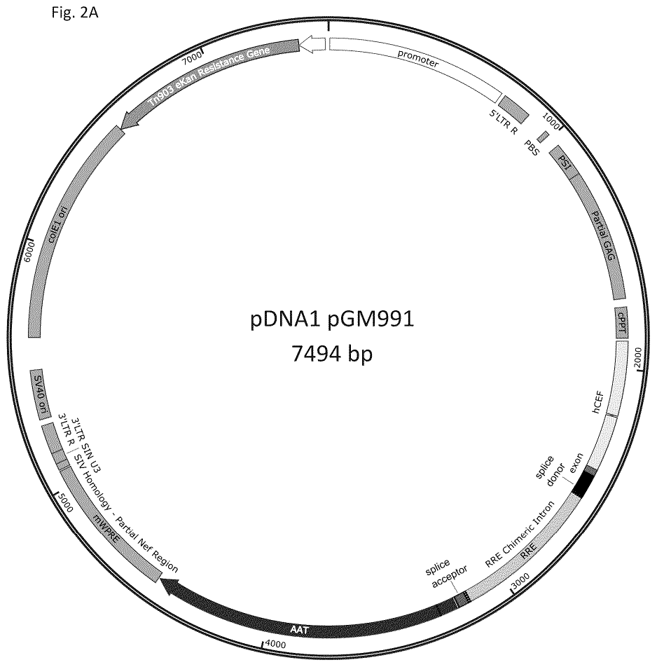

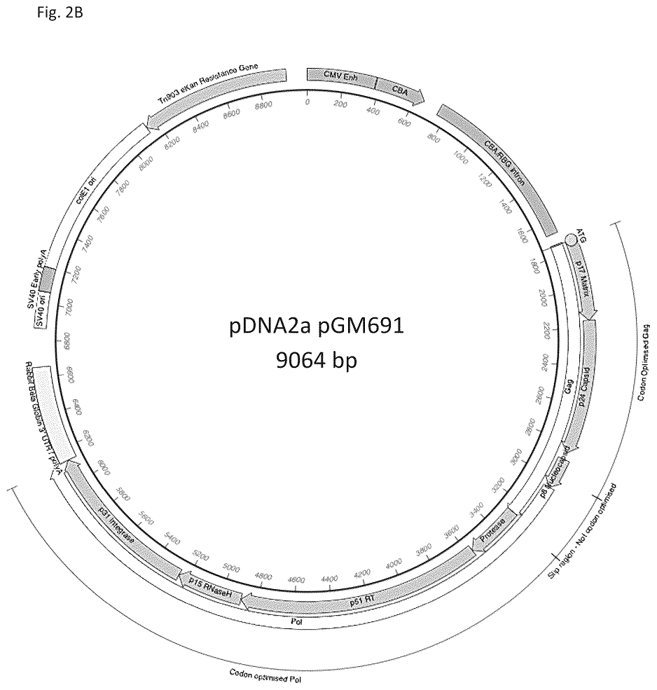

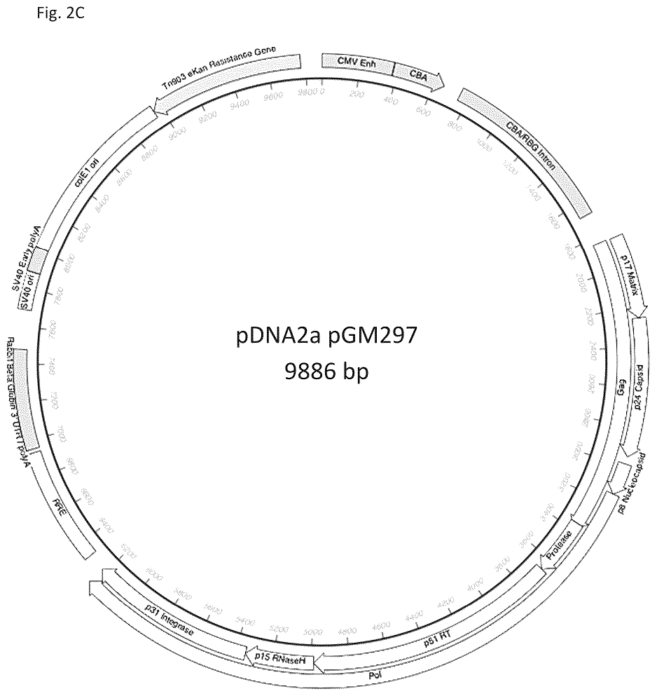

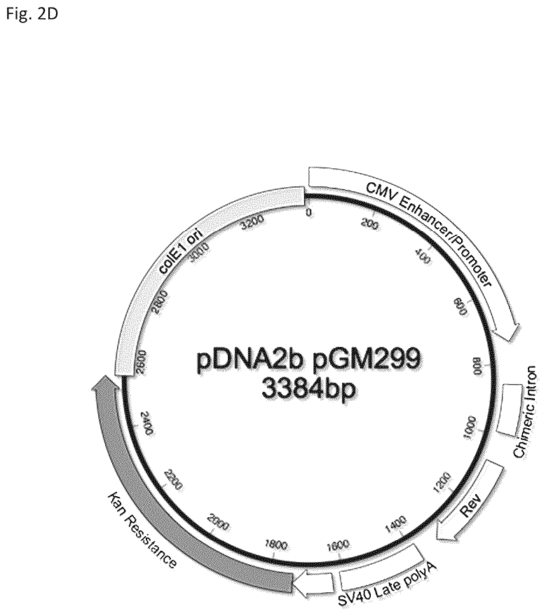

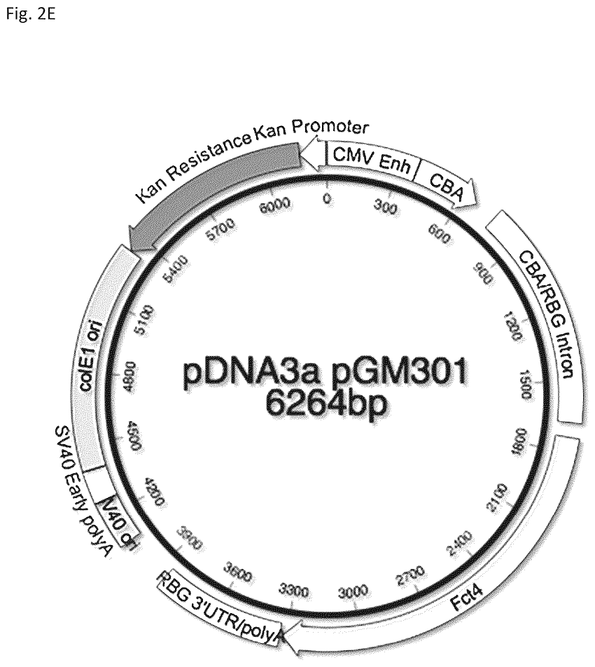

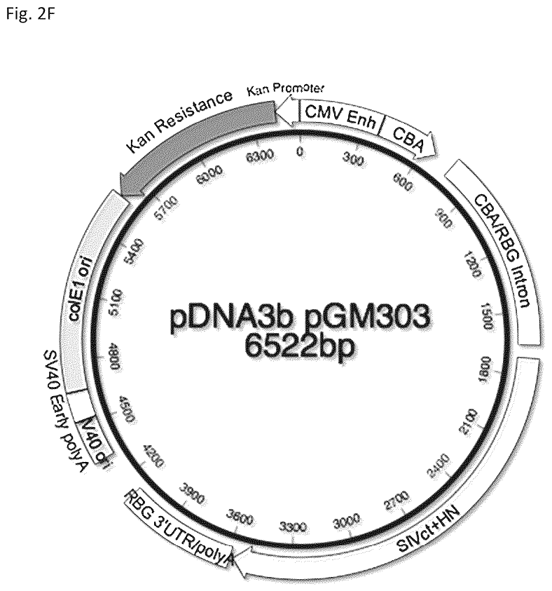

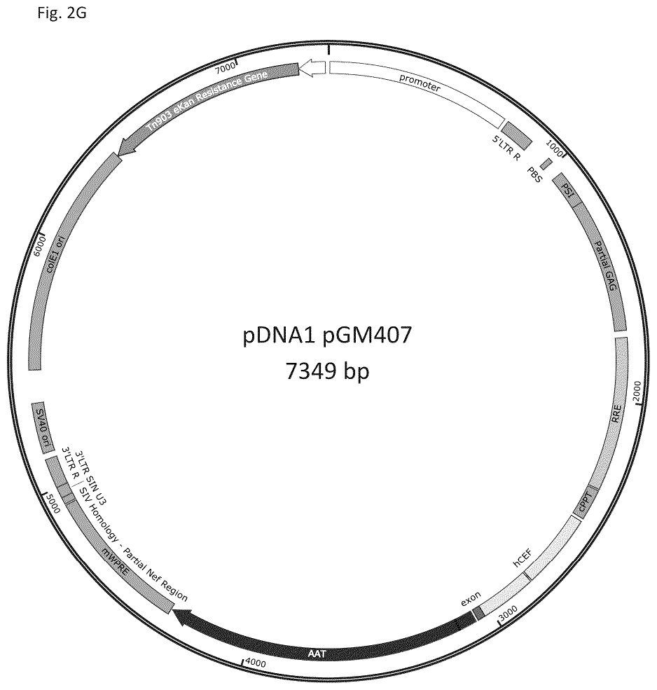

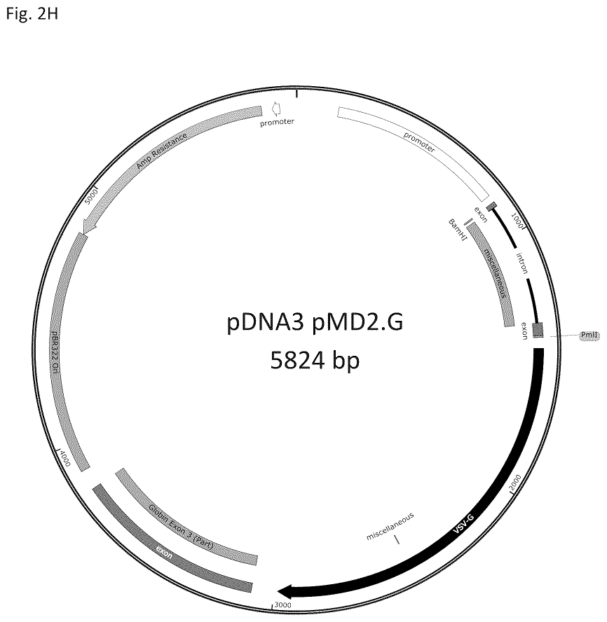

A retroviral/lentiviral (e.g. SIV) vector of the invention typically further comprises a Rev protein. This Rev protein is typically provided by (encoded by) one of the plasmids used in the manufacture of the retroviral/lentiviral (e.g. SIV) vector, as described herein. By way of non-limiting example, the Rev protein may be provided by the Rev plasmid (pDNA2b), wherein separate plasmids are used to provide the Gag-Pol and Rev proteins, or the Rev protein may be provided by the Rev-Gag-Pol plasmid, when a single plasmid is used to provide the Gag-Pol and Rev proteins. An exemplary pDNA2b plasmid, as described herein is pGM299, as shown in FIG. 2D and with a sequence represented by SEQ ID NO: 33. An exemplary Rev protein is the rSIV Rev protein which comprises or consists of the amino acid sequence of SEQ ID NO: 44. This Rev protein is encoded by the pGM299 plasmid.

### Nucleic Acids

The present invention also provides a nucleic acid comprising or consisting of an intron (e.g. a chimeric intron) into which an RRE has been introduced, as described herein. Any intron as described herein may be comprised in a nucleic acid of the invention. Similarly, any RRE as described herein may be comprised in a nucleic acid of the invention. The intron and RRE may each be selected independently, e.g. from those described herein. Any and all disclosure herein in relation to introns and/or RRE in the context of retroviral/lentiviral (e.g. SIV) vectors of the invention applies equally and without reservation to introns and/or RRE in the context of nucleic acids of the invention.

By way of non-limiting example, a nucleic acid of the invention may comprise any intron as described herein, for example a β-globin/IgG chimeric intron comprising or consisting of SEQ ID NO: 4, or a variant thereof, as described herein.

Alternatively or additionally, by way of further non-limiting example, a nucleic acid of the invention may comprise any RRE as described herein, for example a SIV RRE comprising or consisting of SEQ ID NO: 1, or a variant thereof, as described herein.

Preferably, and as exemplified herein, a nucleic acid of the invention comprises or consists of a β-globin/IgG chimeric intron and an SIV RRE. Accordingly, a nucleic acid of the invention may comprise or consist of a β-globin/IgG chimeric intron comprising a SIV RRE which comprise or consist of a nucleic acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 5. Preferably, a nucleic acid of the invention may comprise or consist of a β-globin/IgG chimeric intron comprising a SIV RRE which comprises or consists of nucleic acid sequence having at least 90%, at least 95%, or at least 99% sequence identity to SEQ ID NO: 5. More preferably, a nucleic acid of the invention may comprise or consist of a β-globin/IgG chimeric intron comprising a SIV RRE which consists of nucleic acid sequence having at least 90%, at least 95%, or at least 99% sequence identity to SEQ ID NO: 5. Still more preferably, a nucleic acid of the invention may comprise or consist of a β-globin/IgG chimeric intron comprising a SIV RRE which comprises or consists, particularly consists of, the nucleic acid sequence of SEQ ID NO: 5.

A nucleic acid of the invention may further comprise a transgene, typically which encodes a therapeutic protein as described herein. Any and all disclosure herein in relation to transgenes in the context of retroviral/lentiviral (e.g. SIV) vectors of the invention applies equally and without reservation to transgenes in the context of nucleic acids of the invention. In some preferred embodiments, the therapeutic protein encoded by the transgene is AAT or CFTR.

A nucleic acid of the invention comprising an RRE-comprising intron may exhibit increased expression of the transgene compared with a corresponding nucleic acid lacking said intron. The disclosure herein relating to the increase in transgene expression by a retroviral/lentiviral (e.g. SIV) vector of the invention comprising an RRE-comprising intron applies equally and without reservation to the increase in transgene expression exhibited by a nucleic acid of the invention.

By way of non-limiting example, the increase in expression of the therapeutic protein by a nucleic acid of the invention comprising an RRE-comprising intron may an increase of at least about 5-fold, an increase of at least about 10-fold, an increase of at least about 50-fold, an increase of at least about 100-fold, an increase of at least about 200-fold, an increase of at least about 500-fold, an increase of at least about 600-fold or more, typically compared with expression of the therapeutic protein from a corresponding nucleic acid without the RRE-comprising intron.

A nucleic acid of the invention enables long-term transgene expression, resulting in long-term expression of a therapeutic protein. As described herein, the phrases “long-term expression”, “sustained expression”, “long-lasting expression” and “persistent expression” are used interchangeably. Long-term expression according to the present invention means expression of a therapeutic protein, preferably at therapeutic levels, for at least 45 days, at least 60 days, at least 90 days, at least 120 days, at least 180 days, at least 250 days, at least 360 days, at least 450 days, at least 730 days or more. Preferably long-term expression means expression for at least 90 days, at least 120 days, at least 180 days, at least 250 days, at least 360 days, at least 450 days, at least 720 days or more, more preferably at least 360 days, at least 450 days, at least 720 days or more.

In particular, a nucleic acid of the invention may drive (increased) long-lasting expression of a therapeutic protein in an airway cell in vivo in a patient. Preferably, a nucleic acid of the invention drives expression of a therapeutic protein in an airway cell for at least 45 days, more preferably at least 90 days.

The nucleic acid of the nucleic acid may be as defined herein. The nucleic acid may comprise DNA and/or RNA. Preferably the nucleic acid is DNA.

A nucleic acid of the invention may optionally be codon optimised for expression in a particular cell type, for example, eukaryotic cells (e.g. mammalian cells, yeast cells, insect cells or plants cells) or prokaryotic cells (e.g. E. coli). The term “codon optimised” refers to the replacement of at least one codon within a base polynucleotide sequence with a codon that is preferentially used by the host organism in which the polynucleotide is to be expressed. Typically, the most frequently used codons in the host organism are used in the codon-optimised polynucleotide sequence. Methods of codon optimisation are well known in the art.

It will be understood by a skilled person that numerous different polynucleotides can encode the same polypeptide as a result of the degeneracy of the genetic code. It is also understood that skilled persons may, using routine techniques, make nucleotide substitutions that do not affect the polypeptide sequence encoded by the nucleic acid molecules to reflect the codon usage of any particular host organism in which the polypeptides are to be expressed. Therefore, unless otherwise specified, a nucleic acid that encodes a therapeutic protein of the invention includes all polynucleotide sequences that are degenerate versions of each other and that encode the same amino acid sequence. A nucleic acid cassette of the invention typically comprises a promoter operably linked to the nucleic acid sequence encoding the therapeutic protein. By operably linked, it is meant that the promoter is configured to express the nucleic acid sequence encoding the signal peptide and/or the nucleic acid sequence encoding the therapeutic protein. The disclosure herein in relation to promoters in the context of retroviral/lentiviral (e.g. SIV) vectors of the invention applies equally to nucleic acids of the invention. In some preferred embodiments, the promoter is a hCEF promoter as described herein.

The nucleic acids of the invention may include at least one part of a vector, in particular, regulatory elements. By way of non-limiting example, the promoter (e.g. the hCEFI promoter) within a nucleic acid cassette of the invention may be used to express more than one polypeptide, including one or more therapeutic proteins. Thus, the nucleic acid may comprise a nucleic acid sequence which, when transcribed, gives rise to multiple polypeptides, for instance a transcript may contain multiple open reading frames (ORFs) and also one or more Internal Ribosome Entry Sites (IRES) to allow translation of ORFs after the first ORF. A transcript may be polycistronic, i.e. it may be translated to give a polypeptide which is subsequently cleaved to give a plurality of polypeptides. Alternatively, a nucleic acid of the invention may comprise multiple promoters and hence give rise to a plurality of transcripts and hence a plurality of polypeptides, including a plurality of therapeutic proteins. Nucleic acids may, for instance, express one, two, three, four or more polypeptides via a promoter (e.g. hCEFI) or promoters.

A nucleic acid may comprise one or more translation initiation sequences (TIS). Translation initiation plays an important role in mRNA translation, canonically a methionyl tRNA unique for initiation (Met-tRNAi) identifies the AUG start codon and triggers the downstream translation process. Non-canonical start codons (e.g. CUG for valyl-tRNA)/TIS may also be used.

The nucleic acids of the present invention may comprise at least one termination signal. A “termination signal” or “terminator” is comprised of the DNA sequences involved in specific termination of an RNA transcript by an RNA polymerase. Thus, a termination signal that ends the production of an RNA transcript is contemplated according to the present invention. A terminator may be necessary in vivo to achieve desirable message levels. In eukaryotic systems, a terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, when the nucleic acid is for expression in eukaryotes, a terminator typically comprises a signal for the cleavage of the RNA, and it is preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

The nucleic acids of the invention are capable of expressing the therapeutic protein in a given host cell. Any appropriate host cell may be used, such as mammalian, bacterial, insect, yeast, and/or plant host cells. In addition, cell-free expression systems may be used. Such expression systems and host cells are standard in the art.

Typically the nucleic acid cassettes and vectors of the invention are capable of expressing the therapeutic protein in airway cells, as described herein in relation to retroviral/lentiviral (e.g. SIV) vectors of the invention.

The nucleic acids of the invention may be made using any suitable process known in the art. Thus, the nucleic acids may be made using chemical synthesis techniques. Alternatively, the nucleic acids of the invention may be made using molecular biology techniques.

A nucleic acid of the invention may be used in the production of a retroviral/lentiviral (e.g. SIV) vector, as described herein. By way of non-limiting example, a nucleic acid of the invention may be a plasmid which is used in the manufacture of a retroviral/lentiviral (e.g. SIV) vector. A nucleic acid of the invention may be comprised in a retroviral/lentiviral (e.g. SIV) vector.

A nucleic acid of the invention may be in the form of a DNA vector, such as a DNA plasmid. The vector(s) may be an RNA vector, such as a mRNA vector or a self-amplifying RNA vector. The DNA and/or RNA vector(s) of the invention may be capable of expression in eukaryotic and/or prokaryotic cells.

Typically, the DNA and/or RNA vector(s) are capable of expression in a cell of a subject, for example, a cell of a mammalian or avian subject to be immunised.

Typically the nucleic acids of the invention are capable of expressing the therapeutic protein in airway cells (as described herein).

A non-viral vector of the present invention may be a phage vector, such as an AAV/phage hybrid vector as described in Hajitou et al., Cell 2006; 125(2) pp. 385-398; herein incorporated by reference.

Nucleic acids of the present invention (e.g. DNA or RNA vectors) may be designed in silico, and then synthesised by conventional polynucleotide synthesis techniques.

Non-viral plasmids cannot replicate in the subject to be treated, as they lack the viral genetic material which hijacks the body's normal production machinery. However they are capable of replicating in appropriate host cells, such as yeasts or bacteria including E. coli, and particularly airway cells as defined herein.

The term “plasmid” as used herein refers to a construction comprised of genetic material designed to direct transformation of a targeted cell. The plasmid contains a plasmid backbone. A “plasmid backbone” as used herein contains multiple genetic elements positionally and sequentially oriented with other necessary genetic elements such that the nucleic acid in the nucleic acid can be transcribed and when necessary translated in the transfected cells.

The plasmid backbone can contain one or more unique restriction sites within the backbone. The plasmid may be capable of autonomous replication in a defined host or organism such that the cloned sequence is reproduced. The plasmid can confer some well-defined phenotype on the host organism which is either selectable or readily detected. The plasmid or plasmid backbone may have a linear or circular configuration. The components of a plasmid can contain, but is not limited to, a DNA molecule incorporating: (1) the plasmid backbone; (2) a sequence encoding a signal peptide; (3) a sequence encoding a therapeutic protein; and (4) regulatory elements for transcription, translation, RNA stability and replication

The purpose of the plasmid in human gene therapy for the efficient delivery of nucleic acid sequences to, and expression of therapeutic proteins in, a cell or tissue. In particular, the purpose of the plasmid is to achieve high copy number, avoid potential causes of plasmid instability and provide a means for plasmid selection. As for expression, a nucleic acid of the invention contains the necessary elements for expression of the transgene comprised in the nucleic acid. Expression includes the efficient transcription of an inserted gene, nucleic acid sequence, or nucleic acid within the plasmid.

A DNA plasmid may be CpG-free, or be optimised to reduce CpG dinucleotides as described herein. A DNA plasmid of the invention may be codon-optimised as described herein.

Methods of preparing plasmid DNA are well known in the art. Typically, they are capable of autonomous replication in an appropriate host or producer cell.

Host cells containing (e.g. transformed, transfected, or electroporated with) the plasmid may be prokaryotic or eukaryotic in nature, either stably or transiently transformed, transfected, or electroporated with the plasmid. Suitable host cells include bacterial, yeast, fungal, invertebrate, and mammalian cells. Preferably the host cell is bacterial; more preferably E. coli.

Host cells can then be used in methods for the large scale production of the plasmid. The cells are grown in a suitable culture medium under favourable conditions, and the desired plasmid isolated from the cells, or from the medium in which the cells are grown, by any purification technique well known to those skilled in the art; e.g. see Sambrook et al, supra.

Any appropriate delivery means can be used to deliver a non-viral vector (e.g. plasmid) of the invention to a target cell or patient. Suitable delivery means are known in the art and within the routine skill of one of ordinary skill in the art. Non-limiting examples include the use of cationic lipids, polymers (e.g. polyethyleneimine and poly-L-lysine) and electroporation.

Preferably cationic lipids may be used to deliver non-viral (e.g. plasmid) vectors of the invention to target cells or to a patient. Non-limiting examples of cationic lipids suitable for use according to the invention are GL67A and lipofectamine.

The cationic lipid mixture GL67A is a mixture of three components—GL67 (Cholest-5-en-3-ol (3β)-,3-[(3-aminopropyl)[4-[(3-aminopropyl)amino]butyl]carbamate], (CAS Number: 179075-30-0)), DOPE (1,2-dioleoyl-sn-glycero-3-phosphoethanolamine) and DMPE-PEG5000 (1,2-Dimyristoyl-sn-Glycero-3-Phosphoethanolamine-N-[methoxy (Polyethylene glycol)5000]). These components are formulated at a 1:2:0.05 molar ratio to form GL67A. The composition of GL67A and methods for its production are disclosed in WO2013/061091, as are methods for preparing mixtures of GL67A with exemplary non-viral vectors. The contents of WO2013/061091 are herein incorporated by reference in their entirety.

Lipofectamine consists of a 3:1 mixture of DOSPA (2,3-dioleoyloxy-N-[2(sperminecarboxamido)ethyl]-N,N-dimethyl-1-propaniminium trifluoroacetate) and DOPE.

The invention also provides host cells comprising a nucleic acid (e.g. plasmid) of the invention. Typically a host cell is a mammalian cell, particularly a human cell or cell line. Non-limiting examples of host cells include HEK293 cells (such as HEK293F or HEK293T cells) and 293T/17 cells. Commercial cell lines suitable for the production of virus are also readily available (as described herein).

### Methods of Production

Methods for the production of retroviral/lentiviral (e.g. SIV) vectors of the invention as also described herein.

The present inventors have previously demonstrated that the use of codon-optimised gal-pol genes from SIV does not negatively impact the manufactured titre of a SIV vector pseudotyped with hemagglutinin-neuraminidase (HN) and fusion (F) proteins from a respiratory paramyxovirus, and can even result in an increased titre of the vector. This is described in PCT/GB2022/050524, which is herein incorporated by reference in its entirety. Further, the inventors have shown that retroviral vectors comprising a retroviral/lentiviral RNA sequence comprising (i) codon substitutions and (ii) a reduced number of modified retroviral/lentiviral open reading frames (ORFs) do not negatively impact the manufactured vector titre, transgene expression and/or integration of the retroviral/lentiviral RNA sequence into the host/target cell genome, and can even result in an increase in vector titre, transgene expression and/or integration of the retroviral/lentiviral RNA sequence. This is described in UK Application No. 2212472.1, which is herein incorporated by reference in its entirety.

The present inventors have now shown that retroviral/lentiviral (e.g. SIV) vectors can be produced with the endogenous RRE of the retroviral/lentiviral (e.g. SIV) genome deleted, and an intron with a retroviral/lentiviral (e.g. SIV) inserted therein introduced into the retroviral/lentiviral (e.g. SIV) genome, and that this can increase transgene expression.

Accordingly, the present invention provides a method of producing a retroviral/lentiviral (e.g. SIV) vector from which (i) the endogenous RRE of the retroviral/lentiviral (e.g. SIV) genome has been deleted, and (ii) an intron with a retroviral/lentiviral (e.g. SIV) inserted therein has been introduced. A retroviral/lentiviral (e.g. SIV) may typically be pseudotyped with hemagglutinin-neuraminidase (HN) and fusion (F) proteins from a respiratory paramyxovirus or with VSV-G, and which comprises a promoter and a transgene. Preferably said retroviral/lentiviral (e.g. SIV) vector is a lentiviral vector, with Simian immunodeficiency virus (SIV) vectors being particularly preferred.

The method of the invention may be a scalable GMP-compatible method.

The method of the invention allows the generation of retroviral/lentiviral (e.g. SIV) vectors as described herein, which exhibit high levels of transgene expression. Typically a method of the invention produces retroviral/lentiviral (e.g. SIV) vectors as described herein that exhibit increased transgene expression compared with a corresponding retroviral/lentiviral (e.g. SIV) vector which lacks an RRE-comprising intron according to the invention.

Preferably, the increase in transgene expression by a retroviral/lentiviral (e.g. SIV) vector of the invention may be an increase of at least about 5-fold, an increase of at least about 10-fold, an increase of at least about 50-fold, an increase of at least about 100-fold, an increase of at least about 200-fold, an increase of at least about 500-fold, an increase of at least about 600-fold or more, typically compared with expression of the transgene by a corresponding retroviral/lentiviral (e.g. SIV) vector which lacks an RRE-comprising intron according to the invention produced by the same method. More preferably, the increase in transgene expression by a retroviral/lentiviral (e.g. SIV) vector of the invention may be at least about 10-fold, more preferably at least about 100-fold, even more preferably at least about 500-fold, typically compared with expression of the transgene by a corresponding retroviral/lentiviral (e.g. SIV) vector which lacks an RRE-comprising intron according to the invention produced by the same method. By way of non-limiting example, when the RRE-comprising intron is a β-globulin/IgG chimeric intron comprising an RRE, such as the β-globulin/IgG chimeric RRE-comprising intron of SEQ ID NO: 5, said intron may increase transgene expression by a retroviral/lentiviral (e.g. SIV) vector of the invention comprising said β-globulin/IgG chimeric RRE-comprising intron by at least 600-fold, such as by about 686-fold compared with the expression of the transgene by a corresponding retroviral/lentiviral (e.g. SIV) vector without said β-globulin/IgG chimeric RRE-comprising intron.

A method of the invention typically allows the generation of retroviral/lentiviral (e.g. SIV) vectors comprising a modified retroviral/lentiviral (e.g. SIV) RNA sequence with high levels of vector integration into the host/target cell genome. Alternatively or additionally, a method of the invention may allow the generation of high titre purified retroviral/lentiviral (e.g. SIV) vectors comprising a modified retroviral/lentiviral (e.g. SIV) RNA sequence. These advantageous properties of the vectors and methods of the invention are as described in UK Application No. 2212472.1, which is herein incorporated by reference in its entirety.

The production of retroviral/lentiviral (e.g. SIV) vectors typically employs one or more plasmids which provide the elements needed for the production of the vector: the genome for the retroviral/lentiviral vector, the Gag-Pol, Rev, F and HN. Multiple elements can be provided on a single plasmid. Preferably each element is provided on a separate plasmid, such that there five plasmids, one for each of the vector genome, the Gag-Pol, Rev, F and HN, respectively. Alternatively, a single plasmid may provide the Gag-Pol and Rev elements, and may be referred to as a packaging plasmid (pDNA2). The remaining elements (genome, F and HN) may be provided by separate plasmids (pDNA1, pDNA3a, pDNA3b respectively), such that four plasmids are used for the production of a retroviral/lentiviral (e.g. SIV) vector according to the invention. In the four plasmid methods, pDNA1, pDNA3a and pDNA3b may be as described herein in the context of the five-plasmid method.

For retroviral/lentiviral (e.g. SIV) vectors pseudotyped with another envelope protein, such as VSV-G, rather than F and HN proteins, again a method of the invention typically employs one or more plasmids which provide the elements needed for the production of the vector: the genome for the retroviral/lentiviral vector, the Gag-Pol (pDNA2a), Rev (pDNA2b), and envelope (e.g. VSV-G) (pDNA3). Multiple elements can be provided on a single plasmid. Preferably each element is provided on a separate plasmid, such that there four plasmids, one for each of the vector genome, the Gag-Pol, Rev and envelope (e.g. VSV-G), respectively. In the four plasmid methods for VSV-G pseudotyped retroviral/lentiviral vectors, pDNA1, pDNA2a and pDNA2b may be as described herein in the context of the five-plasmid method for retroviral/lentiviral vectors pseudotyped with F and HN proteins. Alternatively, a single plasmid may provide the Gag-Pol and Rev elements, and may be referred to as a packaging plasmid (pDNA2). The remaining elements (genome and VSV-G) may be provided by separate plasmids (pDNA1 and pDNA3 respectively), such that three plasmids are used for the production of a retroviral/lentiviral (e.g. SIV) vector according to the invention. In the three plasmid methods, pDNA1 may be as described herein in the context of the five/four-plasmid methods.

Preferably, the vector genome plasmid encodes all the genetic material that is packaged into the final retroviral/lentiviral vector, including the transgene. The vector genome plasmid may be designated herein as “pDNA1”, and typically comprises the transgene and the transgene promoter. As described herein, the RRE-comprising intron is typically comprised within the vector genome plasmid. The RRE-comprising intron is typically incorporated from the vector genome plasmid into the retroviral/lentiviral (e.g. SIV) RNA sequence.

The other four plasmids are manufacturing plasmids encoding the Gag-Pol, Rev, F and HN proteins. These plasmids may be designated “pDNA2a”, “pDNA2b”, “pDNA3a” and “pDNA3b” respectively.

Typically, the lentivirus is SIV, such as SIV1, preferably SIV-AGM. The F and HN proteins are derived from a respiratory paramyxovirus, preferably a Sendai virus.

In a specific embodiment relating to AAT, the five plasmids are characterised by FIGS. 2A-2F, thus pDNA1 is the pGM991 plasmid of FIG. 2A, pDNA2a is the pGM691 plasmid of FIG. 2B or the pGM297 plasmid of FIG. 2C, pDNA2b is the pGM299 plasmid of FIG. 2D, pDNA3a is the pGM301 plasmid of FIG. 2E and pDNA3b is the pGM303 plasmid of FIG. 2F, or variants thereof any of these plasmids (as described herein). pGM407 (as shown in FIG. 2G) is an unmodified version of the vector genome plasmid from which pGM991 is derived.

The plasmid as defined in FIG. 2A is represented by SEQ ID NO: 30; the plasmid as defined in FIG. 2B is represented by SEQ ID NO: 31; the plasmid as defined in FIG. 2C is represented by SEQ ID NO: 32; the plasmid as defined in FIG. 2D is represented by SEQ ID NO: 33; the plasmid as defined in FIG. 2E is represented by SEQ ID NO: 34; the plasmid as defined in FIG. 2F is represented by SEQ ID NO: 35; and the plasmid as defined in FIG. 2G is represented by SEQ ID NO: 36. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 30 to 36 are encompassed.

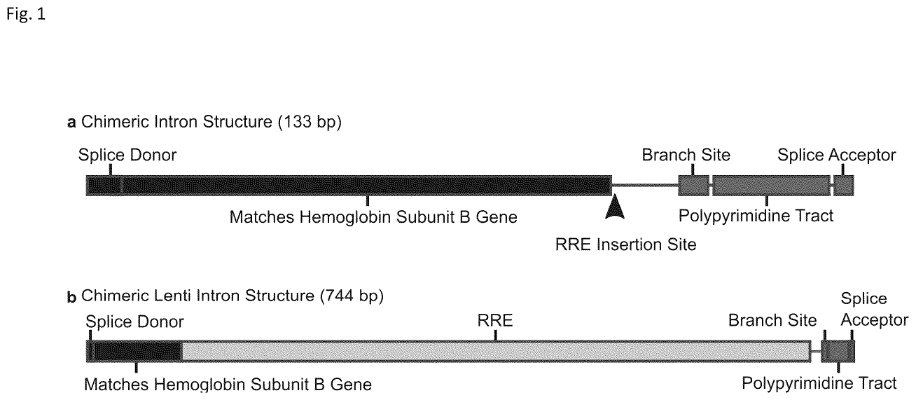

In each of the three-, four- or five-plasmid methods of the invention all of the plasmids contribute to the formation of the final retroviral/lentiviral (e.g. SIV) vector, although only the vector genome plasmid provides nucleic acid sequence comprised in the retroviral/lentiviral (e.g. SIV) RNA sequence. During manufacture of the retroviral/lentiviral (e.g. SIV) vector, the vector genome plasmid (pDNA1) provides the enhancer/promoter, Psi, RRE-comprising intron, cPPT, mWPRE, SIN LTR, SV40 polyA (see FIG. 1A), which are important for virus manufacture. Using pGM991 as a non-limiting example of a pDNA1, the CMV enhancer/promoter, SV40 polyA, colE1 Ori and KanR are involved in manufacture of the retroviral/lentiviral (e.g. SIV) vector of the invention, but are not found in the final retroviral/lentiviral (e.g. SIV) vector. The cPPT (central polypurine tract), RRE-comprising intron (inserted between hCEF and the AAT transgene), hCEF, AAT (transgene) and mWPRE from pGM991 are found in the final retroviral/lentiviral (e.g. SIV) vector. SIN LTR (long terminal repeats, SIN/IN self-inactivating) and Psi (packaging signal) may be found in the final retroviral/lentiviral (e.g. SIV) vector. In contrast, compared with pGM991, pGM407 (from which pGM991 is derived) lacks the RRE-comprising intron, but comprises the endogenous SIV RRE, which is positioned 5′ of the hCEF promoter and between the partial Gag and cPPT sequences.

For other retroviral/lentiviral (e.g. SIV) vectors of the invention, corresponding elements from the other vector genome plasmids (pDNA1) are required for manufacture (but not found in the final vector), or are present in the final retroviral/lentiviral (e.g. SIV) vector.

In a specific embodiment relating to pseudotyping with VSV-G, the four plasmids are characterised by FIGS. 2A-2F, thus pDNA1 is the pGM991 plasmid of FIG. 2A, pDNA2a is the pGM691 plasmid of FIG. 2B or the pGM297 plasmid of FIG. 2C, pDNA2b is the pGM299 plasmid of FIG. 2D, pDNA3 is the pMD2.G plasmid of FIG. 2H, or variants thereof any of these plasmids (as described herein). The plasmid as defined in FIG. 2A is represented by SEQ ID NO: 30; the plasmid as defined in FIG. 2B is represented by SEQ ID NO: 31; the plasmid as defined in FIG. 2C is represented by SEQ ID NO: 32; the plasmid as defined in FIG. 2D is represented by SEQ ID NO: 33; the plasmid as defined in FIG. 2H is represented by SEQ ID NO: 49. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 30 to 33 and 49 are encompassed.

The F and HN proteins from pDNA3a and pDNA3b (preferably Sendai F and HN proteins) or the VSV-G from pDNA3 are important for infection of target cells with the final retroviral/lentiviral (e.g. SIV) vector, i.e. for entry of a patient's epithelial cells (typically lung or nasal cells as described herein). The products of the pDNA2a and pDNA2b plasmids (or pDNA2 if the Gag-Pol and Rev elements are combined in a single plasmid) are important for virus transduction, i.e. for inserting the retroviral/lentiviral (e.g. SIV) DNA into the host's genome. The promoter, regulatory elements (such as WPRE) and transgene are important for transgene expression within the target cell(s).

A method of the invention may comprise or consist of the following steps: (a) growing cells in suspension; (b) transfecting the cells with one or more plasmids; (c) adding a nuclease; (d) harvesting the retrovirus/lentivirus (e.g. SIV); (e) adding trypsin; and (f) purification of the retrovirus/lentivirus (e.g. SIV).

This method may use the three-, four- or five-plasmid system described herein. Thus, for a five-plasmid method, the one or more plasmids may comprise or consist of: a vector genome plasmid pDNA1; a Gag-Pol plasmid (e.g. codon-optimised Gag-Pol plasmid), pDNA2a; a Rev plasmid, pDNA2b; a fusion (F) protein plasmid, pDNA3a; and a hemagglutinin-neuraminidase (HN) plasmid, pDNA3b. The pDNA1 may be pGM991. The pDNA2a may be pGM297 or pGM691, preferably pGM691. The pDNA2b may be pGM299. The pDNA3a may be pGM301. The pDNA3b may be pGM303. Any combination of pDNA1, pDNA2a, pDNA2b, pDNA3a and pDNA3b may be used. Preferably, the pDNA1 is pGM991; the pDNA2a is pGM691; the pDNA2b is pGM299; the pDNA3a is pGM301; and the pDNA3b is pGM303.

For a four-plasmid method, the one or more plasmids may comprise or consist of: a vector genome plasmid pDNA1; a Gag-Pol plasmid (e.g. codon-optimised Gag-Pol plasmid), pDNA2a; a Rev plasmid, pDNA2b; and a VSV-G plasmid, pDNA3. The pDNA1 may be pGM991. The pDNA2a may be pGM297 or pGM691, preferably pGM691. The pDNA2b may be pGM299. The pDNA3 may be pMD2.G. Any combination of pDNA1, pDNA2a, pDNA2b, and pDNA3 may be used. Preferably, the pDNA1 is pGM991; the pDNA2a is pGM691; the pDNA2b is pGM299; the pDNA3a is pGM301; and the pDNA3 is pMD2.G.

Any appropriate ratio of vector genome plasmid: Gag-Pol plasmid: Rev plasmid: F plasmid: HN plasmid may be used to further optimise (increase) the retroviral/lentiviral (e.g. SIV) titre produced. By way of non-limiting example, the ratio of vector genome plasmid: Gag-Pol plasmid: Rev plasmid: F plasmid: HN plasmid may by in the range of 10-40:-4-20:3-12:3-12:3-12, typically 15-20:7-11:4-8:4-8:4-8, such as about 18-22:7-11:4-8:4-8:4-8, 19-21:8-10:5-7:5-7:5-7. Preferably the ratio of vector genome plasmid: Gag-Pol plasmid: Rev plasmid: F plasmid: HN plasmid is about 20:9:6:6:6. Preferably the ratio of vector genome plasmid: Gag-Pol plasmid: Rev plasmid: VSV-G plasmid is about 20:9:6:12.

Steps (a)-(f) of the method are typically carried out sequentially, starting at step (a) and continuing through to step (f). The method may include one or more additional step, such as additional purification steps, buffer exchange, concentration of the retroviral/lentiviral (e.g. SIV) vector after purification, and/or formulation of the retroviral/lentiviral (e.g. SIV) vector after purification (or concentration). Each of the steps may comprise one or more sub-steps. For example, harvesting may involve one or more steps or sub-steps, and/or purification may involve one or more steps or sub-steps.

Any appropriate cell type may be transfected with the one or more plasmids (e.g. the five-, four- or three-plasmids described herein) to produce a retroviral/lentiviral (e.g. SIV) vector of the invention. Typically mammalian cells, particularly human cell lines are used. Non-limiting examples of cells suitable for use in the methods of the invention are HEK293 cells (such as HEK293F or HEK293T cells) and 293T/17 cells. Commercial cell lines suitable for the production of virus are also readily available (e.g. Gibco Viral Production Cells—Catalogue Number A35347 from ThermoFisher Scientific).

The cells may be grown in animal-component free media, including serum-free media. The cells may be grown in a media which contains human components. The cells may be grown in a defined media comprising or consisting of synthetically produced components.

Any appropriate transfection means may be used according to the invention. Selection of appropriate transfection means is within the routine practice of one of ordinary skill in the art. By way of non-limiting example, transfection may be carried out by the use of PEIPro™ Lipofectamine2000™ or Lipofectamine3000™.

Any appropriate nuclease may be used according to the invention. Selection of appropriate nuclease is within the routine practice of one of ordinary skill in the art. Typically the nuclease is an endonuclease. By way of non-limiting example, the nuclease may be Benzonase® or Denarase®. The addition of the nuclease may be at the pre-harvest stage or at the post-harvest stage, or between harvesting steps.

The gag-pol genes used in the production of a retroviral/lentiviral (e.g. SIV) vectors of the invention may be codon-optimised. Thus, the gag-pol genes within the pDNA2a plasmid may be codon-optimised. By way of non-limiting example, codon-optimised gag-pol genes may comprise or consist of the nucleic acid sequence of SEQ ID NO: 37, or a variant thereof (as defined herein). In particular, the codon-optimised gag-pol genes of the invention may comprise or consist of a nucleic acid sequence having at least 80%, at least 85%, at least 90%, at least 95%, at least 96%, at least 97%, at least 98%, at least 99% or more sequence identity to SEQ ID NO: 37, preferably at least 95%, identity to SEQ ID NO: 37. The codon-optimised gag-pol genes may consist of the nucleic acid sequence of SEQ ID NO: 37. The preferred pDNA2a, pGM691, comprises the codon-optimised gag-pol genes of SEQ ID NO: 37.

The gag-pol genes (e.g. SIV gag-pol genes), including codon-optimised gag-pol genes are typically operably linked to a promoter to facilitate expression of the gag-pol proteins. Any suitable promoter may be used, including those described herein in the context of promoters for the transgene. Preferably, the promoter is a CAG promoter, as used on the exemplified pGM691 plasmid. An exemplary CAG promoter is set out in SEQ ID NO: 38. The codon-optimised gag-pol genes of SEQ ID NO: 37 comprise a translational slip, and so do not form a single conventional open reading frame.

Codon-optimised gag-pol genes (or nucleic acids comprising or consisting thereof) and plasmids comprising said genes or nucleic acids are advantageous in the production of retroviral/lentiviral (e.g. SIV) vectors using methods of the invention, as they allow for the production of high titre retroviral/lentiviral (e.g. SIV) vectors. Typically said codon-optimised gag-pol genes (or nucleic acids comprising or consisting thereof) and plasmids comprising said genes or nucleic acids can be used to produces a titre of retroviral/lentiviral (e.g. SIV) vector that is at least equivalent to the titre of retroviral/lentiviral (e.g. SIV) vector produced by a corresponding method which does not use codon-optimised gag-pol genes, as described herein. Codon-optimised gag-pol genes are further disclosed in PCT/GB2022/050524, which is herein incorporated by reference in its entirety.

The invention also provides a retroviral/lentiviral (e.g. SIV) vector obtainable by a method of the invention.

Typically, the retroviral/lentiviral (e.g. SIV) vector obtainable by a method of the invention is produced at a high-titre, as described herein. Titre may be measured in terms of transducing units, as defined here.

Accordingly, the retroviral/lentiviral (e.g. SIV) vectors of the invention, including those obtainable by a method of the invention may optionally be at a titre of at least about 2.5×10⁶ TU/mL, at least about 3.0×10⁶ TU/mL, at least about 3.1×10⁶ TU/mL, at least about 3.2×10⁶ TU/mL, at least about 3.3×10⁶ TU/mL, at least about 3.4×10⁶ TU/mL, at least about 3.5×10⁶ TU/mL, at least about 3.6×10⁶ TU/mL, at least about 3.7×10⁶ TU/mL, at least about 3.8×10⁶ TU/mL, at least about 3.9×10⁶ TU/mL, at least about 4.0×10⁶ TU/mL or more. Preferably the retroviral/lentiviral (e.g. SIV) vector is produced at a titre of at least about 3.0×10⁶ TU/mL, or at least about 3.5×10⁶ TU/mL.

The production of high-titre retroviral/lentiviral (e.g. SIV) vectors may impart other desirable properties on the resulting vector products. For example, without being bound by theory, it is believed that production at high titres without the need for intense concentration by methods such as TFF results in a higher quality vector product than retroviral/lentiviral (e.g. SIV) vectors produced by corresponding methods without the use of codon-optimised gag-pol genes (and optionally a modified vector genome plasmid), because the vectors are exposed to less shear forces which can damage the viral particles and their RNA cargo.

Typically the gag-pol genes (e.g. codon-optimised gag-pol genes) used are matched to the retroviral/lentiviral vector being produced. By way of non-limiting example, when the lentiviral vector is an HIV vector, the codon-optimised gag-pol genes used are HIV gag-pol genes. By way of non-limiting example, when the lentiviral vector is an SIV vector, the codon-optimised gag-pol genes used are SIV gag-pol genes.

Preferably the codon-optimised gag-pol genes used are SIV gag-pol genes.

As described herein, the retroviral/lentiviral (e.g. SIV) vectors of the invention (i) lack the endogenous RRE; and (ii) comprise an RRE-comprising intron. Accordingly, the vector genome plasmid used in the production of a retroviral/lentiviral (e.g. SIV) vector of the invention may be modified to (i) delete the endogenous RRE and (ii) introduce an RRE-comprising intron. Any disclosure herein in relation to retroviral/lentiviral (e.g. SIV) vectors (i) lacking the endogenous RRE and (ii) comprising an RRE-comprising intron, applies equally and without reservation to the vector genome plasmids (pDNA1) described herein, which may be used in the production of retroviral/lentiviral (e.g. SIV) vectors of the invention.

As used herein, the term “trypsin” refers to both trypsin and equivalents thereof. An equivalent enzyme is one with the same or essentially the same cleavage specificity as trypsin. Trypsin cleavage activity may be defined as cleavage C-terminal to arginine or lysine residues, typically exclusively C-terminal to arginine or lysine residues. The trypsin activity may preferably be provided by an animal origin free, recombinant enzyme such as TrypLE Select™. The addition of trypsin may be at the pre-harvest stage or at the post-harvest stage, or between harvesting steps.

Any appropriate purification means may be used to purify the retroviral/lentiviral (e.g. SIV) vector. Non-limiting examples of suitable purification steps include depth/end filtration, tangential flow filtration (TFF) and chromatography. The purification step typically comprises at least on chromatography step. Non-limiting examples of chromatography steps that may be used in accordance with the invention include mixed-mode size exclusion chromatography (SEC) and/or anion exchange chromatography. Elution may be carried out with or without the use of a salt gradient, preferably without.

This method may be used to produce the retroviral/lentiviral (e.g. SIV) vectors of the invention, such as those comprising a CFTR, AAT and/or FVIII gene as described herein. Alternatively, the retroviral/lentiviral (e.g. SIV) vector of the invention comprises any of the above-mentioned genes, or the genes encoding the above-mentioned proteins.

The method, may use any combination of one or more of the specific plasmid constructs provided by FIG. 2A-2F or 2H to provide a retroviral/lentiviral (e.g. SIV) vector of the invention. Particularly the plasmid constructs of FIG. 2A, 2B and 2D-2F or 2A, 2B and 2D or 2H are used.

### Therapeutic Indications

The retroviral/lentiviral (e.g. SIV) vectors and nucleic acids (e.g. plasmids) of the present invention enable high and sustained transgene expression. The retroviral/lentiviral (e.g. SIV) vectors and nucleic acids (e.g. plasmids), and particularly the F/HN-pseudotyped retroviral/lentiviral (e.g. SIV) vectors of the invention are capable of: (i) airway transduction without disruption of epithelial integrity; (ii) persistent gene expression; (iii) lack of chronic toxicity; and (iv) efficient repeat administration. Long term/persistent stable gene expression, preferably at a therapeutically-effective level, may be achieved using repeat doses of a vector of the present invention. Alternatively, a single dose may be used to achieve the desired long-term expression.

Thus, advantageously, the retroviral/lentiviral (e.g. SIV) vectors and nucleic acids (e.g. plasmids) of the present invention can be used in gene therapy. By way of example, the efficient airway cell uptake properties of the retroviral/lentiviral (e.g. SIV) vectors and nucleic acids (e.g. plasmids) of the invention make them highly suitable for treating respiratory tract diseases. The retroviral/lentiviral (e.g. SIV) vectors and nucleic acids (e.g. plasmids) of the invention can also be used in methods of gene therapy to promote secretion of therapeutic proteins. By way of further example, the invention provides secretion of therapeutic proteins into the lumen of the respiratory tract or the circulatory system. Thus, administration of a retroviral/lentiviral (e.g. SIV) vector or nucleic acid (e.g. plasmid) of the invention and its uptake by airway cells may enable the use of the lungs (or nose or airways) as a “factory” to produce a therapeutic protein that is then secreted and enters the general circulation at therapeutic levels, where it can travel to cells/tissues of interest to elicit a therapeutic effect. In contrast to intracellular or membrane proteins, the production of such secreted proteins does not rely on specific disease target cells being transduced, which is a significant advantage and achieves high levels of protein expression. Thus, other diseases which are not respiratory tract diseases, such as cardiovascular diseases and blood disorders, particularly blood clotting deficiencies, can also be treated by the retroviral/lentiviral (e.g. SIV) vectors and nucleic acids (e.g. plasmids) of the present invention.

Retroviral/lentiviral (e.g. SIV) vectors and nucleic acids (e.g. plasmids) of the invention can effectively treat a disease by providing a transgene for the correction of the disease. For example, inserting a functional copy of the CFTR gene to ameliorate or prevent lung disease in CF patients, independent of the underlying mutation. Accordingly, retroviral/lentiviral (e.g. SIV) vectors and nucleic acids (e.g. plasmids) of the invention may be used to treat cystic fibrosis (CF), typically by gene therapy with a CFTR transgene as described herein.

As another example, retroviral/lentiviral (e.g. SIV) vectors and nucleic acids (e.g. plasmids) of the invention may be used to treat Alpha-1 Antitrypsin (AAT) deficiency, typically by gene therapy with a AAT transgene as described herein. AAT is a secreted anti-protease that is produced mainly in the liver and then trafficked to the lung, with smaller amounts also being produced in the lung itself. The main function of AAT is to bind and neutralise/inhibit neutrophil elastase. Gene therapy with AAT according to the present invention is relevant to AAT deficient patient, as well as in other lung diseases such as CF or chronic obstructive pulmonary disease (COPD), and offers the opportunity to overcome some of the problems encountered by conventional enzyme replacement therapy (in which AAT isolated from human blood and administered intravenously every week), providing stable, long-lasting expression in the target tissue (lung/nasal epithelium), ease of administration and unlimited availability.

Transduction with a retroviral/lentiviral (e.g. SIV) vector of the invention or transfection with a nucleic acid (e.g. plasmid) of the invention may lead to secretion of the recombinant protein into the lumen of the lung as well as into the circulation. One benefit of this is that the therapeutic protein reaches the interstitium. AAT gene therapy may therefore also be beneficial in other disease indications, non-limiting examples of which include type 1 and type 2 diabetes, acute myocardial infarction, ischemic heart disease, rheumatoid arthritis, inflammatory bowel disease, transplant rejection, graft versus host (GvH) disease, multiple sclerosis, liver disease, cirrhosis, vasculitides and infections, such as bacterial and/or viral infections.

AAT has numerous other anti-inflammatory and tissue-protective effects, for example in pre-clinical models of diabetes, graft versus host disease and inflammatory bowel disease. The production of AAT in the lung and/or nose following transduction according to the present invention may, therefore, be more widely applicable, including to these indications.

Other examples of diseases that may be treated with gene therapy of a secreted protein according to the present invention include cardiovascular diseases and blood disorders, particularly blood clotting deficiencies such as haemophilia (A, B or C), von Willebrand disease and Factor VII deficiency.

Other examples of diseases or disorders to be treated include Primary Ciliary Dyskinesia (PCD), acute lung injury, Surfactant Protein B (SFTB) deficiency, Pulmonary Alveolar Proteinosis (PAP), Chronic Obstructive Pulmonary Disease (COPD) and/or inflammatory, infectious, immune or metabolic conditions, such as lysosomal storage diseases.

Accordingly, the invention provides a method of treating a disease, the method comprising administering a retroviral/lentiviral (e.g. SIV) vector or nucleic acid (e.g. plasmid) of the invention to a subject. Typically the retroviral/lentiviral (e.g. SIV) vector or nucleic acid (e.g. plasmid) is produced using a method of the present invention. Any disease described herein may be treated according to the invention. In particular, the invention provides a method of treating a lung disease using a retroviral/lentiviral (e.g. SIV) vector or nucleic acid (e.g. plasmid) of the invention. The disease to be treated may be a chronic disease. Preferably, a method of treating CF is provided.

The invention also provides a retroviral/lentiviral (e.g. SIV) vector or nucleic acid (e.g. plasmid) as described herein for use in a method of treating a disease. Typically the retroviral/lentiviral (e.g. SIV) vector or nucleic acid (e.g. plasmid) is produced using a method of the present disclosure. Any disease described herein may be treated according to the invention. In particular, the invention provides a retroviral/lentiviral (e.g. SIV) vector or nucleic acid (e.g. plasmid) of the invention for use in a method of treating a lung disease. The disease to be treated may be a chronic disease. Preferably, a retroviral/lentiviral (e.g. SIV) vector or nucleic acid (e.g. plasmid) for use in treating CF is provided.

The invention also provides the use of a retroviral/lentiviral (e.g. SIV) vector or nucleic acid (e.g. plasmid) as described herein in the manufacture of a medicament for use in a method of treating a disease. Typically the retroviral/lentiviral (e.g. SIV) vector or nucleic acid (e.g. plasmid) is produced using a method of the present disclosure. Any disease described herein may be treated according to the invention. In particular, the invention provides the use of a retroviral/lentiviral (e.g. SIV) vector or nucleic acid (e.g. plasmid) of the invention for the manufacture of a medicament for use in a method of treating a lung disease. The disease to be treated may be a chronic disease. Preferably, the use of a retroviral/lentiviral (e.g. SIV) vector or nucleic acid (e.g. plasmid) in the manufacture of a medicament for use in a method of treating CF is provided.

### Formulation and Administration

The retroviral/lentiviral (e.g. SIV) vectors and/or nucleic acids (e.g. plasmids) of the invention may be administered in any dosage appropriate for achieving the desired therapeutic effect. Appropriate dosages may be determined by a clinician or other medical practitioner using standard techniques and within the normal course of their work. Non-limiting examples of suitable dosages of a retroviral/lentiviral (e.g. SIV) vector include 1×10³ transduction units (TU), 1×10⁹ TU, 1×10¹⁰ TU, 1×10¹¹ TU or more.

The invention also provides compositions comprising the retroviral/lentiviral (e.g. SIV) vectors and/or nucleic acids (e.g. plasmids) described above, and a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long-term storage.

The retroviral/lentiviral (e.g. SIV) vectors and/or nucleic acids (e.g. plasmids) of the invention may be administered by any appropriate route. It may be desired to direct the compositions of the present invention (as described above) to the respiratory system of a subject. Efficient transmission of a therapeutic/prophylactic composition or medicament to the site of infection in the respiratory tract may be achieved by oral or intra-nasal administration, for example, as aerosols (e.g. nasal sprays), or by catheters. Typically the retroviral/lentiviral (e.g. SIV) vectors and/or nucleic acids (e.g. plasmids) of the invention are stable in clinically relevant nebulisers, inhalers (including metered dose inhalers), catheters and aerosols, etc. Typically, therefore, the retroviral/lentiviral (e.g. SIV) vectors and/or nucleic acids (e.g. plasmids) of the invention are formulated for administration to the lungs by any appropriate means, e.g. they may be formulated for intratracheal administration, intranasal administration, aerosol delivery, or direct injection or delivery to the lungs (e.g. delivered by catheter). Other modes of delivery, e.g. intravenous delivery, are also encompassed by the invention.

In some embodiments the nose is a preferred production site for a therapeutic protein using a retroviral/lentiviral (e.g. SIV) vector and/or nucleic acid (e.g. plasmid) of the invention for at least one of the following reasons: (i) extracellular barriers such as inflammatory cells and sputum are less pronounced in the nose; (ii) ease of vector administration; (iii) smaller quantities of vector/nucleic acid required; and (iv) ethical considerations. Thus, transduction of nasal epithelial cells with a retroviral/lentiviral (e.g. SIV) vector or transfection with a nucleic acid (e.g. plasmid) of the invention may result in efficient (high-level) and long-lasting expression of the therapeutic transgene of interest. Accordingly, nasal administration of a retroviral/lentiviral (e.g. SIV) vector or a nucleic acid (e.g. plasmid) of the invention may be preferred.

Formulations for intra-nasal administration may be in the form of nasal droplets or a nasal spray. An intra-nasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

The formulation of pharmaceutical aerosols is routine to those skilled in the art, see for example, Sciarra, J. in Remington's Pharmaceutical Sciences (supra). The agents may be formulated as solution aerosols, dispersion or suspension aerosols of dry powders, emulsions or semisolid preparations. The aerosol may be delivered using any propellant system known to those skilled in the art. The aerosols may be applied to the upper respiratory tract, for example by nasal inhalation, or to the lower respiratory tract or to both. The part of the lung that the medicament is delivered to may be determined by the disorder. Compositions comprising a vector of the invention, in particular where intranasal delivery is to be used, may comprise a humectant. This may help reduce or prevent drying of the mucus membrane and to prevent irritation of the membranes. Suitable humectants include, for instance, sorbitol, mineral oil, vegetable oil and glycerol; soothing agents; membrane conditioners; sweeteners; and combinations thereof. The compositions may comprise a surfactant. Suitable surfactants include non-ionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusieates, bile salts and Octoxynol.

In some cases after an initial administration a subsequent administration of a retroviral/lentiviral (e.g. SIV) vector and/or a nucleic acid (e.g. plasmid) may be performed. The administration may, for instance, be at least a week, two weeks, a month, two months, six months, a year or more after the initial administration. In some instances, a retroviral/lentiviral (e.g. SIV) vector and/or a nucleic acid (e.g. plasmid) of the invention may be administered at least once a week, once a fortnight, once a month, every two months, every six months, annually or at longer intervals. Preferably, administration is every six months, more preferably annually. The retroviral/lentiviral (e.g. SIV) vectors and/or nucleic acids (e.g. plasmids) may, for instance, be administered at intervals dictated by when the effects of the previous administration are decreasing.

Any two or more retroviral/lentiviral (e.g. SIV) vectors and/or nucleic acids (e.g. plasmids) of the invention may be administered separately, sequentially or simultaneously. Thus two or more retroviral/lentiviral (e.g. SIV) vectors and/or nucleic acids (e.g. plasmids), wherein at least one retroviral/lentiviral (e.g. SIV) vector and/or nucleic acid (e.g. plasmid) is a retroviral/lentiviral (e.g. SIV) vector and/or nucleic acid (e.g. plasmid) of the invention, may be administered separately, simultaneously or sequentially. In particular two or more retroviral/lentiviral (e.g. SIV) vectors and/or nucleic acids (e.g. plasmids) of the invention may be administered in such a manner. The two may be administered in the same or different compositions. In a preferred instance, the two retroviral/lentiviral (e.g. SIV) vectors and/or nucleic acids (e.g. plasmids) may be delivered in the same composition.

Methods to Differentiate Between Retroviral/Lentiviral Vectors and Transgene mRNA

Conventional methods for the production of retroviral/lentiviral (e.g. SIV) vectors produce retroviral/lentiviral (e.g. SIV) vectors, which when transcribed, produce an mRNA which is identical in sequence to the retroviral/lentiviral (e.g. SIV) genome. Thus, it is not possible to distinguish between the retroviral/lentiviral (e.g. SIV) vector genome (the prodrug) and the transcribed mRNA (which will then be translated to produce the therapeutic protein).

In contrast, the present invention relates to retroviral/lentiviral (e.g. SIV) vectors which contain an RRE-comprising intron, as described herein. Thus, the retroviral/lentiviral (e.g. SIV) genome (the prodrug) contains the sequence of an RRE-comprising intron. This RRE-comprising intron is spliced out during transcription of the retroviral/lentiviral (e.g. SIV) genome, resulting in mRNA which lacks the RRE-comprising intron, and hence has a different nucleic acid sequence compared with the retroviral/lentiviral (e.g. SIV) genome from which it was derived. The different sequences of the retroviral/lentiviral (e.g. SIV) genome and the transcribed mRNA allow for the development of specific PCR- and in situ hybridisation-based assays that detect and quantify the different nucleic acid sequences, allowing for the retroviral/lentiviral (e.g. SIV) genome and the transcribed mRNA to each be quantified. Thus, the invention provides a means of discriminating between the retroviral/lentiviral (e.g. SIV) vector genome (the prodrug) and the mRNA (first step towards the active therapeutic). This may be useful, for example, during the production of the retroviral/lentiviral (e.g. SIV) vector, during its use in vitro and/or for evaluating clinical efficacy of the retroviral/lentiviral (e.g. SIV) vector.

Accordingly, the present invention provides a method for differentiating between a retroviral/lentiviral (e.g. SIV) vector and a transgene expressed by said retroviral vector, said method comprising or consisting of the steps of (a) transfecting cells with a retroviral/lentiviral (e.g. SIV) vector of the invention; (b) culturing the cells to allow transgene expression by the retroviral/lentiviral (e.g. SIV) vector; and (c) quantifying RNA within the cells; wherein (i) the amount of RNA comprising the intron into which a retroviral/lentiviral (e.g. SIV) RRE has been inserted corresponds to the copy number of the retroviral/lentiviral (e.g. SIV) vector; and (ii) the amount of RNA lacking the intron into which a retroviral/lentiviral (e.g. SIV) RRE has been inserted corresponds to the amount of transgene mRNA.

The present invention provides a method for differentiating between a retroviral/lentiviral (e.g. SIV) vector and mRNA transcribed from said retroviral/lentiviral (e.g. SIV) vector, said method comprising or consisting of the steps of (a) transfecting cells with a retroviral/lentiviral (e.g. SIV) vector of the invention; (b) culturing the cells to allow transcription of the genome of the retroviral/lentiviral (e.g. SIV) vector; and (c) quantifying RNA within the cells; wherein (i) the amount of RNA comprising the intron into which a retroviral/lentiviral (e.g. SIV) RRE has been inserted corresponds to the copy number of the retroviral/lentiviral (e.g. SIV) vector; and (ii) the amount of RNA lacking the intron into which a retroviral/lentiviral (e.g. SIV) RRE has been inserted corresponds to the amount of mRNA transcribed from the retroviral/lentiviral (e.g. SIV) vector genome, and wherein optionally the amount of mRNA transcribed corresponds to the level of transgene expression.

Said methods may involve the quantification of RNA by any appropriate technique, examples of which are known in the art and may be selected by a skilled person without undue burden. Preferably, said method may involve the quantification of RNA by a PCR-based and/or in situ hybridisation-based assay. Such PCR-based methods may comprise the use of two sets of primer pairs. The first primer pair includes one primer which binds to a sequence outside the intron and another primer which binds to a sequence inside the intron. This first primer pair is capable of detecting and quantifying non-spliced retroviral/lentiviral (e.g. SIV) vectors. The second primer pair comprises two primers which bind outside of the intron on either side and, therefore only quantifies spliced retroviral/lentiviral (e.g. SIV) vectors. As the retroviral/lentiviral (e.g. SIV) mRNA is spliced, the second primer pair is specific for the mRNA transcribed from the retroviral/lentiviral (e.g. SIV) vector while the first primer pair will detect retroviral/lentiviral (e.g. SIV) vector genomes and integrated retroviral/lentiviral (e.g. SIV) DNA.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position—Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

A R N D C Q E G H I L K M F P S T W Y V
A 4
R −1 5
N −2 0 6
D −2 −2 1 6
C 0 −3 −3 −3 9
Q −1 1 0 0 −3 5
E −1 0 0 2 −4 2 5
G 0 −2 0 −1 −3 −2 −2 6
H −2 0 1 −1 −3 0 0 −2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5
F −2 −3 −3 −3 −2 −3 −3 −3 −1 0 0 −3 0 6
P −1 −2 −2 −1 −3 −1 −1 −2 −2 −3 −3 −1 −2 −4 7
S 1 −1 1 0 −1 0 0 0 −1 −2 −2 0 −1 −2 −1 4
T 0 −1 0 −1 −1 −1 −1 −2 −2 −1 −1 −1 −1 −2 −1 1 5
W −3 −3 −4 −4 −2 −2 −3 −2 −2 −3 −2 −3 −1 1 −4 −3 −211
Y −2 −2 −2 −3 −2 −1 −2 −3 2 −1 −1 −2 −1 3 −3 −2 −2 2 7
V 0 −3 −3 −3 −1 −2 −2 −3 −3 3 1 −2 1 −1 −2 −2 0 −3 −1 4

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and a -methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labelling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### SEQUENCE INFORMATION

SEQ ID NO: 1 SIV RRE sequence
SEQ ID NO: 2 β-globulin/IgG chimeric intron splice donor site
SEQ ID NO: 3 β-globulin/IgG chimeric intron splice acceptor site
SEQ ID NO: 4 β-globulin/IgG chimeric intron
SEQ ID NO: 5 β-globin/IgG chimeric intron comprising a SIV RRE
SEQ ID NO: 6 Exemplified AAT transgene (SERPINA1)
SEQ ID NO: 7 Complementary strand to the exemplified AAT transgene
SEQ ID NO: 8 Exemplified AAT polypeptide
SEQ ID NO: 9 Exemplified FVIII transgene (N6)
SEQ ID NO: 10 Exemplified FVIII transgene (V3)
SEQ ID NO: 11 Complementary strand to the exemplified FVIII transgene (N6)
SEQ ID NO: 12 Complementary strand to the exemplified FVIII transgene (V3)
SEQ ID NO: 13 Exemplified FVIII polypeptide (N6)
SEQ ID NO: 14 Exemplified FVIII polypeptide (V3)
SEQ ID NO: 15 Exemplified CFTR transgene (soCFTR2)
SEQ ID NO: 16 Exemplified CFTR polypeptide
SEQ ID NO: 17 Exemplified hGM-CSF transgene
SEQ ID NO: 18 Exemplified hGM-CSF polypeptide
SEQ ID NO: 19 Exemplified mGM-CSF transgene
SEQ ID NO: 20 Exemplified mGM-CSF polypeptide
SEQ ID NO: 21 Exemplified Human DCN (Decorin) transgene
SEQ ID NO: 22 Exemplified Human Decorin polypeptide
SEQ ID NO: 23 Exemplified Human TRIM72 transgene
SEQ ID NO: 24 Exemplified Human TRIM72 polypeptide
SEQ ID NO: 25 Exemplified Human ABCA3 (ABCA3) transgene
SEQ ID NO: 26 Exemplified Human ABCA3 polypeptide
SEQ ID NO: 27 Exemplified hCEF promoter
SEQ ID NO: 28 Exemplified CMV promoter
SEQ ID NO: 29 Exemplified EF1a promoter
SEQ ID NO: 30 Plasmid as defined in FIG. 2A (pDNA1 pGM991)
SEQ ID NO: 31 Plasmid as defined in FIG. 2B (pDNA1 pGM691)
SEQ ID NO: 32 Plasmid as defined in FIG. 2C (pDNA2a pGM297)
SEQ ID NO: 33 Plasmid as defined in FIG. 2D (pDNA2b pGM299)
SEQ ID NO: 34 Plasmid as defined in FIG. 2E (pDNA3a pGM301)
SEQ ID NO: 35 Plasmid as defined in FIG. 2F (pDNA3b pGM303)
SEQ ID NO: 36 Plasmid as defined in FIG. 2G (pDNA2a pGM407)
SEQ ID NO: 37 Codon-optimised SIV gag-pol nucleic acid sequence (from pGM691)
SEQ ID NO: 38 Exemplary CAG promoter
SEQ ID NO: 39 Exemplified WPRE component (mWPRE)
SEQ ID NO: 40 Exemplified Human SFTPB transgene
SEQ ID NO: 41 Exemplified Human SFTPB polypeptide
SEQ ID NO: 42 Exemplified Human ADAMTS13 transgene
SEQ ID NO: 43 Exemplified Human ADAMTS13 polypeptide
SEQ ID NO: 44 Exemplified rSIV Rev protein
SEQ ID NO: 45 oIC001 DNA forward primer
SEQ ID NO: 46 oIC002 DNA and RNA reverse primer
SEQ ID NO: 47 oIC105 RNA forward primer
SEQ ID NO: 48 CAGGS promoter comprising a chicken β-actin splice donor and a rabbit β-globulin splice acceptor
SEQ ID NO: 49 Plasmid as defined in FIG. 2H (pDNA3 pMD2.G)
SEQ ID NO: 50 HIV RRE sequence

```
SIV RRE sequence
SEQ ID NO: 1
CCGTTTGTGCTAGGGTTCTTAGGCTTCTTGGGGGCTGCTGGAACTGCAATGGGAGCAGCGGCGACAGC
CCTGACGGTCCAGTCTCAGCATTTGCTTGCTGGGATACTGCAGCAGCAGAAGAATCTGCTGGCGGCTG
TGGAGGCTCAACAGCAGATGTTGAAGCTGACCATTTGGGGTGTTAAAAACCTCAATGCCCGCGTCACA
GCCCTTGAGAAGTACCTAGAGGATCAGGCACGACTAAACTCCTGGGGGTGCGCATGGAAACAAGTATG
TCATACCACAGTGGAGTGGCCCTGGACAAATCGGACTCCGGATTGGCAAAATATGACTTGGTTGGAGT
GGGAAAGACAAATAGCTGATTTGGAAAGCAACATTACGAGACAATTAGTGAAGGCTAGAGAACAAGAG
GAAAAGAATCTAGATGCCTATCAGAAGTTAACTAGTTGGTCAGATTTCTGGTCTTGGTTCGATTTCTC
AAAATGGCTTAACATTTTAAAAATGGGATTTTTAGTAATAGTAGGAATAATAGGGTTAAGATTACTTT
ACACAGTATATGGATGTATAGTGAGGGTTAGGCAGGGATATGTTCCTCTATCTCCACAGATCCATAT
β-globulin/IgGG chimeric intron splice donor site
SEQ ID NO: 2
TGAGTTTAAGGTAAGT
β-globulin/IgGG chimeric intron splice acceptor site
SEQ ID NO: 3
CTCTCCACAG
β-globulin/IgGG chimeric intron
SEQ ID NO: 4
GTAAGTATCAAGGTTACAAGACAGGTTTAAGGAGACCAATAGAAACTGGGCTTGTCGAGACAGAGAAG
ACTCTTGCGTTTCTGATAGGCACCTATTGGTCTTACTGACATCCACTTTGCCTTTCTCTCCACAG
β-globin/IgGG chimeric intron comprising a SIV RRE
SEQ ID NO: 5
GTAAGTATCAAGGTTACAAGACAGGTTTAAGGAGACCAATAGAAACTGGGCTTGTCGAGACAGAGAAG
ACTCTTGCGTTTCTGATAGGCACGCGGCCGCCCGTTTGTGCTAGGGTTCTTAGGCTTCTTGGGGGCTG
CTGGAACTGCAATGGGAGCAGCGGCGACAGCCCTGACGGTCCAGTCTCAGCATTTGCTTGCTGGGATA
CTGCAGCAGCAGAAGAATCTGCTGGCGGCTGTGGAGGCTCAACAGCAGATGTTGAAGCTGACCATTTG
GGGTGTTAAAAACCTCAATGCCCGCGTCACAGCCCTTGAGAAGTACCTAGAGGATCAGGCACGACTAA
ACTCCTGGGGGTGCGCATGGAAACAAGTATGTCATACCACAGTGGAGTGGCCCTGGACAAATCGGACT
CCGGATTGGCAAAATATGACTTGGTTGGAGTGGGAAAGACAAATAGCTGATTTGGAAAGCAACATTAC
GAGACAATTAGTGAAGGCTAGAGAACAAGAGGAAAAGAATCTAGATGCCTATCAGAAGTTAACTAGTT
GGTCAGATTTCTGGTCTTGGTTCGATTTCTCAAAATGGCTTAACATTTTAAAAATGGGATTTTTAGTA
ATAGTAGGAATAATAGGGTTAAGATTACTTTACACAGTATATGGATGTATAGTGAGGGTTAGGCAGGG
ATATGTTCCTCTATCTCCACAGATCCATATGCGGCCGCCTATTGGTCTTACTGACATCCACTTTGCCT
TTCTCTCCACAG
Underlined = β-globin/IgGG chimeric intron
Double underlined = NotI restriction sites
Italicised = SIV RRE sequence
Exemplified AAT transgene (SERPINA1)
SEQ ID NO: 6
atgcccagct ctgtgtcctg gggcattctg ctgctggctg gcctgtgctg tctggtgcct 60
gtgtccctgg ctgaggaccc tcagggggat gctgcccaga aaacagacac ctcccaccat 120
gaccaggacc accccacctt caacaagatc acccccaacc tggcagagtt tgccttcagc 180
ctgtacagac agctggccca ccagagcaac agcaccaaca tctttttcag ccctgtgtcc 240
attgccacag cctttgccat gctgagcctg ggcaccaagg ctgacaccca tgatgagatc 300
ctggaaggcc tgaacttcaa cctgacagag atccctgagg cccagatcca tgagggcttc 360
caggaactgc tgagaaccct gaaccagcca gacagccagc tgcagctgac aacaggcaat 420
gggctgttcc tgtctgaggg cctgaagctg gtggacaagt ttctggaaga tgtgaagaag 480
ctgtaccact ctgaggcctt cacagtgaac tttggggaca cagaagaggc caagaaacag 540
atcaatgact atgtggaaaa gggcacccag ggcaagattg tggaccttgt gaaagagctg 600
gacagggaca ctgtgtttgc ccttgtgaac tacatcttct tcaagggcaa gtgggagagg 660
ccctttgaag tgaaggacac tgaggaagag gacttccatg tggaccaagt gaccacagtg 720
aaggtgccaa tgatgaagag actggggatg ttcaatatcc agcactgcaa gaaactgagc 780
agctgggtgc tgctgatgaa gtacctgggc aatgctacag ccatattctt tctgcctgat 840
gagggcaagc tgcagcacct ggaaaatgag ctgacccatg acatcatcac caaatttctg 900
gaaaatgagg acagaagatc tgccagcctg catctgccca agctgagcat cacaggcaca 960
tatgacctga agtctgtgct gggacagctg ggaatcacca aggtgttcag caatggggca 1020
gacctgagtg gagtgacaga ggaagcccct ctgaagctgt ccaaggctgt gcacaaggca 1080
gtgctgacca ttgatgagaa gggcacagag gctgctgggg ccatgtttct ggaagccatc 1140
cccatgtcca tccccccaga agtgaagttc aacaagccct ttgtgttcct gatgattgag 1200
cagaacacca agagccccct gttcatgggc aaggttgtga accccaccca gaaatga 1257
Complementary strand to the exemplified AAT transgene
SEQ ID NO: 7
tacgggtcga gacacaggac cccgtaagac gacgaccgac cggacacgac agaccacgga 60
cacagggacc gactcctggg agtcccccta cgacgggtct tttgtctgtg gagggtggta 120
ctggtcctgg tggggtggaa gttgttctag tgggggttgg accgtctcaa acggaagtcg 180
gacatgtctg tcgaccgggt ggtctcgttg tcgtggttgt agaaaaagtc gggacacagg 240
taacggtgtc ggaaacggta cgactcggac ccgtggttcc gactgtgggt actactctag 300
gaccttccgg acttgaagtt ggactgtctc tagggactcc gggtctaggt actcccgaag 360
gtccttgacg actcttggga cttggtcggt ctgtcggtcg acgtcgactg ttgtccgtta 420
cccgacaagg acagactccc ggacttcgac cacctgttca aagaccttct acacttcttc 480
gacatggtga gactccggaa gtgtcacttg aaacccctgt gtcttctccg gttctttgtc 540
tagttactga tacacctttt cccgtgggtc ccgttctaac acctggaaca ctttctcgac 600
ctgtccctgt gacacaaacg ggaacacttg atgtagaaga agttcccgtt caccctctcc 660
gggaaacttc acttcctgtg actccttctc ctgaaggtac acctggttca ctggtgtcac 720
ttccacggtt actacttctc tgacccctac aagttatagg tcgtgacgtt ctttgactcg 780
tcgacccacg acgactactt catggacccg ttacgatgtc ggtataagaa agacggacta 840
ctcccgttcg acgtcgtgga ccttttactc gactgggtac tgtagtagtg gtttaaagac 900
cttttactcc tgtcttctag acggtcggac gtagacgggt tcgactcgta gtgtccgtgt 960
atactggact tcagacacga ccctgtcgac ccttagtggt tccacaagtc gttaccccgt 1020
ctggactcac ctcactgtct ccttcgggga gacttcgaca ggttccgaca cgtgttccgt 1080
cacgactggt aactactctt cccgtgtctc cgacgacccc ggtacaaaga ccttcggtag 1140
gggtacaggt aggggggtct tcacttcaag ttgttcggga aacacaagga ctactaactc 1200
gtcttgtggt tctcggggga caagtacccg ttccaacact tggggtgggt ctttact 1257
Exemplified AAT polypeptide
SEQ ID NO: 8
Ala Glu Asp Pro Gln Gly Asp Ala Ala Gln Lys Thr Asp Thr Ser His
1 5 10 15
His Asp Gln Asp His Pro Thr Phe Ala Glu Asp Pro Gln Gly Asp Ala
                        20 25 30
Ala Gln Lys Thr Asp Thr Ser His His Asp Gln Asp His Pro Thr Phe
                35 40 45
Asn Lys Ile Thr Pro Asn Leu Ala Glu Phe Ala Phe Ser Leu Tyr Arg
        50 55 60
Gln Leu Ala His Gln Ser Asn Ser Thr Asn Ile Phe Phe Ser Pro Val
65 70 75 80
Ser Ile Ala Thr Ala Phe Ala Met Leu Ser Leu Gly Thr Lys Ala Asp
                                85 90 95
Thr His Asp Glu Ile Leu Glu Gly Leu Asn Phe Asn Leu Thr Glu Ile
                        100 105 110
Pro Glu Ala Gln Ile His Glu Gly Phe Gln Glu Leu Leu Arg Thr Leu
                115 120 125
Asn Gln Pro Asp Ser Gln Leu Gln Leu Thr Thr Gly Asn Gly Leu Phe
        130 135 140
Leu Ser Glu Gly Leu Lys Leu Val Asp Lys Phe Leu Glu Asp Val Lys
145 150 155 160
Lys Leu Tyr His Ser Glu Ala Phe Thr Val Asn Phe Gly Asp Thr Glu
                                165 170 175
Glu Ala Lys Lys Gln Ile Asn Asp Tyr Val Glu Lys Gly Thr Gln Gly
                      180 185 190
Lys Ile Val Asp Leu Val Lys Glu Leu Asp Arg Asp Thr Val Phe Ala
                195 200 205
Leu Val Asn Tyr Ile Phe Phe Lys Gly Lys Trp Glu Arg Pro Phe Glu
        210 215 220
Val Lys Asp Thr Glu Glu Glu Asp Phe His Val Asp Gln Val Thr Thr
225 230 235 240
Val Lys Val Pro Met Met Lys Arg Leu Gly Met Phe Asn Ile Gln His
                                245 250 255
Cys Lys Lys Leu Ser Ser Trp Val Leu Leu Met Lys Tyr Leu Gly Asn
                        260 265 270
Ala Thr Ala Ile Phe Phe Leu Pro Asp Glu Gly Lys Leu Gln His Leu
              275 280 285
Glu Asn Glu Leu Thr His Asp Ile Ile Thr Lys Phe Leu Glu Asn Glu
        290 295 300
Asp Arg Arg Ser Ala Ser Leu His Leu Pro Lys Leu Ser Ile Thr Gly
305 310 315 320
Thr Tyr Asp Leu Lys Ser Val Leu Gly Gln Leu Gly Ile Thr Lys Val
                                325 330 335
Phe Ser Asn Gly Ala Asp Leu Ser Gly Val Thr Glu Glu Ala Pro Leu
                        340 345 350
Lys Leu Ser Lys Ala Val His Lys Ala Val Leu Thr Ile Asp Glu Lys
                355 365 360
Gly Thr Glu Ala Ala Gly Ala Met Phe Leu Glu Ala Ile Pro Met Ser
        370 375 380
Ile Pro Pro Glu Val Lys Phe Asn Lys Pro Phe Val Phe Leu Met Ile
385 390 395 400
Glu Gln Asn Thr Lys Ser Pro Leu Phe Met Gly Lys Val Val Asn Pro
                                405 410 415
Thr Gln Lys
Exemplified FVIII transgene (N6)
SEQ ID NO: 9
atgcagattg agctgagcac ctgcttcttc ctgtgcctgc tgaggttctg cttctctgcc 60
accaggagat actacctggg ggctgtggag ctgagctggg actacatgca gtctgacctg 120
ggggagctgc ctgtggatgc caggttcccc cccagagtgc ccaagagctt ccccttcaac 180
acctctgtgg tgtacaagaa gaccctgttt gtggagttca ctgaccacct gttcaacatt 240
gccaagccca ggcccccctg gatgggcctg ctgggcccca ccatccaggc tgaggtgtat 300
gacactgtgg tgatcaccct gaagaacatg gccagccacc ctgtgagcct gcatgctgtg 360
ggggtgagct actggaaggc ctctgagggg gctgagtatg atgaccagac cagccagagg 420
gagaaggagg atgacaaggt gttccctggg ggcagccaca cctatgtgtg gcaggtgctg 480
aaggagaatg gccccatggc ctctgacccc ctgtgcctga cctacagcta cctgagccat 540
gtggacctgg tgaaggacct gaactctggc ctgattgggg ccctgctggt gtgcagggag 600
ggcagcctgg ccaaggagaa gacccagacc ctgcacaagt tcatcctgct gtttgctgtg 660
tttgatgagg gcaagagctg gcactctgaa accaagaaca gcctgatgca ggacagggat 720
gctgcctctg ccagggcctg gcccaagatg cacactgtga atggctatgt gaacaggagc 780
ctgcctggcc tgattggctg ccacaggaag tctgtgtact ggcatgtgat tggcatgggc 840
accacccctg aggtgcacag catcttcctg gagggccaca ccttcctggt caggaaccac 900
aggcaggcca gcctggagat cagccccatc accttcctga ctgcccagac cctgctgatg 960
gacctgggcc agttcctgct gttctgccac atcagcagcc accagcatga tggcatggag 1020
gcctatgtga aggtggacag ctgccctgag gagccccagc tgaggatgaa gaacaatgag 1080
gaggctgagg actatgatga tgacctgact gactctgaga tggatgtggt gaggtttgat 1140
gatgacaaca gccccagctt catccagatc aggtctgtgg ccaagaagca ccccaagacc 1200
tgggtgcact acattgctgc tgaggaggag gactgggact atgcccccct ggtgctggcc 1260
cctgatgaca ggagctacaa gagccagtac ctgaacaatg gcccccagag gattggcagg 1320
aagtacaaga aggtcaggtt catggcctac actgatgaaa ccttcaagac cagggaggcc 1380
atccagcatg agtctggcat cctgggcccc ctgctgtatg gggaggtggg ggacaccctg 1440
ctgatcatct tcaagaacca ggccagcagg ccctacaaca tctaccccca tggcatcact 1500
gatgtgaggc ccctgtacag caggaggctg cccaaggggg tgaagcacct gaaggacttc 1560
cccatcctgc ctggggagat cttcaagtac aagtggactg tgactgtgga ggatggcccc 1620
accaagtctg accccaggtg cctgaccaga tactacagca gctttgtgaa catggagagg 1680
gacctggcct ctggcctgat tggccccctg ctgatctgct acaaggagtc tgtggaccag 1740
aggggcaacc agatcatgtc tgacaagagg aatgtgatcc tgttctctgt gtttgatgag 1800
aacaggagct ggtacctgac tgagaacatc cagaggttcc tgcccaaccc tgctggggtg 1860
cagctggagg accctgagtt ccaggccagc aacatcatgc acagcatcaa tggctatgtg 1920
tttgacagcc tgcagctgtc tgtgtgcctg catgaggtgg cctactggta catcctgagc 1980
attggggccc agactgactt cctgtctgtg ttcttctctg gctacacctt caagcacaag 2040
atggtgtatg aggacaccct gaccctgttc cccttctctg gggagactgt gttcatgagc 2100
atggagaacc ctggcctgtg gattctgggc tgccacaact ctgacttcag gaacaggggc 2160
atgactgccc tgctgaaagt ctccagctgt gacaagaaca ctggggacta ctatgaggac 2220
agctatgagg acatctctgc ctacctgctg agcaagaaca atgccattga gcccaggagc 2280
ttcagccaga acagcaggca ccccagcacc aggcagaagc agttcaatgc caccaccatc 2340
cctgagaatg acatagagaa gacagaccca tggtttgccc accggacccc catgcccaag 2400
atccagaatg tgagcagctc tgacctgctg atgctgctga ggcagagccc caccccccat 2460
ggcctgagcc tgtctgacct gcaggaggcc aagtatgaaa ccttctctga tgaccccagc 2520
cctggggcca ttgacagcaa caacagcctg tctgagatga cccacttcag gccccagctg 2580
caccactctg gggacatggt gttcacccct gagtctggcc tgcagctgag gctgaatgag 2640
aagctgggca ccactgctgc cactgagctg aagaagctgg acttcaaagt ctccagcacc 2700
agcaacaacc tgatcagcac catcccctct gacaacctgg ctgctggcac tgacaacacc 2760
agcagcctgg gcccccccag catgcctgtg cactatgaca gccagctgga caccaccctg 2820
tttggcaaga agagcagccc cctgactgag tctgggggcc ccctgagcct gtctgaggag 2880
aacaatgaca gcaagctgct ggagtctggc ctgatgaaca gccaggagag cagctggggc 2940
aagaatgtga gcagcaggga gatcaccagg accaccctgc agtctgacca ggaggagatt 3000
gactatgatg acaccatctc tgtggagatg aagaaggagg actttgacat ctacgacgag 3060
gacgagaacc agagccccag gagcttccag aagaagacca ggcactactt cattgctgct 3120
gtggagaggc tgtgggacta tggcatgagc agcagccccc atgtgctgag gaacagggcc 3180
cagtctggct ctgtgcccca gttcaagaag gtggtgttcc aggagttcac tgatggcagc 3240
ttcacccagc ccctgtacag aggggagctg aatgagcacc tgggcctgct gggcccctac 3300
atcagggctg aggtggagga caacatcatg gtgaccttca ggaaccaggc cagcaggccc 3360
tacagcttct acagcagcct gatcagctat gaggaggacc agaggcaggg ggctgagccc 3420
aggaagaact ttgtgaagcc caatgaaacc aagacctact tctggaaggt gcagcaccac 3480
atggccccca ccaaggatga gtttgactgc aaggcctggg cctacttctc tgatgtggac 3540
ctggagaagg atgtgcactc tggcctgatt ggccccctgc tggtgtgcca caccaacacc 3600
ctgaaccctg cccatggcag gcaggtgact gtgcaggagt ttgccctgtt cttcaccatc 3660
tttgatgaaa ccaagagctg gtacttcact gagaacatgg agaggaactg cagggccccc 3720
tgcaacatcc agatggagga ccccaccttc aaggagaact acaggttcca tgccatcaat 3780
ggctacatca tggacaccct gcctggcctg gtgatggccc aggaccagag gatcaggtgg 3840
tacctgctga gcatgggcag caatgagaac atccacagca tccacttctc tggccatgtg 3900
ttcactgtga ggaagaagga ggagtacaag atggccctgt acaacctgta ccctggggtg 3960
tttgagactg tggagatgct gcccagcaag gctggcatct ggagggtgga gtgcctgatt 4020
ggggagcacc tgcatgctgg catgagcacc ctgttcctgg tgtacagcaa caagtgccag 4080
acccccctgg gcatggcctc tggccacatc agggacttcc agatcactgc ctctggccag 4140
tatggccagt gggcccccaa gctggccagg ctgcactact ctggcagcat caatgcctgg 4200
agcaccaagg agcccttcag ctggatcaag gtggacctgc tggcccccat gatcatccat 4260
ggcatcaaga cccagggggc caggcagaag ttcagcagcc tgtacatcag ccagttcatc 4320
atcatgtaca gcctggatgg caagaagtgg cagacctaca ggggcaacag cactggcacc 4380
ctgatggtgt tctttggcaa tgtggacagc tctggcatca agcacaacat cttcaacccc 4440
cccatcattg ccagatacat caggctgcac cccacccact acagcatcag gagcaccctg 4500
aggatggagc tgatgggctg tgacctgaac agctgcagca tgcccctggg catggagagc 4560
aaggccatct ctgatgccca gatcactgcc agcagctact tcaccaacat gtttgccacc 4620
tggagcccca gcaaggccag gctgcacctg cagggcagga gcaatgcctg gaggccccag 4680
gtcaacaacc ccaaggagtg gctgcaggtg gacttccaga agaccatgaa ggtgactggg 4740
gtgaccaccc agggggtgaa gagcctgctg accagcatgt atgtgaagga gttcctgatc 4800
agcagcagcc aggatggcca ccagtggacc ctgttcttcc agaatggcaa ggtgaaggtg 4860
ttccagggca accaggacag cttcacccct gtggtgaaca gcctggaccc ccccctgctg 4920
accagatacc tgaggattca cccccagagc tgggtgcacc agattgccct gaggatggag 4980
gtgctgggct gtgaggccca ggacctgtac tga 5013
Exemplified FVIII transgene (V3)
SEQ ID NO: 10
atgcagattg agctgagcac ctgcttcttc ctgtgcctgc tgaggttctg cttctctgcc 60
accaggagat actacctggg ggctgtggag ctgagctggg actacatgca gtctgacctg 120
ggggagctgc ctgtggatgc caggttcccc cccagagtgc ccaagagctt ccccttcaac 180
acctctgtgg tgtacaagaa gaccctgttt gtggagttca ctgaccacct gttcaacatt 240
gccaagccca ggcccccctg gatgggcctg ctgggcccca ccatccaggc tgaggtgtat 300
gacactgtgg tgatcaccct gaagaacatg gccagccacc ctgtgagcct gcatgctgtg 360
ggggtgagct actggaaggc ctctgagggg gctgagtatg atgaccagac cagccagagg 420
gagaaggagg atgacaaggt gttccctggg ggcagccaca cctatgtgtg gcaggtgctg 480
aaggagaatg gccccatggc ctctgacccc ctgtgcctga cctacagcta cctgagccat 540
gtggacctgg tgaaggacct gaactctggc ctgattgggg ccctgctggt gtgcagggag 600
ggcagcctgg ccaaggagaa gacccagacc ctgcacaagt tcatcctgct gtttgctgtg 660
tttgatgagg gcaagagctg gcactctgaa accaagaaca gcctgatgca ggacagggat 720
gctgcctctg ccagggcctg gcccaagatg cacactgtga atggctatgt gaacaggagc 780
ctgcctggcc tgattggctg ccacaggaag tctgtgtact ggcatgtgat tggcatgggc 840
accacccctg aggtgcacag catcttcctg gagggccaca ccttcctggt caggaaccac 900
aggcaggcca gcctggagat cagccccatc accttcctga ctgcccagac cctgctgatg 960
gacctgggcc agttcctgct gttctgccac atcagcagcc accagcatga tggcatggag 1020
gcctatgtga aggtggacag ctgccctgag gagccccagc tgaggatgaa gaacaatgag 1080
gaggctgagg actatgatga tgacctgact gactctgaga tggatgtggt gaggtttgat 1140
gatgacaaca gccccagctt catccagatc aggtctgtgg ccaagaagca ccccaagacc 1200
tgggtgcact acattgctgc tgaggaggag gactgggact atgcccccct ggtgctggcc 1260
cctgatgaca ggagctacaa gagccagtac ctgaacaatg gcccccagag gattggcagg 1320
aagtacaaga aggtcaggtt catggcctac actgatgaaa ccttcaagac cagggaggcc 1380
atccagcatg agtctggcat cctgggcccc ctgctgtatg gggaggtggg ggacaccctg 1440
ctgatcatct tcaagaacca ggccagcagg ccctacaaca tctaccccca tggcatcact 1500
gatgtgaggc ccctgtacag caggaggctg cccaaggggg tgaagcacct gaaggacttc 1560
cccatcctgc ctggggagat cttcaagtac aagtggactg tgactgtgga ggatggcccc 1620
accaagtctg accccaggtg cctgaccaga tactacagca gctttgtgaa catggagagg 1680
gacctggcct ctggcctgat tggccccctg ctgatctgct acaaggagtc tgtggaccag 1740
aggggcaacc agatcatgtc tgacaagagg aatgtgatcc tgttctctgt gtttgatgag 1800
aacaggagct ggtacctgac tgagaacatc cagaggttcc tgcccaaccc tgctggggtg 1860
cagctggagg accctgagtt ccaggccagc aacatcatgc acagcatcaa tggctatgtg 1920
tttgacagcc tgcagctgtc tgtgtgcctg catgaggtgg cctactggta catcctgagc 1980
attggggccc agactgactt cctgtctgtg ttcttctctg gctacacctt caagcacaag 2040
atggtgtatg aggacaccct gaccctgttc cccttctctg gggagactgt gttcatgagc 2100
atggagaacc ctggcctgtg gattctgggc tgccacaact ctgacttcag gaacaggggc 2160
atgactgccc tgctgaaagt ctccagctgt gacaagaaca ctggggacta ctatgaggac 2220
agctatgagg acatctctgc ctacctgctg agcaagaaca atgccattga gcccaggagc 2280
ttcagccaga atgccactaa tgtgtctaac aacagcaaca ccagcaatga cagcaatgtg 2340
tctcccccag tgctgaagag gcaccagagg gagatcacca ggaccaccct gcagtctgac 2400
caggaggaga ttgactatga tgacaccatc tctgtggaga tgaagaagga ggactttgac 2460
atctacgacg aggacgagaa ccagagcccc aggagcttcc agaagaagac caggcactac 2520
ttcattgctg ctgtggagag gctgtgggac tatggcatga gcagcagccc ccatgtgctg 2580
aggaacaggg cccagtctgg ctctgtgccc cagttcaaga aggtggtgtt ccaggagttc 2640
actgatggca gcttcaccca gcccctgtac agaggggagc tgaatgagca cctgggcctg 2700
ctgggcccct acatcagggc tgaggtggag gacaacatca tggtgacctt caggaaccag 2760
gccagcaggc cctacagctt ctacagcagc ctgatcagct atgaggagga ccagaggcag 2820
ggggctgagc ccaggaagaa ctttgtgaag cccaatgaaa ccaagaccta cttctggaag 2880
gtgcagcacc acatggcccc caccaaggat gagtttgact gcaaggcctg ggcctacttc 2940
tctgatgtgg acctggagaa ggatgtgcac tctggcctga ttggccccct gctggtgtgc 3000
cacaccaaca ccctgaaccc tgcccatggc aggcaggtga ctgtgcagga gtttgccctg 3060
ttcttcacca tctttgatga aaccaagagc tggtacttca ctgagaacat ggagaggaac 3120
tgcagggccc cctgcaacat ccagatggag gaccccacct tcaaggagaa ctacaggttc 3180
catgccatca atggctacat catggacacc ctgcctggcc tggtgatggc ccaggaccag 3240
aggatcaggt ggtacctgct gagcatgggc agcaatgaga acatccacag catccacttc 3300
tctggccatg tgttcactgt gaggaagaag gaggagtaca agatggccct gtacaacctg 3360
taccctgggg tgtttgagac tgtggagatg ctgcccagca aggctggcat ctggagggtg 3420
gagtgcctga ttggggagca cctgcatgct ggcatgagca ccctgttcct ggtgtacagc 3480
aacaagtgcc agacccccct gggcatggcc tctggccaca tcagggactt ccagatcact 3540
gcctctggcc agtatggcca gtgggccccc aagctggcca ggctgcacta ctctggcagc 3600
atcaatgcct ggagcaccaa ggagcccttc agctggatca aggtggacct gctggccccc 3660
atgatcatcc atggcatcaa gacccagggg gccaggcaga agttcagcag cctgtacatc 3720
agccagttca tcatcatgta cagcctggat ggcaagaagt ggcagaccta caggggcaac 3780
agcactggca ccctgatggt gttctttggc aatgtggaca gctctggcat caagcacaac 3840
atcttcaacc cccccatcat tgccagatac atcaggctgc accccaccca ctacagcatc 3900
aggagcaccc tgaggatgga gctgatgggc tgtgacctga acagctgcag catgcccctg 3960
ggcatggaga gcaaggccat ctctgatgcc cagatcactg ccagcagcta cttcaccaac 4020
atgtttgcca cctggagccc cagcaaggcc aggctgcacc tgcagggcag gagcaatgcc 4080
tggaggcccc aggtcaacaa ccccaaggag tggctgcagg tggacttcca gaagaccatg 4140
aaggtgactg gggtgaccac ccagggggtg aagagcctgc tgaccagcat gtatgtgaag 4200
gagttcctga tcagcagcag ccaggatggc caccagtgga ccctgttctt ccagaatggc 4260
aaggtgaagg tgttccaggg caaccaggac agcttcaccc ctgtggtgaa cagcctggac 4320
ccccccctgc tgaccagata cctgaggatt cacccccaga gctgggtgca ccagattgcc 4380
ctgaggatgg aggtgctggg ctgtgaggcc caggacctgt actga 4425
Complementary strand to the exemplified FVIII transgene (N6)
SEQ ID NO: 11
tacgtctaac tcgactcgtg gacgaagaag gacacggacg actccaagac gaagagacgg 60
tggtcctcta tgatggaccc ccgacacctc gactcgaccc tgatgtacgt cagactggac 120
cccctcgacg gacacctacg gtccaagggg gggtctcacg ggttctcgaa ggggaagttg 180
tggagacacc acatgttctt ctgggacaaa cacctcaagt gactggtgga caagttgtaa 240
cggttcgggt ccggggggac ctacccggac gacccggggt ggtaggtccg actccacata 300
ctgtgacacc actagtggga cttcttgtac cggtcggtgg gacactcgga cgtacgacac 360
ccccactcga tgaccttccg gagactcccc cgactcatac tactggtctg gtcggtctcc 420
ctcttcctcc tactgttcca caagggaccc ccgtcggtgt ggatacacac cgtccacgac 480
ttcctcttac cggggtaccg gagactgggg gacacggact ggatgtcgat ggactcggta 540
cacctggacc acttcctgga cttgagaccg gactaacccc gggacgacca cacgtccctc 600
ccgtcggacc ggttcctctt ctgggtctgg gacgtgttca agtaggacga caaacgacac 660
aaactactcc cgttctcgac cgtgagactt tggttcttgt cggactacgt cctgtcccta 720
cgacggagac ggtcccggac cgggttctac gtgtgacact taccgataca cttgtcctcg 780
gacggaccgg actaaccgac ggtgtccttc agacacatga ccgtacacta accgtacccg 840
tggtggggac tccacgtgtc gtagaaggac ctcccggtgt ggaaggacca gtccttggtg 900
tccgtccggt cggacctcta gtcggggtag tggaaggact gacgggtctg ggacgactac 960
ctggacccgg tcaaggacga caagacggtg tagtcgtcgg tggtcgtact accgtacctc 1020
cggatacact tccacctgtc gacgggactc ctcggggtcg actcctactt cttgttactc 1080
ctccgactcc tgatactact actggactga ctgagactct acctacacca ctccaaacta 1140
ctactgttgt cggggtcgaa gtaggtctag tccagacacc ggttcttcgt ggggttctgg 1200
acccacgtga tgtaacgacg actcctcctc ctgaccctga tacgggggga ccacgaccgg 1260
ggactactgt cctcgatgtt ctcggtcatg gacttgttac cgggggtctc ctaaccgtcc 1320
ttcatgttct tccagtccaa gtaccggatg tgactacttt ggaagttctg gtccctccgg 1380
taggtcgtac tcagaccgta ggacccgggg gacgacatac ccctccaccc cctgtgggac 1440
gactagtaga agttcttggt ccggtcgtcc gggatgttgt agatgggggt accgtagtga 1500
ctacactccg gggacatgtc gtcctccgac gggttccccc acttcgtgga cttcctgaag 1560
gggtaggacg gacccctcta gaagttcatg ttcacctgac actgacacct cctaccgggg 1620
tggttcagac tggggtccac ggactggtct atgatgtcgt cgaaacactt gtacctctcc 1680
ctggaccgga gaccggacta accgggggac gactagacga tgttcctcag acacctggtc 1740
tccccgttgg tctagtacag actgttctcc ttacactagg acaagagaca caaactactc 1800
ttgtcctcga ccatggactg actcttgtag gtctccaagg acgggttggg acgaccccac 1860
gtcgacctcc tgggactcaa ggtccggtcg ttgtagtacg tgtcgtagtt accgatacac 1920
aaactgtcgg acgtcgacag acacacggac gtactccacc ggatgaccat gtaggactcg 1980
taaccccggg tctgactgaa ggacagacac aagaagagac cgatgtggaa gttcgtgttc 2040
taccacatac tcctgtggga ctgggacaag gggaagagac ccctctgaca caagtactcg 2100
tacctcttgg gaccggacac ctaagacccg acggtgttga gactgaagtc cttgtccccg 2160
tactgacggg acgactttca gaggtcgaca ctgttcttgt gacccctgat gatactcctg 2220
tcgatactcc tgtagagacg gatggacgac tcgttcttgt tacggtaact cgggtcctcg 2280
aagtcggtct tgtcgtccgt ggggtcgtgg tccgtcttcg tcaagttacg gtggtggtag 2340
ggactcttac tgtatctctt ctgtctgggt accaaacggg tggcctgggg gtacgggttc 2400
taggtcttac actcgtcgag actggacgac tacgacgact ccgtctcggg gtggggggta 2460
ccggactcgg acagactgga cgtcctccgg ttcatacttt ggaagagact actggggtcg 2520
ggaccccggt aactgtcgtt gttgtcggac agactctact gggtgaagtc cggggtcgac 2580
gtggtgagac ccctgtacca caagtgggga ctcagaccgg acgtcgactc cgacttactc 2640
ttcgacccgt ggtgacgacg gtgactcgac ttcttcgacc tgaagtttca gaggtcgtgg 2700
tcgttgttgg actagtcgtg gtaggggaga ctgttggacc gacgaccgtg actgttgtgg 2760
tcgtcggacc cgggggggtc gtacggacac gtgatactgt cggtcgacct gtggtgggac 2820
aaaccgttct tctcgtcggg ggactgactc agacccccgg gggactcgga cagactcctc 2880
ttgttactgt cgttcgacga cctcagaccg gactacttgt cggtcctctc gtcgaccccg 2940
ttcttacact cgtcgtccct ctagtggtcc tggtgggacg tcagactggt cctcctctaa 3000
ctgatactac tgtggtagag acacctctac ttcttcctcc tgaaactgta gatgctgctc 3060
ctgctcttgg tctcggggtc ctcgaaggtc ttcttctggt ccgtgatgaa gtaacgacga 3120
cacctctccg acaccctgat accgtactcg tcgtcggggg tacacgactc cttgtcccgg 3180
gtcagaccga gacacggggt caagttcttc caccacaagg tcctcaagtg actaccgtcg 3240
aagtgggtcg gggacatgtc tcccctcgac ttactcgtgg acccggacga cccggggatg 3300
tagtcccgac tccacctcct gttgtagtac cactggaagt ccttggtccg gtcgtccggg 3360
atgtcgaaga tgtcgtcgga ctagtcgata ctcctcctgg tctccgtccc ccgactcggg 3420
tccttcttga aacacttcgg gttactttgg ttctggatga agaccttcca cgtcgtggtg 3480
taccgggggt ggttcctact caaactgacg ttccggaccc ggatgaagag actacacctg 3540
gacctcttcc tacacgtgag accggactaa ccgggggacg accacacggt gtggttgtgg 3600
gacttgggac gggtaccgtc cgtccactga cacgtcctca aacgggacaa gaagtggtag 3660
aaactacttt ggttctcgac catgaagtga ctcttgtacc tctccttgac gtcccggggg 3720
acgttgtagg tctacctcct ggggtggaag ttcctcttga tgtccaaggt acggtagtta 3780
ccgatgtagt acctgtggga cggaccggac cactaccggg tcctggtctc ctagtccacc 3840
atggacgact cgtacccgtc gttactcttg taggtgtcgt aggtgaagag accggtacac 3900
aagtgacact ccttcttcct cctcatgttc taccgggaca tgttggacat gggaccccac 3960
aaactctgac acctctacga cgggtcgttc cgaccgtaga cctcccacct cacggactaa 4020
cccctcgtgg acgtacgacc gtactcgtgg gacaaggacc acatgtcgtt gttcacggtc 4080
tggggggacc cgtaccggag accggtgtag tccctgaagg tctagtgacg gagaccggtc 4140
ataccggtca cccgggggtt cgaccggtcc gacgtgatga gaccgtcgta gttacggacc 4200
tcgtggttcc tcgggaagtc gacctagttc cacctggacg accgggggta ctagtaggta 4260
ccgtagttct gggtcccccg gtccgtcttc aagtcgtcgg acatgtagtc ggtcaagtag 4320
tagtacatgt cggacctacc gttcttcacc gtctggatgt ccccgttgtc gtgaccgtgg 4380
gactaccaca agaaaccgtt acacctgtcg agaccgtagt tcgtgttgta gaagttgggg 4440
gggtagtaac ggtctatgta gtccgacgtg gggtgggtga tgtcgtagtc ctcgtgggac 4500
tcctacctcg actacccgac actggacttg tcgacgtcgt acggggaccc gtacctctcg 4560
ttccggtaga gactacgggt ctagtgacgg tcgtcgatga agtggttgta caaacggtgg 4620
acctcggggt cgttccggtc cgacgtggac gtcccgtcct cgttacggac ctccggggtc 4680
cagttgttgg ggttcctcac cgacgtccac ctgaaggtct tctggtactt ccactgaccc 4740
cactggtggg tcccccactt ctcggacgac tggtcgtaca tacacttcct caaggactag 4800
tcgtcgtcgg tcctaccggt ggtcacctgg gacaagaagg tcttaccgtt ccacttccac 4860
aaggtcccgt tggtcctgtc gaagtgggga caccacttgt cggacctggg gggggacgac 4920
tggtctatgg actcctaagt gggggtctcg acccacgtgg tctaacggga ctcctacctc 4980
cacgacccga cactccgggt cctggacatg act 5013
Complementary strand to the exemplified FVIII transgene (V3)
SEQ ID NO: 12
tacgtctaac tcgactcgtg gacgaagaag gacacggacg actccaagac gaagagacgg 60
tggtcctcta tgatggaccc ccgacacctc gactcgaccc tgatgtacgt cagactggac 120
cccctcgacg gacacctacg gtccaagggg gggtctcacg ggttctcgaa ggggaagttg 180
tggagacacc acatgttctt ctgggacaaa cacctcaagt gactggtgga caagttgtaa 240
cggttcgggt ccggggggac ctacccggac gacccggggt ggtaggtccg actccacata 300
ctgtgacacc actagtggga cttcttgtac cggtcggtgg gacactcgga cgtacgacac 360
ccccactcga tgaccttccg gagactcccc cgactcatac tactggtctg gtcggtctcc 420
ctcttcctcc tactgttcca caagggaccc ccgtcggtgt ggatacacac cgtccacgac 480
ttcctcttac cggggtaccg gagactgggg gacacggact ggatgtcgat ggactcggta 540
cacctggacc acttcctgga cttgagaccg gactaacccc gggacgacca cacgtccctc 600
ccgtcggacc ggttcctctt ctgggtctgg gacgtgttca agtaggacga caaacgacac 660
aaactactcc cgttctcgac cgtgagactt tggttcttgt cggactacgt cctgtcccta 720
cgacggagac ggtcccggac cgggttctac gtgtgacact taccgataca cttgtcctcg 780
gacggaccgg actaaccgac ggtgtccttc agacacatga ccgtacacta accgtacccg 840
tggtggggac tccacgtgtc gtagaaggac ctcccggtgt ggaaggacca gtccttggtg 900
tccgtccggt cggacctcta gtcggggtag tggaaggact gacgggtctg ggacgactac 960
ctggacccgg tcaaggacga caagacggtg tagtcgtcgg tggtcgtact accgtacctc 1020
cggatacact tccacctgtc gacgggactc ctcggggtcg actcctactt cttgttactc 1080
ctccgactcc tgatactact actggactga ctgagactct acctacacca ctccaaacta 1140
ctactgttgt cggggtcgaa gtaggtctag tccagacacc ggttcttcgt ggggttctgg 1200
acccacgtga tgtaacgacg actcctcctc ctgaccctga tacgggggga ccacgaccgg 1260
ggactactgt cctcgatgtt ctcggtcatg gacttgttac cgggggtctc ctaaccgtcc 1320
ttcatgttct tccagtccaa gtaccggatg tgactacttt ggaagttctg gtccctccgg 1380
taggtcgtac tcagaccgta ggacccgggg gacgacatac ccctccaccc cctgtgggac 1440
gactagtaga agttcttggt ccggtcgtcc gggatgttgt agatgggggt accgtagtga 1500
ctacactccg gggacatgtc gtcctccgac gggttccccc acttcgtgga cttcctgaag 1560
gggtaggacg gacccctcta gaagttcatg ttcacctgac actgacacct cctaccgggg 1620
tggttcagac tggggtccac ggactggtct atgatgtcgt cgaaacactt gtacctctcc 1680
ctggaccgga gaccggacta accgggggac gactagacga tgttcctcag acacctggtc 1740
tccccgttgg tctagtacag actgttctcc ttacactagg acaagagaca caaactactc 1800
ttgtcctcga ccatggactg actcttgtag gtctccaagg acgggttggg acgaccccac 1860
gtcgacctcc tgggactcaa ggtccggtcg ttgtagtacg tgtcgtagtt accgatacac 1920
aaactgtcgg acgtcgacag acacacggac gtactccacc ggatgaccat gtaggactcg 1980
taaccccggg tctgactgaa ggacagacac aagaagagac cgatgtggaa gttcgtgttc 2040
taccacatac tcctgtggga ctgggacaag gggaagagac ccctctgaca caagtactcg 2100
tacctcttgg gaccggacac ctaagacccg acggtgttga gactgaagtc cttgtccccg 2160
tactgacggg acgactttca gaggtcgaca ctgttcttgt gacccctgat gatactcctg 2220
tcgatactcc tgtagagacg gatggacgac tcgttcttgt tacggtaact cgggtcctcg 2280
aagtcggtct tacggtgatt acacagattg ttgtcgttgt ggtcgttact gtcgttacac 2340
agagggggtc acgacttctc cgtggtctcc ctctagtggt cctggtggga cgtcagactg 2400
gtcctcctct aactgatact actgtggtag agacacctct acttcttcct cctgaaactg 2460
tagatgctgc tcctgctctt ggtctcgggg tcctcgaagg tcttcttctg gtccgtgatg 2520
aagtaacgac gacacctctc cgacaccctg ataccgtact cgtcgtcggg ggtacacgac 2580
tccttgtccc gggtcagacc gagacacggg gtcaagttct tccaccacaa ggtcctcaag 2640
tgactaccgt cgaagtgggt cggggacatg tctcccctcg acttactcgt ggacccggac 2700
gacccgggga tgtagtcccg actccacctc ctgttgtagt accactggaa gtccttggtc 2760
cggtcgtccg ggatgtcgaa gatgtcgtcg gactagtcga tactcctcct ggtctccgtc 2820
ccccgactcg ggtccttctt gaaacacttc gggttacttt ggttctggat gaagaccttc 2880
cacgtcgtgg tgtaccgggg gtggttccta ctcaaactga cgttccggac ccggatgaag 2940
agactacacc tggacctctt cctacacgtg agaccggact aaccggggga cgaccacacg 3000
gtgtggttgt gggacttggg acgggtaccg tccgtccact gacacgtcct caaacgggac 3060
aagaagtggt agaaactact ttggttctcg accatgaagt gactcttgta cctctccttg 3120
acgtcccggg ggacgttgta ggtctacctc ctggggtgga agttcctctt gatgtccaag 3180
gtacggtagt taccgatgta gtacctgtgg gacggaccgg accactaccg ggtcctggtc 3240
tcctagtcca ccatggacga ctcgtacccg tcgttactct tgtaggtgtc gtaggtgaag 3300
agaccggtac acaagtgaca ctccttcttc ctcctcatgt tctaccggga catgttggac 3360
atgggacccc acaaactctg acacctctac gacgggtcgt tccgaccgta gacctcccac 3420
ctcacggact aacccctcgt ggacgtacga ccgtactcgt gggacaagga ccacatgtcg 3480
ttgttcacgg tctgggggga cccgtaccgg agaccggtgt agtccctgaa ggtctagtga 3540
cggagaccgg tcataccggt cacccggggg ttcgaccggt ccgacgtgat gagaccgtcg 3600
tagttacgga cctcgtggtt cctcgggaag tcgacctagt tccacctgga cgaccggggg 3660
tactagtagg taccgtagtt ctgggtcccc cggtccgtct tcaagtcgtc ggacatgtag 3720
tcggtcaagt agtagtacat gtcggaccta ccgttcttca ccgtctggat gtccccgttg 3780
tcgtgaccgt gggactacca caagaaaccg ttacacctgt cgagaccgta gttcgtgttg 3840
tagaagttgg gggggtagta acggtctatg tagtccgacg tggggtgggt gatgtcgtag 3900
tcctcgtggg actcctacct cgactacccg acactggact tgtcgacgtc gtacggggac 3960
ccgtacctct cgttccggta gagactacgg gtctagtgac ggtcgtcgat gaagtggttg 4020
tacaaacggt ggacctcggg gtcgttccgg tccgacgtgg acgtcccgtc ctcgttacgg 4080
acctccgggg tccagttgtt ggggttcctc accgacgtcc acctgaaggt cttctggtac 4140
ttccactgac cccactggtg ggtcccccac ttctcggacg actggtcgta catacacttc 4200
ctcaaggact agtcgtcgtc ggtcctaccg gtggtcacct gggacaagaa ggtcttaccg 4260
ttccacttcc acaaggtccc gttggtcctg tcgaagtggg gacaccactt gtcggacctg 4320
gggggggacg actggtctat ggactcctaa gtgggggtct cgacccacgt ggtctaacgg 4380
gactcctacc tccacgaccc gacactccgg gtcctggaca tgact 4425
Exemplified FVIII polypeptide (N6)
SEQ ID NO: 13
Met Gln Ile Glu Leu Ser Thr Cys Phe Phe Leu Cys Leu Leu Arg Phe
1 5 10 15
Cys Phe Ser Ala Thr Arg Arg Tyr Tyr Leu Gly Ala Val Glu Leu Ser
                        20 25 30
Trp Asp Tyr Met Gln Ser Asp Leu Gly Glu Leu Pro Val Asp Ala Arg
                35 40 45
Phe Pro Pro Arg Val Pro Lys Ser Phe Pro Phe Asn Thr Ser Val Val
        50 55 60
Tyr Lys Lys Thr Leu Phe Val Glu Phe Thr Asp His Leu Phe Asn Ile
65 70 75 80
Ala Lys Pro Arg Pro Pro Trp Met Gly Leu Leu Gly Pro Thr Ile Gln
                                85 90 95
Ala Glu Val Tyr Asp Thr Val Val Ile Thr Leu Lys Asn Met Ala Ser
                        100 105 110
His Pro Val Ser Leu His Ala Val Gly Val Ser Tyr Trp Lys Ala Ser
                115 120 125
Glu Gly Ala Glu Tyr Asp Asp Gln Thr Ser Gln Arg Glu Lys Glu Asp
        130 135 140
Asp Lys Val Phe Pro Gly Gly Ser His Thr Tyr Val Trp Gln Val Leu
145 150 155 160
Lys Glu Asn Gly Pro Met Ala Ser Asp Pro Leu Cys Leu Thr Tyr Ser
                                165 170 175
Tyr Leu Ser His Val Asp Leu Val Lys Asp Leu Asn Ser Gly Leu Ile
                      180 185 190
Gly Ala Leu Leu Val Cys Arg Glu Gly Ser Leu Ala Lys Glu Lys Thr
                195 200 205
Gln Thr Leu His Lys Phe Ile Leu Leu Phe Ala Val Phe Asp Glu Gly
        210 215 220
Lys Ser Trp His Ser Glu Thr Lys Asn Ser Leu Met Gln Asp Arg Asp
225 230 235 240
Ala Ala Ser Ala Arg Ala Trp Pro Lys Met His Thr Val Asn Gly Tyr
                                245 250 255
Val Asn Arg Ser Leu Pro Gly Leu Ile Gly Cys His Arg Lys Ser Val
                        260 265 270
Tyr Trp His Val Ile Gly Met Gly Thr Thr Pro Glu Val His Ser Ile
              275 280 285
Phe Leu Glu Gly His Thr Phe Leu Val Arg Asn His Arg Gln Ala Ser
        290 295 300
Leu Glu Ile Ser Pro Ile Thr Phe Leu Thr Ala Gln Thr Leu Leu Met
305 310 315 320
Asp Leu Gly Gln Phe Leu Leu Phe Cys His Ile Ser Ser His Gln His
                                325 330 335
Asp Gly Met Glu Ala Tyr Val Lys Val Asp Ser Cys Pro Glu Glu Pro
                        340 345 350
Gln Leu Arg Met Lys Asn Asn Glu Glu Ala Glu Asp Tyr Asp Asp Asp
                355 365 360
Leu Thr Asp Ser Glu Met Asp Val Val Arg Phe Asp Asp Asp Asn Ser
        370 375 380
Pro Ser Phe Ile Gln Ile Arg Ser Val Ala Lys Lys His Pro Lys Thr
385 390 395 400
Trp Val His Tyr Ile Ala Ala Glu Glu Glu Asp Trp Asp Tyr Ala Pro
                                405 410 415
Leu Val Leu Ala Pro Asp Asp Arg Ser Tyr Lys Ser Gln Tyr Leu Asn
                        420 425 430
Asn Gly Pro Gln Arg Ile Gly Arg Lys Tyr Lys Lys Val Arg Phe Met
                435 440 445
Ala Tyr Thr Asp Glu Thr Phe Lys Thr Arg Glu Ala Ile Gln His Glu
        450 455 460
Ser Gly Ile Leu Gly Pro Leu Leu Tyr Gly Glu Val Gly Asp Thr Leu
465 470 475 480
Leu Ile Ile Phe Lys Asn Gln Ala Ser Arg Pro Tyr Asn Ile Tyr Pro
                                485 490 495
His Gly Ile Thr Asp Val Arg Pro Leu Tyr Ser Arg Arg Leu Pro Lys
                        500 505 510
Gly Val Lys His Leu Lys Asp Phe Pro Ile Leu Pro Gly Glu Ile Phe
                515 520 525
Lys Tyr Lys Trp Thr Val Thr Val Glu Asp Gly Pro Thr Lys Ser Asp
        530 535 540
Pro Arg Cys Leu Thr Arg Tyr Tyr Ser Ser Phe Val Asn Met Glu Arg
545 550 555 560
Asp Leu Ala Ser Gly Leu Ile Gly Pro Leu Leu Ile Cys Tyr Lys Glu
                                565 570 575
Ser Val Asp Gln Arg Gly Asn Gln Ile Met Ser Asp Lys Arg Asn Val
                        580 585 590
Ile Leu Phe Ser Val Phe Asp Glu Asn Arg Ser Trp Tyr Leu Thr Glu
                595 600 605
Asn Ile Gln Arg Phe Leu Pro Asn Pro Ala Gly Val Gln Leu Glu Asp
        610 615 620
Pro Glu Phe Gln Ala Ser Asn Ile Met His Ser Ile Asn Gly Tyr Val
625 630 635 640
Phe Asp Ser Leu Gln Leu Ser Val Cys Leu His Glu Val Ala Tyr Trp
                                645 650 655
Tyr Ile Leu Ser Ile Gly Ala Gln Thr Asp Phe Leu Ser Val Phe Phe
                        660 665 670
Ser Gly Tyr Thr Phe Lys His Lys Met Val Tyr Glu Asp Thr Leu Thr
                675 680 685
Leu Phe Pro Phe Ser Gly Glu Thr Val Phe Met Ser Met Glu Asn Pro
        690 695 700
Gly Leu Trp Ile Leu Gly Cys His Asn Ser Asp Phe Arg Asn Arg Gly
705 710 715 720
Met Thr Ala Leu Leu Lys Val Ser Ser Cys Asp Lys Asn Thr Gly Asp
                                725 730 735
Tyr Tyr Glu Asp Ser Tyr Glu Asp Ile Ser Ala Tyr Leu Leu Ser Lys
                        740 745 750
Asn Asn Ala Ile Glu Pro Arg Ser Phe Ser Gln Asn Ser Arg His Pro
                755 760 765
Ser Thr Arg Gln Lys Gln Phe Asn Ala Thr Thr Ile Pro Glu Asn Asp
        770 775 780
Ile Glu Lys Thr Asp Pro Trp Phe Ala His Arg Thr Pro Met Pro Lys
785 790 795 800
Ile Gln Asn Val Ser Ser Ser Asp Leu Leu Met Leu Leu Arg Gln Ser
                                805 810 815
Pro Thr Pro His Gly Leu Ser Leu Ser Asp Leu Gln Glu Ala Lys Tyr
                        320 825 830
Glu Thr Phe Ser Asp Asp Pro Ser Pro Gly Ala Ile Asp Ser Asn Asn
                835 840 845
Ser Leu Ser Glu Met Thr His Phe Arg Pro Gln Leu His His Ser Gly
        850 855 860
Asp Met Val Phe Thr Pro Glu Ser Gly Leu Gln Leu Arg Leu Asn Glu
865 870 875 880
Lys Leu Gly Thr Thr Ala Ala Thr Glu Leu Lys Lys Leu Asp Phe Lys
                                885 890 895
Val Ser Ser Thr Ser Asn Asn Leu Ile Ser Thr Ile Pro Ser Asp Asn
                        900 905 910
Leu Ala Ala Gly Thr Asp Asn Thr Ser Ser Leu Gly Pro Pro Ser Met
                915 920 925
Pro Val His Tyr Asp Ser Gln Leu Asp Thr Thr Leu Phe Gly Lys Lys
        930 935 940
Ser Ser Pro Leu Thr Glu Ser Gly Gly Pro Leu Ser Leu Ser Glu Glu
945 950 955 960
Asn Asn Asp Ser Lys Leu Leu Glu Ser Gly Leu Met Asn Ser Gln Glu
                                965 970 975
Ser Ser Trp Gly Lys Asn Val Ser Ser Arg Glu Ile Thr Arg Thr Thr
                        980 985 990
Leu Gln Ser Asp Gln Glu Glu Ile Asp Tyr Asp Asp Thr Ile Ser Val
                995 1000 1005
Glu Met Lys Lys Glu Asp Phe Asp Ile Tyr Asp Glu Asp Glu Asn
        1010 1015 1020
Gln Ser Pro Arg Ser Phe Gln Lys Lys Thr Arg His Tyr Phe Ile
        1025 1030 1035
Ala Ala Val Glu Arg Leu Trp Asp Tyr Gly Met Ser Ser Ser Pro
        1040 1045 1050
His Val Leu Arg Asn Arg Ala Gln Ser Gly Ser Val Pro Gln Phe
        1055 1060 1065
Lys Lys Val Val Phe Gln Glu Phe Thr Asp Gly Ser Phe Thr Gln
        1070 1075 1080
Pro Leu Tyr Arg Gly Glu Leu Asn Glu His Leu Gly Leu Leu Gly
        1085 1090 1095
Pro Tyr Ile Arg Ala Glu Val Glu Asp Asn Ile Met Val Thr Phe
        1100 1105 1110
Arg Asn Gln Ala Ser Arg Pro Tyr Ser Phe Tyr Ser Ser Leu Ile
        1115 1120 1125
Ser Tyr Glu Glu Asp Gln Arg Gln Gly Ala Glu Pro Arg Lys Asn
        1130 1135 1140
Phe Val Lys Pro Asn Glu Thr Lys Thr Tyr Phe Trp Lys Val Gln
        1145 1150 1155
His His Met Ala Pro Thr Lys Asp Glu Phe Asp Cys Lys Ala Trp
        1160 1165 1170
Ala Tyr Phe Ser Asp Val Asp Leu Glu Lys Asp Val His Ser Gly
        1175 1180 1185
Leu Ile Gly Pro Leu Leu Val Cys His Thr Asn Thr Leu Asn Pro
        1190 1195 1200
Ala His Gly Arg Gln Val Thr Val Gln Glu Phe Ala Leu Phe Phe
        1205 1210 1215
Thr Ile Phe Asp Glu Thr Lys Ser Trp Tyr Phe Thr Glu Asn Met
        1220 1225 1230
Glu Arg Asn Cys Arg Ala Pro Cys Asn Ile Gln Met Glu Asp Pro
        1235 1240 1245
Thr Phe Lys Glu Asn Tyr Arg Phe His Ala Ile Asn Gly Tyr Ile
        1250 1255 1260
Met Asp Thr Leu Pro Gly Leu Val Met Ala Gln Asp Gln Arg Ile
        1265 1270 1275
Arg Trp Tyr Leu Leu Ser Met Gly Ser Asn Glu Asn Ile His Ser
        1280 1285 1290
Ile His Phe Ser Gly His Val Phe Thr Val Arg Lys Lys Glu Glu
        1295 1300 1305
Tyr Lys Met Ala Leu Tyr Asn Leu Tyr Pro Gly Val Phe Glu Thr
        1310 1315 1320
Val Glu Met Leu Pro Ser Lys Ala Gly Ile Trp Arg Val Glu Cys
        1325 1330 1335
Leu Ile Gly Glu His Leu His Ala Gly Met Ser Thr Leu Phe Leu
        1340 1345 1350
Val Tyr Ser Asn Lys Cys Gln Thr Pro Leu Gly Met Ala Ser Gly
        1355 1360 1365
His Ile Arg Asp Phe Gln Ile Thr Ala Ser Gly Gln Tyr Gly Gln
        1370 1375 1380
Trp Ala Pro Lys Leu Ala Arg Leu His Tyr Ser Gly Ser Ile Asn
        1385 1390 1395
Ala Trp Ser Thr Lys Glu Pro Phe Ser Trp Ile Lys Val Asp Leu
        1400 1405 1410
Leu Ala Pro Met Ile Ile His Gly Ile Lys Thr Gln Gly Ala Arg
        1415 1420 1425
Gln Lys Phe Ser Ser Leu Tyr Ile Ser Gln Phe Ile Ile Met Tyr
        1430 1435 1440
Ser Leu Asp Gly Lys Lys Trp Gln Thr Tyr Arg Gly Asn Ser Thr
        1445 1450 1455
Gly Thr Leu Met Val Phe Phe Gly Asn Val Asp Ser Ser Gly Ile
        1460 1465 1470
Lys His Asn Ile Phe Asn Pro Pro Ile Ile Ala Arg Tyr Ile Arg
        1475 1480 1485
Leu His Pro Thr His Tyr Ser Ile Arg Ser Thr Leu Arg Met Glu
        1490 1495 1500
Leu Met Gly Cys Asp Leu Asn Ser Cys Ser Met Pro Leu Gly Met
        1505 1510 1515
Glu Ser Lys Ala Ile Ser Asp Ala Gln Ile Thr Ala Ser Ser Tyr
        1520 1525 1530
Phe Thr Asn Met Phe Ala Thr Trp Ser Pro Ser Lys Ala Arg Leu
        1535 1540 1545
His Leu Gln Gly Arg Ser Asn Ala Trp Arg Pro Gln Val Asn Asn
        1550 1555 1560
Pro Lys Glu Trp Leu Gln Val Asp Phe Gln Lys Thr Met Lys Val
        1565 1570 1575
Thr Gly Val Thr Thr Gln Gly Val Lys Ser Leu Leu Thr Ser Met
        1580 1585 1590
Tyr Val Lys Glu Phe Leu Ile Ser Ser Ser Gln Asp Gly His Gln
        1595 1600 1605
Trp Thr Leu Phe Phe Gln Asn Gly Lys Val Lys Val Phe Gln Gly
        1610 1615 1620
Asn Gln Asp Ser Phe Thr Pro Val Val Asn Ser Leu Asp Pro Pro
        1625 1630 1635
Leu Leu Thr Arg Tyr Leu Arg Ile His Pro Gln Ser Trp Val His
        1640 1645 1650
Gln Ile Ala Leu Arg Met Glu Val Leu Gly Cys Glu Ala Gln Asp
        1655 1660 1665
Leu Tyr
        1670
Exemplified FVIII polypeptide (V3)
SEQ ID NO: 14
Met Gln Ile Glu Leu Ser Thr Cys Phe Phe Leu Cys Leu Leu Arg Phe
1 5 10 15
Cys Phe Ser Ala Thr Arg Arg Tyr Tyr Leu Gly Ala Val Glu Leu Ser
                        20 25 30
Trp Asp Tyr Met Gln Ser Asp Leu Gly Glu Leu Pro Val Asp Ala Arg
                35 40 45
Phe Pro Pro Arg Val Pro Lys Ser Phe Pro Phe Asn Thr Ser Val Val
        50 55 60
Tyr Lys Lys Thr Leu Phe Val Glu Phe Thr Asp His Leu Phe Asn Ile
65 70 75 80
Ala Lys Pro Arg Pro Pro Trp Met Gly Leu Leu Gly Pro Thr Ile Gln
                                85 90 95
Ala Glu Val Tyr Asp Thr Val Val Ile Thr Leu Lys Asn Met Ala Ser
                        100 105 110
His Pro Val Ser Leu His Ala Val Gly Val Ser Tyr Trp Lys Ala Ser
                115 120 125
Glu Gly Ala Glu Tyr Asp Asp Gln Thr Ser Gln Arg Glu Lys Glu Asp
        130 135 140
Asp Lys Val Phe Pro Gly Gly Ser His Thr Tyr Val Trp Gln Val Leu
145 150 155 160
Lys Glu Asn Gly Pro Met Ala Ser Asp Pro Leu Cys Leu Thr Tyr Ser
                                165 170 175
Tyr Leu Ser His Val Asp Leu Val Lys Asp Leu Asn Ser Gly Leu Ile
                      180 185 190
Gly Ala Leu Leu Val Cys Arg Glu Gly Ser Leu Ala Lys Glu Lys Thr
                195 200 205
Gln Thr Leu His Lys Phe Ile Leu Leu Phe Ala Val Phe Asp Glu Gly
        210 215 220
Lys Ser Trp His Ser Glu Thr Lys Asn Ser Leu Met Gln Asp Arg Asp
225 230 235 240
Ala Ala Ser Ala Arg Ala Trp Pro Lys Met His Thr Val Asn Gly Tyr
                                245 250 255
Val Asn Arg Ser Leu Pro Gly Leu Ile Gly Cys His Arg Lys Ser Val
                        260 265 270
Tyr Trp His Val Ile Gly Met Gly Thr Thr Pro Glu Val His Ser Ile
              275 280 285
Phe Leu Glu Gly His Thr Phe Leu Val Arg Asn His Arg Gln Ala Ser
        290 295 300
Leu Glu Ile Ser Pro Ile Thr Phe Leu Thr Ala Gln Thr Leu Leu Met
305 310 315 320
Asp Leu Gly Gln Phe Leu Leu Phe Cys His Ile Ser Ser His Gln His
                                325 330 335
Asp Gly Met Glu Ala Tyr Val Lys Val Asp Ser Cys Pro Glu Glu Pro
                        340 345 350
Gln Leu Arg Met Lys Asn Asn Glu Glu Ala Glu Asp Tyr Asp Asp Asp
                355 365 360
Leu Thr Asp Ser Glu Met Asp Val Val Arg Phe Asp Asp Asp Asn Ser
        370 375 380
Pro Ser Phe Ile Gln Ile Arg Ser Val Ala Lys Lys His Pro Lys Thr
385 390 395 400
Trp Val His Tyr Ile Ala Ala Glu Glu Glu Asp Trp Asp Tyr Ala Pro
                                405 410 415
Leu Val Leu Ala Pro Asp Asp Arg Ser Tyr Lys Ser Gln Tyr Leu Asn
                        420 425 430
Asn Gly Pro Gln Arg Ile Gly Arg Lys Tyr Lys Lys Val Arg Phe Met
                435 440 445
Ala Tyr Thr Asp Glu Thr Phe Lys Thr Arg Glu Ala Ile Gln His Glu
        450 455 460
Ser Gly Ile Leu Gly Pro Leu Leu Tyr Gly Glu Val Gly Asp Thr Leu
465 470 475 480
Leu Ile Ile Phe Lys Asn Gln Ala Ser Arg Pro Tyr Asn Ile Tyr Pro
                                485 490 495
His Gly Ile Thr Asp Val Arg Pro Leu Tyr Ser Arg Arg Leu Pro Lys
                        500 505 510
Gly Val Lys His Leu Lys Asp Phe Pro Ile Leu Pro Gly Glu Ile Phe
                515 520 525
Lys Tyr Lys Trp Thr Val Thr Val Glu Asp Gly Pro Thr Lys Ser Asp
        530 535 540
Pro Arg Cys Leu Thr Arg Tyr Tyr Ser Ser Phe Val Asn Met Glu Arg
545 550 555 560
Asp Leu Ala Ser Gly Leu Ile Gly Pro Leu Leu Ile Cys Tyr Lys Glu
                                565 570 575
Ser Val Asp Gln Arg Gly Asn Gln Ile Met Ser Asp Lys Arg Asn Val
                        580 585 590
Ile Leu Phe Ser Val Phe Asp Glu Asn Arg Ser Trp Tyr Leu Thr Glu
                595 600 605
Asn Ile Gln Arg Phe Leu Pro Asn Pro Ala Gly Val Gln Leu Glu Asp
        610 615 620
Pro Glu Phe Gln Ala Ser Asn Ile Met His Ser Ile Asn Gly Tyr Val
625 630 635 640
Phe Asp Ser Leu Gln Leu Ser Val Cys Leu His Glu Val Ala Tyr Trp
                                645 650 655
Tyr Ile Leu Ser Ile Gly Ala Gln Thr Asp Phe Leu Ser Val Phe Phe
                        660 665 670
Ser Gly Tyr Thr Phe Lys His Lys Met Val Tyr Glu Asp Thr Leu Thr
                675 680 685
Leu Phe Pro Phe Ser Gly Glu Thr Val Phe Met Ser Met Glu Asn Pro
        690 695 700
Gly Leu Trp Ile Leu Gly Cys His Asn Ser Asp Phe Arg Asn Arg Gly
705 710 715 720
Met Thr Ala Leu Leu Lys Val Ser Ser Cys Asp Lys Asn Thr Gly Asp
                                725 730 735
Tyr Tyr Glu Asp Ser Tyr Glu Asp Ile Ser Ala Tyr Leu Leu Ser Lys
                        740 745 750
Asn Asn Ala Ile Glu Pro Arg Ser Phe Ser Gln Asn Ala Thr Asn Val
                755 760 765
Ser Asn Asn Ser Asn Thr Ser Asn Asp Ser Asn Val Ser Pro Pro Val
        770 775 780
Leu Lys Arg His Gln Arg Glu Ile Thr Arg Thr Thr Leu Gln Ser Asp
785 790 795 800
Gln Glu Glu Ile Asp Tyr Asp Asp Thr Ile Ser Val Glu Met Lys Lys
                                805 810 815
Glu Asp Phe Asp Ile Tyr Asp Glu Asp Glu Asn Gln Ser Pro Arg Ser
                        820 825 830
Phe Gln Lys Lys Thr Arg His Tyr Phe Ile Ala Ala Val Glu Arg Leu
                835 840 845
Trp Asp Tyr Gly Met Ser Ser Ser Pro His Val Leu Arg Asn Arg Ala
        850 855 860
Gln Ser Gly Ser Val Pro Gln Phe Lys Lys Val Val Phe Gln Glu Phe
865 870 875 880
Thr Asp Gly Ser Phe Thr Gln Pro Leu Tyr Arg Gly Glu Leu Asn Glu
                                885 890 895
His Leu Gly Leu Leu Gly Pro Tyr Ile Arg Ala Glu Val Glu Asp Asn
                        900 905 910
Ile Met Val Thr Phe Arg Asn Gln Ala Ser Arg Pro Tyr Ser Phe Tyr
              915 920 925
Ser Ser Leu Ile Ser Tyr Glu Glu Asp Gln Arg Gln Gly Ala Glu Pro
        930 935 940
Arg Lys Asn Phe Val Lys Pro Asn Glu Thr Lys Thr Tyr Phe Trp Lys
945 950 955 960
Val Gln His His Met Ala Pro Thr Lys Asp Glu Phe Asp Cys Lys Ala
                                965 970 975
Trp Ala Tyr Phe Ser Asp Val Asp Leu Glu Lys Asp Val His Ser Gly
                        980 985 990
Leu Ile Gly Pro Leu Leu Val Cys His Thr Asn Thr Leu Asn Pro Ala
                995 1000 1005
His Gly Arg Gln Val Thr Val Gln Glu Phe Ala Leu Phe Phe Thr
        1010 1015 1020
Ile Phe Asp Glu Thr Lys Ser Trp Tyr Phe Thr Glu Asn Met Glu
        1025 1030 1035
Arg Asn Cys Arg Ala Pro Cys Asn Ile Gln Met Glu Asp Pro Thr
        1040 1045 1050
Phe Lys Glu Asn Tyr Arg Phe His Ala Ile Asn Gly Tyr Ile Met
        1055 1060 1065
Asp Thr Leu Pro Gly Leu Val Met Ala Gln Asp Gln Arg Ile Arg
        1070 1075 1080
Trp Tyr Leu Leu Ser Met Gly Ser Asn Glu Asn Ile His Ser Ile
        1085 1090 1095
His Phe Ser Gly His Val Phe Thr Val Arg Lys Lys Glu Glu Tyr
        1100 1105 1110
Lys Met Ala Leu Tyr Asn Leu Tyr Pro Gly Val Phe Glu Thr Val
        1115 1120 1125
Glu Met Leu Pro Ser Lys Ala Gly Ile Trp Arg Val Glu Cys Leu
        1130 1135 1140
Ile Gly Glu His Leu His Ala Gly Met Ser Thr Leu Phe Leu Val
        1145 1150 1155
Tyr Ser Asn Lys Cys Gln Thr Pro Leu Gly Met Ala Ser Gly His
        1160 1165 1170
Ile Arg Asp Phe Gln Ile Thr Ala Ser Gly Gln Tyr Gly Gln Trp
        1175 1180 1185
Ala Pro Lys Leu Ala Arg Leu His Tyr Ser Gly Ser Ile Asn Ala
        1190 1195 1200
Trp Ser Thr Lys Glu Pro Phe Ser Trp Ile Lys Val Asp Leu Leu
        1205 1210 1215
Ala Pro Met Ile Ile His Gly Ile Lys Thr Gln Gly Ala Arg Gln
        1220 1225 1230
Lys Phe Ser Ser Leu Tyr Ile Ser Gln Phe Ile Ile Met Tyr Ser
        1235 1240 1245
Leu Asp Gly Lys Lys Trp Gln Thr Tyr Arg Gly Asn Ser Thr Gly
        1250 1255 1260
Thr Leu Met Val Phe Phe Gly Asn Val Asp Ser Ser Gly Ile Lys
        1265 1270 1275
His Asn Ile Phe Asn Pro Pro Ile Ile Ala Arg Tyr Ile Arg Leu
        1280 1285 1290
His Pro Thr His Tyr Ser Ile Arg Ser Thr Leu Arg Met Glu Leu
        1295 1300 1305
Met Gly Cys Asp Leu Asn Ser Cys Ser Met Pro Leu Gly Met Glu
        1310 1315 1320
Ser Lys Ala Ile Ser Asp Ala Gln Ile Thr Ala Ser Ser Tyr Phe
        1325 1330 1335
Thr Asn Met Phe Ala Thr Trp Ser Pro Ser Lys Ala Arg Leu His
        1340 1345 1350
Leu Gln Gly Arg Ser Asn Ala Trp Arg Pro Gln Val Asn Asn Pro
        1355 1360 1365
Lys Glu Trp Leu Gln Val Asp Phe Gln Lys Thr Met Lys Val Thr
        1370 1375 1380
Gly Val Thr Thr Gln Gly Val Lys Ser Leu Leu Thr Ser Met Tyr
        1385 1390 1395
Val Lys Glu Phe Leu Ile Ser Ser Ser Gln Asp Gly His Gln Trp
        1400 1405 1410
Thr Leu Phe Phe Gln Asn Gly Lys Val Lys Val Phe Gln Gly Asn
        1415 1420 1425
Gln Asp Ser Phe Thr Pro Val Val Asn Ser Leu Asp Pro Pro Leu
        1430 1435 1440
Leu Thr Arg Tyr Leu Arg Ile His Pro Gln Ser Trp Val His Gln
        1445 1450 1455
Ile Ala Leu Arg Met Glu Val Leu Gly Cys Glu Ala Gln Asp Leu
        1460 1465 1470
Tyr
Exemplified CFTR transgene (soCFTR2)
SEQ ID NO: 15
gctagccac atgcagagaa gccctctgga gaaggcctct gtggtgagca agctgttctt 60
cagctggacc aggcccatcc tgaggaaggg ctacaggcag agactggagc tgtctgacat 120
ctaccagatc ccctctgtgg actctgctga caacctgtct gagaagctgg agagggagtg 180
ggatagagag ctggccagca agaagaaccc caagctgatc aatgccctga ggagatgctt 240
cttctggaga ttcatgttct atggcatctt cctgtacctg ggggaagtga ccaaggctgt 300
gcagcctctg ctgctgggca gaatcattgc cagctatgac cctgacaaca aggaggagag 360
gagcattgcc atctacctgg gcattggcct gtgcctgctg ttcattgtga ggaccctgct 420
gctgcaccct gccatctttg gcctgcacca cattggcatg cagatgagga ttgccatgtt 480
cagcctgatc tacaagaaaa ccctgaagct gtccagcaga gtgctggaca agatcagcat 540
tggccagctg gtgagcctgc tgagcaacaa cctgaacaag tttgatgagg gcctggccct 600
ggcccacttt gtgtggattg cccctctgca ggtggccctg ctgatgggcc tgatttggga 660
gctgctgcag gcctctgcct tttgtggcct gggcttcctg attgtgctgg ccctgtttca 720
ggctggcctg ggcaggatga tgatgaagta cagggaccag agggcaggca agatcagtga 780
gaggctggtg atcacctctg agatgattga gaacatccag tctgtgaagg cctactgttg 840
ggaggaagct atggagaaga tgattgaaaa cctgaggcag acagagctga agctgaccag 900
gaaggctgcc tatgtgagat acttcaacag ctctgccttc ttcttctctg gcttctttgt 960
ggtgttcctg tctgtgctgc cctatgccct gatcaagggg atcatcctga gaaagatttt 1020
caccaccatc agcttctgca ttgtgctgag gatggctgtg accagacagt tcccctgggc 1080
tgtgcagacc tggtatgaca gcctgggggc catcaacaag atccaggact tcctgcagaa 1140
gcaggagtac aagaccctgg agtacaacct gaccaccaca gaagtggtga tggagaatgt 1200
gacagccttc tgggaggagg gctttgggga gctgtttgag aaggccaagc agaacaacaa 1260
caacagaaag accagcaatg gggatgactc cctgttcttc tccaacttct ccctgctggg 1320
cacacctgtg ctgaaggaca tcaacttcaa gattgagagg gggcagctgc tggctgtggc 1380
tggatctaca ggggctggca agaccagcct gctgatgatg atcatggggg agctggagcc 1440
ttctgagggc aagatcaagc actctggcag gatcagcttt tgcagccagt tcagctggat 1500
catgcctggc accatcaagg agaacatcat ctttggagtg agctatgatg agtacagata 1560
caggagtgtg atcaaggcct gccagctgga ggaggacatc agcaagtttg ctgagaagga 1620
caacattgtg ctgggggagg gaggcattac actgtctggg ggccagagag ccagaatcag 1680
cctggccagg gctgtgtaca aggatgctga cctgtacctg ctggactccc cctttggcta 1740
cctggatgtg ctgacagaga aggagatttt tgagagctgt gtgtgcaagc tgatggccaa 1800
caagaccaga atcctggtga ccagcaagat ggagcacctg aagaaggctg acaagatcct 1860
gatcctgcat gagggcagca gctacttcta tgggaccttc tctgagctgc agaacctgca 1920
gcctgacttc agctctaagc tgatgggctg tgacagcttt gaccagttct ctgctgagag 1980
gaggaacagc atcctgacag agaccctgca cagattcagc ctggagggag atgcccctgt 2040
gagctggaca gagaccaaga agcagagctt caagcagaca ggggagtttg gggagaagag 2100
gaagaactcc atcctgaacc ccatcaacag catcaggaag ttcagcattg tgcagaaaac 2160
ccccctgcag atgaatggca ttgaggaaga ttctgatgag cccctggaga ggagactgag 2220
cctggtgcct gattctgagc agggagaggc catcctgcct aggatctctg tgatcagcac 2280
aggccctaca ctgcaggcca gaaggaggca gtctgtgctg aacctgatga cccactctgt 2340
gaaccagggc cagaacatcc acaggaaaac cacagcctcc accaggaaag tgagcctggc 2400
ccctcaggcc aatctgacag agctggacat ctacagcagg aggctgtctc aggagacagg 2460
cctggagatt tctgaggaga tcaatgagga ggacctgaaa gagtgcttct ttgatgacat 2520
ggagagcatc cctgctgtga ccacctggaa cacctacctg agatacatca cagtgcacaa 2580
gagcctgatc tttgtgctga tctggtgcct ggtgatcttc ctggctgaag tggctgcctc 2640
tctggtggtg ctgtggctgc tgggaaacac cccactgcag gacaagggca acagcaccca 2700
cagcaggaac aacagctatg ctgtgatcat cacctccacc tccagctact atgtgttcta 2760
catctatgtg ggagtggctg ataccctgct ggctatgggc ttctttagag gcctgcccct 2820
ggtgcacaca ctgatcacag tgagcaagat cctccaccac aagatgctgc actctgtgct 2880
gcaggctcct atgagcaccc tgaataccct gaaggctggg ggcatcctga acagattctc 2940
caaggatatt gccatcctgg atgacctgct gcctctcacc atctttgact tcatccagct 3000
gctgctgatt gtgattgggg ccattgctgt ggtggcagtg ctgcagccct acatctttgt 3060
ggccacagtg cctgtgattg tggccttcat catgctgagg gcctactttc tgcagacctc 3120
ccagcagctg aagcagctgg agtctgaggg cagaagcccc atcttcaccc acctggtgac 3180
aagcctgaag ggcctgtgga ccctgagagc ctttggcagg cagccctact ttgagaccct 3240
gttccacaag gccctgaacc tgcacacage caactggttc ctctacctgt ccaccctgag 3300
atggttccag atgagaattg agatgatctt tgtcatcttc ttcattgctg tgaccttcat 3360
cagcattctg accacaggag agggagaggg cagagtgggc attatcctga ccctggccat 3420
gaacatcatg agcacactgc agtgggcagt gaacagcagc attgatgtgg acagcctgat 3480
gaggagtgtg agcagagtgt tcaagttcat tgatatgccc acagagggca agcctaccaa 3540
gagcaccaag ccctacaaga atggccagct gagcaaagtg atgatcattg agaacagcca 3600
tgtgaagaag gatgatatct ggcccagtgg aggccagatg acagtgaagg acctgacagc 3660
caagtacaca gaggggggca atgctatcct ggagaacatc tccttcagca tctcccctgg 3720
ccagagagtg ggactgctgg gaagaacagg ctctggcaag tctaccctgc tgtctgcctt 3780
cctgaggctg ctgaacacag agggagagat ccagattgat ggagtgtcct gggacagcat 3840
cacactgcag cagtggagga aggcctttgg tgtgatcccc cagaaagtgt tcatcttcag 3900
tggcaccttc aggaagaacc tggaccccta tgagcagtgg tctgaccagg agatttggaa 3960
agtggctgat gaagtgggcc tgagaagtgt gattgagcag ttccctggca agctggactt 4020
tgtcctggtg gatgggggct gtgtgctgag ccatggccac aagcagctga tgtgcctggc 4080
cagatcagtg ctgagcaagg ccaagatcct gctgctggat gagccttctg cccacctgga 4140
tcctgtgacc taccagatca tcaggaggac cctcaagcag gcctttgctg actgcacagt 4200
catcctgtgt gagcacagga ttgaggccat gctggagtgc cagcagttcc tggtgattga 4260
ggagaacaaa gtgaggcagt atgacagcat ccagaagctg ctgaatgaga ggagcctgtt 4320
caggcaggcc atcagcccct ctgatagagt gaagctgttc ccccacagga acagctccaa 4380
gtgcaagagc aagccccaga ttgctgccct gaaggaggag acagaggagg aagtgcagga 4440
caccaggctg tgagggccc 4459
Exemplified CFTR polypeptide
SEQ ID NO: 16
MQRSPLEKASVVSKLFFSWTRPILRKGYRQRLELSDIYQIPSVDSADNLSEKLEREWDRELASKKNPKLINALRR
CFFWRFMFYGIFLYLGEVTKAVQPLLLGRIIASYDPDNKEERSIAIYLGIGLCLLFIVRTLLLHPAIFGLHHIGM
QMRIAMFSLIYKKTLKLSSRVLDKISIGQLVSLLSNNLNKFDEGLALAHFVWIAPLQVALLMGLIWELLQASAFC
GLGFLIVLALFQAGLGRMMMKYRDQRAGKISERLVITSEMIENIQSVKAYCWEEAMEKMIENLRQTELKLTRKAA
YVRYFNSSAFFFSGFFVVFLSVLPYALIKGIILRKIFTTISFCIVLRMAVTRQFPWAVQTWYDSLGAINKIQDFL
QKQEYKTLEYNLTTTEVVMENVTAFWEEGFGELFEKAKQNNNNRKTSNGDDSLFFSNFSLLGTPVLKDINFKIER
GQLLAVAGSTGAGKTSLLMMIMGELEPSEGKIKHSGRISFCSQFSWIMPGTIKENIIFGVSYDEYRYRSVIKACQ
LEEDISKFAEKDNIVLGEGGITLSGGQRARISLARAVYKDADLYLLDSPFGYLDVLTEKEIFESCVCKLMANKTR
ILVTSKMEHLKKADKILILHEGSSYFYGTFSELQNLQPDFSSKLMGCDSFDQFSAERRNSILTETLHRFSLEGDA
PVSWTETKKQSFKQTGEFGEKRKNSILNPINSIRKFSIVQKTPLQMNGIEEDSDEPLERRLSLVPDSEQGEAILP
RISVISTGPTLQARRRQSVLNLMTHSVNQGQNIHRKTTASTRKVSLAPQANLTELDIYSRRLSQETGLEISEEIN
EEDLKECFFDDMESIPAVTTWNTYLRYITVHKSLIFVLIWCLVIFLAEVAASLVVLWLLGNTPLQDKGNSTHSRN
NSYAVIITSTSSYYVFYIYVGVADTLLAMGFFRGLPLVHTLITVSKILHHKMLHSVLQAPMSTLNTLKAGGILNR
FSKDIAILDDLLPLTIFDFIQLLLIVIGAIAVVAVLQPYIFVATVPVIVAFIMLRAYFLQTSQQLKQLESEGRSP
IFTHLVTSLKGLWTLRAFGRQPYFETLFHKALNLHTANWFLYLSTLRWFQMRIEMIFVIFFIAVTFISILTTGEG
EGRVGIILTLAMNIMSTLQWAVNSSIDVDSLMRSVSRVFKFIDMPTEGKPTKSTKPYKNGQLSKVMIIENSHVKK
DDIWPSGGQMTVKDLTAKYTEGGNAILENISFSISPGQRVGLLGRTGSGKSTLLSAFLRLLNTEGEIQIDGVSWD
SITLQQWRKAFGVIPQKVFIFSGTFRKNLDPYEQWSDQEIWKVADEVGLRSVIEQFPGKLDFVLVDGGCVLSHGH
KQLMCLARSVLSKAKILLLDEPSAHLDPVTYQIIRRTLKQAFADCTVILCEHRIEAMLECQQFLVIEENKVRQYD
SIQKLLNERSLFRQAISPSDRVKLFPHRNSSKCKSKPQIAALKEETEEEVQDTRL
Exemplified hGM-CSF transgene
SEQ ID NO: 17
ATGTGGCTGCAGAGCCTGCTGCTCTTGGGCACTGTGGCCTGCAGCATCTCTGCACCCGCCCGCTCGCCCAGCCCC
AGCACGCAGCCCTGGGAGCATGTGAATGCCATCCAGGAGGCCCGGCGTCTCCTGAACCTGAGTAGAGACACTGCT
GCTGAGATGAATGAAACAGTAGAAGTCATCTCAGAAATGTTTGACCTCCAGGAGCCGACCTGCCTACAGACCCGC
CTGGAGCTGTACAAGCAGGGCCTGCGGGGCAGCCTCACCAAGCTCAAGGGCCCCTTGACCATGATGGCCAGCCAC
TACAAGCAGCACTGCCCTCCAACCCCGGAAACTTCCTGTGCAACCCAGATTATCACCTTTGAAAGTTTCAAAGAG
AACCTGAAGGACTTTCTGCTTGTCATCCCCTTTGACTGCTGGGAGCCAGTCCAGGAGTGA
Exemplified hGM-CSF polypeptide
SEQ ID NO: 18
MWLQSLLLLGTVACSISAPARSPSPSTQPWEHVNAIQEARRLLNLSRDTAAEMNETVEVISEMFDLQEPTCLQTR
LELYKQGLRGSLTKLKGPLTMMASHYKQHCPPTPETSCATQIITFESFKENLKDFLLVIPFDCWEPVQE
Exemplified mGM-CSF transgene
SEQ ID NO: 19
ATGTGGCTGCAGAACCTGCTGTTCCTGGGCATTGTGGTGTACAGCCTGTCTGCCCCTACAAGATCCCCTATCACA
GTGACCAGACCTTGGAAACATGTGGAAGCCATCAAAGAGGCCCTGAATCTGCTGGATGACATGCCTGTGACACTG
AATGAAGAGGTGGAAGTGGTGTCCAATGAGTTCAGCTTCAAGAAACTGACCTGTGTGCAGACCAGGCTGAAGATT
TTTGAGCAGGGCCTGAGAGGCAACTTCACCAAGCTGAAAGGGGCTCTGAACATGACAGCCAGCTACTACCAGACC
TACTGTCCTCCTACACCTGAGACAGACTGTGAAACCCAAGTGACCACCTATGCTGACTTCATTGACAGCCTCAAG
ACCTTCCTGACAGACATCCCCTTTGAGTGCAAGAAACCTGGCCAGAAGTGA
Exemplified mGM-CSF polypeptide
SEQ ID NO: 20
MWLQNLLFLGIVVYSLSAPTRSPITVTRPWKHVEAIKEALNLLDDMPVTLNEEVEVVSNEFSFKKLTCVQTRLKI
FEQGLRGNFTKLKGALNMTASYYQTYCPPTPETDCETQVTTYADFIDSLKTFLTDIPFECKKPGQK
Exemplified Human DCN (Decorin) transgene
SEQ ID NO: 21
ATGAAGGCCACTATCATCCTCCTTCTGCTTGCACAAGTTTCCTGGGCTGGACCGTTTCAACAGAGAGGCTTATTT
GACTTTATGCTAGAAGATGAGGCTTCTGGGATAGGCCCAGAAGTTCCTGATGACCGCGACTTCGAGCCCTCCCTA
GGCCCAGTGTGCCCCTTCCGCTGTCAATGCCATCTTCGAGTGGTCCAGTGTTCTGATTTGGGTCTGGACAAAGTG
CCAAAGGATCTTCCCCCTGACACAACTCTGCTAGACCTGCAAAACAACAAAATAACCGAAATCAAAGATGGAGAC
TTTAAGAACCTGAAGAACCTTCACGCATTGATTCTTGTCAACAATAAAATTAGCAAAGTTAGTCCTGGAGCATTT
ACACCTTTGGTGAAGTTGGAACGACTTTATCTGTCCAAGAATCAGCTGAAGGAATTGCCAGAAAAAATGCCCAAA
ACTCTTCAGGAGCTGCGTGCCCATGAGAATGAGATCACCAAAGTGCGAAAAGTTACTTTCAATGGACTGAACCAG
ATGATTGTCATAGAACTGGGCACCAATCCGCTGAAGAGCTCAGGAATTGAAAATGGGGCTTTCCAGGGAATGAAG
AAGCTCTCCTACATCCGCATTGCTGATACCAATATCACCAGCATTCCTCAAGGTCTTCCTCCTTCCCTTACGGAA
TTACATCTTGATGGCAACAAAATCAGCAGAGTTGATGCAGCTAGCCTGAAAGGACTGAATAATTTGGCTAAGTTG
GGATTGAGTTTCAACAGCATCTCTGCTGTTGACAATGGCTCTCTGGCCAACACGCCTCATCTGAGGGAGCTTCAC
TTGGACAACAACAAGCTTACCAGAGTACCTGGTGGGCTGGCAGAGCATAAGTACATCCAGGTTGTCTACCTTCAT
AACAACAATATCTCTGTAGTTGGATCAAGTGACTTCTGCCCACCTGGACACAACACCAAAAAGGCTTCTTATTCG
GGTGTGAGTCTTTTCAGCAACCCGGTCCAGTACTGGGAGATACAGCCATCCACCTTCAGATGTGTCTACGTGCGC
TCTGCCATTCAACTCGGAAACTATAAGTAA
Exemplified Human Decorin polypeptide
SEQ ID NO: 22
MKATIILLLLAQVSWAGPFQQRGLFDFMLEDEASGIGPEVPDDRDFEPSLGPVCPFRCQCHLRVVQCSDLGLDKV
PKDLPPDTTLLDLQNNKITEIKDGDFKNLKNLHALILVNNKISKVSPGAFTPLVKLERLYLSKNQLKELPEKMPK
TLQELRAHENEITKVRKVTFNGLNQMIVIELGTNPLKSSGIENGAFQGMKKLSYIRIADTNITSIPQGLPPSLTE
LHLDGNKISRVDAASLKGLNNLAKLGLSFNSISAVDNGSLANTPHLRELHLDNNKLTRVPGGLAEHKYIQVVYLH
NNNISVVGSSDFCPPGHNTKKASYSGVSLFSNPVQYWEIQPSTFRCVYVRSAIQLGNYK
Exemplified Human TRIM72 transgene
SEQ ID NO: 23
ATGTCGGCTGCGCCCGGCCTCCTGCACCAGGAGCTGTCCTGCCCGCTGTGCCTGCAGCTGTTCGACGCGCCCGTG
ACAGCCGAGTGCGGCCACAGTTTCTGCCGCGCCTGCCTAGGCCGCGTGGCCGGGGAGCCGGCGGCGGATGGCACC
GTTCTCTGCCCCTGCTGCCAGGCCCCCACGCGGCCGCAGGCACTCAGCACCAACCTGCAGCTGGCGCGCCTGGTG
GAGGGGCTGGCCCAGGTGCCGCAGGGCCACTGCGAGGAGCACCTGGACCCGCTGAGCATCTACTGCGAGCAGGAC
CGCGCGCTGGTGTGCGGAGTGTGCGCCTCACTCGGCTCGCACCGCGGTCATCGCCTCCTGCCTGCCGCCGAGGCC
CACGCACGCCTCAAGACACAGCTGCCACAGCAGAAACTGCAGCTGCAGGAGGCATGCATGCGCAAGGAGAAGAGT
GTGGCTGTGCTGGAGCATCAGCTGGTGGAGGTGGAGGAGACAGTGCGTCAGTTCCGGGGGGCCGTGGGGGAGCAG
CTGGGCAAGATGCGGGTGTTCCTGGCTGCACTGGAGGGCTCCTTGGACCGCGAGGCAGAGCGTGTACGGGGTGAG
GCAGGGGTCGCCTTGCGCCGGGAGCTGGGGAGCCTGAACTCTTACCTGGAGCAGCTGCGGCAGATGGAGAAGGTC
CTGGAGGAGGTGGCGGACAAGCCGCAGACTGAGTTCCTCATGAAATACTGCCTGGTGACCAGCAGGCTGCAGAAG
ATCCTGGCAGAGTCTCCCCCACCCGCCCGTCTGGACATCCAGCTGCCAATTATCTCAGATGACTTCAAATTCCAG
GTGTGGAGGAAGATGTTCCGGGCTCTGATGCCAGCGCTGGAGGAGCTGACCTTTGACCCGAGCTCTGCGCACCCG
AGCCTGGTGGTGTCTTCCTCTGGCCGCCGCGTGGAGTGCTCGGAGCAGAAGGCGCCGCCGGCCGGGGAGGACCCG
CGCCAGTTCGACAAGGCGGTGGCGGTGGTGGCGCACCAGCAGCTCTCCGAGGGCGAGCACTACTGGGAGGTGGAT
GTTGGCGACAAGCCGCGCTGGGCGCTGGGCGTGATCGCGGCCGAGGCCCCCCGCCGCGGGCGCCTGCACGCGGTG
CCCTCGCAGGGCCTGTGGCTGCTGGGGCTGCGCGAGGGCAAGATCCTGGAGGCACACGTGGAGGCCAAGGAGCCG
CGCGCTCTGCGCAGCCCCGAGAGGCGGCCCACGCGCATTGGCCTTTACCTGAGCTTCGGCGACGGCGTCCTCTCC
TTCTACGATGCCAGCGACGCCGACGCGCTCGTGCCGCTTTTTGCCTTCCACGAGCGCCTGCCCAGGCCCGTGTAC
CCCTTCTTCGACGTGTGCTGGCACGACAAGGGCAAGAATGCCCAGCCGCTGCTGCTCGTGGGTCCCGAAGGCGCC
GAGGCCTGA
Exemplified Human TRIM72 polypeptide
SEQ ID NO: 24
MSAAPGLLHQELSCPLCLQLFDAPVTAECGHSFCRACLGRVAGEPAADGTVLCPCCQAPTRPQALSTNLQLARLV
EGLAQVPQGHCEEHLDPLSIYCEQDRALVCGVCASLGSHRGHRLLPAAEAHARLKTQLPQQKLQLQEACMRKEKS
VAVLEHQLVEVEETVRQFRGAVGEQLGKMRVFLAALEGSLDREAERVRGEAGVALRRELGSLNSYLEQLRQMEKV
LEEVADKPQTEFLMKYCLVTSRLQKILAESPPPARLDIQLPIISDDFKFQVWRKMFRALMPALEELTFDPSSAHP
SLVVSSSGRRVECSEQKAPPAGEDPRQFDKAVAVVAHQQLSEGEHYWEVDVGDKPRWALGVIAAEAPRRGRLHAV
PSQGLWLLGLREGKILEAHVEAKEPRALRSPERRPTRIGLYLSFGDGVLSFYDASDADALVPLFAFHERLPRPVY
PFFDVCWHDKGKNAQPLLLVGPEGAEA
Exemplified Human ABCA3 (ABCA3) transgene
SEQ ID NO: 25
ATGGCTGTGCTCAGGCAGCTGGCGCTCCTCCTCTGGAAGAACTACACCCTGCAGAAGCGGAAGGTCCTGGTGACG
GTCCTGGAACTCTTCCTGCCATTGCTGTTTTCTGGGATCCTCATCTGGCTCCGCTTGAAGATTCAGTCGGAAAAT
GTGCCCAACGCCACCATCTACCCGGGCCAGTCCATCCAGGAGCTGCCTCTGTTCTTCACCTTCCCTCCGCCAGGA
GACACCTGGGAGCTTGCCTACATCCCTTCTCACAGTGACGCTGCCAAGACCGTCACTGAGACAGTGCGCAGGGCA
CTTGTGATCAACATGCGAGTGCGCGGCTTTCCCTCCGAGAAGGACTTTGAGGACTACATTAGGTACGACAACTGC
TCGTCCAGCGTGCTGGCCGCCGTGGTCTTCGAGCACCCCTTCAACCACAGCAAGGAGCCCCTGCCGCTGGCGGTG
AAATATCACCTACGGTTCAGTTACACACGGAGAAATTACATGTGGACCCAAACAGGCTCCTTTTTCCTGAAAGAG
ACAGAAGGCTGGCACACTACTTCCCTTTTCCCGCTTTTCCCAAACCCAGGACCAAGGGAACCTACATCCCCTGAT
GGCGGAGAACCTGGGTACATCCGGGAAGGCTTCCTGGCCGTGCAGCATGCTGTGGACCGGGCCATCATGGAGTAC
CATGCCGATGCCGCCACACGCCAGCTGTTCCAGAGACTGACGGTGACCATCAAGAGGTTCCCGTACCCGCCGTTC
ATCGCAGACCCCTTCCTCGTGGCCATCCAGTACCAGCTGCCCCTGCTGCTGCTGCTCAGCTTCACCTACACCGCG
CTCACCATTGCCCGTGCTGTCGTGCAGGAGAAGGAAAGGAGGCTGAAGGAGTACATGCGCATGATGGGGCTCAGC
AGCTGGCTGCACTGGAGTGCCTGGTTCCTCTTGTTCTTCCTCTTCCTCCTCATCGCCGCCTCCTTCATGACCCTG
CTCTTCTGTGTCAAGGTGAAGCCAAATGTAGCCGTGCTGTCCCGCAGCGACCCCTCCCTGGTGCTCGCCTTCCTG
CTGTGCTTCGCCATCTCTACCATCTCCTTCAGCTTCATGGTCAGCACCTTCTTCAGCAAAGCCAACATGGCAGCA
GCCTTCGGAGGCTTCCTCTACTTCTTCACCTACATCCCCTACTTCTTCGTGGCCCCTCGGTACAACTGGATGACT
CTGAGCCAGAAGCTCTGCTCCTGCCTCCTGTCTAATGTCGCCATGGCAATGGGAGCCCAGCTCATTGGGAAATTT
GAGGCGAAAGGCATGGGCATCCAGTGGCGAGACCTCCTGAGTCCCGTCAACGTGGACGACGACTTCTGCTTCGGG
CAGGTGCTGGGGATGCTGCTGCTGGACTCTGTGCTCTATGGCCTGGTGACCTGGTACATGGAGGCCGTCTTCCCA
GGGCAGTTCGGCGTGCCTCAGCCCTGGTACTTCTTCATCATGCCCTCCTATTGGTGTGGGAAGCCAAGGGCGGTT
GCAGGGAAGGAGGAAGAAGACAGTGACCCCGAGAAAGCACTCAGAAACGAGTACTTTGAAGCCGAGCCAGAGGAC
CTGGTGGCGGGGATCAAGATCAAGCACCTGTCCAAGGTGTTCAGGGTGGGAAATAAGGACAGGGCGGCCGTCAGA
GACCTGAACCTCAACCTGTACGAGGGACAGATCACCGTCCTGCTGGGCCACAACGGTGCCGGGAAGACCACCACC
CTCTCCATGCTCACAGGTCTCTTTCCCCCCACCAGTGGACGGGCATACATCAGCGGGTATGAAATTTCCCAGGAC
ATGGTTCAGATCCGGAAGAGCCTGGGCCTGTGCCCGCAGCACGACATCCTGTTTGACAACTTGACAGTCGCAGAG
CACCTTTATTTCTACGCCCAGCTGAAGGGCCTGTCACGTCAGAAGTGCCCTGAAGAAGTCAAGCAGATGCTGCAC
ATCATCGGCCTGGAGGACAAGTGGAACTCACGGAGCCGCTTCCTGAGCGGGGGCATGAGGCGCAAGCTCTCCATC
GGCATCGCCCTCATCGCAGGCTCCAAGGTGCTGATACTGGACGAGCCCACCTCGGGCATGGACGCCATCTCCAGG
AGGGCCATCTGGGATCTTCTTCAGCGGCAGAAAAGTGACCGCACCATCGTGCTGACCACCCACTTCATGGACGAG
GCTGACCTGCTGGGAGACCGCATCGCCATCATGGCCAAGGGGGAGCTGCAGTGCTGCGGGTCCTCGCTGTTCCTC
AAGCAGAAATACGGTGCCGGCTATCACATGACGCTGGTGAAGGAGCCGCACTGCAACCCGGAAGACATCTCCCAG
CTGGTCCACCACCACGTGCCCAACGCCACGCTGGAGAGCAGCGCTGGGGCCGAGCTGTCTTTCATCCTTCCCAGA
GAGAGCACGCACAGGTTTGAAGGTCTCTTTGCTAAACTGGAGAAGAAGCAGAAAGAGCTGGGCATTGCCAGCTTT
GGGGCATCCATCACCACCATGGAGGAAGTCTTCCTTCGGGTCGGGAAGCTGGTGGACAGCAGTATGGACATCCAG
GCCATCCAGCTCCCTGCCCTGCAGTACCAGCACGAGAGGCGCGCCAGCGACTGGGCTGTGGACAGCAACCTCTGT
GGGGCCATGGACCCCTCCGACGGCATTGGAGCCCTCATCGAGGAGGAGCGCACCGCTGTCAAGCTCAACACTGGG
CTCGCCCTGCACTGCCAGCAATTCTGGGCCATGTTCCTGAAGAAGGCCGCATACAGCTGGCGCGAGTGGAAAATG
GTGGCGGCACAGGTCCTGGTGCCTCTGACCTGCGTCACCCTGGCCCTCCTGGCCATCAACTACTCCTCGGAGCTC
TTCGACGACCCCATGCTGAGGCTGACCTTGGGCGAGTACGGCAGAACCGTCGTGCCCTTCTCAGTTCCCGGGACC
TCCCAGCTGGGTCAGCAGCTGTCAGAGCATCTGAAAGACGCACTGCAGGCTGAGGGACAGGAGCCCCGCGAGGTG
CTCGGTGACCTGGAGGAGTTCTTGATCTTCAGGGCTTCTGTGGAGGGGGGCGGCTTTAATGAGCGGTGCCTTGTG
GCAGCGTCCTTCAGAGATGTGGGAGAGCGCACGGTCGTCAACGCCTTGTTCAACAACCAGGCGTACCACTCTCCA
GCCACTGCCCTGGCCGTCGTGGACAACCTTCTGTTCAAGCTGCTGTGCGGGCCTCACGCCTCCATTGTGGTCTCC
AACTTCCCCCAGCCCCGGAGCGCCCTGCAGGCTGCCAAGGACCAGTTTAACGAGGGCCGGAAGGGATTCGACATT
GCCCTCAACCTGCTCTTCGCCATGGCATTCTTGGCCAGCACGTTCTCCATCCTGGCGGTCAGCGAGAGGGCCGTG
CAGGCCAAGCATGTGCAGTTTGTGAGTGGAGTCCACGTGGCCAGTTTCTGGCTCTCTGCTCTGCTGTGGGACCTC
ATCTCCTTCCTCATCCCCAGTCTGCTGCTGCTGGTGGTGTTTAAGGCCTTCGACGTGCGTGCCTTCACGCGGGAC
GGCCACATGGCTGACACCCTGCTGCTGCTCCTGCTCTACGGCTGGGCCATCATCCCCCTCATGTACCTGATGAAC
TTCTTCTTCTTGGGGGCGGCCACTGCCTACACGAGGCTGACCATCTTCAACATCCTGTCAGGCATCGCCACCTTC
CTGATGGTCACCATCATGCGCATCCCAGCTGTAAAACTGGAAGAACTTTCCAAAACCCTGGATCACGTGTTCCTG
GTGCTGCCCAACCACTGTCTGGGGATGGCAGTCAGCAGTTTCTACGAGAACTACGAGACGCGGAGGTACTGCACC
TCCTCCGAGGTCGCCGCCCACTACTGCAAGAAATATAACATCCAGTACCAGGAGAACTTCTATGCCTGGAGCGCC
CCGGGGGTCGGCCGGTTTGTGGCCTCCATGGCCGCCTCAGGGTGCGCCTACCTCATCCTGCTCTTCCTCATCGAG
ACCAACCTGCTTCAGAGACTCAGGGGCATCCTCTGCGCCCTCCGGAGGAGGCGGACACTGACAGAATTATACACC
CGGATGCCTGTGCTTCCTGAGGACCAAGATGTAGCGGACGAGAGGACCCGCATCCTGGCCCCCAGTCCGGACTCC
CTGCTCCACACACCTCTGATTATCAAGGAGCTCTCCAAGGTGTACGAGCAGCGGGTGCCCCTCCTGGCCGTGGAC
AGGCTCTCCCTCGCGGTGCAGAAAGGGGAGTGCTTCGGCCTGCTGGGCTTCAATGGAGCCGGGAAGACCACGACT
TTCAAAATGCTGACCGGGGAGGAGAGCCTCACTTCTGGGGATGCCTTTGTCGGGGGTCACAGAATCAGCTCTGAT
GTCGGAAAGGTGCGGCAGCGGATCGGCTACTGCCCGCAGTTTGATGCCTTGCTGGACCACATGACAGGCCGGGAG
ATGCTGGTCATGTACGCTCGGCTCCGGGGCATCCCTGAGCGCCACATCGGGGCCTGCGTGGAGAACACTCTGCGG
GGCCTGCTGCTGGAGCCACATGCCAACAAGCTGGTCAGGACGTACAGTGGTGGTAACAAGCGGAAGCTGAGCACC
GGCATCGCCCTGATCGGAGAGCCTGCTGTCATCTTCCTGGACGAGCCGTCCACTGGCATGGACCCCGTGGCCCGG
CGCCTGCTTTGGGACACCGTGGCACGAGCCCGAGAGTCTGGCAAGGCCATCATCATCACCTCCCACAGCATGGAG
GAGTGTGAGGCCCTGTGCACCCGGCTGGCCATCATGGTGCAGGGGCAGTTCAAGTGCCTGGGCAGCCCCCAGCAC
CTCAAGAGCAAGTTCGGCAGCGGCTACTCCCTGCGGGCCAAGGTGCAGAGTGAAGGGCAACAGGAGGCGCTGGAG
GAGTTCAAGGCCTTCGTGGACCTGACCTTTCCAGGCAGCGTCCTGGAAGATGAGCACCAAGGCATGGTCCATTAC
CACCTGCCGGGCCGTGACCTCAGCTGGGCGAAGGTTTTCGGTATTCTGGAGAAAGCCAAGGAAAAGTACGGCGTG
GACGACTACTCCGTGAGCCAGATCTCGCTGGAACAGGTCTTCCTGAGCTTCGCCCACCTGCAGCCGCCCACCGCA
GAGGAGGGGCGATGA
Exemplified Human ABCA3 polypeptide
SEQ ID NO: 26
MAVLRQLALLLWKNYTLQKRKVLVTVLELFLPLLFSGILIWLRLKIQSENVPNATIYPGQSIQELPLFFTFPPPG
DTWELAYIPSHSDAAKTVTETVRRALVINMRVRGFPSEKDFEDYIRYDNCSSSVLAAVVFEHPFNHSKEPLPLAV
KYHLRFSYTRRNYMWTQTGSFFLKETEGWHTTSLFPLFPNPGPREPTSPDGGEPGYIREGFLAVQHAVDRAIMEY
HADAATRQLFQRLTVTIKRFPYPPFIADPFLVAIQYQLPLLLLLSFTYTALTIARAVVQEKERRLKEYMRMMGLS
SWLHWSAWFLLFFLFLLIAASFMTLLFCVKVKPNVAVLSRSDPSLVLAFLLCFAISTISFSFMVSTFFSKANMAA
AFGGFLYFFTYIPYFFVAPRYNWMTLSQKLCSCLLSNVAMAMGAQLIGKFEAKGMGIQWRDLLSPVNVDDDFCFG
QVLGMLLLDSVLYGLVTWYMEAVFPGQFGVPQPWYFFIMPSYWCGKPRAVAGKEEEDSDPEKALRNEYFEAEPED
LVAGIKIKHLSKVFRVGNKDRAAVRDLNLNLYEGQITVLLGHNGAGKTTTLSMLTGLFPPTSGRAYISGYEISQD
MVQIRKSLGLCPQHDILFDNLTVAEHLYFYAQLKGLSRQKCPEEVKQMLHIIGLEDKWNSRSRFLSGGMRRKLSI
GIALIAGSKVLILDEPTSGMDAISRRAIWDLLQRQKSDRTIVLTTHFMDEADLLGDRIAIMAKGELQCCGSSLFL
KQKYGAGYHMTLVKEPHCNPEDISQLVHHHVPNATLESSAGAELSFILPRESTHRFEGLFAKLEKKQKELGIASF
GASITTMEEVFLRVGKLVDSSMDIQAIQLPALQYQHERRASDWAVDSNLCGAMDPSDGIGALIEEERTAVKLNTG
LALHCQQFWAMFLKKAAYSWREWKMVAAQVLVPLTCVTLALLAINYSSELFDDPMLRLTLGEYGRTVVPFSVPGT
SQLGQQLSEHLKDALQAEGQEPREVLGDLEEFLIFRASVEGGGFNERCLVAASFRDVGERTVVNALFNNQAYHSP
ATALAVVDNLLFKLLCGPHASIVVSNFPQPRSALQAAKDQFNEGRKGFDIALNLLFAMAFLASTFSILAVSERAV
QAKHVQFVSGVHVASFWLSALLWDLISFLIPSLLLLVVFKAFDVRAFTRDGHMADTLLLLLLYGWAIIPLMYLMN
FFFLGAATAYTRLTIFNILSGIATFLMVTIMRIPAVKLEELSKTLDHVFLVLPNHCLGMAVSSFYENYETRRYCT
SSEVAAHYCKKYNIQYQENFYAWSAPGVGRFVASMAASGCAYLILLFLIETNLLQRLRGILCALRRRRTLTELYT
RMPVLPEDQDVADERTRILAPSPDSLLHTPLIIKELSKVYEQRVPLLAVDRLSLAVQKGECFGLLGFNGAGKTTT
FKMLTGEESLTSGDAFVGGHRISSDVGKVRQRIGYCPQFDALLDHMTGREMLVMYARLRGIPERHIGACVENTLR
GLLLEPHANKLVRTYSGGNKRKLSTGIALIGEPAVIFLDEPSTGMDPVARRLLWDTVARARESGKAIIITSHSME
ECEALCTRLAIMVQGQFKCLGSPQHLKSKFGSGYSLRAKVQSEGQQEALEEFKAFVDLTFPGSVLEDEHQGMVHY
HLPGRDLSWAKVFGILEKAKEKYGVDDYSVSQISLEQVFLSFAHLQPPTAEEGR
Exemplified hCEF promoter
SEQ ID NO: 27
agatctgtta cataacttat ggtaaatggc ctgcctggct gactgcccaa tgacccctgc 60
ccaatgatgt caataatgat gtatgttccc atgtaatgcc aatagggact ttccattgat 120
gtcaatgggt ggagtattta tggtaactgc ccacttggca gtacatcaag tgtatcatat 180
gccaagtatg ccccctattg atgtcaatga tggtaaatgg cctgcctggc attatgccca 240
gtacatgacc ttatgggact ttcctacttg gcagtacatc tatgtattag tcattgctat 300
taccatggga attcactagt ggagaagagc atgcttgagg gctgagtgcc cctcagtggg 360
cagagagcac atggcccaca gtccctgaga agttgggggg aggggtgggc aattgaactg 420
gtgcctagag aaggtggggc ttgggtaaac tgggaaagtg atgtggtgta ctggctccac 480
ctttttcccc agggtggggg agaaccatat ataagtgcag tagtctctgt gaacattcaa 540
gcttctgcct tctccctcct gtgagtttgc tagc 574
Exemplified CMV promoter
SEQ ID NO: 28
ccgcggagat ctcaatattg gccattagcc atattattca ttggttatat agcataaatc 60
aatattggct attggccatt gcatacgttg tatctatatc ataatatgta catttatatt 120
ggctcatgtc caatatgacc gccatgttgg cattgattat tgactagtta ttaatagtaa 180
tcaattacgg ggtcattagt tcatagccca tatatggagt tccgcgttac ataacttacg 240
gtaaatggcc cgcctggctg accgcccaac gacccccgcc cattgacgtc aataatgacg 300
tatgttccca tagtaacgcc aatagggact ttccattgac gtcaatgggt ggagtattta 360
cggtaaactg cccacttggc agtacatcaa gtgtatcata tgccaagtcc gccccctatt 420
gacgtcaatg acggtaaatg gcccgcctgg cattatgccc agtacatgac cttacgggac 480
tttcctactt ggcagtacat ctacgtatta gtcatcgcta ttaccatggt gatgcggttt 540
tggcagtaca ccaatgggcg tggatagcgg tttgactcac ggggatttcc aagtctccac 600
cccattgacg tcaatgggag tttgttttgg caccaaaatc aacgggactt tccaaaatgt 660
cgtaataacc ccgccccgtt gacgcaaatg ggcggtaggc gtgtacggtg ggaggtctat 720
ataagcagag ctcgtttagt gaaccgtcag atcactagaa gctttattgc ggtagtttat 780
cacagttaaa ttgctaacgc agtcagtgct tctgacacaa cagtctcgaa cttaagctgc 840
agaagttggt cgtgaggcac tgggcaggct agc 873
Exemplified EF1a promoter
SEQ ID NO: 29
agatccatat ccgcggcaat tttaaaagaa agggaggaat agggggacag acttcagcag 60
agagactaat taatataata acaacacaat tagaaataca acatttacaa accaaaattc 120
aaaaaatttt aaattttaga gccgcggaga tcccgtgagg ctccggtgcc cgtcagtggg 180
cagagcgcac atcgcccaca gtccccgaga agttgggggg aggggtcggc aattgaaccg 240
gtgcctagag aaggtggcgc ggggtaaact gggaaagtga tgtcgtgtac tggctccgcc 300
tttttcccga gggtggggga gaaccgtata taagtgcagt agtcgccgtg aacgttcttt 360
ttcgcaacgg gtttgccgcc agaacacagg ctagc 395
Plasmid as defined in FIG. 2A (pDNA1 pGM991)
SEQ ID NO: 30
GGTACCTCAATATTGGCCATTAGCCATATTATTCATTGGTTATATAGCATAAATCAATATTGGCTATTGGCCATT
GCATACGTTGTATCTATATCATAATATGTACATTTATATTGGCTCATGTCCAATATGACCGCCATGTTGGCATTG
ATTATTGACTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTACGGTAAACTGCCCACTTGGC
AGTACATCAAGTGTATCATATGCCAAGTCCGCCCCCTATTGACGTCAATGACGGTAAATGGCCCGCCTGGCATTA
TGCCCAGTACATGACCTTACGGGACTTTCCTACTTGGCAGTACATCTACGTATTAGTCATCGCTATTACCATGGT
GATGCGGTTTTGGCAGTACACCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAACAACTGCGATCGCCC
GCCCCGTTGACGCAAATGGGCGGTAGGCGTGTACGGTGGGAGGTCTATATAAGCAGAGCTCGCTGGCTTGTAACT
CAGTCTCTTACTAGGAGACCAGCTTGAGCCTGGGTGTTCGCTGGTTAGCCTAACCTGGTTGGCCACCAGGGGTAA
GGACTCCTTGGCTTAGAAAGCTAATAAACTTGCCTGCATTAGAGCTTATCTGAGTCAAGTGTCCTCATTGACGCC
TCACTCTCTTGAACGGGAATCTTCCTTACTGGGTTCTCTCTCTGACCCAGGCGAGAGAAACTCCAGCAGTGGCGC
CCGAACAGGGACTTGAGTGAGAGTGTAGGCACGTACAGCTGAGAAGGCGTCGGACGCGAAGGAAGCGCGGGGTGC
GACGCGACCAAGAAGGAGACTTGGTGAGTAGGCTTCTCGAGTGCCGGGAAAAAGCTCGAGCCTAGTTAGAGGACT
AGGAGAGGCCGTAGCCGTAACTACTCTTGGGCAAGTAGGGCAGGCGGTGGGTACGCAATGGGGGGGGCTACCTCA
GCACTAAATAGGAGACAATTAGACCAATTTGAGAAAATACGACTTCGCCCGAACGGAAAGAAAAAGTACCAAATT
AAACATTTAATATGGGCAGGCAAGGAGATGGAGCGCTTCGGCCTCCATGAGAGGTTGTTGGAGACAGAGGAGGGG
TGTAAAAGAATCATAGAAGTCCTCTACCCCCTAGAACCAACAGGATCGGAGGGCTTAAAAAGTCTGTTCAATCTT
GTGTGCGTGCTATATTGCTTGCACAAGGAACAGAAAGTGAAAGACACAGAGGAAGCAGTAGCAACAGTAAGACAA
CACTGCCATCTAGTGGAAAAAGAAAAAAGTGCAACAGAGACATCTAGTGGACAAAAGAAAAATGACAAGGGAATA
GCAGCGCCACCTGGTGGCAGTCAGAATTTTCCAGCGCAACAACAAGGAAATGCCTGGGTACATGTACCCTTGTCA
CCGCGCACCTTAAATGCGTGGGTAAAAGCAGTAGAGGAGAAAAAATTTGGAGCAGAAATAGTACCCATTTTTTTG
TTTCAAGCCCTATCGAATTCCCGCGGCAATTTTAAAAGAAAGGGAGGAATAGGGGGACAGACTTCAGCAGAGAGA
CTAATTAATATAATAACAACACAATTAGAAATACAACATTTACAAACCAAAATTCAAAAAATTTTAAATTTTAGA
GCCGCGGAGATCTGTTACATAACTTATGGTAAATGGCCTGCCTGGCTGACTGCCCAATGACCCCTGCCCAATGAT
GTCAATAATGATGTATGTTCCCATGTAATGCCAATAGGGACTTTCCATTGATGTCAATGGGTGGAGTATTTATGG
TAACTGCCCACTTGGCAGTACATCAAGTGTATCATATGCCAAGTATGCCCCCTATTGATGTCAATGATGGTAAAT
GGCCTGCCTGGCATTATGCCCAGTACATGACCTTATGGGACTTTCCTACTTGGCAGTACATCTATGTATTAGTCA
TTGCTATTACCATGGGAATTCACTAGTGGAGAAGAGCATGCTTGAGGGCTGAGTGCCCCTCAGTGGGCAGAGAGC
ACATGGCCCACAGTCCCTGAGAAGTTGGGGGGAGGGGTGGGCAATTGAACTGGTGCCTAGAGAAGGTGGGGCTTG
GGTAAACTGGGAAAGTGATGTGGTGTACTGGCTCCACCTTTTTCCCCAGGGTGGGGGAGAACCATATATAAGTGC
AGTAGTCTCTGTGAACATTCCTGCCTTCTCCCTCCTGTGAGTTTAAGGTAAGTATCAAGGTTACAAGACAGGTTT
AAGGAGACCAATAGAAACTGGGCTTGTCGAGACAGAGAAGACTCTTGCGTTTCTGATAGGCACGCGGCCGC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]








GCGGCCGCCTATTGGTCTTACTGACATCCACTTTGCCTTTCTCTCCACAGCTAGCCACCATGCCCAGC
TCTGTGTCCTGGGGCATTCTGCTGCTGGCTGGCCTGTGCTGTCTGGTGCCTGTGTCCCTGGCTGAGGACCCTCAG
GGGGATGCTGCCCAGAAAACAGACACCTCCCACCATGACCAGGACCACCCCACCTTCAACAAGATCACCCCCAAC
CTGGCAGAGTTTGCCTTCAGCCTGTACAGACAGCTGGCCCACCAGAGCAACAGCACCAACATCTTTTTCAGCCCT
GTGTCCATTGCCACAGCCTTTGCCATGCTGAGCCTGGGCACCAAGGCTGACACCCATGATGAGATCCTGGAAGGC
CTGAACTTCAACCTGACAGAGATCCCTGAGGCCCAGATCCATGAGGGCTTCCAGGAACTGCTGAGAACCCTGAAC
CAGCCAGACAGCCAGCTGCAGCTGACAACAGGCAATGGGCTGTTCCTGTCTGAGGGCCTGAAGCTGGTGGACAAG
TTTCTGGAAGATGTGAAGAAGCTGTACCACTCTGAGGCCTTCACAGTGAACTTTGGGGACACAGAAGAGGCCAAG
AAACAGATCAATGACTATGTGGAAAAGGGCACCCAGGGCAAGATTGTGGACCTTGTGAAAGAGCTGGACAGGGAC
ACTGTGTTTGCCCTTGTGAACTACATCTTCTTCAAGGGCAAGTGGGAGAGGCCCTTTGAAGTGAAGGACACTGAG
GAAGAGGACTTCCATGTGGACCAAGTGACCACAGTGAAGGTGCCAATGATGAAGAGACTGGGGATGTTCAATATC
CAGCACTGCAAGAAACTGAGCAGCTGGGTGCTGCTGATGAAGTACCTGGGCAATGCTACAGCCATATTCTTTCTG
CCTGATGAGGGCAAGCTGCAGCACCTGGAAAATGAGCTGACCCATGACATCATCACCAAATTTCTGGAAAATGAG
GACAGAAGATCTGCCAGCCTGCATCTGCCCAAGCTGAGCATCACAGGCACATATGACCTGAAGTCTGTGCTGGGA
CAGCTGGGAATCACCAAGGTGTTCAGCAATGGGGCAGACCTGAGTGGAGTGACAGAGGAAGCCCCTCTGAAGCTG
TCCAAGGCTGTGCACAAGGCAGTGCTGACCATTGATGAGAAGGGCACAGAGGCTGCTGGGGCCATGTTTCTGGAA
GCCATCCCCATGTCCATCCCCCCAGAAGTGAAGTTCAACAAGCCCTTTGTGTTCCTGATGATTGAGCAGAACACC
AAGAGCCCCCTGTTCATGGGCAAGGTTGTGAACCCCACCCAGAAATGAGGGCCCAATCAACCTCTGGATTACAAA
ATTTGTGAAAGATTGACTGGTATTCTTAACTATGTTGCTCCTTTTACGCTATGTGGATACGCTGCTTTAATGCCT
TTGTATCATGCTATTGCTTCCCGTATGGCTTTCATTTTCTCCTCCTTGTATAAATCCTGGTTGCTGTCTCTTTAT
GAGGAGTTGTGGCCCGTTGTCAGGCAACGTGGCGTGGTGTGCACTGTGTTTGCTGACGCAACCCCCACTGGTTGG
GGCATTGCCACCACCTGTCAGCTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACAATTCCGTGGTGTTGTCGGGGAAA
TCATCGTCCTTTCCTTGGCTGCTCGCCTGTGTTGCCACCTGGATTCTGCGCGGGACGTCCTTCTGCTACGTCCCT
TCGGCCCTCAATCCAGCGGACCTTCCTTCCCGCGGCCTGCTGCCGGCTCTGCGGCCTCTTCCGCGTCTTCGCCTT
CGCCCTCAGACGAGTCGGATCTCCCTTTGGGCCGCCTCCCCGCAAGCTTCGCACTTTTTAAAAGAAAAGGGAGGA
CTGGATGGGATTTATTACTCCGATAGGACGCTGGCTTGTAACTCAGTCTCTTACTAGGAGACCAGCTTGAGCCTG
GGTGTTCGCTGGTTAGCCTAACCTGGTTGGCCACCAGGGGTAAGGACTCCTTGGCTTAGAAAGCTAATAAACTTG
CCTGCATTAGAGCTCTTACGCGTCCCGGGCTCGAGATCCGCATCTCAATTAGTCAGCAACCATAGTCCCGCCCCT
AACTCCGCCCATCCCGCCCCTAACTCCGCCCAGTTCCGCCCATTCTCCGCCCCATGGCTGACTAATTTTTTTTAT
TTATGCAGAGGCCGAGGCCGCCTCGGCCTCTGAGCTATTCCAGAAGTAGTGAGGAGGCTTTTTTGGAGGCCTAGG
CTTTTGCAAAAAGCTAACTTGTTTATTGCAGCTTATAATGGTTACAAATAAAGCAATAGCATCACAAATTTCACA
AATAAAGCATTTTTTTCACTGCATTCTAGTTGTGGTTTGTCCAAACTCATCAATGTATCTTATCATGTCTGTCCG
CTTCCTCGCTCACTGACTCGCTGCGCTCGGTCGTTCGGCTGCGGCGAGCGGTATCAGCTCACTCAAAGGCGGTAA
TACGGTTATCCACAGAATCAGGGGATAACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGAGCATCACAAAAATCGACGCTCAA
GTCAGAGGTGGCGAAACCCGACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGCGTGGCGCTTTCTCATAGCT
CACGCTGTAGGTATCTCAGTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAGTGGTGGCCTAACT
ACGGCTACACTAGAAGAACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATTAAAAATGAAGTTTTAAATCAA
TCTAAAGTATATATGAGTAAACTTGGTCTGACAGTTAGAAAAACTCATCGAGCATCAAATGAAACTGCAATTTAT
TCATATCAGGATTATCAATACCATATTTTTGAAAAAGCCGTTTCTGTAATGAAGGAGAAAACTCACCGAGGCAGT
TCCATAGGATGGCAAGATCCTGGTATCGGTCTGCGATTCCGACTCGTCCAACATCAATACAACCTATTAATTTCC
CCTCGTCAAAAATAAGGTTATCAAGTGAGAAATCACCATGAGTGACGACTGAATCCGGTGAGAATGGCAACAGCT
TATGCATTTCTTTCCAGACTTGTTCAACAGGCCAGCCATTACGCTCGTCATCAAAATCACTCGCATCAACCAAAC
CGTTATTCATTCGTGATTGCGCCTGAGCGAGACGAAATACGCGATCGCTGTTAAAAGGACAATTACAAACAGGAA
TCGAATGCAACCGGCGCAGGAACACTGCCAGCGCATCAACAATATTTTCACCTGAATCAGGATATTCTTCTAATA
CCTGGAATGCTGTTTTTCCGGGGATCGCAGTGGTGAGTAACCATGCATCATCAGGAGTACGGATAAAATGCTTGA
TGGTCGGAAGAGGCATAAATTCCGTCAGCCAGTTTAGTCTGACCATCTCATCTGTAACATCATTGGCAACGCTAC
CTTTGCCATGTTTCAGAAACAACTCTGGCGCATCGGGCTTCCCATACAATCGATAGATTGTCGCACCTGATTGCC
CGACATTATCGCGAGCCCATTTATACCCATATAAATCAGCATCCATGTTGGAATTTAATCGCGGCCTAGAGCAAG
ACGTTTCCCGTTGAATATGGCTCATAACACCCCTTGTATTACTGTTTATGTAAGCAGACAGTTTTATTGTTCATG
ATGATATATTTTTATCTTGTGCAATGTAACATCAGAGATTTTGAGACACAACAATTGGTCGACGGATCC
hCEF Promoter is underlined
β-globulin/IgGG chimeric intron comprising SIV RRE Intron is italicised
SIV RRE is italicised and double-underlined
Plasmid as defined in FIG. 2B (pDNA1 pGM691)
SEQ ID NO: 31
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tgggggggtg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggctg ccttcggggg ggacggggca gggcggggtt cggcttctgg cgtgtgaccg 1620
gcggctctag agcctctgct aaccatgttc atgccttctt ctttttccta cagctcctgg 1680
gcaacgtgct ggttattgtg ctgtctcatc attttggcaa agaattgctc gagccaccat 1740
gggagctgcc acatctgccc tgaatagacg gcagctggac cagttcgaga agatcagact 1800
gcggcccaac ggcaagaaga agtaccagat caagcacctg atctgggccg gcaaagagat 1860
ggaaagattc ggcctgcacg agcggctgct ggaaaccgag gaaggctgca agagaattat 1920
cgaggtgctg taccctctgg aacctaccgg ctctgagggc ctgaagtccc tgttcaatct 1980
cgtgtgcgtg ctgtactgcc tgcacaaaga acagaaagtg aaggacaccg aagaggccgt 2040
ggccacagtt agacagcact gccacctggt ggaaaaagag aagtccgcca cagagacaag 2100
cagcggccag aagaagaacg acaagggaat tgctgcccct cctggcggca gccagaattt 2160
tcctgctcag cagcagggaa acgcctgggt gcacgttcca ctgagcccta gaacactgaa 2220
tgcctgggtc aaagccgtgg aagagaagaa gtttggcgcc gagatcgtgc ccatgttcca 2280
ggctctgtct gagggctgca ccccttacga catcaaccag atgctgaacg tgctgggaga 2340
tcaccagggc gctctgcaga tcgtgaaaga gatcatcaac gaagaggctg cccagtggga 2400
cgtgacacat ccattgcctg ctggacctct gccagccgga caactgagag atcctagagg 2460
ctctgatatc gccggcacca ccagctctgt gcaagagcag ctggaatgga tctacaccgc 2520
caatcctaga gtggacgtgg gcgccatcta cagaagatgg atcatcctgg gcctgcagaa 2580
atgcgtgaag atgtacaacc ccgtgtccgt gctggacatc agacagggac ccaaagagcc 2640
cttcaaggac tacgtggacc ggttctataa ggccattaga gccgagcagg ccagcggcga 2700
agtgaagcag tggatgacag agagcctgct gatccagaac gccaatccag actgcaaagt 2760
gatcctgaaa ggcctgggca tgcaccccac actggaagag atgctgacag cctgtcaagg 2820
cgttggcggc ccttcttaca aagccaaagt gatggccgag atgatgcaga ccatgcagaa 2880
ccagaacatg gtgcagcaag gcggccctaa gagacagagg cctcctctga gatgctacaa 2940
ctgcggcaag ttcggccaca tgcagagaca gtgtcctgag cctaggaaaa caaaatgtct 3000
aaagtgtgga aaattgggac acctagcaaa agactgcagg ggacaggtga attttttagg 3060
gtatggacgg tggatggggg caaaaccgag aaattttccc gccgctactc ttggagcgga 3120
accgagtgcg cctcctccac cgagcggcac caccccatac gacccagcaa agaagctcct 3180
gcagcaatat gcagagaaag ggaaacaact gagggagcaa aagaggaatc caccggcaat 3240
gaatccggat tggaccgagg gatattcttt gaactccctc tttggagaag accaataaag 3300
accgtgtaca tcgagggcgt gcccatcaag gctctgctgg atacaggcgc cgacgacacc 3360
atcatcaaag agaacgacct gcagctgagc ggcccttgga ggcctaagat cattggagga 3420
atcggcggag gcctgaacgt caaagagtac aacgaccggg aagtgaagat cgaggacaag 3480
atcctgaggg gcacaatcct gctgggcgcc acacctatca acatcatcgg cagaaatctg 3540
ctggcccctg ccggcgctag actggttatg ggacagctct ctgagaagat ccccgtgaca 3600
cccgtgaagc tgaaagaagg cgctagagga ccttgtgtgc gacagtggcc tctgagcaaa 3660
gagaagattg aggccctgca agaaatctgt agccagctgg aacaagaggg caagatcagc 3720
agagttggcg gcgagaacgc ctacaatacc cctatcttct gcatcaagaa aaaggacaag 3780
agccagtggc ggatgctggt ggactttaga gagctgaaca aggctaccca ggacttcttc 3840
gaggtgcagc tgggaattcc tcatcctgcc ggcctgcgga agatgagaca gatcacagtg 3900
ctggatgtgg gcgacgccta ctacagcatc cctctggacc ccaacttcag aaagtacacc 3960
gccttcacaa tccccaccgt gaacaatcaa ggccctggca tcagatacca gttcaactgc 4020
ctgcctcaag gctggaaggg cagccccacc atttttcaga ataccgccgc cagcatcctg 4080
gaagaaatca agagaaacct gcctgctctg accatcgtgc agtacatgga cgatctgtgg 4140
gtcggaagcc aagagaatga gcacacccac gacaagctgg tggaacagct gagaacaaag 4200
ctgcaggcct ggggcctcga aacccctgag aagaaggtgc agaaagaacc tccttacgag 4260
tggatgggct acaagctgtg gcctcacaag tgggagctga gccggattca gctcgaagag 4320
aaggacgagt ggaccgtgaa cgacatccag aaactcgtgg gcaagctgaa ttgggcagcc 4380
cagctgtatc ccggcctgag gaccaagaac atctgcaagc tgatccgggg aaagaagaac 4440
ctgctggaac tggtcacatg gacacctgag gccgaggccg aatatgccga gaatgccgaa 4500
atcctgaaaa ccgagcaaga ggggacctac tacaagcctg gcattccaat cagagctgcc 4560
gtgcagaaac tggaaggcgg ccagtggtcc taccagttta agcaagaagg ccaggtcctg 4620
aaagtgggca agtacaccaa gcagaagaac acccacacca acgagctgag gacactggct 4680
ggcctggtcc agaaaatctg caaagaggcc ctggtcattt ggggcatcct gcctgttctg 4740
gaactgccca ttgagcggga agtgtgggaa cagtggtggg ccgattactg gcaagtgtct 4800
tggatccccg agtgggactt cgtgtctacc cctcctctgc tgaaactgtg gtacaccctg 4860
acaaaagagc ccattcctaa agaggacgtc tactacgttg acggcgcctg caaccggaac 4920
tccaaagaag gcaaggccgg ctacatcagc cagtacggca agcagagagt ggaaaccctg 4980
gaaaacacca ccaaccagca ggccgagctg accgccatta agatggccct ggaagatagc 5040
ggccccaatg tgaacatcgt gaccgactct cagtacgcca tgggaatcct gacagcccag 5100
cctacacaga gcgatagccc tctggttgag cagatcattg ccctgatgat tcagaagcag 5160
caaatctacc tgcagtgggt gcccgctcac aaaggcatcg gcggaaacga agagatcgat 5220
aagctggtgt ccaagggaat cagacgggtg ctgttcctgg aaaagattga agaggcccaa 5280
gaggaacacg agcgctacca caacaactgg aagaatctgg ccgacaccta cggactgccc 5340
cagatcgtgg ccaaagaaat cgtggctatg tgccccaagt gtcagatcaa gggcgaacct 5400
gtgcacggcc aagtggatgc ttctcctggc acatggcaga tggactgtac ccacctggaa 5460
ggcaaagtgg tcatcgtggc tgtgcacgtg gcctccggct ttattgaggc cgaagtgatc 5520
cccagagaga caggcaaaga aaccgccaag ttcctgctga agatcctgtc cagatggccc 5580
atcacacagc tgcacaccga caacggccct aacttcacat ctcaagaggt ggccgccatc 5640
tgttggtggg gaaagattga gcacacaacc ggcattccct acaatccaca gagccagggc 5700
agcatcgagt ccatgaacaa gcagctcaaa gagattatcg gcaagatccg ggacgactgc 5760
cagtacacag aaacagccgt gctgatggcc tgtcacatcc acaacttcaa gcggaaaggc 5820
ggcatcggag gacagacatc tgccgagaga ctgatcaata tcatcaccac tcagctggaa 5880
atccagcacc tccagaccaa gatccagaag attctgaact tccgggtgta ctaccgcgag 5940
ggcagagatc ctgtttggaa aggcccagca cagctgatct ggaaaggcga aggtgccgtg 6000
gtgctgaagg atggctctga tctgaaggtg gtgcccagac ggaaggccaa gattatcaag 6060
gattacgagc ccaaacagcg cgtgggcaat gaaggcgacg ttgagggcac aagaggcagc 6120
gacaattgaa attcactcct caggtgcagg ctgcctatca gaaggtggtg gctggtgtgg 6180
ccaatgccct ggctcacaaa taccactgag atctttttcc ctctgccaaa aattatgggg 6240
acatcatgaa gccccttgag catctgactt ctggctaata aaggaaattt attttcattg 6300
caatagtgtg ttggaatttt ttgtgtctct cactcggaag gacatatggg agggcaaatc 6360
atttaaaaca tcagaatgag tatttggttt agagtttggc aacatatgcc catatgctgg 6420
ctgccatgaa caaaggttgg ctataaagag gtcatcagta tatgaaacag ccccctgctg 6480
tccattcctt attccataga aaagccttga cttgaggtta gatttttttt atattttgtt 6540
ttgtgttatt tttttcttta acatccctaa aattttcctt acatgtttta ctagccagat 6600
ttttcctcct ctcctgacta ctcccagtca tagctgtccc tcttctctta tggagatccc 6660
tcgacctgca gcccaagctt ggcgtaatca tggtcatagc tgtttcctgt gtgaaattgt 6720
tatccgctca caattccaca caacatacga gccggaagca taaagtgtaa agcctggggt 6780
gcctaatgag tgagctaact cacattaatt gcgttgcgct cactgcccgc tttccagtcg 6840
ggaaacctgt cgtgccagcg gatccgcatc tcaattagtc agcaaccata gtcccgcccc 6900
taactccgcc catcccgccc ctaactccgc ccagttccgc ccattctccg ccccatggct 6960
gactaatttt ttttatttat gcagaggccg aggccgcctc ggcctctgag ctattccaga 7020
agtagtgagg aggctttttt ggaggcctag gcttttgcaa aaagctaact tgtttattgc 7080
agcttataat ggttacaaat aaagcaatag catcacaaat ttcacaaata aagcattttt 7140
ttcactgcat tctagttgtg gtttgtccaa actcatcaat gtatottatc atgtctgtcc 7200
gcttcctcgc tcactgactc gctgcgctcg gtcgttcggc tgcggcgagc ggtatcagct 7260
cactcaaagg cggtaatacg gttatccaca gaatcagggg ataacgcagg aaagaacatg 7320
tgagcaaaag gccagcaaaa ggccaggaac cgtaaaaagg ccgcgttgct ggcgtttttc 7380
cataggctcc gcccccctga cgagcatcac aaaaatcgac gctcaagtca gaggtggcga 7440
aacccgacag gactataaag ataccaggcg tttccccctg gaagctccct cgtgcgctct 7500
cctgttccga ccctgccgct taccggatac ctgtccgcct ttctcccttc gggaagcgtg 7560
gcgctttctc atagctcacg ctgtaggtat ctcagttcgg tgtaggtcgt tcgctccaag 7620
ctgggctgtg tgcacgaacc ccccgttcag cccgaccgct gcgccttatc cggtaactat 7680
cgtcttgagt ccaacccggt aagacacgac ttatcgccac tggcagcagc cactggtaac 7740
aggattagca gagcgaggta tgtaggcggt gctacagagt tcttgaagtg gtggcctaac 7800
tacggctaca ctagaagaac agtatttggt atctgcgctc tgctgaagcc agttaccttc 7860
ggaaaaagag ttggtagctc ttgatccggc aaacaaacca ccgctggtag cggtggtttt 7920
tttgtttgca agcagcagat tacgcgcaga aaaaaaggat ctcaagaaga tcctttgatc 7980
ttttctacgg ggtctgacgc tcagtggaac gaaaactcac gttaagggat tttggtcatg 8040
agattatcaa aaaggatctt cacctagatc cttttaaatt aaaaatgaag ttttaaatca 8100
atctaaagta tatatgagta aacttggtct gacagttaga aaaactcatc gagcatcaaa 8160
tgaaactgca atttattcat atcaggatta tcaataccat atttttgaaa aagccgtttc 8220
tgtaatgaag gagaaaactc accgaggcag ttccatagga tggcaagatc ctggtatcgg 8280
tctgcgattc cgactcgtcc aacatcaata caacctatta atttcccctc gtcaaaaata 8340
aggttatcaa gtgagaaatc accatgagtg acgactgaat ccggtgagaa tggcaacagc 8400
ttatgcattt ctttccagac ttgttcaaca ggccagccat tacgctcgtc atcaaaatca 8460
ctcgcatcaa ccaaaccgtt attcattcgt gattgcgcct gagcgagacg aaatacgcga 8520
tcgctgttaa aaggacaatt acaaacagga atcgaatgca accggcgcag gaacactgcc 8580
agcgcatcaa caatattttc acctgaatca ggatattctt ctaatacctg gaatgctgtt 8640
tttccgggga tcgcagtggt gagtaaccat gcatcatcag gagtacggat aaaatgcttg 8700
atggtcggaa gaggcataaa ttccgtcagc cagtttagtc tgaccatctc atctgtaaca 8760
tcattggcaa cgctaccttt gccatgtttc agaaacaact ctggcgcatc gggcttccca 8820
tacaatcgat agattgtcgc acctgattgc ccgacattat cgcgagccca tttataccca 8880
tataaatcag catccatgtt ggaatttaat cgcggcctag agcaagacgt ttcccgttga 8940
atatggctca taacacccct tgtattactg tttatgtaag cagacagttt tattgttcat 9000
gatgatatat ttttatcttg tgcaatgtaa catcagagat tttgagacac aacaattggt 9060
cgac 9064
Plasmid as defined in FIG. 2C (pDNA2a pGM297)
SEQ ID NO: 32
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tgggggggtg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggctg ccttcggggg ggacggggca gggcggggtt cggcttctgg cgtgtgaccg 1620
gcggctctag agcctctgct aaccatgttc atgccttctt ctttttccta cagctcctgg 1680
gcaacgtgct ggttattgtg ctgtctcatc attttggcaa agaattgctc gagactagtg 1740
acttggtgag taggcttcga gcctagttag aggactagga gaggccgtag ccgtaactac 1800
tctgggcaag tagggcaggc ggtgggtacg caatgggggc ggctacctca gcactaaata 1860
ggagacaatt agaccaattt gagaaaatac gacttcgccc gaacggaaag aaaaagtacc 1920
aaattaaaca tttaatatgg gcaggcaagg agatggagcg cttcggcctc catgagaggt 1980
tgttggagac agaggagggg tgtaaaagaa tcatagaagt cctctacccc ctagaaccaa 2040
caggatcgga gggcttaaaa agtctgttca atcttgtgtg cgtactatat tgcttgcaca 2100
aggaacagaa agtgaaagac acagaggaag cagtagcaac agtaagacaa cactgccatc 2160
tagtggaaaa agaaaaaagt gcaacagaga catctagtgg acaaaagaaa aatgacaagg 2220
gaatagcagc gccacctggt ggcagtcaga attttccagc gcaacaacaa ggaaatgcct 2280
gggtacatgt acccttgtca ccgcgcacct taaatgcgtg ggtaaaagca gtagaggaga 2340
aaaaatttgg agcagaaata gtacccatgt ttcaagccct atcagaaggc tgcacaccct 2400
atgacattaa tcagatgctt aatgtgctag gagatcatca aggggcatta caaatagtga 2460
aagagatcat taatgaagaa gcagcccagt gggatgtaac acacccacta cccgcaggac 2520
ccctaccagc aggacagctc agggaccctc gcggctcaga tatagcaggg accaccagct 2580
cagtacaaga acagttagaa tggatctata ctgctaaccc ccgggtagat gtaggtgcca 2640
tctaccggag atggattatt ctaggacttc aaaagtgtgt caaaatgtac aacccagtat 2700
cagtcctaga cattaggcag ggacctaaag agcccttcaa ggattatgtg gacagatttt 2760
acaaggcaat tagagcagaa caagcctcag gggaagtgaa acaatggatg acagaatcat 2820
tactcattca aaatgctaat ccagattgta aggtcatcct gaagggccta ggaatgcacc 2880
ccacccttga agaaatgtta acggcttgtc agggggtagg aggcccaagc tacaaagcaa 2940
aagtaatggc agaaatgatg cagaccatgc aaaatcaaaa catggtgcag cagggaggtc 3000
caaaaagaca aagaccccca ctaagatgtt ataattgtgg aaaatttggc catatgcaaa 3060
gacaatgtcc ggaaccaagg aaaacaaaat gtctaaagtg tggaaaattg ggacacctag 3120
caaaagactg caggggacag gtgaattttt tagggtatgg acggtggatg ggggcaaaac 3180
cgagaaattt tcccgccgct actcttggag cggaaccgag tgcgcctcct ccaccgagcg 3240
gcaccacccc atacgaccca gcaaagaagc tcctgcagca atatgcagag aaagggaaac 3300
aactgaggga gcaaaagagg aatccaccgg caatgaatcc ggattggacc gagggatatt 3360
ctttgaactc cctctttgga gaagaccaat aaagacagtg tatatagaag gggtccccat 3420
taaggcactg ctagacacag gggcagatga caccataatt aaagaaaatg atttacaatt 3480
atcaggtcca tggagaccca aaattatagg gggcatagga ggaggcctta atgtaaaaga 3540
atataacgac agggaagtaa aaatagaaga taaaattttg agaggaacaa tattgttagg 3600
agcaactccc attaatataa taggtagaaa tttgctggcc ccggcaggtg cccggttagt 3660
aatgggacaa ttatcagaaa aaattcctgt cacacctgtc aaattgaagg aaggggctcg 3720
gggaccctgt gtaagacaat ggcctctctc taaagagaag attgaagctt tacaggaaat 3780
atgttcccaa ttagagcagg aaggaaaaat cagtagagta ggaggagaaa atgcatacaa 3840
taccccaata ttttgcataa agaagaagga caaatcccag tggaggatgc tagtagactt 3900
tagagagtta aataaggcaa cccaagattt ctttgaagtg caattaggga taccccaccc 3960
agcaggatta agaaagatga gacagataac agttttagat gtaggagacg cctattattc 4020
cataccattg gatccaaatt ttaggaaata tactgctttt actattccca cagtgaataa 4080
tcagggaccc gggattaggt atcaattcaa ctgtctcccg caagggtgga aaggatctcc 4140
tacaatcttc caaaatacag cagcatccat tttggaggag ataaaaagaa acttgccagc 4200
actaaccatt gtacaataca tggatgattt atgggtaggt tctcaagaaa atgaacacac 4260
ccatgacaaa ttagtagaac agttaagaac aaaattacaa gcctggggct tagaaacccc 4320
agaaaagaag gtgcaaaaag aaccacctta tgagtggatg ggatacaaac tttggcctca 4380
caaatgggaa ctaagcagaa tacaactgga ggaaaaagat gaatggactg tcaatgacat 4440
ccagaagtta gttgggaaac taaattgggc agcacaattg tatccaggtc ttaggaccaa 4500
gaatatatgc aagttaatta gaggaaagaa aaatctgtta gagctagtga cttggacacc 4560
tgaggcagaa gctgaatatg cagaaaatgc agagattctt aaaacagaac aggaaggaac 4620
ctattacaaa ccaggaatac ctattagggc agcagtacag aaattggaag gaggacagtg 4680
gagttaccaa ttcaaacaag aaggacaagt cttgaaagta ggaaaataca ccaagcaaaa 4740
gaacacccat acaaatgaac ttcgcacatt agctggttta gtgcagaaga tttgcaaaga 4800
agctctagtt atttggggga tattaccagt tctagaactc ccgatagaaa gagaggtatg 4860
ggaacaatgg tgggcggatt actggcaggt aagctggatt cccgaatggg attttgtcag 4920
caccccacct ttgctcaaac tatggtacac attaacaaaa gaacccatac ccaaggagga 4980
cgtttactat gtagatggag catgcaacag aaattcaaaa gaaggaaaag caggatacat 5040
ctcacaatac ggaaaacaga gagtagaaac attagaaaac actaccaatc agcaagcaga 5100
attaacagct ataaaaatgg ctttggaaga cagtgggcct aatgtgaaca tagtaacaga 5160
ctctcaatat gcaatgggaa ttttgacagc acaacccaca caaagtgatt caccattagt 5220
agagcaaatt atagccttaa tgatacaaaa gcaacaaata tatttgcagt gggtaccagc 5280
acataaagga ataggaggaa atgaggagat agataaatta gtgagtaaag gcattagaag 5340
agttttattc ttagaaaaaa tagaagaagc tcaagaagag catgaaagat atcataataa 5400
ttggaaaaac ctagcagata catatgggct tccacaaata gtagcaaaag agatagtggc 5460
catgtgtcca aaatgtcaga taaagggaga accagtgcat ggacaagtgg atgcctcacc 5520
tggaacatgg cagatggatt gtactcatct agaaggaaaa gtagtcatag ttgcggtcca 5580
tgtagccagt ggattcatag aagcagaagt catacctagg gaaacaggaa aagaaacggc 5640
aaagtttcta ttaaaaatac tgagtagatg gcctataaca cagttacaca cagacaatgg 5700
gcctaacttt acctcccaag aagtggcagc aatatgttgg tggggaaaaa ttgaacatac 5760
aacaggtata ccatataacc cccaatctca aggatcaata gaaagcatga acaaacaatt 5820
aaaagagata attgggaaaa taagagatga ttgccaatat acagagacag cagtactgat 5880
ggcttgccat attcacaatt ttaaaagaaa gggaggaata gggggacaga cttcagcaga 5940
gagactaatt aatataataa caacacaatt agaaatacaa catttacaaa ccaaaattca 6000
aaaaatttta aattttagag tctactacag agaagggaga gaccctgtgt ggaaaggacc 6060
agcacaatta atctggaaag gggaaggagc agtggtcctc aaggacggaa gtgacctaaa 6120
ggttgtacca agaaggaaag ctaaaattat taaggattat gaacccaaac aaagagtggg 6180
taatgagggt gacgtggaag gtaccagggg atctgataac taaatggcag ggaatagtca 6240
gatattggat gagacaaaga aatttgaaat ggaactatta tatgcatcag ctggcggccg 6300
cgaattcact agtgattccc gtttgtgcta gggttcttag gcttcttggg ggctgctgga 6360
actgcaatgg gagcagcggc gacagccctg acggtccagt ctcagcattt gcttgctggg 6420
atactgcagc agcagaagaa tctgctggcg gctgtggagg ctcaacagca gatgttgaag 6480
ctgaccattt ggggtgttaa aaacctcaat gcccgcgtca cagcccttga gaagtaccta 6540
gaggatcagg cacgactaaa ctcctggggg tgcgcatgga aacaagtatg tcataccaca 6600
gtggagtggc cctggacaaa tcggactccg gattggcaaa atatgacttg gttggagtgg 6660
gaaagacaaa tagctgattt ggaaagcaac attacgagac aattagtgaa ggctagagaa 6720
caagaggaaa agaatctaga tgcctatcag aagttaacta gttggtcaga tttctggtct 6780
tggttcgatt tctcaaaatg gcttaacatt ttaaaaatgg gatttttagt aatagtagga 6840
ataatagggt taagattact ttacacagta tatggatgta tagtgagggt taggcaggga 6900
tatgttcctc tatctccaca gatccatatc caatcgaatt cccgcggccg caattcactc 6960
ctcaggtgca ggctgcctat cagaaggtgg tggctggtgt ggccaatgcc ctggctcaca 7020
aataccactg agatcttttt ccctctgcca aaaattatgg ggacatcatg aagccccttg 7080
agcatctgac ttctggctaa taaaggaaat ttattttcat tgcaatagtg tgttggaatt 7140
ttttgtgtct ctcactcgga aggacatatg ggagggcaaa tcatttaaaa catcagaatg 7200
agtatttggt ttagagtttg gcaacatatg cccatatgct ggctgccatg aacaaaggtt 7260
ggctataaag aggtcatcag tatatgaaac agccccctgc tgtccattcc ttattccata 7320
gaaaagcctt gacttgaggt tagatttttt ttatattttg ttttgtgtta tttttttctt 7380
taacatccct aaaattttcc ttacatgttt tactagccag atttttcctc ctctcctgac 7440
tactcccagt catagctgtc cctcttctct tatggagatc cctcgacctg cagcccaagc 7500
ttggcgtaat catggtcata gctgtttcct gtgtgaaatt gttatccgct cacaattcca 7560
cacaacatac gagccggaag cataaagtgt aaagcctggg gtgcctaatg agtgagctaa 7620
ctcacattaa ttgcgttgcg ctcactgccc gctttccagt cgggaaacct gtcgtgccag 7680
cggatccgca tctcaattag tcagcaacca tagtcccgcc cctaactccg cccatcccgc 7740
ccctaactcc gcccagttcc gcccattctc cgccccatgg ctgactaatt ttttttattt 7800
atgcagaggc cgaggccgcc tcggcctctg agctattcca gaagtagtga ggaggctttt 7860
ttggaggcct aggcttttgc aaaaagctaa cttgtttatt gcagcttata atggttacaa 7920
ataaagcaat agcatcacaa atttcacaaa taaagcattt ttttcactgc attctagttg 7980
tggtttgtcc aaactcatca atgtatctta tcatgtctgt ccgcttcctc gctcactgac 8040
tcgctgcgct cggtcgttcg gctgcggcga gcggtatcag ctcactcaaa ggcggtaata 8100
cggttatcca cagaatcagg ggataacgca ggaaagaaca tgtgagcaaa aggccagcaa 8160
aaggccagga accgtaaaaa ggccgcgttg ctggcgtttt tccataggct ccgcccccct 8220
gacgagcatc acaaaaatcg acgctcaagt cagaggtggc gaaacccgac aggactataa 8280
agataccagg cgtttccccc tggaagctcc ctcgtgcgct ctcctgttcc gaccctgccg 8340
cttaccggat acctgtccgc ctttctccct tcgggaagcg tggcgctttc tcatagctca 8400
cgctgtaggt atctcagttc ggtgtaggtc gttcgctcca agctgggctg tgtgcacgaa 8460
ccccccgttc agcccgaccg ctgcgcctta tccggtaact atcgtcttga gtccaacccg 8520
gtaagacacg acttatcgcc actggcagca gccactggta acaggattag cagagcgagg 8580
tatgtaggcg gtgctacaga gttcttgaag tggtggccta actacggcta cactagaaga 8640
acagtatttg gtatctgcgc tctgctgaag ccagttacct tcggaaaaag agttggtagc 8700
tcttgatccg gcaaacaaac caccgctggt agcggtggtt tttttgtttg caagcagcag 8760
attacgcgca gaaaaaaagg atctcaagaa gatcctttga tcttttctac ggggtctgac 8820
gctcagtgga acgaaaactc acgttaaggg attttggtca tgagattatc aaaaaggatc 8880
ttcacctaga tccttttaaa ttaaaaatga agttttaaat caatctaaag tatatatgag 8940
taaacttggt ctgacagtta gaaaaactca tcgagcatca aatgaaactg caatttattc 9000
atatcaggat tatcaatacc atatttttga aaaagccgtt tctgtaatga aggagaaaac 9060
tcaccgaggc agttccatag gatggcaaga tcctggtatc ggtctgcgat tccgactcgt 9120
ccaacatcaa tacaacctat taatttcccc tcgtcaaaaa taaggttatc aagtgagaaa 9180
tcaccatgag tgacgactga atccggtgag aatggcaaca gottatgcat ttctttccag 9240
acttgttcaa caggccagcc attacgctcg tcatcaaaat cactcgcatc aaccaaaccg 9300
ttattcattc gtgattgcgc ctgagcgaga cgaaatacgc gatcgctgtt aaaaggacaa 9360
ttacaaacag gaatcgaatg caaccggcgc aggaacactg ccagcgcatc aacaatattt 9420
tcacctgaat caggatattc ttctaatacc tggaatgctg tttttccggg gatcgcagtg 9480
gtgagtaacc atgcatcatc aggagtacgg ataaaatgct tgatggtcgg aagaggcata 9540
aattccgtca gccagtttag tctgaccatc tcatctgtaa catcattggc aacgctacct 9600
ttgccatgtt tcagaaacaa ctctggcgca tcgggcttcc catacaatcg atagattgtc 9660
gcacctgatt gcccgacatt atcgcgagcc catttatacc catataaatc agcatccatg 9720
ttggaattta atcgcggcct agagcaagac gtttcccgtt gaatatggct cataacaccc 9780
cttgtattac tgtttatgta agcagacagt tttattgttc atgatgatat atttttatct 9840
tgtgcaatgt aacatcagag attttgagac acaacaattg gtcgac 9886
Plasmid as defined in FIG. 2D (pDNA2b pGM299)
SEQ ID NO: 33
tcaatattgg ccattagcca tattattcat tggttatata gcataaatca atattggcta 60
ttggccattg catacgttgt atctatatca taatatgtac atttatattg gctcatgtcc 120
aatatgaccg ccatgttggc attgattatt gactagttat taatagtaat caattacggg 180
gtcattagtt catagcccat atatggagtt ccgcgttaca taacttacgg taaatggccc 240
gcctggctga ccgcccaacg acccccgccc attgacgtca ataatgacgt atgttcccat 300
agtaacgcca atagggactt tccattgacg tcaatgggtg gagtatttac ggtaaactgc 360
ccacttggca gtacatcaag tgtatcatat gccaagtccg ccccctattg acgtcaatga 420
cggtaaatgg cccgcctggc attatgccca gtacatgacc ttacgggact ttcctacttg 480
gcagtacatc tacgtattag tcatcgctat taccatggtg atgcggtttt ggcagtacac 540
caatgggcgt ggatagcggt ttgactcacg gggatttcca agtctccacc ccattgacgt 600
caatgggagt ttgttttggc accaaaatca acgggacttt ccaaaatgtc gtaataaccc 660
cgccccgttg acgcaaatgg gcggtaggcg tgtacggtgg gaggtctata taagcagagc 720
tcgtttagtg aaccgtcaga tcactagaag ctttattgcg gtagtttatc acagttaaat 780
tgctaacgca gtcagtgctt ctgacacaac agtctcgaac ttaagctgca gaagttggtc 840
gtgaggcact gggcaggtaa gtatcaaggt tacaagacag gtttaaggag accaatagaa 900
actgggcttg tcgagacaga gaagactctt gcgtttctga taggcaccta ttggtcttac 960
tgacatccac tttgcctttc tctccacagg tgtccactcc cagttcaatt acagctctta 1020
aggctagagt acttaatacg actcactata ggctagcctc gagaattcga ttatgcccct 1080
aggaccagaa gaaagaagat tgcttcgctt gatttggctc ctttacagca ccaatccata 1140
tccaccaagt ggggaaggga cggccagaca acgccgacga gccaggagaa ggtggagaca 1200
acagcaggat caaattagag tottggtaga aagactccaa gagcaggtgt atgcagttga 1260
ccgcctggct gacgaggctc aacacttggc tatacaacag ttgcctgacc ctcctcattc 1320
agcttagaat cactagtgaa ttcacgcgtg gtacctctag agtcgacccg ggcggccgct 1380
tcgagcagac atgataagat acattgatga gtttggacaa accacaacta gaatgcagtg 1440
aaaaaaatgc tttatttgtg aaatttgtga tgctattgct ttatttgtaa ccattataag 1500
ctgcaataaa caagttaaca acaacaattg cattcatttt atgtttcagg ttcaggggga 1560
gatgtgggag gttttttaaa gcaagtaaaa cctctacaaa tgtggtaaaa tcgataagga 1620
tccgtcgacc aattgttgtg tctcaaaatc tctgatgtta cattgcacaa gataaaaata 1680
tatcatcatg aacaataaaa ctgtctgctt acataaacag taatacaagg ggtgttatga 1740
gccatattca acgggaaacg tcttgctcta ggccgcgatt aaattccaac atggatgctg 1800
atttatatgg gtataaatgg gctcgcgata atgtcgggca atcaggtgcg acaatctatc 1860
gattgtatgg gaagcccgat gcgccagagt tgtttctgaa acatggcaaa ggtagcgttg 1920
ccaatgatgt tacagatgag atggtcagac taaactggct gacggaattt atgcctcttc 1980
cgaccatcaa gcattttatc cgtactcctg atgatgcatg gttactcacc actgcgatcc 2040
ccggaaaaac agcattccag gtattagaag aatatcctga ttcaggtgaa aatattgttg 2100
atgcgctggc agtgttcctg cgccggttgc attcgattcc tgtttgtaat tgtcctttta 2160
acagcgatcg cgtatttcgt ctcgctcagg cgcaatcacg aatgaataac ggtttggttg 2220
atgcgagtga ttttgatgac gagcgtaatg gctggcctgt tgaacaagtc tggaaagaaa 2280
tgcataagct gttgccattc tcaccggatt cagtcgtcac tcatggtgat ttctcacttg 2340
ataaccttat ttttgacgag gggaaattaa taggttgtat tgatgttgga cgagtcggaa 2400
tcgcagaccg ataccaggat cttgccatcc tatggaactg cctcggtgag ttttctcctt 2460
cattacagaa acggcttttt caaaaatatg gtattgataa tcctgatatg aataaattgc 2520
agtttcattt gatgctcgat gagtttttct aactgtcaga ccaagtttac tcatatatac 2580
tttagattga tttaaaactt catttttaat ttaaaaggat ctaggtgaag atcctttttg 2640
ataatctcat gaccaaaatc ccttaacgtg agttttcgtt ccactgagcg tcagaccccg 2700
tagaaaagat caaaggatct tottgagatc ctttttttct gcgcgtaatc tgctgcttgc 2760
aaacaaaaaa accaccgcta ccagcggtgg tttgtttgcc ggatcaagag ctaccaactc 2820
tttttccgaa ggtaactggc ttcagcagag cgcagatacc aaatactgtt cttctagtgt 2880
agccgtagtt aggccaccac ttcaagaact ctgtagcacc gcctacatac ctcgctctgc 2940
taatcctgtt accagtggct gctgccagtg gcgataagtc gtgtcttacc gggttggact 3000
caagacgata gttaccggat aaggcgcagc ggtcgggctg aacggggggt tcgtgcacac 3060
agcccagctt ggagcgaacg acctacaccg aactgagata cctacagcgt gagctatgag 3120
aaagcgccac gcttcccgaa gggagaaagg cggacaggta tccggtaagc ggcagggtcg 3180
gaacaggaga gcgcacgagg gagcttccag ggggaaacgc ctggtatctt tatagtcctg 3240
tcgggtttcg ccacctctga cttgagcgtc gatttttgtg atgctcgtca ggggggcgga 3300
gcctatggaa aaacgccagc aacgcggcct ttttacggtt cctggccttt tgctggcctt 3360
ttgctcacat ggctcgacag atct 3384
Plasmid as defined in FIG. 2E (pDNA3a pGM301)
SEQ ID NO: 34
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tgggggggtg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggctg ccttcggggg ggacggggca gggcggggtt cggcttctgg cgtgtgaccg 1620
gcggctctag agcctctgct aaccatgttc atgccttctt ctttttccta cagctcctgg 1680
gcaacgtgct ggttattgtg ctgtctcatc attttggcaa agaattcgat tgccatggca 1740
acatatatcc agagagtaca gtgcatctca acatcactac tggttgttct caccacattg 1800
gtctcgtgtc agattcccag ggataggctc tctaacatag gggtcatagt cgatgaaggg 1860
aaatcactga agatagctgg atcccacgaa tcgaggtaca tagtactgag tctagttccg 1920
ggggtagact ttgagaatgg gtgcggaaca gcccaggtta tccagtacaa gagcctactg 1980
aacaggctgt taatcccatt gagggatgcc ttagatcttc aggaggctct gataactgtc 2040
accaatgata cgacacaaaa tgccggtgct ccccagtcga gattcttcgg tgctgtgatt 2100
ggtactatcg cacttggagt ggcgacatca gcacaaatca ccgcagggat tgcactagcc 2160
gaagcgaggg aggccaaaag agacatagcg ctcatcaaag aatcgatgac aaaaacacac 2220
aagtctatag aactgctgca aaacgctgtg ggggaacaaa ttcttgctct aaagacactc 2280
caggatttcg tgaatgatga gatcaaaccc gcaataagcg aattaggctg tgagactgct 2340
gccttaagac tgggtataaa attgacacag cattactccg agctgttaac tgcgttcggc 2400
tcgaatttcg gaaccatcgg agagaagagc ctcacgctgc aggcgctgtc ttcactttac 2460
tctgctaaca ttactgagat tatgaccaca atcaggacag ggcagtctaa catctatgat 2520
gtcatttata cagaacagat caaaggaacg gtgatagatg tggatctaga gagatacatg 2580
gtcaccctgt ctgtgaagat ccctattctt tctgaagtcc caggtgtgct catacacaag 2640
gcatcatcta tttcttacaa catagacggg gaggaatggt atgtgactgt ccccagccat 2700
atactcagtc gtgcttcttt cttagggggt gcagacataa ccgattgtgt tgagtccaga 2760
ttgacctata tatgccccag ggatcccgca caactgatac ctgacagcca gcaaaagtgt 2820
atcctggggg acacaacaag gtgtcctgtc acaaaagttg tggacagcct tatccccaag 2880
tttgcttttg tgaatggggg cgttgttgct aactgcatag catccacatg tacctgcggg 2940
acaggccgaa gaccaatcag tcaggatcgc tctaaaggtg tagtattcct aacccatgac 3000
aactgtggtc ttataggtgt caatggggta gaattgtatg ctaaccggag agggcacgat 3060
gccacttggg gggtccagaa cttgacagtc ggtcctgcaa ttgctatcag acccgttgat 3120
atttctctca accttgctga tgctacgaat ttcttgcaag actctaaggc tgagcttgag 3180
aaagcacgga aaatcctctc ggaggtaggt agatggtaca actcaagaga gactgtgatt 3240
acgatcatag tagttatggt cgtaatattg gtggtcatta tagtgatcat catcgtgctt 3300
tatagactca gaaggtgaaa tcactagtga attcactcct caggtgcagg ctgcctatca 3360
gaaggtggtg gctggtgtgg ccaatgccct ggctcacaaa taccactgag atctttttcc 3420
ctctgccaaa aattatgggg acatcatgaa gccccttgag catctgactt ctggctaata 3480
aaggaaattt attttcattg caatagtgtg ttggaatttt ttgtgtctct cactcggaag 3540
gacatatggg agggcaaatc atttaaaaca tcagaatgag tatttggttt agagtttggc 3600
aacatatgcc catatgctgg ctgccatgaa caaaggttgg ctataaagag gtcatcagta 3660
tatgaaacag ccccctgctg tccattcctt attccataga aaagccttga cttgaggtta 3720
gatttttttt atattttgtt ttgtgttatt tttttcttta acatccctaa aattttcctt 3780
acatgtttta ctagccagat ttttcctcct ctcctgacta ctcccagtca tagctgtccc 3840
tcttctctta tggagatccc tcgacctgca gcccaagctt ggcgtaatca tggtcatagc 3900
tgtttcctgt gtgaaattgt tatccgctca caattccaca caacatacga gccggaagca 3960
taaagtgtaa agcctggggt gcctaatgag tgagctaact cacattaatt gcgttgcgct 4020
cactgcccgc tttccagtcg ggaaacctgt cgtgccagcg gatccgcatc tcaattagtc 4080
agcaaccata gtcccgcccc taactccgcc catcccgccc ctaactccgc ccagttccgc 4140
ccattctccg ccccatggct gactaatttt ttttatttat gcagaggccg aggccgcctc 4200
ggcctctgag ctattccaga agtagtgagg aggctttttt ggaggcctag gcttttgcaa 4260
aaagctaact tgtttattgc agcttataat ggttacaaat aaagcaatag catcacaaat 4320
ttcacaaata aagcattttt ttcactgcat tctagttgtg gtttgtccaa actcatcaat 4380
gtatcttatc atgtctgtcc gcttcctcgc tcactgactc gctgcgctcg gtcgttcggc 4440
tgcggcgagc ggtatcagct cactcaaagg cggtaatacg gttatccaca gaatcagggg 4500
ataacgcagg aaagaacatg tgagcaaaag gccagcaaaa ggccaggaac cgtaaaaagg 4560
ccgcgttgct ggcgtttttc cataggctcc gcccccctga cgagcatcac aaaaatcgac 4620
gctcaagtca gaggtggcga aacccgacag gactataaag ataccaggcg tttccccctg 4680
gaagctccct cgtgcgctct cctgttccga ccctgccgct taccggatac ctgtccgcct 4740
ttctcccttc gggaagcgtg gcgctttctc atagctcacg ctgtaggtat ctcagttcgg 4800
tgtaggtcgt tcgctccaag ctgggctgtg tgcacgaacc ccccgttcag cccgaccgct 4860
gcgccttatc cggtaactat cgtcttgagt ccaacccggt aagacacgac ttatcgccac 4920
tggcagcagc cactggtaac aggattagca gagcgaggta tgtaggcggt gctacagagt 4980
tcttgaagtg gtggcctaac tacggctaca ctagaagaac agtatttggt atctgcgctc 5040
tgctgaagcc agttaccttc ggaaaaagag ttggtagctc ttgatccggc aaacaaacca 5100
ccgctggtag cggtggtttt tttgtttgca agcagcagat tacgcgcaga aaaaaaggat 5160
ctcaagaaga tcctttgatc ttttctacgg ggtctgacgc tcagtggaac gaaaactcac 5220
gttaagggat tttggtcatg agattatcaa aaaggatctt cacctagatc cttttaaatt 5280
aaaaatgaag ttttaaatca atctaaagta tatatgagta aacttggtct gacagttaga 5340
aaaactcatc gagcatcaaa tgaaactgca atttattcat atcaggatta tcaataccat 5400
atttttgaaa aagccgtttc tgtaatgaag gagaaaactc accgaggcag ttccatagga 5460
tggcaagatc ctggtatcgg tctgcgattc cgactcgtcc aacatcaata caacctatta 5520
atttcccctc gtcaaaaata aggttatcaa gtgagaaatc accatgagtg acgactgaat 5580
ccggtgagaa tggcaacagc ttatgcattt ctttccagac ttgttcaaca ggccagccat 5640
tacgctcgtc atcaaaatca ctcgcatcaa ccaaaccgtt attcattcgt gattgcgcct 5700
gagcgagacg aaatacgcga tcgctgttaa aaggacaatt acaaacagga atcgaatgca 5760
accggcgcag gaacactgcc agcgcatcaa caatattttc acctgaatca ggatattctt 5820
ctaatacctg gaatgctgtt tttccgggga tcgcagtggt gagtaaccat gcatcatcag 5880
gagtacggat aaaatgcttg atggtcggaa gaggcataaa ttccgtcagc cagtttagtc 5940
tgaccatctc atctgtaaca tcattggcaa cgctaccttt gccatgtttc agaaacaact 6000
ctggcgcatc gggcttccca tacaatcgat agattgtcgc acctgattgc ccgacattat 6060
cgcgagccca tttataccca tataaatcag catccatgtt ggaatttaat cgcggcctag 6120
agcaagacgt ttcccgttga atatggctca taacacccct tgtattactg tttatgtaag 6180
cagacagttt tattgttcat gatgatatat ttttatcttg tgcaatgtaa catcagagat 6240
tttgagacac aacaattggt cgac 6264
Plasmid as defined in FIG. 2F (pDNA3b pGM303)
SEQ ID NO: 35
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tgggggggtg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggggc agggcggggt tcggcttctg gcgtgtgacc ggcggctcta gagcctctgc 1620
taaccatgtt catgccttct tctttttcct acagctcctg ggcaacgtgc tggttattgt 1680
gctgtctcat cattttggca aagaattcct cgagcatgtg gtctgagtta aaaatcagga 1740
gcaacgacgg aggtgaagga ccagaggacg ccaacgaccc ccggggaaag ggggtgcaac 1800
acatccatat ccagccatct ctacctgttt atggacagag ggttagggat ggtgataggg 1860
gcaaacgtga ctcgtactgg tctacttctc ctagtggtag caccacaaaa ccagcatcag 1920
gttgggagag gtcaagtaaa gccgacacat ggttgctgat tctctcattc acccagtggg 1980
ctttgtcaat tgccacagtg atcatctgta tcataatttc tgctagacaa gggtatagta 2040
tgaaagagta ctcaatgact gtagaggcat tgaacatgag cagcagggag gtgaaagagt 2100
cacttaccag tctaataagg caagaggtta tagcaagggc tgtcaacatt cagagctctg 2160
tgcaaaccgg aatcccagtc ttgttgaaca aaaacagcag ggatgtcatc cagatgattg 2220
ataagtcgtg cagcagacaa gagctcactc agcactgtga gagtacgatc gcagtccacc 2280
atgccgatgg aattgcccca cttgagccac atagtttctg gagatgccct gtcggagaac 2340
cgtatcttag ctcagatcct gaaatctcat tgctgcctgg tccgagcttg ttatctggtt 2400
ctacaacgat ctctggatgt gttaggctcc cttcactctc aattggcgag gcaatctatg 2460
cctattcatc aaatctcatt acacaaggtt gtgctgacat agggaaatca tatcaggtcc 2520
tgcagctagg gtacatatca ctcaattcag atatgttccc tgatcttaac cccgtagtgt 2580
cccacactta tgacatcaac gacaatcgga aatcatgctc tgtggtggca accgggacta 2640
ggggttatca gctttgctcc atgccgactg tagacgaaag aaccgactac tctagtgatg 2700
gtattgagga tctggtcctt gatgtcctgg atctcaaagg gagaactaag tctcaccggt 2760
atcgcaacag cgaggtagat cttgatcacc cgttctctgc actatacccc agtgtaggca 2820
acggcattgc aacagaaggc tcattgatat ttcttgggta tggtggacta accacccctc 2880
tgcagggtga tacaaaatgt aggacccaag gatgccaaca ggtgtcgcaa gacacatgca 2940
atgaggctct gaaaattaca tggctaggag ggaaacaggt ggtcagcgtg atcatccagg 3000
tcaatgacta tctctcagag aggccaaaga taagagtcac aaccattcca atcactcaaa 3060
actatctcgg ggcggaaggt agattattaa aattgggtga tcgggtgtac atctatacaa 3120
gatcatcagg ctggcactct caactgcaga taggagtact tgatgtcagc caccctttga 3180
ctatcaactg gacacctcat gaagccttgt ctagaccagg aaataaagag tgcaattggt 3240
acaataagtg tccgaaggaa tgcatatcag gcgtatacac tgatgcttat ccattgtccc 3300
ctgatgcagc taacgtcgct accgtcacgc tatatgccaa tacatcgcgt gtcaacccaa 3360
caatcatgta ttctaacact actaacatta taaatatgtt aaggataaag gatgttcaat 3420
tagaggctgc atataccacg acatcgtgta tcacgcattt tggtaaaggc tactgctttc 3480
acatcatcga gatcaatcag aagagcctga ataccttaca gccgatgctc tttaagacta 3540
gcatccctaa attatgcaag gccgagtctt aagcggccgc gcatgcgaat tcactcctca 3600
ggtgcaggct gcctatcaga aggtggtggc tggtgtggcc aatgccctgg ctcacaaata 3660
ccactgagat ctttttccct ctgccaaaaa ttatggggac atcatgaagc cccttgagca 3720
tctgacttct ggctaataaa ggaaatttat tttcattgca atagtgtgtt ggaatttttt 3780
gtgtctctca ctcggaagga catatgggag ggcaaatcat ttaaaacatc agaatgagta 3840
tttggtttag agtttggcaa catatgccca tatgctggct gccatgaaca aaggttggct 3900
ataaagaggt catcagtata tgaaacagcc ccctgctgtc tattccttat tccatagaaa 3960
agccttgact tgaggttaga ttttttttat attttgtttt gtgttatttt tttctttaac 4020
atccctaaaa ttttccttac atgttttact agccagattt ttcctcctct cctgactact 4080
cccagtcata gctgtccctc ttctcttatg gagatccctc gacctgcagc ccaagcttgg 4140
cgtaatcatg gtcatagctg tttcctgtgt gaaattgtta tccgctcaca attccacaca 4200
acatacgagc cggaagcata aagtgtaaag cctggggtgc ctaatgagtg agctaactca 4260
cattaattgc gttgcgctca ctgcccgctt tccagtcggg aaacctgtcg tgccagcgga 4320
tccgcatctc aattagtcag caaccatagt cccgccccta actccgccca tcccgcccct 4380
aactccgccc agttccgccc attctccgcc ccatggctga ctaatttttt ttatttatgc 4440
agaggccgag gccgcctcgg cctctgagct attccagaag tagtgaggag gcttttttgg 4500
aggcctaggc ttttgcaaaa agctaacttg tttattgcag cttataatgg ttacaaataa 4560
agcaatagca tcacaaattt cacaaataaa gcattttttt cactgcattc tagttgtggt 4620
ttgtccaaac tcatcaatgt atcttatcat gtctgtccgc cttcctgctc actgactcgc 4680
tgcgctcggt cgttcggctg cggcgagcgg tatcagctca ctcaaaggcg gtaatacggt 4740
tatccacaga atcaggggat aacgcaggaa agaacatgtg agcaaaaggc cagcaaaagg 4800
ccaggaaccg taaaaaggcc gcgttgctgg cgtttttcca taggctccgc ccccctgacg 4860
agcatcacaa aaatcgacgc tcaagtcaga ggtggcgaaa cccgacagga ctataaagat 4920
accaggcgtt tccccctgga agctccctcg tgcgctctcc tgttccgacc ctgccgotta 4980
ccggatacct gtccgccttt ctcccttcgg gaagcgtggc gctttctcat agctcacgct 5040
gtaggtatct cagttcggtg taggtcgttc gctccaagct gggctgtgtg cacgaacccc 5100
ccgttcagcc cgaccgctgc gccttatccg gtaactatcg tcttgagtcc aacccggtaa 5160
gacacgactt atcgccactg gcagcagcca ctggtaacag gattagcaga gcgaggtatg 5220
taggcggtgc tacagagttc ttgaagtggt ggcctaacta cggctacact agaagaacag 5280
tatttggtat ctgcgctctg ctgaagccag ttaccttcgg aaaaagagtt ggtagctctt 5340
gatccggcaa acaaaccacc gctggtagcg gtggtttttt tgtttgcaag cagcagatta 5400
cgcgcagaaa aaaaggatct caagaagatc ctttgatctt ttctacgggg tctgacgctc 5460
agtggaacga aaactcacgt taagggattt tggtcatgag attatcaaaa aggatcttca 5520
cctagatcct tttaaattaa aaatgaagtt ttaaatcaat ctaaagtata tatgagtaaa 5580
cttggtctga cagttagaaa aactcatcga gcatcaaatg aaactgcaat ttattcatat 5640
caggattatc aataccatat ttttgaaaaa gccgtttctg taatgaagga gaaaactcac 5700
cgaggcagtt ccataggatg gcaagatcct ggtatcggtc tgcgattccg actcgtccaa 5760
catcaataca acctattaat ttcccctcgt caaaaataag gttatcaagt gagaaatcac 5820
catgagtgac gactgaatcc ggtgagaatg gcaacagctt atgcatttct ttccagactt 5880
gttcaacagg ccagccatta cgctcgtcat caaaatcact cgcatcaacc aaaccgttat 5940
tcattcgtga ttgcgcctga gcgagacgaa atacgcgatc gctgttaaaa ggacaattac 6000
aaacaggaat cgaatgcaac cggcgcagga acactgccag cgcatcaaca atattttcac 6060
ctgaatcagg atattcttct aatacctgga atgctgtttt tccggggatc gcagtggtga 6120
gtaaccatgc atcatcagga gtacggataa aatgcttgat ggtcggaaga ggcataaatt 6180
ccgtcagcca gtttagtctg accatctcat ctgtaacatc attggcaacg ctacctttgc 6240
catgtttcag aaacaactct ggcgcatcgg gcttcccata caatcgatag attgtcgcac 6300
ctgattgccc gacattatcg cgagcccatt tatacccata taaatcagca tccatgttgg 6360
aatttaatcg cggcctagag caagacgttt cccgttgaat atggctcata acaccccttg 6420
tattactgtt tatgtaagca gacagtttta ttgttcatga tgatatattt ttatcttgtg 6480
caatgtaaca tcagagattt tgagacacaa caattggtcg ac 6522
Plasmid as defined in FIG. 2G (pDNA2a pGM407)
SEQ ID NO: 36
ggtacctcaa tattggccat tagccatatt attcattggt tatatagcat aaatcaatat 60
tggctattgg ccattgcata cgttgtatct atatcataat atgtacattt atattggctc 120
atgtccaata tgaccgccat gttggcattg attattgact agttattaat agtaatcaat 180
tacggggtca ttagttcata gcccatatat ggagttccgc gttacataac ttacggtaaa 240
tggcccgcct ggctgaccgc ccaacgaccc ccgcccattg acgtcaataa tgacgtatgt 300
tcccatagta acgccaatag ggactttcca ttgacgtcaa tgggtggagt atttacggta 360
aactgcccac ttggcagtac atcaagtgta tcatatgcca agtccgcccc ctattgacgt 420
caatgacggt aaatggcccg cctggcatta tgcccagtac atgaccttac gggactttcc 480
tacttggcag tacatctacg tattagtcat cgctattacc atggtgatgc ggttttggca 540
gtacaccaat gggcgtggat agcggtttga ctcacgggga tttccaagtc tccaccccat 600
tgacgtcaat gggagtttgt tttggcacca aaatcaacgg gactttccaa aatgtcgtaa 660
caactgcgat cgcccgcccc gttgacgcaa atgggcggta ggcgtgtacg gtgggaggtc 720
tatataagca gagctcgctg gcttgtaact cagtctctta ctaggagacc agcttgagcc 780
tgggtgttcg ctggttagcc taacctggtt ggccaccagg ggtaaggact ccttggctta 840
gaaagctaat aaacttgcct gcattagagc ttatctgagt caagtgtcct cattgacgcc 900
tcactctctt gaacgggaat cttccttact gggttctctc tctgacccag gcgagagaaa 960
ctccagcagt ggcgcccgaa cagggacttg agtgagagtg taggcacgta cagctgagaa 1020
ggcgtcggac gcgaaggaag cgcggggtgc gacgcgacca agaaggagac ttggtgagta 1080
ggcttctcga gtgccgggaa aaagctcgag cctagttaga ggactaggag aggccgtagc 1140
cgtaactact cttgggcaag tagggcaggc ggtgggtacg caatgggggc ggctacctca 1200
gcactaaata ggagacaatt agaccaattt gagaaaatac gacttcgccc gaacggaaag 1260
aaaaagtacc aaattaaaca tttaatatgg gcaggcaagg agatggagcg cttcggcctc 1320
catgagaggt tgttggagac agaggagggg tgtaaaagaa tcatagaagt cctctacccc 1380
ctagaaccaa caggatcgga gggcttaaaa agtctgttca atcttgtgtg cgtgctatat 1440
tgcttgcaca aggaacagaa agtgaaagac acagaggaag cagtagcaac agtaagacaa 1500
cactgccatc tagtggaaaa agaaaaaagt gcaacagaga catctagtgg acaaaagaaa 1560
aatgacaagg gaatagcagc gccacctggt ggcagtcaga attttccagc gcaacaacaa 1620
ggaaatgcct gggtacatgt acccttgtca ccgcgcacct taaatgcgtg ggtaaaagca 1680
gtagaggaga aaaaatttgg agcagaaata gtacccattt ttttgtttca agccctatcg 1740
aattcccgtt tgtgctaggg ttcttaggct tcttgggggc tgctggaact gcaatgggag 1800
cagcggcgac agccctgacg gtccagtctc agcatttgct tgctgggata ctgcagcagc 1860
agaagaatct gctggcggct gtggaggctc aacagcagat gttgaagctg accatttggg 1920
gtgttaaaaa cctcaatgcc cgcgtcacag cccttgagaa gtacctagag gatcaggcac 1980
gactaaactc ctgggggtgc gcatggaaac aagtatgtca taccacagtg gagtggccct 2040
ggacaaatcg gactccggat tggcaaaata tgacttggtt ggagtgggaa agacaaatag 2100
ctgatttgga aagcaacatt acgagacaat tagtgaaggc tagagaacaa gaggaaaaga 2160
atctagatgc ctatcagaag ttaactagtt ggtcagattt ctggtcttgg ttcgatttct 2220
caaaatggct taacatttta aaaatgggat ttttagtaat agtaggaata atagggttaa 2280
gattacttta cacagtatat ggatgtatag tgagggttag gcagggatat gttcctctat 2340
ctccacagat ccatatccgc ggcaatttta aaagaaaggg aggaataggg ggacagactt 2400
cagcagagag actaattaat ataataacaa cacaattaga aatacaacat ttacaaacca 2460
aaattcaaaa aattttaaat tttagagccg cggagatctg ttacataact tatggtaaat 2520
ggcctgcctg gctgactgcc caatgacccc tgcccaatga tgtcaataat gatgtatgtt 2580
cccatgtaat gccaataggg actttccatt gatgtcaatg ggtggagtat ttatggtaac 2640
tgcccacttg gcagtacatc aagtgtatca tatgccaagt atgcccccta ttgatgtcaa 2700
tgatggtaaa tggcctgcct ggcattatgc ccagtacatg accttatggg actttcctac 2760
ttggcagtac atctatgtat tagtcattgc tattaccatg ggaattcact agtggagaag 2820
agcatgcttg agggctgagt gcccctcagt gggcagagag cacatggccc acagtccctg 2880
agaagttggg gggaggggtg ggcaattgaa ctggtgccta gagaaggtgg ggcttgggta 2940
aactgggaaa gtgatgtggt gtactggctc cacctttttc cccagggtgg gggagaacca 3000
tatataagtg cagtagtctc tgtgaacatt caagcttctg ccttctccct cctgtgagtt 3060
tgctagccac catgcccagc tctgtgtcct ggggcattct gctgctggct ggcctgtgct 3120
gtctggtgcc tgtgtccctg gctgaggacc ctcaggggga tgctgcccag aaaacagaca 3180
cctcccacca tgaccaggac caccccacct tcaacaagat cacccccaac ctggcagagt 3240
ttgccttcag cctgtacaga cagctggccc accagagcaa cagcaccaac atctttttca 3300
gccctgtgtc cattgccaca gcctttgcca tgctgagcct gggcaccaag gctgacaccc 3360
atgatgagat cctggaaggc ctgaacttca acctgacaga gatccctgag gcccagatcc 3420
atgagggctt ccaggaactg ctgagaaccc tgaaccagcc agacagccag ctgcagctga 3480
caacaggcaa tgggctgttc ctgtctgagg gcctgaagct ggtggacaag tttctggaag 3540
atgtgaagaa gctgtaccac tctgaggcct tcacagtgaa ctttggggac acagaagagg 3600
ccaagaaaca gatcaatgac tatgtggaaa agggcaccca gggcaagatt gtggaccttg 3660
tgaaagagct ggacagggac actgtgtttg cccttgtgaa ctacatcttc ttcaagggca 3720
agtgggagag gccctttgaa gtgaaggaca ctgaggaaga ggacttccat gtggaccaag 3780
tgaccacagt gaaggtgcca atgatgaaga gactggggat gttcaatatc cagcactgca 3840
agaaactgag cagctgggtg ctgctgatga agtacctggg caatgctaca gccatattct 3900
ttctgcctga tgagggcaag ctgcagcacc tggaaaatga gctgacccat gacatcatca 3960
ccaaatttct ggaaaatgag gacagaagat ctgccagcct gcatctgccc aagctgagca 4020
tcacaggcac atatgacctg aagtctgtgc tgggacagct gggaatcacc aaggtgttca 4080
gcaatggggc agacctgagt ggagtgacag aggaagcccc tctgaagctg tccaaggctg 4140
tgcacaaggc agtgctgacc attgatgaga agggcacaga ggctgctggg gccatgtttc 4200
tggaagccat ccccatgtcc atccccccag aagtgaagtt caacaagccc tttgtgttcc 4260
tgatgattga gcagaacacc aagagccccc tgttcatggg caaggttgtg aaccccaccc 4320
agaaatgagg gcccaatcaa cctctggatt acaaaatttg tgaaagattg actggtattc 4380
ttaactatgt tgctcctttt acgctatgtg gatacgctgc tttaatgcct ttgtatcatg 4440
ctattgcttc ccgtatggct ttcattttct cctccttgta taaatcctgg ttgctgtctc 4500
tttatgagga gttgtggccc gttgtcaggc aacgtggcgt ggtgtgcact gtgtttgctg 4560
acgcaacccc cactggttgg ggcattgcca ccacctgtca gctcctttcc gggactttcg 4620
ctttccccct ccctattgcc acggcggaac tcatcgccgc ctgccttgcc cgctgctgga 4680
caggggctcg gctgttgggc actgacaatt ccgtggtgtt gtcggggaaa tcatcgtcct 4740
ttccttggct gctcgcctgt gttgccacct ggattctgcg cgggacgtcc ttctgctacg 4800
tcccttcggc cctcaatcca gcggaccttc cttcccgcgg cctgctgccg gctctgcggc 4860
ctcttccgcg tcttcgcctt cgccctcaga cgagtcggat ctccctttgg gccgcctccc 4920
cgcaagcttc gcacttttta aaagaaaagg gaggactgga tgggatttat tactccgata 4980
ggacgctggc ttgtaactca gtctcttact aggagaccag cttgagcctg ggtgttcgct 5040
ggttagccta acctggttgg ccaccagggg taaggactcc ttggcttaga aagctaataa 5100
acttgcctgc attagagctc ttacgcgtcc cgggctcgag atccgcatct caattagtca 5160
gcaaccatag tcccgcccct aactccgccc atcccgcccc taactccgcc cagttccgcc 5220
cattctccgc cccatggctg actaattttt tttatttatg cagaggccga ggccgcctcg 5280
gcctctgagc tattccagaa gtagtgagga ggcttttttg gaggcctagg cttttgcaaa 5340
aagctaactt gtttattgca gcttataatg gttacaaata aagcaatagc atcacaaatt 5400
tcacaaataa agcatttttt tcactgcatt ctagttgtgg tttgtccaaa ctcatcaatg 5460
tatcttatca tgtctgtccg cttcctcgct cactgactcg ctgcgctcgg tcgttcggct 5520
gcggcgagcg gtatcagctc actcaaaggc ggtaatacgg ttatccacag aatcagggga 5580
taacgcagga aagaacatgt gagcaaaagg ccagcaaaag gccaggaacc gtaaaaaggc 5640
cgcgttgctg gcgtttttcc ataggctccg cccccctgac gagcatcaca aaaatcgacg 5700
ctcaagtcag aggtggcgaa acccgacagg actataaaga taccaggcgt ttccccctgg 5760
aagctccctc gtgcgctctc ctgttccgac cctgccgctt accggatacc tgtccgcctt 5820
tctcccttcg ggaagcgtgg cgctttctca tagctcacgc tgtaggtatc tcagttcggt 5880
gtaggtcgtt cgctccaagc tgggctgtgt gcacgaaccc cccgttcagc ccgaccgctg 5940
cgccttatcc ggtaactatc gtcttgagtc caacccggta agacacgact tatcgccact 6000
ggcagcagcc actggtaaca ggattagcag agcgaggtat gtaggcggtg ctacagagtt 6060
cttgaagtgg tggcctaact acggctacac tagaagaaca gtatttggta tctgcgctct 6120
gctgaagcca gttaccttcg gaaaaagagt tggtagctct tgatccggca aacaaaccac 6180
cgctggtagc ggtggttttt ttgtttgcaa gcagcagatt acgcgcagaa aaaaaggatc 6240
tcaagaagat cctttgatct tttctacggg gtctgacgct cagtggaacg aaaactcacg 6300
ttaagggatt ttggtcatga gattatcaaa aaggatcttc acctagatcc ttttaaatta 6360
aaaatgaagt tttaaatcaa tctaaagtat atatgagtaa acttggtctg acagttagaa 6420
aaactcatcg agcatcaaat gaaactgcaa tttattcata tcaggattat caataccata 6480
tttttgaaaa agccgtttct gtaatgaagg agaaaactca ccgaggcagt tccataggat 6540
ggcaagatcc tggtatcggt ctgcgattcc gactcgtcca acatcaatac aacctattaa 6600
tttcccctcg tcaaaaataa ggttatcaag tgagaaatca ccatgagtga cgactgaatc 6660
cggtgagaat ggcaacagct tatgcatttc tttccagact tgttcaacag gccagccatt 6720
acgctcgtca tcaaaatcac tcgcatcaac caaaccgtta ttcattcgtg attgcgcctg 6780
agcgagacga aatacgcgat cgctgttaaa aggacaatta caaacaggaa tcgaatgcaa 6840
ccggcgcagg aacactgcca gcgcatcaac aatattttca cctgaatcag gatattcttc 6900
taatacctgg aatgctgttt ttccggggat cgcagtggtg agtaaccatg catcatcagg 6960
agtacggata aaatgcttga tggtcggaag aggcataaat tccgtcagcc agtttagtct 7020
gaccatctca tctgtaacat cattggcaac gctacctttg ccatgtttca gaaacaactc 7080
tggcgcatcg ggcttcccat acaatcgata gattgtcgca cctgattgcc cgacattatc 7140
gcgagcccat ttatacccat ataaatcagc atccatgttg gaatttaatc gcggcctaga 7200
gcaagacgtt tcccgttgaa tatggctcat aacacccctt gtattactgt ttatgtaagc 7260
agacagtttt attgttcatg atgatatatt tttatcttgt gcaatgtaac atcagagatt 7320
ttgagacaca acaattggtc gacggatcc 7349
Codon-optimised SIV gag-pol nucleic acid sequence (from pGM691)
SEQ ID NO: 37
atgggagctg ccacatctgc cctgaataga cggcagctgg accagttcga gaagatcaga 60
ctgcggccca acggcaagaa gaagtaccag atcaagcacc tgatctgggc cggcaaagag 120
atggaaagat tcggcctgca cgagcggctg ctggaaaccg aggaaggctg caagagaatt 180
atcgaggtgc tgtaccctct ggaacctacc ggctctgagg gcctgaagtc cctgttcaat 240
ctcgtgtgcg tgctgtactg cctgcacaaa gaacagaaag tgaaggacac cgaagaggcc 300
gtggccacag ttagacagca ctgccacctg gtggaaaaag agaagtccgc cacagagaca 360
agcagcggcc agaagaagaa cgacaaggga attgctgccc ctcctggcgg cagccagaat 420
tttcctgctc agcagcaggg aaacgcctgg gtgcacgttc cactgagccc tagaacactg 480
aatgcctggg tcaaagccgt ggaagagaag aagtttggcg ccgagatcgt gcccatgttc 540
caggctctgt ctgagggctg caccccttac gacatcaacc agatgctgaa cgtgctggga 600
gatcaccagg gcgctctgca gatcgtgaaa gagatcatca acgaagaggc tgcccagtgg 660
gacgtgacac atccattgcc tgctggacct ctgccagccg gacaactgag agatcctaga 720
ggctctgata tcgccggcac caccagctct gtgcaagagc agctggaatg gatctacacc 780
gccaatccta gagtggacgt gggcgccatc tacagaagat ggatcatcct gggcctgcag 840
aaatgcgtga agatgtacaa ccccgtgtcc gtgctggaca tcagacaggg acccaaagag 900
cccttcaagg actacgtgga ccggttctat aaggccatta gagccgagca ggccagcggc 960
gaagtgaagc agtggatgac agagagcctg ctgatccaga acgccaatcc agactgcaaa 1020
gtgatcctga aaggcctggg catgcacccc acactggaag agatgctgac agcctgtcaa 1080
ggcgttggcg gcccttctta caaagccaaa gtgatggccg agatgatgca gaccatgcag 1140
aaccagaaca tggtgcagca aggcggccct aagagacaga ggcctcctct gagatgctac 1200
aactgcggca agttcggcca catgcagaga cagtgtcctg agcctaggaa aacaaaatgt 1260
ctaaagtgtg gaaaattggg acacctagca aaagactgca ggggacaggt gaatttttta 1320
gggtatggac ggtggatggg ggcaaaaccg agaaattttc ccgccgctac tcttggagcg 1380
gaaccgagtg cgcctcctcc accgagcggc accaccccat acgacccagc aaagaagctc 1440
ctgcagcaat atgcagagaa agggaaacaa ctgagggagc aaaagaggaa tccaccggca 1500
atgaatccgg attggaccga gggatattct ttgaactccc tctttggaga agaccaataa 1560
agaccgtgta catcgagggc gtgcccatca aggctctgct ggatacaggc gccgacgaca 1620
ccatcatcaa agagaacgac ctgcagctga gcggcccttg gaggcctaag atcattggag 1680
gaatcggcgg aggcctgaac gtcaaagagt acaacgaccg ggaagtgaag atcgaggaca 1740
agatcctgag gggcacaatc ctgctgggcg ccacacctat caacatcatc ggcagaaatc 1800
tgctggcccc tgccggcgct agactggtta tgggacagct ctctgagaag atccccgtga 1860
cacccgtgaa gctgaaagaa ggcgctagag gaccttgtgt gcgacagtgg cctctgagca 1920
aagagaagat tgaggccctg caagaaatct gtagccagct ggaacaagag ggcaagatca 1980
gcagagttgg cggcgagaac gcctacaata cccctatctt ctgcatcaag aaaaaggaca 2040
agagccagtg gcggatgctg gtggacttta gagagctgaa caaggctacc caggacttct 2100
tcgaggtgca gctgggaatt cctcatcctg ccggcctgcg gaagatgaga cagatcacag 2160
tgctggatgt gggcgacgcc tactacagca tccctctgga ccccaacttc agaaagtaca 2220
ccgccttcac aatccccacc gtgaacaatc aaggccctgg catcagatac cagttcaact 2280
gcctgcctca aggctggaag ggcagcccca ccatttttca gaataccgcc gccagcatcc 2340
tggaagaaat caagagaaac ctgcctgctc tgaccatcgt gcagtacatg gacgatctgt 2400
gggtcggaag ccaagagaat gagcacaccc acgacaagct ggtggaacag ctgagaacaa 2460
agctgcaggc ctggggcctc gaaacccctg agaagaaggt gcagaaagaa cctccttacg 2520
agtggatggg ctacaagctg tggcctcaca agtgggagct gagccggatt cagctcgaag 2580
agaaggacga gtggaccgtg aacgacatcc agaaactcgt gggcaagctg aattgggcag 2640
cccagctgta tcccggcctg aggaccaaga acatctgcaa gctgatccgg ggaaagaaga 2700
acctgctgga actggtcaca tggacacctg aggccgaggc cgaatatgcc gagaatgccg 2760
aaatcctgaa aaccgagcaa gaggggacct actacaagcc tggcattcca atcagagctg 2820
ccgtgcagaa actggaaggc ggccagtggt cctaccagtt taagcaagaa ggccaggtcc 2880
tgaaagtggg caagtacacc aagcagaaga acacccacac caacgagctg aggacactgg 2940
ctggcctggt ccagaaaatc tgcaaagagg ccctggtcat ttggggcatc ctgcctgttc 3000
tggaactgcc cattgagcgg gaagtgtggg aacagtggtg ggccgattac tggcaagtgt 3060
cttggatccc cgagtgggac ttcgtgtcta cccctcctct gctgaaactg tggtacaccc 3120
tgacaaaaga gcccattcct aaagaggacg tctactacgt tgacggcgcc tgcaaccgga 3180
actccaaaga aggcaaggcc ggctacatca gccagtacgg caagcagaga gtggaaaccc 3240
tggaaaacac caccaaccag caggccgagc tgaccgccat taagatggcc ctggaagata 3300
gcggccccaa tgtgaacatc gtgaccgact ctcagtacgc catgggaatc ctgacagccc 3360
agcctacaca gagcgatagc cctctggttg agcagatcat tgccctgatg attcagaagc 3420
agcaaatcta cctgcagtgg gtgcccgctc acaaaggcat cggcggaaac gaagagatcg 3480
ataagctggt gtccaaggga atcagacggg tgctgttcct ggaaaagatt gaagaggccc 3540
aagaggaaca cgagcgctac cacaacaact ggaagaatct ggccgacacc tacggactgc 3600
cccagatcgt ggccaaagaa atcgtggcta tgtgccccaa gtgtcagate aagggcgaac 3660
ctgtgcacgg ccaagtggat gcttctcctg gcacatggca gatggactgt acccacctgg 3720
aaggcaaagt ggtcatcgtg gctgtgcacg tggcctccgg ctttattgag gccgaagtga 3780
tccccagaga gacaggcaaa gaaaccgcca agttcctgct gaagatcctg tccagatggc 3840
ccatcacaca gctgcacacc gacaacggcc ctaacttcac atctcaagag gtggccgcca 3900
tctgttggtg gggaaagatt gagcacacaa ccggcattcc ctacaatcca cagagccagg 3960
gcagcatcga gtccatgaac aagcagctca aagagattat cggcaagatc cgggacgact 4020
gccagtacac agaaacagcc gtgctgatgg cctgtcacat ccacaacttc aagcggaaag 4080
gcggcatcgg aggacagaca tctgccgaga gactgatcaa tatcatcacc actcagctgg 4140
aaatccagca cctccagacc aagatccaga agattctgaa cttccgggtg tactaccgcg 4200
agggcagaga tcctgtttgg aaaggcccag cacagctgat ctggaaaggc gaaggtgccg 4260
tggtgctgaa ggatggctct gatctgaagg tggtgcccag acggaaggcc aagattatca 4320
aggattacga gcccaaacag cgcgtgggca atgaaggcga cgttgagggc acaagaggca 4380
gcgacaattg a 4391
Exemplary CAG promoter
SEQ ID NO: 38
attgattatt gactagttat taatagtaat caattacggg gtcattagtt catagcccat 60
atatggagtt ccgcgttaca taacttacgg taaatggccc gcctggctga ccgcccaacg 120
acccccgccc attgacgtca ataatgacgt atgttcccat agtaacgcca atagggactt 180
tccattgacg tcaatgggtg gagtatttac ggtaaactgc ccacttggca gtacatcaag 240
tgtatcatat gccaagtacg ccccctattg acgtcaatga cggtaaatgg cccgcctggc 300
attatgccca gtacatgacc ttatgggact ttcctacttg gcagtacatc tacgtattag 360
tcatcgctat taccatggtc gaggtgagcc ccacgttctg cttcactctc cccatctccc 420
ccccctcccc acccccaatt ttgtatttat ttatttttta attattttgt gcagcgatgg 480
gggcgggggg gggggggggg cgcgcgccag gcggggcggg gcggggcgag gggcggggcg 540
gggcgaggcg gagaggtgcg gcggcagcca atcagagcgg cgcgctccga aagtttcctt 600
ttatggcgag gcggcggcgg cggcggccct ataaaaagcg aagcgcgcgg cgggcgggag 660
tcgctgcgcg ctgccttcgc cccgtgcccc gctccgccgc cgcctcgcgc cgcccgcccc 720
ggctctgact gaccgcgtta ctcccacagg tgagcgggcg ggacggccct tctcctccgg 780
gctgtaatta gcgcttggtt taatgacggc ttgtttcttt tctgtggctg cgtgaaagcc 840
ttgaggggct ccgggagggc cctttgtgcg gggggagcgg ctcggggggt gcgtgcgtgt 900
gtgtgtgcgt ggggagcgcc gcgtgcggct ccgcgctgcc cggcggctgt gagcgctgcg 960
ggcgcggcgc ggggctttgt gcgctccgca gtgtgcgcga ggggagcgcg gccgggggcg 1020
gtgccccgcg gtgcgggggg ggctgcgagg ggaacaaagg ctgcgtgcgg ggtgtgtgcg 1080
tgggggggtg agcagggggt gtgggcgcgt cggtcgggct gcaacccccc ctgcaccccc 1140
ctccccgagt tgctgagcac ggcccggctt cgggtgcggg gctccgtacg gggcgtggcg 1200
cggggctcgc cgtgccgggc ggggggtggc ggcaggtggg ggtgccgggc ggggcggggc 1260
cgcctcgggc cggggagggc tcgggggagg ggcgcggcgg cccccggagc gccggcggct 1320
gtcgaggcgc ggcgagccgc agccattgcc ttttatggta atcgtgcgag agggcgcagg 1380
gacttccttt gtcccaaatc tgtgcggagc cgaaatctgg gaggcgccgc cgcaccccct 1440
ctagcgggcg cggggcgaag cggtgcggcg ccggcaggaa ggaaatgggc ggggagggcc 1500
ttcgtgcgtc gccgcgccgc cgtccccttc tccctctcca gcctcggggc tgtccgcggg 1560
gggacggctg ccttcggggg ggacggggca gggcggggtt cggcttctgg cgtgtgaccg 1620
gcggctctag agcctctgct aaccatgttc atgccttctt ctttttccta cagctcctgg 1680
gcaacgtgct ggttattgtg ctgtctcatc attttggcaa agaattgctc gagccacc 1738
Exemplified WPRE component (mWPRE)
SEQ ID NO: 39
gggcccaatc aacctctgga ttacaaaatt tgtgaaagat tgactggtat tottaactat 60
gttgctcctt ttacgctatg tggatacgct gctttaatgc ctttgtatca tgctattgct 120
tcccgtatgg ctttcatttt ctcctccttg tataaatcct ggttgctgtc tctttatgag 180
gagttgtggc ccgttgtcag gcaacgtggc gtggtgtgca ctgtgtttgc tgacgcaacc 240
cccactggtt ggggcattgc caccacctgt cagctccttt ccgggacttt cgctttcccc 300
ctccctattg ccacggcgga actcatcgcc gcctgccttg cccgctgctg gacaggggct 360
cggctgttgg gcactgacaa ttccgtggtg ttgtcgggga aatcatcgtc ctttccttgg 420
ctgctcgcct gtgttgccac ctggattctg cgcgggacgt ccttctgcta cgtcccttcg 480
gccctcaatc cagcggacct tccttcccgc ggcctgctgc cggctctgcg gcctcttccg 540
cgtcttcgcc ttcgccctca gacgagtcgg atctcccttt gggccgcctc cccgcaagct 600
Exemplified Human SFTPB transgene
SEQ ID NO: 40
ATGCACCAAGCAGGGTACCCAGGCTGCAGAGGTGCCATGGCTGAGTCACACCTGCTGCAGTGGCTGCTGCTGCTG
CTGCCCACGCTCTGTGGCCCAGGCACTGCTGCCTGGACCACCTCATCCTTGGCCTGTGCCCAGGGCCCTGAGTTC
TGGTGCCAAAGCCTGGAGCAAGCATTGCAGTGCAGAGCCCTAGGGCATTGCCTACAGGAAGTCTGGGGACATGTG
GGAGCCGATGACCTATGCCAAGAGTGTGAGGACATCGTCCACATCCTTAACAAGATGGCCAAGGAGGCCATTTTC
CAGGACACGATGAGGAAGTTCCTGGAGCAGGAGTGCAACGTCCTCCCCTTGAAGCTGCTCATGCCCCAGTGCAAC
CAAGTGCTTGACGACTACTTCCCCCTGGTCATCGACTACTTCCAGAACCAGACTGACTCAAACGGCATCTGTATG
CACCTGGGCCTGTGCAAATCCCGGCAGCCAGAGCCAGAGCAGGAGCCAGGGATGTCAGACCCCCTGCCCAAACCT
CTGCGGGACCCTCTGCCAGACCCTCTGCTGGACAAGCTCGTCCTCCCTGTGCTGCCCGGGGCCCTCCAGGCGAGG
CCTGGGCCTCACACACAGGATCTCTCCGAGCAGCAATTCCCCATTCCTCTCCCCTATTGCTGGCTCTGCAGGGCT
CTGATCAAGCGGATCCAAGCCATGATTCCCAAGGGTGCGCTAGCTGTGGCAGTGGCCCAGGTGTGCCGCGTGGTA
CCTCTGGTGGCGGGCGGCATCTGCCAGTGCCTGGCTGAGCGCTACTCCGTCATCCTGCTCGACACGCTGCTGGGC
CGCATGCTGCCCCAGCTGGTCTGCCGCCTCGTCCTCCGGTGCTCCATGGATGACAGCGCTGGCCCAAGGTCGCCG
ACAGGAGAATGGCTGCCGCGAGACTCTGAGTGCCACCTCTGCATGTCCGTGACCACCCAGGCCGGGAACAGCAGC
GAGCAGGCCATACCACAGGCAATGCTCCAGGCCTGTGTTGGCTCCTGGCTGGACAGGGAAAAGTGCAAGCAATTT
GTGGAGCAGCACACGCCCCAGCTGCTGACCCTGGTGCCCAGGGGCTGGGATGCCCACACCACCTGCCAGGCCCTC
GGGGTGTGTGGGACCATGTCCAGCCCTCTCCAGTGTATCCACAGCCCCGACCTTTGA
Exemplified Human SFTPB polypeptide
SEQ ID NO: 41
MAESHLLQWLLLLLPTLCGPGTAAWTTSSLACAQGPEFWCQSLEQALQCRALGHCLQEVWGHVGADDLCQECEDI
VHILNKMAKEAIFQDTMRKFLEQECNVLPLKLLMPQCNQVLDDYFPLVIDYFQNQTDSNGICMHLGLCKSRQPEP
EQEPGMSDPLPKPLRDPLPDPLLDKLVLPVLPGALQARPGPHTQDLSEQQFPIPLPYCWLCRALIKRIQAMIPKG
ALAVAVAQVCRVVPLVAGGICQCLAERYSVILLDTLLGRMLPQLVCRLVLRCSMDDSAGPRSPTGEWLPRDSECH
LCMSVTTQAGNSSEQAIPQAMLQACVGSWLDREKCKQFVEQHTPQLLTLVPRGWDAHTTCQALGVCGTMSSPLQC
IHSPDL
Exemplified Human ADAMTS13 transgene
SEQ ID NO: 42
ATGCACCAGCGTCACCCCCGGGCAAGATGCCCTCCCCTCTGTGTGGCCGGAATCCTTGCCTGTGGCTTTCTCCTG
GGCTGCTGGGGACCCTCCCATTTCCAGCAGAGTTGTCTTCAGGCTTTGGAGCCACAGGCCGTGTCTTCTTACTTG
AGCCCTGGTGCTCCCTTAAAAGGCCGCCCTCCTTCCCCTGGCTTCCAGAGGCAGAGGCAGAGGCAGAGGCGGGCT
GCAGGCGGCATCCTACACCTGGAGCTGCTGGTGGCCGTGGGCCCCGATGTCTTCCAGGCTCACCAGGAGGACACA
GAGCGCTATGTGCTCACCAACCTCAACATCGGGGCAGAACTGCTTCGGGACCCGTCCCTGGGGGCTCAGTTTCGG
GTGCACCTGGTGAAGATGGTCATTCTGACAGAGCCTGAGGGTGCTCCAAATATCACAGCCAACCTCACCTCGTCC
CTGCTGAGCGTCTGTGGGTGGAGCCAGACCATCAACCCTGAGGACGACACGGATCCTGGCCATGCTGACCTGGTC
CTCTATATCACTAGGTTTGACCTGGAGTTGCCTGATGGTAACCGGCAGGTGCGGGGCGTCACCCAGCTGGGCGGT
GCCTGCTCCCCAACCTGGAGCTGCCTCATTACCGAGGACACTGGCTTCGACCTGGGAGTCACCATTGCCCATGAG
ATTGGGCACAGCTTCGGCCTGGAGCACGACGGCGCGCCCGGCAGCGGCTGCGGCCCCAGCGGACACGTGATGGCT
TCGGACGGCGCCGCGCCCCGCGCCGGCCTCGCCTGGTCCCCCTGCAGCCGCCGGCAGCTGCTGAGCCTGCTCAGC
GCAGGACGGGCGCGCTGCGTGTGGGACCCGCCGCGGCCTCAACCCGGGTCCGCGGGGCACCCGCCGGATGCGCAG
CCTGGCCTCTACTACAGCGCCAACGAGCAGTGCCGCGTGGCCTTCGGCCCCAAGGCTGTCGCCTGCACCTTCGCC
AGGGAGCACCTGGATATGTGCCAGGCCCTCTCCTGCCACACAGACCCGCTGGACCAAAGCAGCTGCAGCCGCCTC
CTCGTTCCTCTCCTGGATGGGACAGAATGTGGCGTGGAGAAGTGGTGCTCCAAGGGTCGCTGCCGCTCCCTGGTG
GAGCTGACCCCCATAGCAGCAGTGCATGGGCGCTGGTCTAGCTGGGGTCCCCGAAGTCCTTGCTCCCGCTCCTGC
GGAGGAGGTGTGGTCACCAGGAGGCGGCAGTGCAACAACCCCAGACCTGCCTTTGGGGGGCGTGCATGTGTTGGT
GCTGACCTCCAGGCCGAGATGTGCAACACTCAGGCCTGCGAGAAGACCCAGCTGGAGTTCATGTCGCAACAGTGC
GCCAGGACCGACGGCCAGCCGCTGCGCTCCTCCCCTGGCGGCGCCTCCTTCTACCACTGGGGTGCTGCTGTACCA
CACAGCCAAGGGGATGCTCTGTGCAGACACATGTGCCGGGCCATTGGCGAGAGCTTCATCATGAAGCGTGGAGAC
AGCTTCCTCGATGGGACCCGGTGTATGCCAAGTGGCCCCCGGGAGGACGGGACCCTGAGCCTGTGTGTGTCGGGC
AGCTGCAGGACATTTGGCTGTGATGGTAGGATGGACTCCCAGCAGGTATGGGACAGGTGCCAGGTGTGTGGTGGG
GACAACAGCACGTGCAGCCCACGGAAGGGCTCTTTCACAGCTGGCAGAGCGAGAGAATATGTCACGTTTCTGACA
GTTACCCCCAACCTGACCAGTGTCTACATTGCCAACCACAGGCCTCTCTTCACACACTTGGCGGTGAGGATCGGA
GGGCGCTATGTCGTGGCTGGGAAGATGAGCATCTCCCCTAACACCACCTACCCCTCCCTCCTGGAGGATGGTCGT
GTCGAGTACAGAGTGGCCCTCACCGAGGACCGGCTGCCCCGCCTGGAGGAGATCCGCATCTGGGGACCCCTCCAG
GAAGATGCTGACATCCAGGTTTACAGGCGGTATGGCGAGGAGTATGGCAACCTCACCCGCCCAGACATCACCTTC
ACCTACTTCCAGCCTAAGCCACGGCAGGCCTGGGTGTGGGCCGCTGTGCGTGGGCCCTGCTCGGTGAGCTGTGGG
GCAGGGCTGCGCTGGGTAAACTACAGCTGCCTGGACCAGGCCAGGAAGGAGTTGGTGGAGACTGTCCAGTGCCAA
GGGAGCCAGCAGCCACCAGCGTGGCCAGAGGCCTGCGTGCTCGAACCCTGCCCTCCCTACTGGGCGGTGGGAGAC
TTCGGCCCATGCAGCGCCTCCTGTGGGGGTGGCCTGCGGGAGCGGCCAGTGCGCTGCGTGGAGGCCCAGGGCAGC
CTCCTGAAGACATTGCCCCCAGCCCGGTGCAGAGCAGGGGCCCAGCAGCCAGCTGTGGCGCTGGAAACCTGCAAC
CCCCAGCCCTGCCCTGCCAGGTGGGAGGTGTCAGAGCCCAGCTCATGCACATCAGCTGGTGGAGCAGGCCTGGCC
TTGGAGAACGAGACCTGTGTGCCAGGGGCAGATGGCCTGGAGGCTCCAGTGACTGAGGGGCCTGGCTCCGTAGAT
GAGAAGCTGCCTGCCCCTGAGCCCTGTGTCGGGATGTCATGTCCTCCAGGCTGGGGCCATCTGGATGCCACCTCT
GCAGGGGAGAAGGCTCCCTCCCCATGGGGCAGCATCAGGACGGGGGCTCAAGCTGCACACGTGTGGACCCCTGCG
GCAGGGTCGTGCTCCGTCTCCTGCGGGCGAGGTCTGATGGAGCTGCGTTTCCTGTGCATGGACTCTGCCCTCAGG
GTGCCTGTCCAGGAAGAGCTGTGTGGCCTGGCAAGCAAGCCTGGGAGCCGGCGGGAGGTCTGCCAGGCTGTCCCG
TGCCCTGCTCGGTGGCAGTACAAGCTGGCGGCCTGCAGCGTGAGCTGTGGGAGAGGGGTCGTGCGGAGGATCCTG
TATTGTGCCCGGGCCCATGGGGAGGACGATGGTGAGGAGATCCTGTTGGACACCCAGTGCCAGGGGCTGCCTCGC
CCGGAACCCCAGGAGGCCTGCAGCCTGGAGCCCTGCCCACCTAGGTGGAAAGTCATGTCCCTTGGCCCATGTTCG
GCCAGCTGTGGCCTTGGCACTGCTAGACGCTCGGTGGCCTGTGTGCAGCTCGACCAAGGCCAGGACGTGGAGGTG
GACGAGGCGGCCTGTGCGGCGCTGGTGCGGCCCGAGGCCAGTGTCCCCTGTCTCATTGCCGACTGCACCTACCGC
TGGCATGTTGGCACCTGGATGGAGTGCTCTGTTTCCTGTGGGGATGGCATCCAGCGCCGGCGTGACACCTGCCTC
GGACCCCAGGCCCAGGCGCCTGTGCCAGCTGATTTCTGCCAGCACTTGCCCAAGCCGGTGACTGTGCGTGGCTGC
TGGGCTGGGCCCTGTGTGGGACAGGGTGCCTGTGGCAGGCAGCACCTTGAGCCAACAGGAACCATTGACATGCGA
GGCCCAGGGCAGGCAGACTGTGCAGTGGCCATTGGGCGGCCCCTCGGGGAGGTGGTGACCCTCCGCGTCCTTGAG
AGTTCTCTCAACTGCAGTGCGGGGGACATGTTGCTGCTTTGGGGCCGGCTCACCTGGAGGAAGATGTGCAGGAAG
CTGTTGGACATGACTTTCAGCTCCAAGACCAACACGCTGGTGGTGAGGCAGCGCTGCGGGCGGCCAGGAGGTGGG
GTGCTGCTGCGGTATGGGAGCCAGCTTGCTCCTGAAACCTTCTACAGAGAATGTGACATGCAGCTCTTTGGGCCC
TGGGGTGAAATCGTGAGCCCCTCGCTGAGTCCAGCCACGAGTAATGCAGGGGGCTGCCGGCTCTTCATTAATGTG
GCTCCGCACGCACGGATTGCCATCCATGCCCTGGCCACCAACATGGGCGCTGGGACCGAGGGAGCCAATGCCAGC
TACATCTTGATCCGGGACACCCACAGCTTGAGGACCACAGCGTTCCATGGGCAGCAGGTGCTCTACTGGGAGTCA
GAGAGCAGCCAGGCTGAGATGGAGTTCAGCGAGGGCTTCCTGAAGGCTCAGGCCAGCCTGCGGGGCCAGTACTGG
ACCCTCCAATCATGGGTACCGGAGATGCAGGACCCTCAGTCCTGGAAGGGAAAGGAAGGAACCTGA
Exemplified Human ADAMTS13 polypeptide
SEQ ID NO: 43
MHQRHPRARCPPLCVAGILACGFLLGCWGPSHFQQSCLQALEPQAVSSYLSPGAPLKGRPPSPGFQRQRQRORRA
AGGILHLELLVAVGPDVFQAHQEDTERYVLTNLNIGAELLRDPSLGAQFRVHLVKMVILTEPEGAPNITANLTSS
LLSVCGWSQTINPEDDTDPGHADLVLYITRFDLELPDGNRQVRGVTQLGGACSPTWSCLITEDTGFDLGVTIAHE
IGHSFGLEHDGAPGSGCGPSGHVMASDGAAPRAGLAWSPCSRRQLLSLLSAGRARCVWDPPRPQPGSAGHPPDAQ
PGLYYSANEQCRVAFGPKAVACTFAREHLDMCQALSCHTDPLDQSSCSRLLVPLLDGTECGVEKWCSKGRCRSLV
ELTPIAAVHGRWSSWGPRSPCSRSCGGGVVTRRRQCNNPRPAFGGRACVGADLQAEMCNTQACEKTQLEFMSQQC
ARTDGQPLRSSPGGASFYHWGAAVPHSQGDALCRHMCRAIGESFIMKRGDSFLDGTRCMPSGPREDGTLSLCVSG
SCRTFGCDGRMDSQQVWDRCQVCGGDNSTCSPRKGSFTAGRAREYVTFLTVTPNLTSVYIANHRPLFTHLAVRIG
GRYVVAGKMSISPNTTYPSLLEDGRVEYRVALTEDRLPRLEEIRIWGPLQEDADIQVYRRYGEEYGNLTRPDITF
TYFQPKPRQAWVWAAVRGPCSVSCGAGLRWVNYSCLDQARKELVETVQCQGSQQPPAWPEACVLEPCPPYWAVGD
FGPCSASCGGGLRERPVRCVEAQGSLLKTLPPARCRAGAQQPAVALETCNPQPCPARWEVSEPSSCTSAGGAGLA
LENETCVPGADGLEAPVTEGPGSVDEKLPAPEPCVGMSCPPGWGHLDATSAGEKAPSPWGSIRTGAQAAHVWTPA
AGSCSVSCGRGLMELRFLCMDSALRVPVQEELCGLASKPGSRREVCQAVPCPARWQYKLAACSVSCGRGVVRRIL
YCARAHGEDDGEEILLDTQCQGLPRPEPQEACSLEPCPPRWKVMSLGPCSASCGLGTARRSVACVQLDQGQDVEV
DEAACAALVRPEASVPCLIADCTYRWHVGTWMECSVSCGDGIQRRRDTCLGPQAQAPVPADFCQHLPKPVTVRGC
WAGPCVGQGACGRQHLEPTGTIDMRGPGQADCAVAIGRPLGEVVTLRVLESSLNCSAGDMLLLWGRLTWRKMCRK
LLDMTFSSKTNTLVVRQRCGRPGGGVLLRYGSQLAPETFYRECDMQLFGPWGEIVSPSLSPATSNAGGCRLFINV
APHARIAIHALATNMGAGTEGANASYILIRDTHSLRTTAFHGQQVLYWESESSQAEMEFSEGFLKAQASLRGQYW
TLQSWVPEMQDPQSWKGKEGT
Exemplified rSIV Rev protein
SEQ ID NO: 44
MPLGPEERRLLRLIWLLYSTNPYPPSGEGTARQRRRARRRWRQQQDQIRVLVERLQEQVYAVDRLADEAQHLAIQ
QLPDPPHSA
oIC001 DNA forward primer
SEQ ID NO: 45
GGGAAAGTGATGTGGTGTACTG
oIC002 DNA and RNA reverse primer
SEQ ID NO: 46
CAATGGACACAGGGCTGAAA
oIC105 RNA forward primer
SEQ ID NO: 47
TGCCTTCTCCCTCCTGT
CAGGS promoter comprising a chicken β-actin splice donor and a rabbit
β-globulin splice acceptor
SEQ ID NO: 48
ggagtcgctgcgttgccttcgccccgtgccccgctccgcgccgcctcgcgccgcccgccccggctctgactgacc
gcgttactcccacaggtgagcgggcgggacggcccttctcctccgggctgtaattagcgcttggtttaatgacgg
ctcgtttcttttctgtggctgcgtgaaagccttaaagggctccgggagggccctttgtgcgggggggagcggctc
ggggggtgcgtgcgtgtgtgtgtgcgtggggagcgccgcgtgcggcccgcgctgcccggcggctgtgagcgctgc
gggcgcggcgcggggctttgtgcgctccgcgtgtgcgcgaggggagcgcggccgggggcggtgccccgcggtgcg
ggggggctgcgaggggaacaaaggctgcgtgcggggtgtgtgcgtgggggggtgagcagggggtgtgggcgcggc
ggtcgggctgtaacccccccctgcacccccctccccgagttgctgagcacggcccggcttcgggtgcggggctcc
gtgcggggcgtggcgcggggctcgccgtgccgggcggggggtggcggcaggtgggggtgccgggcggggcggggc
cgcctcgggccggggagggctcgggggaggggcgcggcggccccggagcgccggcggctgtcgaggcgcggcgag
ccgcagccattgccttttatggtaatcgtgcgagagggcgcaagggacttcctttgtcccaaatctgtgcggagc
cgaaatctgggaggcgccgccgcaccccctctagcgggcgcggggcgaagcggtgcggcgccggcaggaaggaaa
tgggcggggagggccttcgtgcgtcgccgcgccgccgtccccttctccctctccagcctcggggctgtccgcggg
gggacggctgccttcgggggggacggggcagggcggggttcggcttctggcgtgtgaccggcagctctagagcct
ctgctaaccatgttcatgccttcttctttttcctacag
Plasmid as defined in FIG. 2H (pDNA3 pMD2.G)
SEQ ID NO: 49
GGATCCCCTGAGGGGGCCCCCATGGGCTAGAGGATCCGGCCTCGGCCTCTGCATAAATAAAAAAAATTAGTCAGC
CATGAGCTTGGCCCATTGCATACGTTGTATCCATATCATAATATGTACATTTATATTGGCTCATGTCCAACATTA
CCGCCATGTTGACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGGAGTATTTACGG
TAAACTGCCCACTTGGCAGTACATCAAGTGTATCATATGCCAAGTACGCCCCCTATTGACGTCAATGACGGTAAA
TGGCCCGCCTGGCATTATGCCCAGTACATGACCTTATGGGACTTTCCTACTTGGCAGTACATCTACGTATTAGTC
ATCGCTATTACCATGGTGATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGGGACTTTCCAAAATGTCGT
AACAACTCCGCCCCATTGACGCAAATGGGCGGTAGGCGTGTACGGTGGGAGGTCTATATAAGCAGAGCTCGTTTA
GTGAACCGTCAGATCGCCTGGAGACGCCATCCACGCTGTTTTGACCTCCATAGAAGACACCGGGACCGATCCAGC
CTCCCCTCGAAGCTTACATGTGGTACCGAGCTCGGATCCTGAGAACTTCAGGGTGAGTCTATGGGACCCTTGATG
TTTTCTTTCCCCTTCTTTTCTATGGTTAAGTTCATGTCATAGGAAGGGGAGAAGTAACAGGGTACACATATTGAC
CAAATCAGGGTAATTTTGCATTTGTAATTTTAAAAAATGCTTTCTTCTTTTAATATACTTTTTTGTTTATCTTAT
TTCTAATACTTTCCCTAATCTCTTTCTTTCAGGGCAATAATGATACAATGTATCATGCCTCTTTGCACCATTCTA
AAGAATAACAGTGATAATTTCTGGGTTAAGGCAATAGCAATATTTCTGCATATAAATATTTCTGCATATAAATTG
TAACTGATGTAAGAGGTTTCATATTGCTAATAGCAGCTACAATCCAGCTACCATTCTGCTTTTATTTTATGGTTG
GGATAAGGCTGGATTATTCTGAGTCCAAGCTAGGCCCTTTTGCTAATCATGTTCATACCTCTTATCTTCCTCCCA
CAGCTCCTGGGCAACGTGCTGGTCTGTGTGCTGGCCCATCACTTTGGCAAAGCACGTGAGATCTGAATTCAACAG
AGATCGATCTGTTTCCTTGACACTATGAAGTGCCTTTTGTACTTAGCCTTTTTATTCATTGGGGTGAATTGCAAG
TTCACCATAGTTTTTCCACACAACCAAAAAGGAAACTGGAAAAATGTTCCTTCTAATTACCATTATTGCCCGTCA
AGCTCAGATTTAAATTGGCATAATGACTTAATAGGCACAGCCATACAAGTCAAAATGCCCAAGAGTCACAAGGCT
ATTCAAGCAGACGGTTGGATGTGTCATGCTTCCAAATGGGTCACTACTTGTGATTTCCGCTGGTATGGACCGAAG
TATATAACACAGTCCATCCGATCCTTCACTCCATCTGTAGAACAATGCAAGGAAAGCATTGAACAAACGAAACAA
GGAACTTGGCTGAATCCAGGCTTCCCTCCTCAAAGTTGTGGATATGCAACTGTGACGGATGCCGAAGCAGTGATT
GTCCAGGTGACTCCTCACCATGTGCTGGTTGATGAATACACAGGAGAATGGGTTGATTCACAGTTCATCAACGGA
AAATGCAGCAATTACATATGCCCCACTGTCCATAACTCTACAACCTGGCATTCTGACTATAAGGTCAAAGGGCTA
TGTGATTCTAACCTCATTTCCATGGACATCACCTTCTTCTCAGAGGACGGAGAGCTATCATCCCTGGGAAAGGAG
GGCACAGGGTTCAGAAGTAACTACTTTGCTTATGAAACTGGAGGCAAGGCCTGCAAAATGCAATACTGCAAGCAT
TGGGGAGTCAGACTCCCATCAGGTGTCTGGTTCGAGATGGCTGATAAGGATCTCTTTGCTGCAGCCAGATTCCCT
GAATGCCCAGAAGGGTCAAGTATCTCTGCTCCATCTCAGACCTCAGTGGATGTAAGTCTAATTCAGGACGTTGAG
AGGATCTTGGATTATTCCCTCTGCCAAGAAACCTGGAGCAAAATCAGAGCGGGTCTTCCAATCTCTCCAGTGGAT
CTCAGCTATCTTGCTCCTAAAAACCCAGGAACCGGTCCTGCTTTCACCATAATCAATGGTACCCTAAAATACTTT
GAGACCAGATACATCAGAGTCGATATTGCTGCTCCAATCCTCTCAAGAATGGTCGGAATGATCAGTGGAACTACC
ACAGAAAGGGAACTGTGGGATGACTGGGCACCATATGAAGACGTGGAAATTGGACCCAATGGAGTTCTGAGGACC
AGTTCAGGATATAAGTTTCCTTTATACATGATTGGACATGGTATGTTGGACTCCGATCTTCATCTTAGCTCAAAG
GCTCAGGTGTTCGAACATCCTCACATTCAAGACGCTGCTTCGCAACTTCCTGATGATGAGAGTTTATTTTTTGGT
GATACTGGGCTATCCAAAAATCCAATCGAGCTTGTAGAAGGTTGGTTCAGTAGTTGGAAAAGCTCTATTGCCTCT
TTTTTCTTTATCATAGGGTTAATCATTGGACTATTCTTGGTTCTCCGAGTTGGTATCCATCTTTGCATTAAATTA
AAGCACACCAAGAAAAGACAGATTTATACAGACATAGAGATGAACCGACTTGGAAAGTAACTCAAATCCTGCACA
ACAGATTCTTCATGTTTGGACCAAATCAACTTGTGATACCATGCTCAAAGAGGCCTCAATTATATTTGAGTTTTT
AATTTTTATGGAATTCACCCCACCAGTGCAGGCTGCCTATCAGAAAGTGGTGGCTGGTGTGGCTAATGCCCTGGC
CCACAAGTTTCACTAAGCTCGCTTCCTTGCTGTCCAATTTCTATTAAAGGTTCCTTGGTTCCCTAAGTCCAACTA
CTAAACTGGGGGATATTATGAAGGGCCTTGAGCATCTGGATTCTGCCTAATAAAAAACATTTATTTTCATTGCAA
TGATGTATTTAAATTATTTCTGAATATTTTACTAAAAAGGGAATGTGGGAGGTCAGTGCATTTAAAACATAAAGA
AATGAAGAGCTAGTTCAAACCTTGGGAAAATACACTATATCTTAAACTCCATGAAAGAAGGTGAGGCTGCAAACA
GCTAATGCACATTGGCAACAGCCCTGATGCCTATGCCTTATTCATCCCTCAGAAAAGGATTCAAGTAGAGGCTTG
ATTTGGAGGTTAAAGTTTGGCTATGCTGTATTTTACATTACTTATTGTTTTAGCTGTCCTCATGAATGTCTTTTC
ACTACCCATTTGCTTATCCTGCATCTCTCAGCCTTGACTCCACTCAGTTCTCTTGCTTAGAGATACCACCTTTCC
CCTGAAGTGTTCCTTCCATGTTTTACGGCGAGATGGTTTCTCCTCGCCTGGCCACTCAGCCTTAGTTGTCTCTGT
TGTCTTATAGAGGTCTACTTGAAGAAGGAAAAACAGGGGGCATGGTTTGACTGTCCTGTGAGCCCTTCTTCCCTG
CCTCCCCCACTCACAGTGACCCGGAATCCCTCGACATGGCAGTCTAGCACTAGTGCGGCCGCAGATCTGCTTCCT
CGCTCACTGACTCGCTGCGCTCGGTCGTTCGGCTGCGGCGAGCGGTATCAGCTCACTCAAAGGCGGTAATACGGT
TATCCACAGAATCAGGGGATAACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAATCGACGCTCAAGTCAGA
GGTGGCGAAACCCGACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCTCAATGCTCACGCT
GTAGGTATCTCAGTTCGGTGTAGGTCGTTCGCTCCAAGCTGGGCTGTGTGCACGAACCCCCCGTTCAGCCCGACC
GCTGCGCCTTATCCGGTAACTATCGTCTTGAGTCCAACCCGGTAAGACACGACTTATCGCCACTGGCAGCAGCCA
CTGGTAACAGGATTAGCAGAGCGAGGTATGTAGGCGGTGCTACAGAGTTCTTGAAGTGGTGGCCTAACTACGGCT
ACACTAGAAGGACAGTATTTGGTATCTGCGCTCTGCTGAAGCCAGTTACCTTCGGAAAAAGAGTTGGTAGCTCTT
GATCCGGCAAACAAACCACCGCTGGTAGCGGTGGTTTTTTTGTTTGCAAGCAGCAGATTACGCGCAGAAAAAAAG
GATCTCAAGAAGATCCTTTGATCTTTTCTACGGGGTCTGACGCTCAGTGGAACGAAAACTCACGTTAAGGGATTT
TGGTCATGAGATTATCAAAAAGGATCTTCACCTAGATCCTTTTAAATTAAAAATGAAGTTTTAAATCAATCTAAA
GTATATATGAGTAAACTTGGTCTGACAGTTACCAATGCTTAATCAGTGAGGCACCTATCTCAGCGATCTGTCTAT
TTCGTTCATCCATAGTTGCCTGACTCCCCGTCGTGTAGATAACTACGATACGGGAGGGCTTACCATCTGGCCCCA
GTGCTGCAATGATACCGCGAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTGCCGGGAAGCTAGAGTAA
GTAGTTCGCCAGTTAATAGTTTGCGCAACGTTGTTGCCATTGCTACAGGCATCGTGGTGTCACGCTCGTCGTTTG
GTATGGCTTCATTCAGCTCCGGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCACTCATGGTTATGGCAGCAC
TGCATAATTCTCTTACTGTCATGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGATAATACCGCGCCACATAGCAGAA
CTTTAAAAGTGCTCATCATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGAATACTCATACTCTTCCTTT
TTCAATATTATTGAAGCATTTATCAGGGTTATTGTCTCATGAGCGGATACATATTTGAATGTATTTAGAAAAATA
AACAAATAGGGGTTCCGCGCACATTTCCCCGAAAAGTGCCACCTGACGT
HIV RRE sequence
SEQ ID NO: 50
agtagcacccaccaaggcaaagagaagagtggtgcagagagaaaaaagagcagtgggaataggagctttgttcct
tgggttcttgggagcagcaggaagcactatgggcgcagcgtcaatgacgctgacggtacaggccagacaattatt
gtctggtatagtgcagcagcagaacaatttgctgagggctattgaggcgcaacagcatctgttgcaactcacagt
ctggggcatcaagcagctccaggcaagaatcctggctgtggaaagatacctaaaggatcaacagctcctggggat
ttggggttgctctggaaaactcatttgcaccactgctgtgccttggaatgctagt
```

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1—Design and Production of a β-Globin/IgG Chimeric Intron and Insertion into an SIV Vector Genome Plasmid

A functional β-globin/IgG chimeric intron comprising an SIV RRE was designed using SnapGene® Software (version 5.3.2, Insightful Science, San Diego, CA, USA; available at www.snapgene.com). To begin, the chimeric intron sequence of the pCI Mammalian Expression Vector (Cat. E1731, Promega, Madison, WI, USA) was analysed using the Basic Local Alignment Search Tool (BLAST, National Centre for Biotechnology Information, Bethesda, MD, USA; https://blast.ncbi.nlm.nih.gov/Blast.cgi). A region homologous to the Hemoglobin Subunit B gene, as well as the splice donor, branch site, polypyrimidine tract, and splice acceptor were mapped onto the chimeric intron.

The Rev Response Element (RRE) from r.SIV (Griesenbach et al. (2012) American Journal of Respiratory and Critical Care Medicine 186, 846-856). was moved from upstream of the promoter and inserted in silico within the intron, 5′ of the region homologous to Hemoglobin, and 3′ to the splice acceptor site. The resulting lentiviral genome plasmid backbone, with the RRE moved from between the partial GAG and cPPT elements to within an intron, was synthesized as a DNA Ultramer with a 5′ KpnI and a3′ NheI restriction site by IDT (IDT, Newark, NJ, USA). The synthesized Ultramer was double digested with KpnI-HF and NheI restriction enzymes (Cat. #R3142 and R3131, New England BioLabs, Ipswich, MA, USA) following manufacture's protocols, size separated on a 1% agarose gel, purified with a Monarch® DNA gel extraction kit (Cat. #T1020, New England BioLabs), and ligated with T4 DNA ligase (Cat. #M0202, New England BioLabs) following manufacture's protocols. Ligation products were heat shock transformed into 5a competent Escherichia coli (Cat. #2987, New England BioLabs), plated on LB plates with 50 μg/mL of kanamycin, and incubated overnight at 37 °C. Individual colonies were picked, grown up in 5 mL liquid LB cultures with 50 μg/mL kanamycin. Clones were MiniPrepped using a Monarch® Plasmid MiniPrep Kit (Cat. #T010, New England BioLabs) and screened by PCR, restriction digest, and sanger sequencing. Glycerol stocks were made of a successful clone by combining 500 μL of E. coli in liquid culture with 500 μL of 50% glycerol, and storing immediately at −80 °C.

FIG. 1 shows a schematic of the resulting β-globin/IgG chimeric intron comprising an SIV RRE. FIG. 2G shows a plasmid map of the pGM407 SIV vector genome plasmid, which comprises an AAT transgene under the control of a hCEF promoter. The pGM407 plasmid lacks an intron and comprises a SIV RRE. FIG. 2A shows a plasmid map of a plasmid derived from pGM407 which has had the SIV RRE deleted and the β-globin/IgG chimeric intron comprising an SIV RRE of FIG. 1 inserted between with hCEF promoter and the AAT transgene, as described above.

### Example 2—Transfection of HEK293T Cells with a Plasmid Comprising a β-Globin/IgG Chimeric Intron with an Inserted SIV RRE Increases AAT Transgene Expression

HEK293T cells were transfected with a plasmid lacking the endogenous SIV RRE and comprising (i) an AAT transgene under the control of an hCEF promoter and (ii) a β-globin/IgG chimeric intron with an inserted SIV RRE as produced in Example 1 (pGM991).

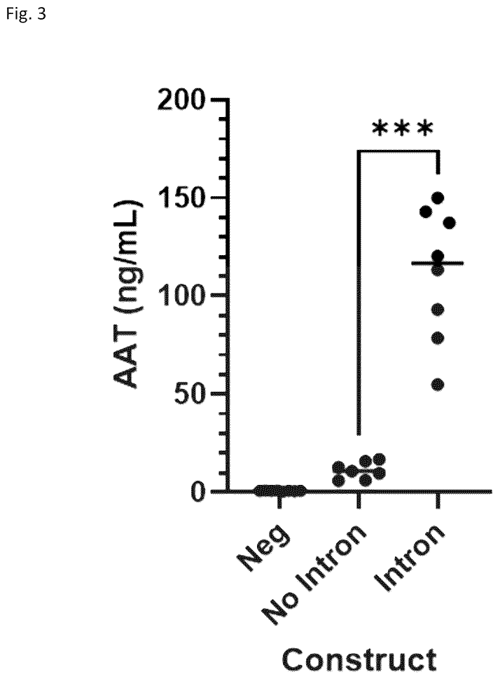

As shown in FIG. 3, transfection of HEK293T cells with pGM991 significantly increased AAT expression compared with transfection of HEK293T cells with a corresponding plasmid comprising the endogenous SIV RRE and lacking the β-globin/IgG chimeric intron with an inserted SIV RRE (pGM407). In particular, an increase in AAT expression of 10.6-fold was observed on transfection with the plasmid lacking the endogenous SIV RRE and comprising the β-globin/IgG chimeric intron with an inserted SIV RRE compared with transfection with a corresponding plasmid comprising the endogenous SIV RRE and lacking said intron/RRE.

PCR of DNA extracted from the transfected HEK293T cells was carried out using primers that bind to either side of the intron:

| Primer | SEQ ID | | |
|---|---|---|---|
| Name | NO: | Sequence | Assays |
| oIC001 | 45 | GGGAAAGTGAT | DNA forward |
| | | GTGGTGTACTG | primer |
| oIC002 | 46 | CAATGGACACA | DNA and RNA |
| | | GGGCTGAAA | reverse primer |

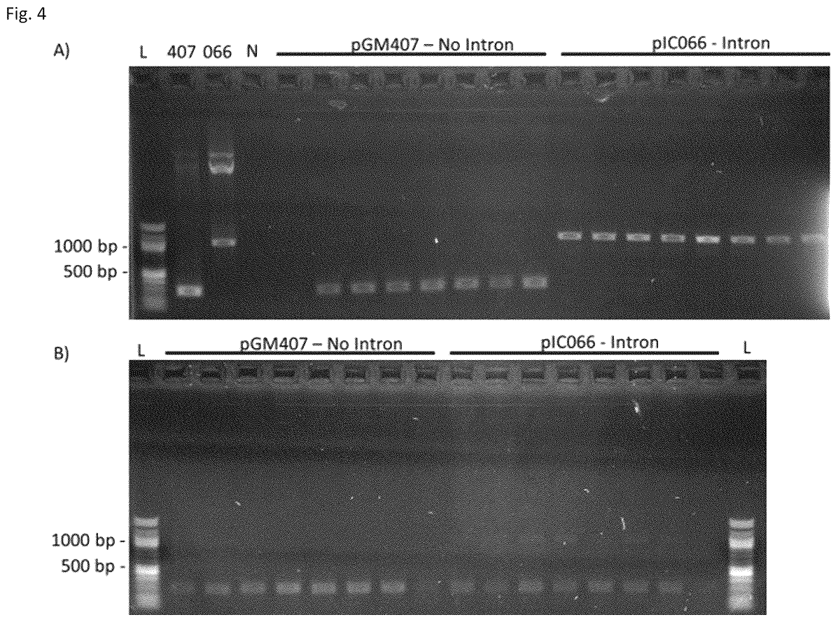

As shown in FIG. 4A, the intron in pGM991 was present and a large 1127 bp product was detected. In contrast, pGM407 produced a smaller product of 371 bp.

RNA was extracted from the HEK293T cells transfected with either pGM991 or pGM407. RT-PCR was carried out on the extracted RNA using primers which bind to either side of the intron:

| Primer | SEQ ID | | |
|---|---|---|---|
| Name | NO: | | Assays |
| oIC002 | 46 | CAATGGACAC | DNA and RNA |
| | | AGGGCTGAAA | reverse primer |
| oIC105 | 47 | TGCCTTCTC | RNA forward |
| | | CCTCCTGT | primer (can be |
| | | | used for DNA) |

As shown in FIG. 4B, the intron was spliced successfully by the transfected HEK293T cells, such that both pGM991 and pGM407 produced the same 277 bp RNA transcript.

### Example 3—SIV Vector Comprising a β-Globin/IgG Chimeric Intron with an Inserted SIV RRE is Correctly Packaged and Spliced

HEK293T cells were transduced with (a) a VSV-G pseudotyped SIV vector (SIV.VSV-G) lacking the endogenous SIV RRE and comprising (i) an AAT transgene under the control of an hCEF promoter and (ii) a β-globin/IgG chimeric intron with an inserted SIV RRE as produced in Example 1 (vGM291); or (b) a SIV.VSV-G vector comprising the endogenous SIV RRE and lacking this chimeric intron-RRE (vGM290). Non-transduced cells were used as controls.

PCR of DNA extracted from the transduced HEK293T was carried out using primers which bind to either side of the intron:

| Primer | SEQ ID | | |
|---|---|---|---|
| Name | NO: | Sequence | Assays |
| oIC001 | 45 | GGGAAAGTGAT | DNA forward |
| | | GTGGTGTACTG | primer |
| oIC002 | 46 | CAATGGACACA | DNA and RNA |
| | | GGGCTGAAA | reverse primer |

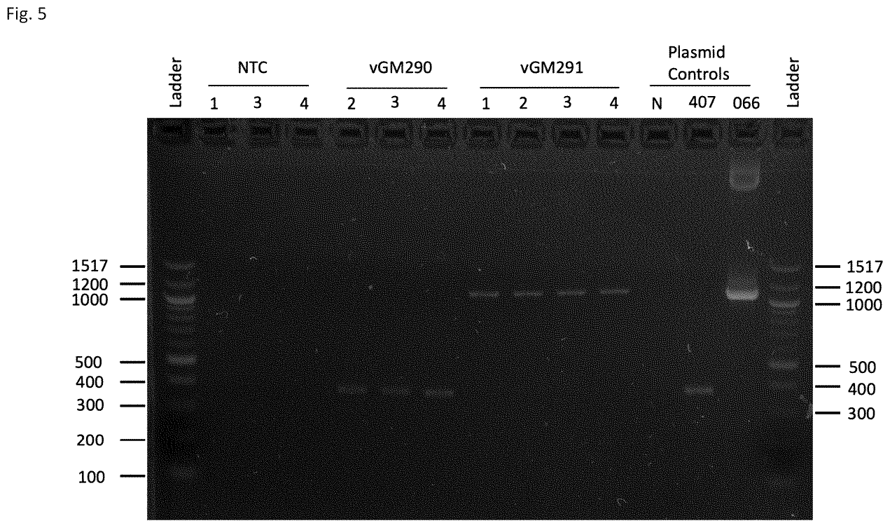

As shown in FIG. 5, vGM291 was properly packaged to produce SIV.VSV-G particles, with an 1127 bp product being detected. In contrast, vGM290 produced a smaller product of 371 bp.

RNA was extracted from the transduced HEK293T cells transduced with either vGM291; or vGM290. RT-PCR was carried out on the extracted RNA using primers which bind to either side of the intron:

| Primer | SEQ ID | | |
|---|---|---|---|
| Name | NO: | Sequence | Assays |
| oIC002 | 46 | CAATGGACAC | DNA and RNA |
| | | AGGGCTGAAA | reverse primer |
| oIC105 | 47 | TGCCTTCTCC | RNA forward |
| | | CTCCTGT | primer (can be |
| | | | used for DNA) |

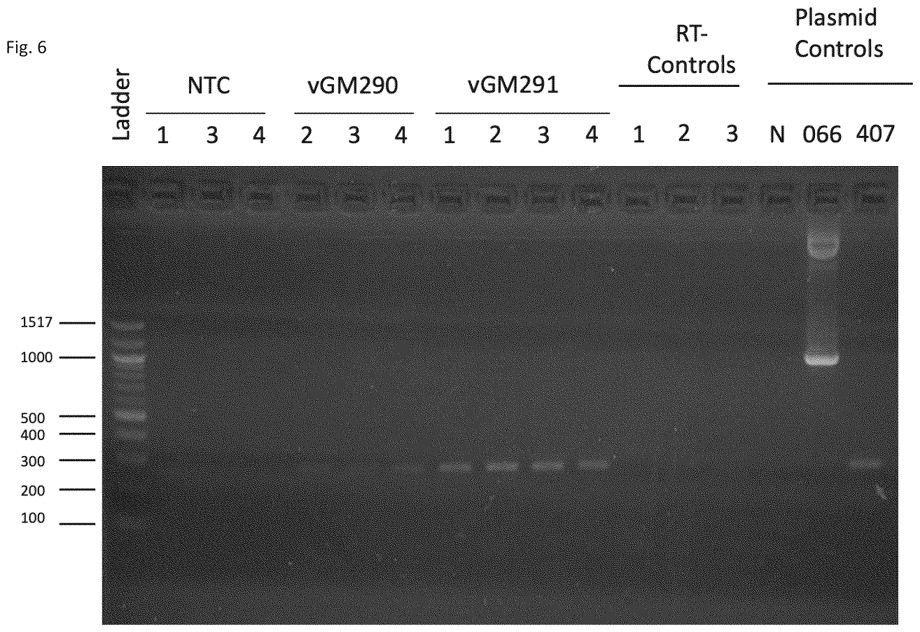

As shown in FIG. 6, the intron was spliced successfully by the transduced HEK293T cells, such that both vGM290 and vGM291 produced the same 277 bp RNA transcript.

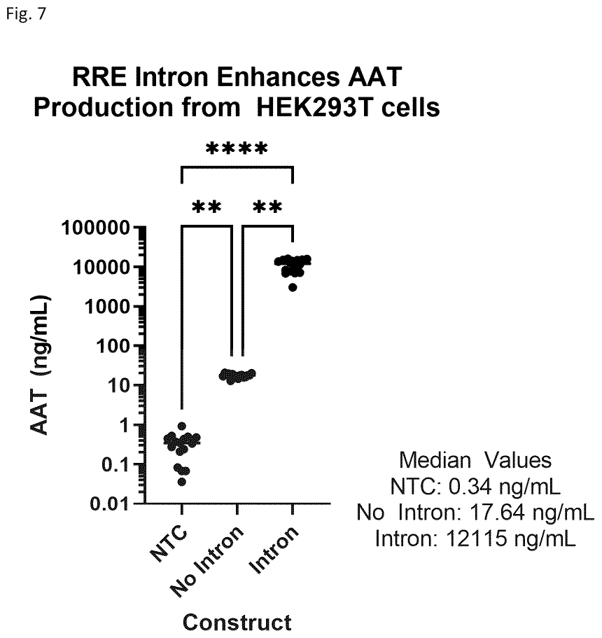

The effect of inclusion of the β-globin/IgG chimeric intron with an inserted SIV RRE on AAT transgene expression was also investigated. As shown in FIG. 7, transduction of HEK293T cells with this SIV.VSV-G vector significantly increased AAT expression compared with transduction of HEK293T cells with a corresponding SIV.VSV-G vector comprising the endogenous SIV RRE and lacking the β-globin/IgG chimeric intron with an inserted SIV RRE (vGM290). In particular, an increase in AAT expression of 686-fold was observed on transduction with vGM291 compared with transduction with vGM290.

### Example 4—SIV Vector Comprising a β-Globin/IgG Chimeric Intron with an Inserted SIV RRE Increases AAT Transgene Transcription

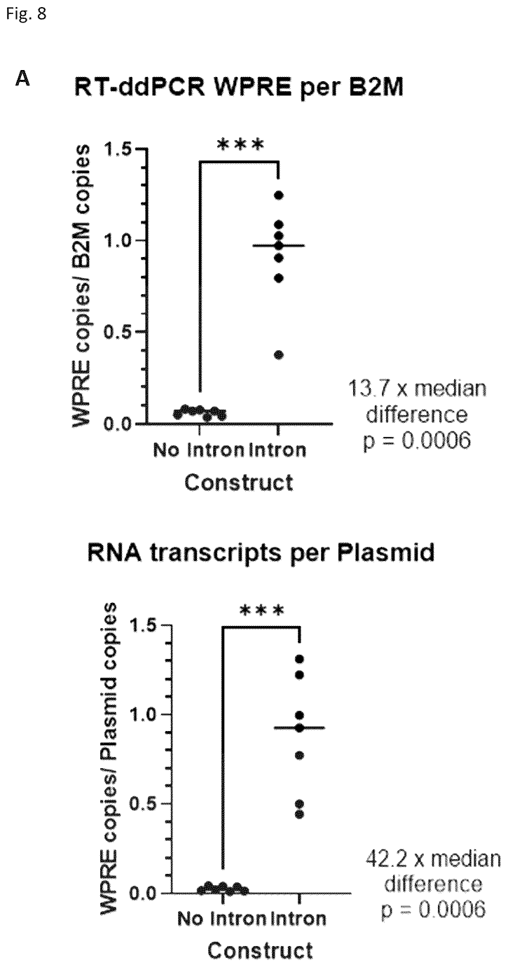

Furthermore, the effect of the intron on transgene transcription was investigated. Reverse transcription droplet digital PCR (RT-ddPCR) was carried out on RNA extracted from HEK293T cells transfected with plasmids as described in Example 2. Amount of mRNA was quantified by the number of WPRE copies using RT-ddPCR, standardised to the housekeeping gene @3-2-microglobulin (B2M). As shown in FIG. 8A, use of the pGM991 plasmid comprising the @3-globin/IgG chimeric intron with an inserted SIV RRE significantly increased the amount of mRNA produced by the HEK293T cells compared with use of the pGM407 plasmid which lacks the @3-globin/IgG chimeric intron with an inserted SIV RRE. Furthermore, the amount of mRNA produced per plasmid copy was also calculated, for which plasmid copy number was quantified by ddPCR from DNA extracted from transfected HEK293T cells. As shown in FIG. 8B, use of the pGM991 plasmid significantly increased the number of mRNA copies per plasmid copy compared to the pGM407 plasmid.

### Example 5—SIV.FN/H Vector Comprising a β-Globin/IgG Chimeric Intron with an Inserted SIV RRE Increases AAT Transgene Transcription

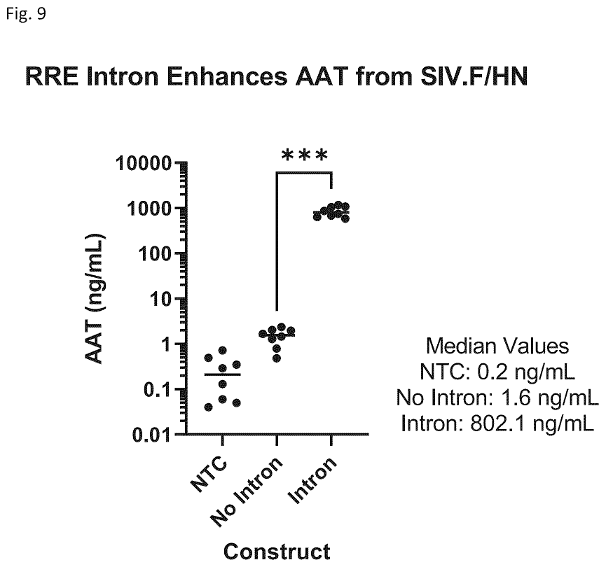

The experiment in Example 3 was repeated with an F/HN pseudotyped SIV vector, rather than a VSV-G pseudotyped SIV vector. As shown in FIG. 9, transduction of HEK293T cells with a SIV.F/HN vector containing the β-globin/IgG chimeric intron (vGM295) significantly increased AAT expression compared to HEK293T cells transduced with a corresponding SIV.F/HN vector comprising the endogenous SIV RRE and lacking the β-globin/IgG chimeric intron (vGM294). In particular, an increase in AAT expression of 501-fold was observed on transduction with vGM295 compared to transduction with vGM294.

Thus, the use of the β-globin/IgG chimeric intron with an inserted SIV RRE facilitates the maturation of a stable mRNA molecule, resulting in increased mRNA transcript levels, and consequently increased transgene expression, and this effect is observed in multiple different pseudotyped SIV vectors. Whilst this is exemplified with AAT, it is expected that similar advantages will be achieved using other transgenes.

1. A retroviral vector comprising an intron; wherein:
(a) the endogenous Rev response element (RRE) of the retroviral genome is deleted; and
(b) a retroviral RRE is inserted into the intron within 100 bp 5′ of the splice acceptor's branch site of the intron.

2. A retroviral vector according to claim 1, wherein the retroviral RRE is inserted within 20 bp 5′ of the splice acceptor's branch site of the intron.

3. A retroviral vector according to claim 1 or 2, wherein:
(a) the intron is a chimeric intron, optionally selected from a β-globin/IgG chimeric intron or a chimeric intron from the CAGGS promoter; or
(b) the intron is a viral intron, optionally selected from SV40 intron, CMV Intron A and adenovirus tripartite leader sequence intron.

4. A retroviral vector comprising a chimeric intron; wherein:
(a) the endogenous RRE of the retroviral genome is deleted; and
(b) a retroviral RRE is inserted into the chimeric intron.

5. A retroviral vector according to any one of the preceding claims, wherein the retroviral RRE inserted into the intron is the endogenous RRE of the retroviral genome.

6. A retroviral vector according to any one of the preceding claims, wherein the RRE is a Simian immunodeficiency virus (SIV) RRE.

7. A retroviral vector according to claim 6, wherein the RRE comprises or consists of a nucleic acid sequence having at least 90% identity to SEQ ID NO: 1.

8. A retroviral vector according to any one of the preceding claims, wherein the intron is less than 1,000 bp in length, preferably less than 800 bp in length.

9. A retroviral vector according to any one of claims 3 to 8, wherein the chimeric intron is a β-globin/IgG chimeric intron or a chimeric intron from the CAGGS promoter.

10. A retroviral vector according to claim 9, wherein the chimeric intron is a β-globin/IgG chimeric intron and the RRE is inserted between (i) a splice donor site comprising or consisting of a nucleic acid sequence of TGAGTTTAAGGTAAGT (SEQ ID NO: 2); and (ii) a splice acceptor site comprising or consisting of a nucleic acid sequence of CTCTCCACAG (SEQ ID NO: 3).

11. A retroviral vector according to claim 9 or 10, wherein the β-globin/IgG chimeric intron comprises or consists of a nucleic acid sequence having at least 90% identity to SEQ ID NO: 4.

12. A retroviral vector according to any one of the preceding claims, wherein the intron is a β-globin/IgG chimeric intron and the RRE is an SIV RRE, and optionally wherein the chimeric intron comprising the RRE comprises or consists of a nucleic acid sequence having at least 90% identity to SEQ ID NO: 5.

13. A retroviral vector according to any one of the preceding claims, wherein the intron is between a promoter and a transgene operably linked to said promoter, wherein optionally the promoter is selected the group consisting of a cytomegalovirus (CMV) promoter, elongation factor 1a (EF1a) promoter, and a hybrid human CMV enhancer/EF1a (hCEF) promoter, preferably a hCEF promoter.

14. A retroviral vector according to any one of claim 13, wherein the transgene encodes a therapeutic protein, wherein optionally said therapeutic protein is selected from:
(a) a secreted therapeutic protein, optionally Alpha-1 Antitrypsin (AAT), Factor VIII, Surfactant Protein B (SFTPB), ADAMTS13, Factor VII, Factor IX, Factor X, Factor XI, von Willebrand Factor, Granulocyte-Macrophage Colony-Stimulating Factor (GM-CSF), decorin, Surfactant Protein C (SP-C), an anti-inflammatory protein and a monoclonal antibody against an infectious agent; or
(b) CFTR, ABCA3, DNAH5, DNAH11, DNAI1, DNAI2, CSF2RA, CSF2RB and TRIM-72.

15. A retroviral vector according to any one of the preceding claims, wherein the retroviral vector is a lentiviral vector.

16. A retroviral vector according to claim 15, wherein the lentiviral vector is selected from the group consisting of a Human immunodeficiency virus (HIV) vector, a Simian immunodeficiency virus (SIV) vector, a Feline immunodeficiency virus (FIV) vector, an Equine infectious anaemia virus (EIAV) vector, and a Visna/maedi virus vector.

17. A retroviral vector according to any one of the preceding claims, which is pseudotyped with haemagglutinin-neuraminidase (HN) and fusion (F) proteins from a respiratory paramyxovirus or G glycoprotein from Vesicular Stomatitis Virus (G-VSV).

18. A retroviral vector according to any one of claims 13 to 17, which increases transgene expression by at least about 2-fold, preferably at least about 5-fold, more preferably at least about 10-fold compared with a corresponding vector which lacks a intron into which a retroviral RRE has been inserted.

19. A nucleic acid comprising or consisting of a intron into which a retroviral RRE has been inserted, wherein optionally (i) the intron; and/or (ii) the RRE are as defined in any one of claims 1 to 12.

20. A plasmid comprising a nucleic acid according to claim 19.

21. A retroviral vector according to any one of claims 1 to 18, a nucleic acid according to claim 19 or a plasmid according to claim 20, which is codon-optimised.

22. A composition comprising a retroviral vector according to any one of claims 1 to 18 or 21, a nucleic acid according to claim 19 or 21 or a plasmid according to claim 20 or 21, and a pharmaceutically-acceptable carrier.

23. A host cell comprising a retroviral vector according to any one of claims 1 to 18 or 21, a nucleic acid according to claim 19 or 21 or a plasmid according to claim 20 or 21.

24. A retroviral vector according to any one of claims 1 to 18 or 21, a nucleic acid according to claim 19 or 21, a plasmid according to claim 20 or 21, or a composition according to claim 22 for use in a method of treatment.

25. A method of producing a retroviral vector, said method comprising the following steps:
(a) growing cells in suspension;
(b) transfecting the cells with one or more plasmids;
(c) adding a nuclease;
(d) harvesting the lentivirus;
(e) adding trypsin; and
(f) purification;
wherein the one or more plasmids comprises a vector genome plasmid which comprises a nucleic acid as defined in claim 19, and optionally (i) a promoter as defined in claim 13 and/or (ii) a transgene as defined in claim 14.

26. A method of differentiating between a retroviral vector and a transgene expressed by said retroviral vector, said method comprising the steps of:
(a1) transducing cells with a retroviral vector as defined in any one of claims 1 to 18;
(b1) culturing the cells to allow transgene expression by the retrovirus; and
(c1) quantifying RNA within the cells; or
(a2) quantifying RNA within cells of a sample obtained from a patient who has undergone treatment with a retroviral vector according to any one of claims 1 to 18 or 21, a nucleic acid according to claim 19 or 21, a plasmid according to claim 20 or 21, or a composition according to claim 23;
wherein:
(i) the amount of RNA comprising the chimeric intron into which a retroviral RRE has been inserted corresponds to the copy number of the retroviral vector; and
(ii) the amount of RNA lacking the chimeric intron into which a retroviral RRE has been inserted corresponds to the amount of transgene mRNA;
wherein optionally RNA is quantified by a PCR-based or in situ hybridisation-based assay.
